(19) United States
(12) Patent Application Publication
(10) Pub. No.: US 2026/0207578 A1
(43) Pub. Date: Jul. 23, 2026

(54) COMPOUND BASED ON SULFUR/OXYGEN SUBSTITUTED GLUTARIMIDE-BASED ISOINDOLINONE SKELETON AND USE THEREOF

(71) Applicant: Gluetacs Therapeutics (Shanghai) Co., LTD., Shanghai (CN)

(72) Inventors: Xiaobao Yang, Shanghai (CN); Renhong Sun, Shanghai (CN); Yuedong Zhou, Shanghai (CN); Baoyin Zhao, Shanghai (CN); Chaowei Ren, Shanghai (CN)

(73) Assignee: Gluetacs Therapeutics (Shanghai) Co., LTD., Shanghai (CN)

(21) Appl. No.: 19/143,339

(22) PCT Filed: Dec. 26, 2023
(86) PCT No.: PCT/CN2023/141787

§ 371 Date: Jun. 25, 2025

(30) Foreign Application Priority Data
Dec. 27, 2022 (CN) 202211684679.1

(51) Int. Cl. A61K 31/4545; A61K 31/454; A61K 31/55; A61P 19/02; C07D 401/14; C07D 405/14; C07D 409/14

(52) U.S. Cl. CPC A61K 31/4545; A61K 31/454; A61K 31/55; A61P 19/02; C07D 401/14; C07D 405/14; C07D 409/14

(57) ABSTRACT

The present disclosure relates to the technical field of molecular glue degraders, and particularly to a sulfur/oxygen substituted glutarimide-isoindolinone skeleton-based compound of Formula I, and uses thereof. The present disclosure further pertains to a compound of Formula A and its uses.

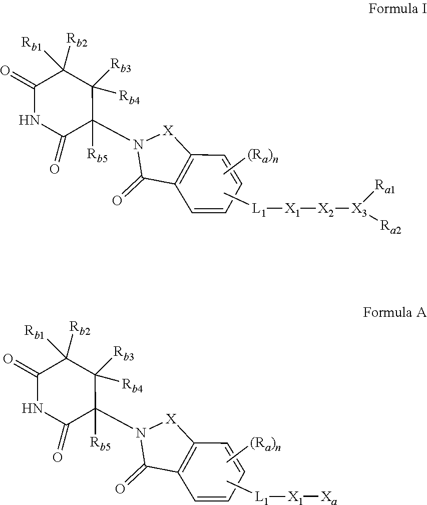

### TECHNICAL FIELD

The present disclosure relates to the technical field of molecular glue degraders, and particularly to a sulfur/oxygen-substituted glutarimidyl-isoindolinone scaffold-based compound and its applications.

### BACKGROUND

Although Thalidomide, lenalidomide and other phthalimide immunomodulatory drugs (IMiDs) have shown significant efficacy in the treatment of multiple myeloma and autoimmune diseases, it was not until 2010 that the E3 ubiquitin ligase cereblon (CRBN) was identified as a direct target of IMiDs. Subsequent research confirmed that these drugs function as molecular glue, inducing the interaction between transcription factors IKZF1/3 and CRBN protein, ultimately leading to their ubiquitination and degradation. Compared with traditional small molecule inhibitors, molecular glue protein degraders have natural mechanism advantages: the former works by occupying the functional domain of the target protein for a long time to inhibit its function, while the protein degraders directly degrade and eliminate the entire target protein, often having a much greater efficacy than traditional small molecule inhibitors. The protein degraders may target “non-drugable” targets and have low requirements for binding affinity, catalytic capacity and low concentration, which may overcome the clinical drug resistance problem of traditional small molecules. Moreover, molecular glue degraders usually have small molecular weights and desirable druggability, so the research and development of such drugs have received great attention. Based on the CRBN E3 ubiquitin ligase and molecular glue degradation mechanism, a series of compounds have been developed, such as pomalidomide, which has been launched and CC-122 of Bristol-Myers Squibb (BMS), which is undergoing clinical trials, CC-220, CC-90009, CC-99282 and CC-92480, DKY709 from Novartis and CFT7455 from C4 Therapeutics. The degradation substrates of these molecular glues have also expanded from the initially discovered transcription factors IKZF1/3 to include casein kinase 1a (CK1α), zinc finger protein 91 (ZFP91) and translation factor GSPT1. The degradation of these protein substrates enables the molecular glues to exert therapeutic efficacy against diseases or disorders associated with said substrates, including but not limited to immunomodulation, anti-inflammatory, and anti-tumor effects. Currently, the number of identified molecular glue candidate compounds is quite limited. Moreover, there is a wide variety of pathogenic proteins that may theoretically serve as degradation substrates for the development of molecular glues. Therefore, it is necessary to design and develop more molecular glues through rationalization and diversification approaches to degrade and eliminate more pathogenic proteins, and to apply them to the treatment of diseases associated with these pathogenic proteins.

The present disclosure develops more novel, highly efficient, low-toxic, stable, and cost-effective molecular glue degraders based on the fused phenyl-glutarimide skeleton through rational design to meet the demands of clinical applications.

### SUMMARY

The objects of the present disclosure are to provide a sulfur/oxygen-substituted glutarimidyl-isoindolinone scaffold-based compound and its applications, aiming to address the limitations in the prior art.

To achieve the above objectives and other related goals, in one aspect, the present disclosure provides a compound of Formula I:

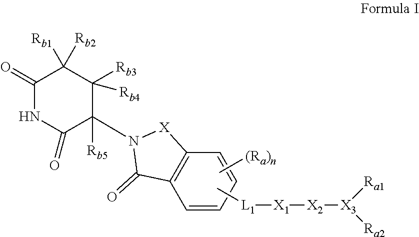

or salts, enantiomers, stereoisomers, polymorphs or solvates thereof, wherein
   X is selected from the group consisting of CO or CH₂;
   L₁ is selected from the group consisting of S or O;
   X₁ is selected from the group consisting of unsubstituted or substituted linear or branched alkylene, or X₁ is absent;
   X₂ represents the structure of Formula II:

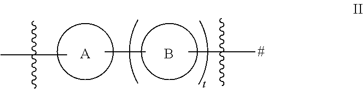

wherein ring A and ring B are each independently selected from the group consisting of unsubstituted or substituted arylene, unsubstituted or substituted heteroarylene, unsubstituted or substituted heterocyclylene, or unsubstituted or substituted cycloalkylene;
t represents an integer of 0, 1 or 2; and
symbol # indicates the point of attachment to X₃;
X₃ is selected from the group consisting of CR₁, N, or CO, wherein R₁ is selected from the group consisting of hydrogen, deuterium, halogen, nitro, cyano, hydroxy, mercapto, trifluoromethoxy, unsubstituted or substituted amino, unsubstituted or substituted silyl, unsubstituted or substituted boryl, unsubstituted or substituted linear or branched alkyl, or unsubstituted or substituted cycloalkyl;
Rₐ₁ is selected from the group consisting of hydrogen, deuterium, unsubstituted or substituted aryl, unsubstituted or substituted heteroaryl, unsubstituted or substituted cycloalkyl, or unsubstituted or substituted heterocyclyl;
Rₐ₂ is selected from the group consisting of hydrogen, deuterium, unsubstituted or substituted aryl, unsubstituted or substituted heteroaryl, unsubstituted or substituted cycloalkyl, or unsubstituted or substituted heterocyclyl, or Rₐ₂ is absent; and when X₃ represents CO, Rₐ₂ is absent and Rₐ₁ is neither hydrogen nor deuterium, and when Rₐ₂ represents hydrogen or deuterium, X₃ represents CR₁ or N, and Rₐ₁ is neither hydrogen nor deuterium;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl;
R_{b1}, R_{b2}, R_{b3}, R_{b4}, and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl; and
n is an integer of 0, 1, 2 or 3, and when n is an integer of 2 or 3, each Rₐ is independently identical to or different from each other.

In another aspect, the present disclosure provides a method for preparing the compound or salts, enantiomers, stereoisomers, polymorphs or solvates thereof of the present disclosure, comprising:

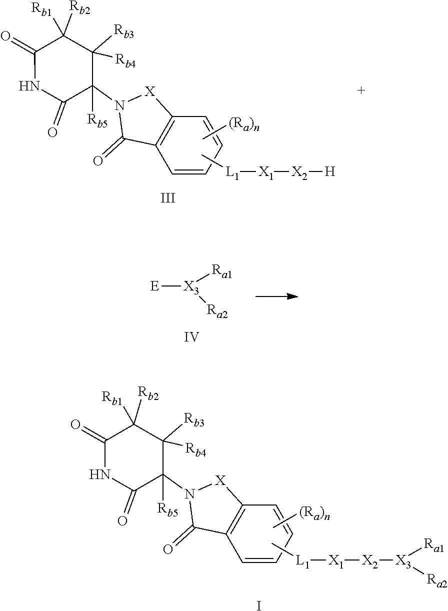

wherein (1) E-X₃ represents CO, Rₐ₂ represents hydrogen; or (2) E-X₃ represents halogen-CH, MsO-CH, TsO-CH, or NsO-CH; or (3) E-X₃ represents CO, Rₐ₂ represents hydroxy.

In a further aspect, the present disclosure provides the compound or salts, enantiomers, stereoisomers, polymorphs or solvates thereof of the present disclosure, for use as a medicament.

In a further aspect, the present disclosure provides a pharmaceutical composition comprising the compound or salts, enantiomers, stereoisomers, polymorphs or solvates thereof of the present disclosure, and at least one pharmaceutically acceptable salt, and at least one pharmaceutically acceptable carrier or excipient.

In a further aspect, the present disclosure provides use of the compound or salts, enantiomers, stereoisomers, polymorphs or solvates thereof of the present disclosure, or the pharmaceutical composition of the present disclosure for the manufacture of a medicament for the treatment of a disease or disorder associated with cereblon protein.

In a further aspect, the present disclosure provides a method for treating or preventing a disease or disorder associated with cereblon protein, comprising administering to a subject a therapeutically effective amount of the compounds or salts, enantiomers, stereoisomers, polymorphs or solvates thereof of the present disclosure, or the pharmaceutical composition of the present disclosure.

In another aspect, the present disclosure provides a compound of Formula A:

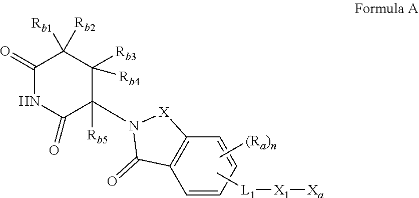

or salts, enantiomers, stereoisomers, polymorphs or solvates thereof, wherein
   X is selected from the group consisting of CO or CH₂;
   L₁ is selected from the group consisting of S or O;
   X₁ is selected from the group consisting of unsubstituted or substituted linear or branched alkylene, or X₁ is absent;
   Xₐ represents the structure of Formula A1:

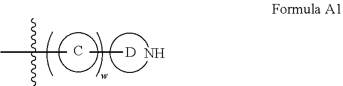

wherein ring C is selected from the group consisting of unsubstituted or substituted arylene, unsubstituted or substituted heteroarylene, unsubstituted or substituted heterocyclylene, or unsubstituted or substituted cycloalkylene, and w represents an integer of 0 or 1; and
ring D represents unsubstituted or substituted nitrogen-containing heterocyclyl;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl;
R_{b1}, R_{b2}, R_{b3}, R_{b4} and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl; and
n is an integer of 0, 1, 2 or 3, and when n is an integer of 2 or 3, each Rₐ is independently identical to or different from each other.

In a further aspect, the present disclosure provides the compound of Formula A or salts, enantiomers, stereoisomers, polymorphs or solvates thereof of the present disclosure, for use as a medicament.

In a further aspect, the present disclosure provides a pharmaceutical composition comprising the compound of Formula A or salts, enantiomers, stereoisomers, polymorphs or solvates thereof of the present disclosure, and at least one pharmaceutically acceptable salt, and at least one pharmaceutically acceptable carrier or excipient.

In a further aspect, the present disclosure provides use of the compound of Formula A or salts, enantiomers, stereoisomers, polymorphs or solvates thereof of the present disclosure, or the pharmaceutical composition of the present disclosure for the manufacture of a medicament for the treatment of a disease or disorder associated with cereblon protein.

In a further aspect, the present disclosure provides a method for treating or preventing a disease or disorder associated with cereblon protein, comprising administering to a subject a therapeutically effective amount of the compound of Formula A or salts, enantiomers, stereoisomers, polymorphs or solvates thereof of the present disclosure, or the pharmaceutical composition of the present disclosure.

Compared with the prior art, the advantageous effects of the present invention are as follows:

The compounds provided by the present invention feature a sulfur/oxygen-substituted glutarimidyl-isoindolinone scaffold. Based on this scaffold, rationally designed novel compounds have been developed as molecular glues, exhibiting high efficacy, low toxicity, excellent stability, and cost-effectiveness. These compounds are suitable for preparing immunomodulatory drugs, anti-inflammatory agents, and antitumor medications to meet clinical needs.

### DESCRIPTION OF DRAWINGS

FIG. 1 shows the Western blot analysis results of the compounds of the present disclosure. The compounds of the present disclosure effectively degrade substrate proteins (such as Wee1, IKZF1, IKZF2, IKZF3, GSPT1, and CK1α) in human primary peripheral blood mononuclear cells (hPBMCs).

### DETAILED DESCRIPTION OF THE INVENTION

Through extensive research, the present inventors have developed novel compounds based on a sulfur/oxygen-substituted glutarimidyl-isoindolinone scaffold. These compounds serve as molecular glues with the following advantages: structural novelty, high efficacy and low toxicity, excellent stability and cost-effectiveness. They are suitable for manufacturing medicament for treating CRBN (cereblon protein)-associated diseases or disorders, leading to the completion of the present invention.

### Definitions

Unless otherwise specified, the following words, phrases and symbols used herein generally have the meanings as described below.

In general, the nomenclature used herein (e.g., the IUPAC nomenclature) and the laboratory procedures described below (including those used in cell culture, organic chemistry, analytical chemistry, and pharmacology, etc.) are those well-known and commonly used in the art. Unless otherwise defined, all scientific and technical terms used herein in connection with the present disclosure described herein have the same meaning as commonly understood by one skill in the art. In addition, the use of the word “a” or “an” when used in conjunction with the term “comprising” or a noun in the claims and/or the specification may mean “one”, but it is also consistent with the meaning of “one or more”, “at least one”, and “one or more than one”. Similarly, the terms “another” or “other” may mean at least a second or more.

It should be understood that whenever the term “comprise” or “include” is used herein to describe various aspects, other similar aspects described by “consisting of” and/or “consisting essentially of” are also provided.

Herein, a bond interrupted by a wavy line shows the point of attachment of the depicted group to the rest of the molecule. For example, the group L₃ depicted below

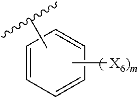

shows the point of attachment of the group L₃ to the group L₂ of the compound of Formula I.

Salts or pharmaceutically acceptable salts, enantiomers, stereoisomers, solvates, polymorphs of the compounds of Formula I of the present disclosure are also encompassed within the scope of the present disclosure.

As used herein, the term “enantiomer” shall be construed to mean, when a specific compound (or generic structure) is designated as the (R)- or (S)-enantiomer/having an absolute (R)- or (S)-configuration, the corresponding compound (or generic structure) in an enriched, particularly substantially pure enantiomeric form.

As used herein, the term “stereoisomer” refers to a compound with the same chemical structural formula, but a different arrangement of atoms or groups in space. Stereoisomers include enantiomers, diastereomers, conformational isomers (rotational isomers), geometric isomers (cis/trans isomers), atropisomers, and so on.

As used herein, the term “polymorph” refers to solid crystalline forms of the compounds of the present disclosure or complexes thereof. Different polymorphs of the same compound exhibit distinct physical, chemical, and/or spectroscopic properties. Physical property variations include but are not limited to stability (e.g., thermal or photostability), compressibility and density (critical for formulation and manufacturing), and dissolution rate (may affect bioavailability). Stability differences may cause variations in chemical reactivity (e.g., differential oxidation, as evidenced by faster discoloration observed in one polymorph compared to another), mechanical properties (e.g., conversion of tablets containing a kinetically preferred polymorph into a more thermodynamically stable crystalline form during storage), or both (e.g., tablets containing one polymorph being more susceptible to degradation under high humidity conditions). The additional physical properties of polymorphs may affect their processing behavior. For example, one polymorph may be more prone to solvate formation due to e.g., its distinct crystal habit or particle size distribution, or may present greater filtration or washing challenges compared to another polymorph.

As used herein, the term “salts or pharmaceutically acceptable salts” of the present disclosure refer to non-toxic inorganic or organic acid and/or base addition salts. Examples include: sulfates, hydrohalides (including hydrochlorides and hydrobromates), maleates, sulfonates, citrates, lactates, lactobionates, L-tartrates, fumarates, L-malates, L-lactates, α-ketoglutarates, hippurates, D-glucuronates, D-gluconates, α-D-glucoheptates, glycolates, mucates, L-ascorbates, orotates, picrates, glycinates, alaninates, argininates, cinnamates, laurates, pamoates, sebacates, benzenesulfonates, methanesulfonates, ethanesulfonates, ethanedisulfonates, formates, acetates, 2,2-dichloroacetates, trimethylacetates, propionates, valerates, palmitates, triphenylacetates, 2-ethyl-butanedioates, iodates, nicotiniates, L-pyroglutamates, L-prolinates, ferulates, 2-hydroxyethanesulfonates, nitrates, gentisates, cholates, salicylate, terephthalates, glutarates, adipates, stearates, oleoates, undecylenates, camphorates, camphorsulfonates, dodecyl sulfonates, phosphates, thiocyanates, dihydrophosphates, pyrophosphates, metaphosphates, oxalates, carbonates, malonates, benzoates, mandelates, succinates, pyruvates, p-chlorobenzenesulfonates, 1,5-naphthalenedisulfonates, 3-hydroxy-2-naphthoates, 1-hydroxy-2-naphthoates, 2-naphthalenesulfonates, glycolates, or p-toluenesulfonates, etc.

As used herein, the term “solvate” refers to a compound of the present disclosure or its pharmaceutically acceptable salt that incorporates either stoichiometric or non-stoichiometric amounts of solvent molecules bound through non-covalent intermolecular forces. Preferably, the solvent is volatile, non-toxic, and suitable for administration in minimal doses to humans. Examples of solvents include, but are not limited to water, isopropanol, ethanol, methanol, DMSO, ethyl acetate, acetic acid, and ethanolamine. The term “hydrate” refers to a complex where the solvent molecule is water.

As used herein, the wording “optionally substituted with . . . ” and “unsubstituted or substituted” may be used interchangeably. The term “substituted” generally indicates that one or more hydrogens in the referenced structure are replaced by the same or different specific substituents. By way of example, substituents may optionally be selected from the group consisting of: deuterium, halogen, cyano, nitro, hydroxy, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkylaryl, alkylamino, aralkylamino, heteroarylamino, arylamino, arylphosphino, heteroaryl, heterocyclyl, acenaphthenyl, and oxo; or the substituents may be formed by linking two or more of the aforementioned exemplary substituents. For example, “substituents formed by linking two or more of the aforementioned exemplary substituents” may include biaryl groups (e.g., biphenyl) and biheteroaryl groups (e.g., bipyridyl).

As used herein, the term “unsubstituted or substituted amino” may be represented by the formula —NY₁Y₂, wherein Y₁ and Y₂ may each independently be hydrogen, deuterium, alkenyl, alkyl (e.g., C₁-C₃ alkyl), cycloalkyl (e.g., C₃₋₂₀ cycloalkyl), heterocyclyl (e.g., 4- to 20-membered heterocyclyl), aryl (e.g., C₆₋₂₀ aryl), or heteroaryl (e.g., 5- to 20-membered heteroaryl). Non-limiting examples of the unsubstituted or substituted amino groups include amino, N-methylamino, N-ethylamino, N-phenylamino, N,N-dimethylamino, N,N-diethylamino, etc.

As used herein, the term “unsubstituted or substituted silyl” may be represented by the formula —SiYₐY_{b}Y_{c}, wherein Yₐ, Y_{b} and Y_{c} may each independently be hydrogen, deuterium, halogen, alkoxy, halogenated alkoxy, alkenyl, alkyl (e.g., C₁-C₃ alkyl), cycloalkyl (e.g., C₃₋₂₀ cycloalkyl), heterocyclyl (e.g., 4- to 20-membered heterocyclyl), aryl (e.g., C₆₋₂₀ aryl), or heteroaryl (e.g., 5- to 20-membered heteroaryl). Non-limiting examples of the unsubstituted or substituted silyl include trimethylsilyl, triethylsilyl, tert-butyldimethylsilyl, vinyldimethylsilyl, propyldimethylsilyl, triphenylsilyl, diphenylsilyl, phenylsilyl, etc.

As used herein, the term “unsubstituted or substituted boryl” may be represented by the formula —BY_{d}Yₑ, wherein Y_{d} and Yₑ may each independently be hydrogen, deuterium, alkoxy, halogenated alkoxy, alkenyl, alkyl (e.g., C₁-C₃ alkyl), cycloalkyl (e.g., C₃₋₂₀ cycloalkyl), heterocyclyl (e.g., 4- to 20-membered heterocyclyl), aryl (e.g., C₆₋₂₀ aryl), or heteroaryl (e.g., 5- to 20-membered heteroaryl). Non-limiting examples of the unsubstituted or substituted boryl include trimethylboryl, triethylboryl, tert-butyldimethylboryl, triphenylboryl, phenylboryl, etc.

As used herein, the term “ . . . is absent”, used alone or in combination, means that the referenced group serves as a bond linker, with the two adjacent groups being directly connected. For example, the expression “X₁ is absent” means that X₁ is a bond linker. In other words, when X₁ is absent, group L₁ of the compound of Formula I is directly connected to group X₂. Alternatively, for example, the expression “when X₃ represents CO, Rₐ₂ is absent, and Rₐ₁ is neither hydrogen nor deuterium” means that the group

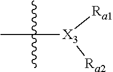

of the compound of Formula I represents

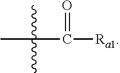

As used herein, the term “aryl”, used alone or in combination, refers to a monovalent aromatic hydrocarbon group containing from 5 to 30 carbon atoms and optionally one or more fused rings, such as C₅-C₃₀ aryl, C₅-C₂₅ aryl, C₅-C₂₀ aryl, C₅-C₁₅ aryl etc. Aryl may be monocyclic or polycyclic aryl. In some embodiments, monocyclic aryl includes but is not limited to phenyl, biphenyl, terphenyl, quaterphenyl, and quinquephenyl. Polycyclic aryl includes but is not limited to naphthyl, anthryl, phenanthryl, pyrenyl, perylenyl, and fluorenyl etc. Fluorenyl may be substituted (e.g., 9,9′-dimethylfluorenyl and 9,9′-diphenylfluorenyl etc.; furthermore, two of the substituents may combine with each other to form a spirocyclic structure such as 9,9′-spirobifluorenyl. As used herein, “aryl” refers to an optionally substituted aryl group. A substituted aryl refers to an aryl group substituted one or more times (e.g., 1-4, 1-3, or 1-2 times) with a substituent(s). For example, aryl is mono-, di-, or tri-substituted with a substituent(s) optionally selected from the group consisting of e.g., deuterated C₁-C₆ alkyl, deuterated C₃-C₆cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, cycloalkyl, alkenyl, aryl, aralkyl, arylalkenyl, alkylaryl, aralkylamino, heteroarylamino, arylamino, arylphosphino, heteroaryl, heterocyclyl, acenaphthenyl, and any combination thereof.

As used herein, the term “arylene”, used alone or in combination, refers to a divalent aromatic hydrocarbon group containing from 5 to 30 carbon atoms and optionally one or more fused rings, such as C₅-C₃₀ arylene, C₅-C₂₅ arylene, C₅-C₂₀ arylene, C₅-C₁₅ arylene etc. C₅-C₃₀ arylene may be monocyclic arylene or polycyclic arylene. Monocyclic arylene includes but is not limited to phenylene, biphenylene, terphenylene, quaterphenylene, and quinquephenylene. Polycyclic arylene includes but is not limited to naphthylene, anthrylene, phenanthrylene, pyrenylene, perylenylene, and fluorenylene etc. Fluorenylene may be substituted (e.g., 9,9′-dimethylfluorenylene and 9,9′-diphenylfluorenylene); furthermore, two of the substituents may combine with each other to form a spirocyclic structure such as 9,9′-spirobifluorenylene. As used herein, “arylene” refers to an optionally substituted arylene group. A substituted arylene refers to an arylene group substituted one or more times (e.g., 1-4, 1-3, or 1-2 times) with a substituent(s). For example, arylene is mono-, di-, or tri-substituted with a substituent(s) optionally selected from the group consisting of e.g., deuterated C₁-C₆ alkyl, deuterated C₃-C₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, cycloalkyl, alkenyl, aryl, aralkyl, arylalkenyl, alkylaryl, aralkylamino, heteroarylamino, arylamino, arylphosphino, heteroaryl, heterocyclyl, acenaphthenyl, and any combination thereof.

As used herein, the term “heteroaryl”, used alone or in combination, refers to a 5- to 30-membered (optionally 5- to 20-membered, 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered) monovalent monocyclic, bicyclic, or polycyclic heteroaryl containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroaryl groups include bicyclic, tricyclic or tetracyclic heteroaryl groups, which contain one aromatic ring having one or more heteroatoms independently selected from O, S and N, and the remaining rings which may be a saturated, partially unsaturated or aromatic ring and may be carbocyclic ring or contain one or more heteroatoms independently selected from O, S and N. Heteroaryl includes, but is not limited to, furanyl, oxazolyl, isoxazolyl, oxadiazolyl, thienyl, thiazolyl, isothiazolyl, thiadiazolyl, pyrrolyl, imidazolyl, pyrazolyl, pyridyl, pyrimidinyl, pyridazinyl, pyrazinyl, triazolyl, tetrazolyl, pyranyl, thiopyranyl, thiazinyl, triazinyl, tetrazinyl, indolyl, indolinyl, isoindolyl, isoindolinyl, benzofuranyl, isobenzofuranyl, benzothienyl, indazolyl, quinolinyl, isoquinolinyl, quinazolinyl, quinoxalinyl, cinnolinyl, naphthyridinyl, acridinyl, xanthenyl, phenanthridinyl, diazanaphthyl, triazaindenyl, indolizinyl, phthalazinyl, pyrazolopyridinyl, pyrazolopyrimidinyl, pyridopyrimidinyl, pyridopyrazinyl, pyrazinopyrazinyl, benzothiazolyl, benzoxazolyl, benzimidazolyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, pyrrolopyridinyl, pyrrolothiazolyl, imidazothiazolyl, pyrenyl, dinaphthofuranyl, naphthobenzofuranyl, dinaphthothienyl, or naphthobenzothienyl etc. The heteroaryl group may be unsubstituted or substituted. A substituted heteroaryl refers to heteroaryl substituted one or more times (e.g., 1-4, 1-3, or 1-2 times) by a substituent(s) optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, aryl, aralkyl, arylalkenyl, alkylaryl, aralkylamino, heteroarylamino, arylamino, arylphosphino, heteroaryl, heterocyclyl, acenaphthenyl, or any combination thereof.

As used herein, the term “heteroarylene”, used alone or in combination, refers to a 5- to 30-membered (optionally 5- to 20-membered, 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered) divalent monocyclic, bicyclic, or polycyclic heteroaryl containing one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroarylene groups include bicyclic, tricyclic or tetracyclic heteroarylene groups, which contain one aromatic ring having one or more heteroatoms independently selected from O, S and N, and the remaining rings which may be a saturated, partially unsaturated or aromatic ring and may be carbocyclic ring or contain one or more heteroatoms independently selected from O, S and N. Heteroarylene includes, but is not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, tetrazolylene, pyranylene, thiopyranylene, thiazinylene, triazinylene, tetrazinylene, indolylene, indolinylene, isoindolylene, isoindolinylene, benzofuranylene, isobenzofuranylene, benzothienylene, indazolylene, quinolinylene, isoquinolinylene, quinazolinylene, quinoxalinylene, cinnolinylene, naphthyridinylene, acridinylene, xanthenylene, phenanthridinylene, diazanaphthylene, triazaindenylene, indolizinylene, phthalazinylene, pyrazolopyridylene, pyrazolopyrimidinylene, pyridopyrimidinylene, pyridopyrazinylene, pyrazinopyrazinylene, benzothiazolylene, benzoxazolylene, benzimidazolylene, dibenzothienylene, dibenzofuranylene, carbazolylene, benzocarbazolylene, dibenzocarbazolylene, indolocarbazolylene, indenocarbazolylene, phenazinylene, imidazopyridinylene, phenanthridinylene, phenanthrolinylene, phenothiazinylene, imidazopyridinylene, imidazophenanthridinylene, pyrrolopyridinylene, pyrrolothiazolylene, imidazothiazolylene, benzimidazoquinazolinylene, benzimidazophenanthridinylene, pyrenylene, dinaphthofuranylene, naphthobenzofuranylene, dinaphthothienylene, or naphthobenzothienylene etc. The heteroarylene group may be unsubstituted or substituted. A substituted heteroarylene refers to heteroarylene substituted one or more times (e.g., 1-4, 1-3, or 1-2 times) by a substituent(s) optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, aryl, aralkyl, arylalkenyl, alkylaryl, aralkylamino, heteroarylamino, arylamino, arylphosphino, heteroaryl, heterocyclyl, acenaphthenyl, or any combination thereof.

As used herein, the term “heterocyclyl”, used alone or in combination, refers to a monocyclic, bicyclic, tricyclic, or polycyclic saturated or partially unsaturated (i.e., containing one or more double bonds without full conjugation) cycloalkyl group containing one or more heteroatoms (e.g., 1 to 5, 1 to 4, 1 to 3, 1 to 2, or 1 heteroatom) independently selected from sulfur, oxygen, and nitrogen, with no specific limitation on the number of carbon atoms. Representative examples include, but are not limited to, 4- to 30-membered heterocyclyl, 4- to 25-membered heterocyclyl, 4- to 20-membered heterocyclyl, 4- to 15-membered heterocyclyl, 4- to 10-membered heterocyclyl, 10- to 15-membered heterocyclyl, 7- to 15-membered heterocyclyl, and 5- to 15-membered heterocyclyl. Bicyclic, tricyclic, and polycyclic heterocyclyl include bridged heterocyclyl (e.g., 5- to 20-membered, 5- to 15-membered, 5- to 11-membered, 5- to 10-membered, 7- to 20-membered, 7- to 15-membered, 7- to 9-membered bridged heterocyclyl), fused heterocyclyl (e.g., 5- to 20-membered, 5- to 15-membered, 5- to 11-membered, 5- to 10-membered, 7- to 20-membered, 7- to 15-membered, 7- to 9-membered fused heterocyclyl) and spiro-heterocyclyl (e.g., 5- to 20-membered, 5- to 15-membered, 5- to 11-membered, 5- to 10-membered, 7- to 20-membered, 7- to 15-membered, 7- to 9-membered spiro-heterocyclyl). By way of example, the heterocyclyl group may contain 1-3 heteroatoms, 1-2 heteroatoms, or 1 heteroatom, with each heteroatom independently selected from the group consisting of O, N, and S. In additional embodiments, the heterocyclyl includes, but is not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, azacyclooctyl, diazacycloheptanyl (e.g., 1,4-diazacycloheptanyl, 4,5-diazacycloheptanyl, or 1,3-diazacycloheptanyl etc.), diazacyclooctyl, azabicyclohexyl (e.g., 3-azabicyclo[3.1.0]hexyl etc.), azabicycloheptanyl (e.g., 3-azabicyclo[3.1.1]heptanyl, 3-azabicyclo[3.2.0]heptanyl, 6-azabicyclo[3.1.1]heptan-3-yl etc.), diazabicycloheptanyl (e.g., 2,5-diazabicyclo[2.2.1]heptan-2-yl, 3,6-diazabicyclo[3.1.1]heptan-3-yl), azabicyclooctyl (e.g., cis-7-azabicyclo[3.3.0]octanyl, 3-azabicyclo[3.2.1]octan-8-yl etc.), diazabicyclooctyl (e.g., 3,8-diazabicyclo[3.2.1]octan-8-yl, 3,8-diazabicyclo[3.2.1]octan-3-yl, 2,5-diazabicyclo[2.2.2]octan-2-yl), spiro-heterocyclyl (e.g., 5- to 15-membered or 7- to 15-membered spiro-heterocyclyl, 7-azaspiro[3.5]nonanyl, 3-azaspiro[5,5]undecanyl etc.). The heterocyclyl may be unsubstituted or substituted as explicitly defined (e.g., mono-, di-, tri-, or poly-substituted) by a substituent(s) optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, alkenyl, aryl, aralkyl, arylalkenyl, alkylaryl, aralkylamino, heteroarylamino, arylamino, arylphosphino, heteroaryl, heterocyclyl, acenaphthenyl, and any combination thereof.

As used herein, the term “nitrogen-containing heterocyclyl”, used alone or in combination, refers to 4- to 30-membered (e.g., 4- to 20-membered, 4- to 15-membered, 4- to 14-membered, 4- to 13-membered, 4- to 12-membered, 4- to 11-membered, 4- to 10-membered, 4- to 9-membered, 4- to 8-membered, 4- to 7-membered, 4- to 6-membered, 4- to 5-membered, 4- to 9-membered, 5- to 20-membered, 5- to 15-membered, 7- to 20-membered, or 7- to 15-membered) saturated or partially unsaturated (i.e., containing one or more double bonds without full conjugation) monocyclic, bicyclic, tricyclic, or polycyclic cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Examples of “nitrogen-containing heterocyclyl” include, but are not limited to, nitrogen-containing monocyclic heterocyclyl (e.g., 4- to 30-membered or 4- to 20-membered nitrogen-containing monocyclic heterocyclyl), nitrogen-containing bridged heterocyclyl (e.g., 5- to 20-membered nitrogen-containing bridged heterocyclyl, or 7- to 20-membered nitrogen-containing bridged heterocyclyl), nitrogen-containing fused heterocyclyl (e.g., 5- to 20-membered or 7- to 20-membered nitrogen-containing fused heterocyclyl) and nitrogen-containing spiro-heterocyclyl (e.g., 5- to 20-membered or 7- to 20-membered nitrogen-containing spiro-heterocyclyl). Representative examples of nitrogen-containing monocyclic heterocyclyl include, but are not limited to, azetidinyl, pyrrolidinyl, imidazolidinyl, pyrazolidyl, oxazolidinyl, thiazolidinyl, piperidinyl, piperazinyl, morpholinyl, thiomorpholinyl, azacycloheptanyl, azacyclooctyl, diazacycloheptanyl (e.g., 1,4-diazacycloheptan-1-yl) and diazacyclooctyl. Examples of nitrogen-containing bridged heterocyclyl, nitrogen-containing fused heterocyclyl and nitrogen-containing spiro-heterocyclyl include, but are not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, 2,5-diazabicyclo[2.2.2]octan-2-yl, and azaspiro-heterocyclyl (e.g., 5- to 20-membered azaspiro-heterocyclyl, such as 3-azaspiro[5.5]undecan-3-yl). The nitrogen-containing heterocyclyl may be unsubstituted or substituted as explicitly defined (e.g., mono-, di-, tri-, or poly-substituted) by a substituent(s) optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, alkenyl, aryl, aralkyl, arylalkenyl, alkylaryl, aralkylamino, heteroarylamino, arylamino, arylphosphino, alkyl, cycloalkyl, heteroaryl, heterocyclyl, acenaphthenyl, and any combination thereof.

As used herein, the term “heterocyclylene”, used alone or in combination, refers to a monocyclic, bicyclic, tricyclic, or polycyclic saturated or partially unsaturated (i.e., containing one or more double bonds without full conjugation) divalent cycloalkyl group containing one or more heteroatoms (e.g., 1 to 5, 1 to 4, 1 to 3, 1 to 2, or 1 heteroatom) independently selected from sulfur, oxygen, and nitrogen, with no specific limitation on the number of carbon atoms. Representative examples include, but are not limited to, 4- to 30-membered heterocyclylene, 4- to 25-membered heterocyclylene, 4- to 20-membered heterocyclylene, 4- to 15-membered heterocyclylene, 4- to 10-membered heterocyclylene, 10- to 15-membered heterocyclylene, 7- to 15-membered heterocyclylene, and 5- to 15-membered heterocyclylene etc. Bicyclic, tricyclic, and polycyclic heterocyclylene include bridged heterocyclylene (e.g., 5- to 20-membered, 5- to 15-membered, 5- to 11-membered, 5- to 10-membered, 7- to 20-membered, 7- to 15-membered, 7- to 9-membered bridged heterocyclylene), fused heterocyclylene (e.g., 5- to 20-membered, 5- to 15-membered, 5- to 11-membered, 5- to 10-membered, 7- to 20-membered, 7- to 15-membered, 7 to 9-membered fused heterocyclylene) and spiro-heterocyclylene (e.g., 5- to 20-membered, 5- to 15-membered, 5- to 11-membered, 5- to 10-membered, 7- to 20-membered, 7- to 15-membered, 7- to 9-membered spiro-heterocyclylene). By way of example, the heterocyclylene group may contain 1-3 heteroatoms, 1-2 heteroatoms, or 1 heteroatom, with each heteroatom independently selected from the group consisting of O, N, and S. In additional embodiments, the heterocyclylene includes, but is not limited to, azetidinylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, azacyclooctylene, or diazacycloheptanylene (e.g., 1,4-diazacycloheptanylene 4,5-diazacycloheptanylene, or 1,3-diazacycloheptanylene etc.), diazacyclooctylene, azabicyclohexylene (e.g., 3-azabicyclo[3.1.0]hexylene etc.), azabicycloheptanylene (e.g., 3-azabicyclo[3.1.1]heptanylene, 3-azabicyclo[3.2.0]heptanylene, 6-azabicyclo[3.1.1]heptanylene etc.), azabicyclooctylene (e.g., cis-7-azabicyclo[3.3.0]octanylene, 3-azabicyclo[3.2.1]octanylene etc.), diazabicyclooctylene (e.g., 3,8-diazabicyclo[3.2.1]octanylene, 2,5-diazabicyclo[2.2.2]octanylene), or spiro-heterocyclylene (e.g., 5- to 15-membered or 7- to 15-membered spiro-heterocyclylene, 7-azaspiro[3.5]nonanylene, 3-azaspiro[5,5]undecanylene etc.). The heterocyclylene may be unsubstituted or substituted as explicitly defined (e.g., mono-, di-, tri-, or poly-substituted) by a substituent(s) optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, alkenyl, aryl, aralkyl, arylalkenyl, alkylaryl, aralkylamino, heteroarylamino, arylamino, arylphosphino, alkyl, cycloalkyl, heteroaryl, heterocyclyl, acenaphthenyl, and any combination thereof.

As used herein, the term “cycloalkyl”, used alone or in combination, refers to a saturated or partially unsaturated (i.e., containing one or more double bonds without full conjugation) monocyclic or bicyclic or polycyclic cyclic hydrocarbon radical, which in some embodiments contains from 3 to 20 carbon atoms (i.e., C₃₋₂₀ cycloalkyl), or from 3 to 15 carbon atoms (i.e., C₃₋₁₅ cycloalkyl), or from 3 to 12 carbon atoms (i.e., C₃₋₁₂ cycloalkyl), or from 3 to 11 carbon atoms (i.e., C₃₋₁₁ cycloalkyl), or from 3 to 10 carbon atoms (i.e., C₃₋₁₀ cycloalkyl), or from 3 to 8 carbon atoms (i.e., C₃₋₈ cycloalkyl), or from 3 to 7 carbon atoms (i.e., C₃₋₇ cycloalkyl), or from 3 to 6 carbon atoms (i.e., C₃₋₆ cycloalkyl), or from 4 to 20 carbon atoms (i.e., C₄₋₂₀ cycloalkyl), or from 4 to 15 carbon atoms (i.e., C₄₋₁₅ cycloalkyl), or from 5 to 20 carbon atoms (i.e., C₅₋₂₀ cycloalkyl), or from 5 to 15 carbon atoms (i.e., C₅₋₁₅ cycloalkyl). The term “C₃-C₂₀cycloalkyl” refers to a saturated or partially unsaturated (i.e., containing one or more double bonds without full conjugation) monocyclic or bicyclic or polycyclic cyclic hydrocarbon radical contains from 3 to 20 carbon atoms. The term “cycloalkyl” includes monocyclic, bicyclic, tricyclic and polycyclic cycloalkyl. The bicyclic, tricyclic and polycyclic cycloalkyl include bridged cycloalkyl (e.g., C₅₋₂₀ bridged cycloalkyl, C₅₋₁₅ bridged cycloalkyl, C₇₋₂₀ bridged cycloalkyl, C₇₋₁₅ bridged cycloalkyl, C₇₋₁₅ bridged cycloalkyl), fused cycloalkyl (e.g., C₅₋₂₀ fused cycloalkyl, C₅₋₁₅ fused cycloalkyl, C₇₋₂₀ fused cycloalkyl, C₇₋₁₅ fused cycloalkyl, C₃₋₇ fused cycloalkyl) and spiro-cycloalkyl (e.g., C₅₋₂₀ spiro-cycloalkyl, C₅₋₁₅ spiro-cycloalkyl, C₇₋₂₀ spiro-cycloalkyl, C₇₋₁₅ spiro-cycloalkyl, C₇₋₁ spiro-cycloalkyl). Representative examples of cycloalkyl include, but are not limited to, cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₅₋₂₀ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), adamantanyl, noradamantanyl, bornyl, norbornyl (also named as bicyclo[2.2.1]heptyl by the IUPAC system) and cubanyl. As used herein, the “cycloalkyl” is optionally substituted. The substituents of the substituted “cycloalkyl” may preferably be one or more (e.g., 1-5, 1-4, 1-3, 1-2, or 1) substituents selected from the group consisting of trifluoromethyl, mercapto, hydroxy, amino, halogen, cyano, C₁-C₃ alkoxy, trifluoromethyl, heterocyclyl, deuterium, nitro, carbonyl, ester group, imide group, amino, phosphine oxide group, deuterated alkoxy, trifluoromethoxy, aryloxy, alkylthio, arylthio, alkylsulfonyl, arylsulfonyl, silyl, boryl, alkyl (e.g., C₁-C₃ alkyl), deuterated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo, or any combination thereof.

As used herein, the term “cycloalkylene”, used alone or in combination, refers to a saturated or partially unsaturated (i.e., containing one or more double bonds without full conjugation) divalent monocyclic or bicyclic or polycyclic cyclic hydrocarbon radical, which in some embodiments contains from 3 to 20 carbon atoms (i.e., C₃₋₂₀ cycloalkylene), or from 3 to 15 carbon atoms (i.e., C₃₋₁₅ cycloalkylene), or from 3 to 12 carbon atoms (i.e., C₃₋₁₂ cycloalkylene), or from 3 to 11 carbon atoms (i.e., C₃₋₁₁ cycloalkylene), or from 3 to 10 carbon atoms (i.e., C₃₋₁₀ cycloalkylene), or from 3 to 8 carbon atoms (i.e., C₃₋₈ cycloalkylene), or from 3 to 7 carbon atoms (i.e., C₃₋₇ cycloalkylene), or from 3 to 6 carbon atoms (i.e., C₃₋₆ cycloalkylene), or from 4 to 20 carbon atoms (i.e., C₄₋₂₀ cycloalkylene), or from 4 to 15 carbon atoms (i.e., C₄₋₁₅ cycloalkylene), or from 5 to 20 carbon atoms (i.e., C₅₋₂₀ cycloalkylene), or from 5 to 15 carbon atoms (i.e., C₅₋₁₅ cycloalkylene). The term “C₃-C₂₀ cycloalkylene” refers to a saturated or partially unsaturated (i.e., containing one or more double bonds without full conjugation) divalent monocyclic or bicyclic or polycyclic cyclic hydrocarbon radical contains from 3 to 20 carbon atoms. The term “cycloalkylene” includes monocyclic, bicyclic, tricyclic and polycyclic cycloalkylene. The bicyclic, tricyclic and polycyclic cycloalkylene include bridged cycloalkylene (e.g., C₅-20 bridged cycloalkylene, C₅₋₁₅ bridged cycloalkylene, C₇₋₂₀ bridged cycloalkylene, C₇₋₁₅ bridged cycloalkylene, C₇₋₁ bridged cycloalkylene), fused cycloalkylene (e.g., C₅₋₂₀ fused cycloalkylene, C₅₋₁₅ fused cycloalkylene, C₇₋₂₀ fused cycloalkylene, C₇₋₁₅ fused cycloalkylene, C₇-1 fused cycloalkylene) and spiro-cycloalkylene (e.g., C₅₋₂₀ spiro-cycloalkylene, C₅₋₁₅ spiro-cycloalkylene, C₇₋₂₀ spiro-cycloalkylene, C₇₋₁₅ spiro-cycloalkylene, C₇₋₁₁ spiro-cycloalkylene). Representative examples of cycloalkylene include, but are not limited to, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₅₋₂₀ spiro-cycloalkylene, such as spiro[3.3]heptanylene, spiro[2.5]octanylene, spiro[3.5]nonylene, spiro[4.4]nonylene, spiro[4.5]decylene, or spiro[5.5]undecylene), adamantanylene, noradamantanylene, bornylene, norbornylene (also named as bicyclo[2.2.1]heptanylene by the IUPAC system) and cubanylene. The cycloalkylene may be unsubstituted or substituted as explicitly defined. The substituents of the substituted “cycloalkylene” may preferably be one or more (e.g., 1-5, 1-4, 1-3, 1-2, or 1) substituents selected from the group consisting of trifluoromethyl, mercapto, hydroxy, amino, halogen, cyano, C₁-C₃ alkoxy, trifluoromethyl, heterocyclyl, deuterium, nitro, carbonyl, ester group, imide group, phosphine oxide group, deuterated alkoxy, trifluoromethoxy, aryloxy, alkylthio, arylthio, alkylsulfonyl, arylsulfonyl, silyl, boryl, alkyl (e.g., C₁-C₃ alkyl), deuterated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo, or any combination thereof.

As used herein, the term “alkyl”, used alone or in combination, refers to a linear or branched alkyl group, with no specific limitation on the number of carbon atoms. For example, it may be C₁-C₃₀, C₁-C₂₅, C₁-C₂₀, C₁-C₁₅, C₁-C₁₀, C₁-C₅ alkyl, etc. By way of example, alkyl includes but is not limited to methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, etc. As used herein, the “alkyl” is optionally substituted by one or more substituents optionally selected from the group consisting of D (i.e., deuterium), halogen, hydroxy, cyano, C₁-C₃ alkoxy, trifluoromethyl, heterocyclyl (e.g., 4- to 8-membered heterocyclyl), nitro, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl (e.g., C₁-C₃ alkyl), deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkylaryl, alkylamino, aralkylamino, heteroarylamino, arylamino, arylphosphino, heteroaryl, acenaphthenyl, oxo, or any combination thereof.

As used herein, the term “alkylene”, used alone or in combination, refers to a linear or branched divalent saturated hydrocarbon group composed of carbon and hydrogen atoms, with no specific limitation on the number of carbon atoms. For example, it may be C₁-C₃₀, C₁-C₂₅, C₁-C₂₀, C₁-C₁₅, C₁-C₁₀, C₁-C₅ alkylebe, etc. By way of example, alkylene includes but is not limited to methylene, ethylene, propylene, isopropylene, butylene, n-butylene, isobutylene, tert-butylene, sec-butylene, 1-methyl-butylene, 1-ethyl-butylene, pentylene, n-pentylene, isopentylene, neopentylene, tert-pentylene, hexylene, n-hexylene, 1-methylpentylene, 2-methylpentylene, 4-methyl-2-pentylene, 3,3-dimethylbutylene, 2-ethylbutylene, heptylene, n-heptylene, 1-methylhexylene, cyclopentylmethylene, cyclohexylmethylene, octylene, n-octylene, tert-octylene, 1-methylheptylene, 2-ethylhexylene, 2-propylpentylene, nonylene, 2,2-dimethylheptylene, 1-ethyl-propylene, 1,1-dimethyl-propylene, isohexylene, 4-methylhexylene, 5-methylhexylene, etc. As used herein, the “alkylene” is optionally substituted by one or more substituents optionally selected from the group consisting of D (i.e., deuterium), halogen, hydroxy, cyano, C₁-C₃ alkoxy, trifluoromethyl, heterocyclyl (e.g., 4- to 8-membered heterocyclyl), nitro, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo, or any combination thereof.

As used herein, the term “alkoxy”, used alone or in combination, refers to —O-alkyl. Optionally, the alkyl portion of the alkoxy group may contain 1-10 (or 1-6, 1-5, 1-4, or 1-3) carbon atoms. Alkoxy includes, but is not limited to, e.g., methoxy, ethoxy, propoxy, isopropoxy, n-butoxy, isobutoxy, tert-butoxy, sec-butoxy, n-pentyloxy, neopentyloxy, isopentyloxy, n-hexyloxy, 3,3-dimethylbutoxy, 2-ethylbutoxy, n-octyloxy, n-nonyloxy, n-decyloxy, etc.

As used herein, the term “halogen atom” or “halogen”, used alone or in combination, refers to fluorine, chlorine, bromine, or iodine.

As used herein, the term “halogenated alkyl”, used alone or in combination, refers to a linear or branched alkyl in which one or more hydrogen atoms are each replaced with a halogen atom. Examples of the halogenated alkyl include, but are not limited to, —CF₃, —CHF₂, —CH₂F, —CBr₃, —CHBr₂, —CH₂Br, —CCl₃, —CHC12, —CH₂Cl, —C₁₃, —CHI₂, —CH₂I, —CH₂—CF 3, —CH₂—CHF₂, —CH₂—CH₂F, —CH₂—CBr₃, —CH₂—CHBr₂, —CH₂—CH₂Br, —CH₂—CCl₃, —CH₂—CHC12, —CH₂—CH₂Cl, —CH₂—C₁₃, —CH₂—CHI₂, —CH₂—CH₂I etc. Herein, the alkyl and halogen are as defined above.

As used herein, the term “halogenated alkoxy”, used alone or in combination, refers to a alkoxy group in which one or more hydrogen atoms are each replaced with a halogen atom. Examples of the halogenated alkoxy include, but are not limited to, trifluoromethoxy etc. Herein, the alkoxy and halogen are as defined above.

As used herein, the term “pharmaceutically acceptable carrier” generally refers to carriers used for the administration of therapeutic agents, which do not themselves induce the production of antibodies harmful to the individual receiving the composition and exhibit no excessive toxicity upon administration. These carriers are well-known to those skilled in the art, e.g., relevant information on pharmaceutically acceptable carriers disclosed in Remington's Pharmaceutical Sciences (Mack Pub. Co., N. J, 1991). Specifically, the carrier may comprise, but is not limited to, one or more selected from the group consisting of saline, buffers, glucose, water, glycerol, ethanol, adjuvants, and combinations thereof.

As used herein, the term “pharmaceutically acceptable excipient” refers to a pharmaceutically acceptable material, such as a filler, stabilizer, dispersant, suspending agent, diluent, excipient, thickener, solvent, or encapsulating material, with which the useful compounds according to the present disclosure are carried or transported into or administered to a patient so that they may perform their intended function. Generally, such constructs are carried or transported from one organ or part of the body to another organ or part of the body. The excipient is compatible with the other ingredients of the formulation, including the compounds useful in the present disclosure, and is not harmful to the patient, and the excipient must be “acceptable”. Some examples of materials that may be used as pharmaceutically acceptable excipients include sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; and other common non-toxic compatible substances used in pharmaceutical formulations.

### Compounds

In one aspect, the present disclosure provides an embodiment 1): the compound of Formula I:

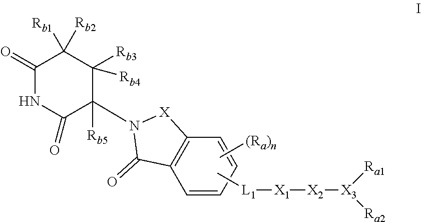

or salts, enantiomers, stereoisomers, polymorphs or solvates thereof,
   wherein X is selected from the group consisting of CO or CH₂;
   L₁ is selected from the group consisting of S or O;
   X₁ is selected from the group consisting of unsubstituted or substituted linear or branched alkylene, or X₁ is absent;
   X₂ represents the structure of Formula II:

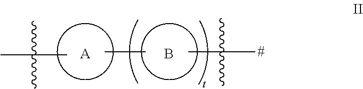

wherein ring A and ring B are each independently selected from the group consisting of unsubstituted or substituted arylene, unsubstituted or substituted heteroarylene, unsubstituted or substituted heterocyclylene, or unsubstituted or substituted cycloalkylene;
t represents an integer of 0, 1 or 2; and
symbol # indicates the point of attachment to X₃;
X₃ is selected from the group consisting of CR₁, N, or CO, wherein R₁ is selected from the group consisting of hydrogen, deuterium, halogen, nitro, cyano, hydroxy, mercapto, trifluoromethoxy, unsubstituted or substituted amino, unsubstituted or substituted silyl, unsubstituted or substituted boryl, unsubstituted or substituted linear or branched alkyl, or unsubstituted or substituted cycloalkyl;
Rₐ₁ is selected from the group consisting of hydrogen, deuterium, unsubstituted or substituted aryl, unsubstituted or substituted heteroaryl, unsubstituted or substituted cycloalkyl, or unsubstituted or substituted heterocyclyl;
Rₐ₂ is selected from the group consisting of hydrogen, deuterium, unsubstituted or substituted aryl, unsubstituted or substituted heteroaryl, unsubstituted or substituted cycloalkyl, or unsubstituted or substituted heterocyclyl, or Rₐ₂ is absent; and when X₃ represents CO, Rₐ₂ is absent and Rₐ₁ is neither hydrogen nor deuterium, and when Rₐ₂ represents hydrogen or deuterium, X₃ represents CR₁ or N and Rai is neither hydrogen nor deuterium;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl; R_{b1}, R_{b2}, R_{b3}, R_{b4} and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl;
n is an integer of 0, 1, 2 or 3, and when n is an integer of 2 or 3, each Rₐ is independently identical to or different from each other.

Embodiment 2): Pertaining to the compound of Formula I or salts, enantiomers, stereoisomers, polymorphs or solvates thereof as described in embodiment 1), wherein X in Formula I is CO.

Embodiment 3): Pertaining to the compound of Formula I or salts, enantiomers, stereoisomers, polymorphs or solvates thereof as described in embodiment 1), wherein X in Formula I is CH₂.

Embodiment 4): Pertaining to the compound of Formula I or salts, enantiomers, stereoisomers, polymorphs or solvates thereof as described in embodiment 1), wherein L₁ in Formula I is S.

Embodiment 5): Pertaining to the compound of Formula I or salts, enantiomers, stereoisomers, polymorphs or solvates thereof as described in embodiment 1), wherein L₁ in Formula I is O.

Embodiment 6): Pertaining to the compound of Formula I or salts, enantiomers, stereoisomers, polymorphs or solvates thereof as described in any one of embodiments 1)-5), wherein X₁ is unsubstituted or substituted linear or branched alkylene. When X₁ is unsubstituted or substituted linear or branched alkylene, the X₁ represents unsubstituted or substituted linear or branched C₁-C₅alkylene, or unsubstituted or substituted linear or branched C₁-C₃ alkylene.

Embodiment 7): Pertaining to the compound of Formula I or salts, enantiomers, stereoisomers, polymorphs or solvates thereof as described in any one of embodiments 1)-6), wherein when X₁ is unsubstituted or substituted linear or branched C₁-C₅ alkylene, the linear or branched C₁-C₅ alkylene may be methylene, ethylene, propylene, n-propylene, isopropylene, butylene, n-butylene, isobutylene, tert-butylene, sec-butylene, 1-methyl-butylene, 1-ethyl-butylene, pentylene, n-pentylene, isopentylene, neopentylene, or tert-pentylene.

Embodiment 8): Pertaining to the compound of Formula I or salts, enantiomers, stereoisomers, polymorphs or solvates thereof as described in any one of embodiments 1)-7), wherein when X₁ is unsubstituted or substituted linear or branched C₁-C₃ alkylene, the linear or branched alkylene may be e.g., methylene, ethylene, propylene, or isopropylene.

Embodiment 9): Pertaining to the compound of Formula I or salts, enantiomers, stereoisomers, polymorphs or solvates thereof as described in any one of embodiments 1)-8), wherein the X₁ is selected from the group consisting of methylene.

Embodiment 10): Pertaining to the compound of Formula I or salts, enantiomers, stereoisomers, polymorphs or solvates thereof as described in any one of embodiments 1)-8), wherein the substituents of the substituted X₁ is optionally selected from the group consisting of deuterium, halogen, cyano, nitro, hydroxy, mercapto, carbonyl, ester group, imide group, amino, phosphine oxide group, alkoxy, halogenated alkoxy, aryloxy, alkylthio, arylthio, alkylsulfonyl, arylsulfonyl, silyl, boryl, alkyl, cycloalkyl, alkenyl, aryl, aralkyl, arylalkenyl, alkylaryl, alkylamino, aralkylamino, heteroarylamino, arylamino, arylphosphino, heteroaryl, and acenaphthenyl etc., or any combination thereof. Optionally, the substituents of the substituted X₁ may be e.g., one or more substituents selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl), nitro, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo or any combination thereof. Further optionally, the substituents of the substituted X₁ may be e.g., selected from the group consisting of deuterium, halogen, nitro, mercapto, hydroxy, cyano, C₁-C₃ alkyl, C₁-C₃ alkoxy, halogenated C₁-C₃ alkoxy, halogenated C₁-C₃ alkyl, amino, silyl, boryl, or any combination thereof. Further optionally, the substituents of the substituted X₁ may be e.g., selected from the group consisting of deuterium, F, Cl, methoxy, methyl, hydroxy, or any combination thereof.

Embodiment 11): Pertaining to the compound of Formula I or salts, enantiomers, stereoisomers, polymorphs or solvates thereof as described in any one of embodiments 1)-5), wherein X₁ is absent.

Embodiment 12): Pertaining to the compound of Formula I or salts, enantiomers, stereoisomers, polymorphs or solvates thereof as described in any one of embodiments 1)-11), wherein X₂ represents the structure of Formula II:

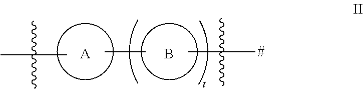

wherein ring A is selected from the group consisting of unsubstituted or substituted C₅-C₃₀ arylene, unsubstituted or substituted 5- to 30-membered heteroarylene, unsubstituted or substituted 4- to 30-membered heterocyclylene, or unsubstituted or substituted C₃-C₃₀ cycloalkylene; preferably, ring A is selected from the group consisting of unsubstituted or substituted C₅-C₂₀ arylene, unsubstituted or substituted 5- to 20-membered heteroarylene, unsubstituted or substituted 4- to 20-membered heterocyclylene, or unsubstituted or substituted C₃-C₂₀ cycloalkylene;
ring B is selected from the group consisting of unsubstituted or substituted C₅-C₃₀ arylene, unsubstituted or substituted 5- to 30-membered heteroarylene, unsubstituted or substituted 4- to 30-membered heterocyclylene, or unsubstituted or substituted C₃-C₃₀ cycloalkylene; preferably, ring B is selected from the group consisting of unsubstituted or substituted C₅-C₂₀ arylene, unsubstituted or substituted 5- to 20-membered heteroarylene, unsubstituted or substituted 4- to 20-membered heterocyclylene, or unsubstituted or substituted C₃-C₂₀ cycloalkylene;
t represents an integer of 0, 1 or 2; preferably, t represents an integer of 0 or 1; and
symbol # indicates the point of attachment to X₃.

Embodiment 13): Pertaining to the compound of Formula I or salts, enantiomers, stereoisomers, polymorphs or solvates thereof as described in any one of embodiments 1)-12), wherein X₂ represents the structure of Formula II:

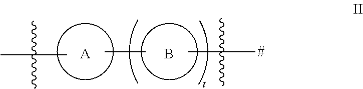

wherein ring A is selected from the group consisting of unsubstituted or substituted C₅-C₁₅ arylene, unsubstituted or substituted 5- to 15-membered heteroarylene, unsubstituted or substituted 4- to 15-membered heterocyclylene, or unsubstituted or substituted C₃-C₁₅ cycloalkylene, wherein the substituents of the substituted ring A are optionally one or more substituents selected from the group consisting of halogen, mercapto, hydroxy, amino, cyano, C₁-C₆ alkyl, C₁-C₆ alkoxy, halogenated C₁-C₆ alkyl or any combination thereof;
ring B is selected from the group consisting of unsubstituted or substituted C₅-C₁₅ arylene, unsubstituted or substituted 5- to 15-membered heteroarylene, unsubstituted or substituted 4- to 15-membered heterocyclylene, or unsubstituted or substituted C₃-C₁₅ cycloalkylene; wherein the substituents of the substituted ring B are optionally one or more substituents selected from the group consisting of halogen, mercapto, hydroxy, amino, cyano, C₁-C₆ alkyl, C₁-C₆ alkoxy, halogenated C₁-C₆ alkyl, or any combination thereof;
t represents an integer of 0, 1 or 2; preferably, t represents an integer of 0 or 1; and
symbol # indicates the point of attachment to X₃.

Embodiment 14): Pertaining to the compound of Formula I or salts, enantiomers, stereoisomers, polymorphs or solvates thereof as described in any one of embodiments 1)-13), wherein ring A and ring B are each independently selected from the group consisting of:
azetidinylene, azacyclopentylene, azacyclohexylene, azacycloheptanylene, azacyclooctylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, azacyclooctylene, diazacycloheptanylene, diazacyclooctylene, bridged heterocyclylene (e.g., 4- to 15-membered bridged heterocyclylene, 5- to 15-membered bridged heterocyclylene, and 7- to 15-membered bridged heterocyclylene, such as azabicyclohexylene, azabicycloheptanylene or azabicyclooctylene), or spiro-heterocyclylene (e.g., 4- to 15-membered spiro-heterocyclylene, 5- to 15-membered spiro-heterocyclylene, and 7- to 15-membered spiro-heterocyclylene, such as 3-azaspiro[5.5]undecanylene or 7-azaspiro[3.5]nonanylene); or
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene; or
cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₃-C₁₅ spiro-cycloalkylene or C₅-C₁₅ spiro-cycloalkylene or C₇-C₁₅ spiro-cycloalkylene, such as spiro[3.3]heptylene, spiro[2.5]octylene, spiro[3.5]nonylene, spiro[4.4]nonylene, spiro[4.5]decylene, or spiro[5.5]undecylene), or bridged cycloalkylene (e.g., C₃-C₁₅ bridged cycloalkylene or C₅-C₁₅ bridged cycloalkylene or C₇-C₁₅ bridged cycloalkylene, adamantanylene, noradamantanylene, bornylene, bicyclo[2.2.1]heptylene, 2-oxobicyclo[2.2.1]heptylene, or bicyclo[2.2.1]heptentylene); or
phenylene or naphthylene;
wherein ring A and ring B are each independently optionally substituted by one or more substituents selected from the group consisting of deuterium, halogen, nitro, mercapto, hydroxy, cyano, C₁-C₆ alkyl, C₁-C₆ alkoxy, halogenated C₁-C₆ alkoxy, halogenated C₁-C₆ alkyl, unsubstituted or substituted amino, unsubstituted or substituted silyl, unsubstituted or substituted boryl or any combination thereof.

Embodiment 15): Pertaining to the compound of Formula I or salts, enantiomers, stereoisomers, polymorphs or solvates thereof as described in any one of embodiments 12)-14), wherein X₂ represents optionally substituted nitrogen-containing heterocyclyl, optionally substituted aryl-NH₂, optionally substituted heteroaryl-NH₂, or optionally substituted cycloalkyl-NH₂. Further, X₂ is selected from the group consisting of

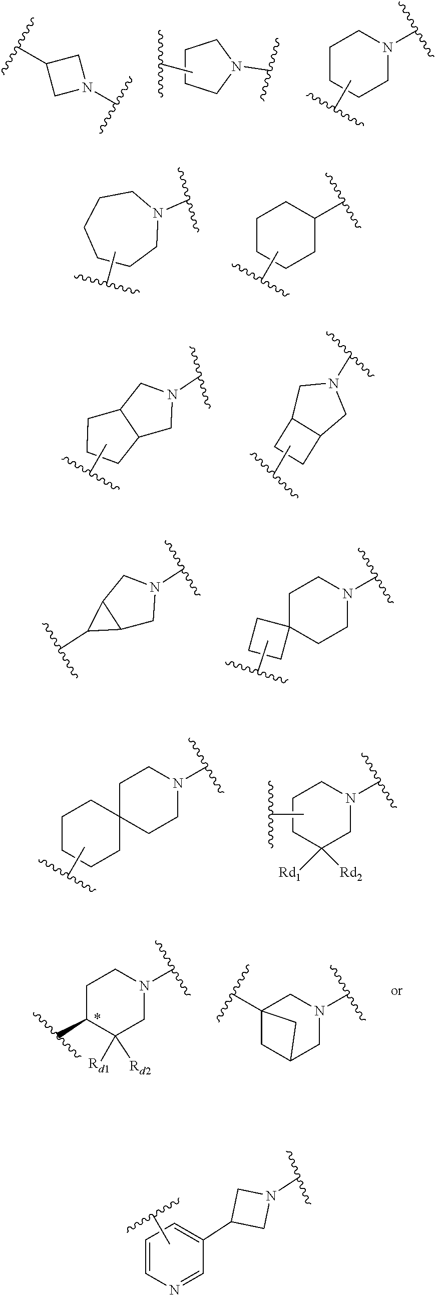

Embodiment 16): Pertaining to the compound of Formula I or salts, enantiomers, stereoisomers, polymorphs or solvates thereof as described in embodiment 15), wherein R_{d1} and R_{d2} are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, and unsubstituted or substituted linear or branched C₁-C₅ alkyl; and R_{d1} and R_{d2} are not hydrogen at the same time. In specific embodiments, optionally, the halogen may be e.g., F, Cl, Br or I. Optionally, the C₁-C₆ alkoxy includes but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, and 2-ethylbutoxy etc. Optionally, the halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino etc.

Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc. Optionally, the linear or branched C₁-C₅ alkyl includes but is not limited to methyl, ethyl, propyl, isopropyl, butyl, n-butyl, isobutyl, tert-butyl, sec-butyl, 1-methyl-butyl, 1-ethyl-butyl, pentyl, n-pentyl, isopentyl, neopentyl, and tert-pentyl etc., wherein said linear or branched C₁-C₅ alkyl is optionally substituted with one or more substituents optioanlly selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl) or any combination thereof.

Embodiment 17): Pertaining to the compound of Formula I or salts, enantiomers, stereoisomers, polymorphs or solvates thereof as described in any one of embodiments 1)-16), wherein X₃ is CR₁; and R₁ is selected from the group consisting of hydrogen, deuterium, halogen, nitro, cyano, hydroxy, mercapto, trifluoromethoxy, unsubstituted or substituted amino, unsubstituted or substituted silyl, unsubstituted or substituted boryl, unsubstituted or substituted linear or branched alkyl, or unsubstituted or substituted cycloalkyl.

Embodiment 18): Pertaining to the compound of Formula I or salts, enantiomers, stereoisomers, polymorphs or solvates thereof as described in embodiment 17), wherein R¹ is selected from the group consisting of hydrogen, deuterium, F, Cl, Br, I, nitro, cyano, hydroxy, mercapto, trifluoromethoxy, unsubstituted or substituted amino, unsubstituted or substituted silyl, unsubstituted or substituted boryl, unsubstituted or substituted linear or branched C₁-C₁₀ alkyl, or unsubstituted or substituted C₃-C₁₅ cycloalkyl.

Embodiment 19): Pertaining to the compound of Formula I or salts, enantiomers, stereoisomers, polymorphs or solvates thereof as described in embodiment 17), wherein when X₃ is selected from the group consisting of CR₁, R₁ is hydrogen.

Embodiment 20): Pertaining to the compound of Formula I or salts, enantiomers, stereoisomers, polymorphs or solvates thereof as described in embodiment 17), wherein when X₃ is selected from the group consisting of CR₁, R₁ is deuterium.

Embodiment 21): Pertaining to the compound of Formula I or salts, enantiomers, stereoisomers, polymorphs or solvates thereof as described in embodiment 17), wherein when X₃ is selected from the group consisting of CR₁, R₁ is halogen. Optionally, when R₁ is halogen, the halogen is F, Cl, Br, or I.

Embodiment 22): Pertaining to the compound of Formula I or salts, enantiomers, stereoisomers, polymorphs or solvates thereof as described in embodiment 17), wherein when X₃ is selected from the group consisting of CR₁, R₁ is nitro.

Embodiment 23): Pertaining to the compound of Formula I or salts, enantiomers, stereoisomers, polymorphs or solvates thereof as described in embodiment 17), wherein when X₃ is selected from the group consisting of CR₁, R₁ is cyano.

Embodiment 24): Pertaining to the compound of Formula I or salts, enantiomers, stereoisomers, polymorphs or solvates thereof as described in embodiment 17), wherein when X₃ is selected from the group consisting of CR₁, R₁ is amino.

Embodiment 25): Pertaining to the compound of Formula I or salts, enantiomers, stereoisomers, polymorphs or solvates thereof as described in embodiment 17), wherein when X₃ is selected from the group consisting of CR₁, R₁ is hydroxy.

Embodiment 26): Pertaining to the compound of Formula I or salts, enantiomers, stereoisomers, polymorphs or solvates thereof as described in embodiment 17), wherein when X₃ is selected from the group consisting of CR₁, R₁ is mercapto.

Embodiment 27): Pertaining to the compound of Formula I or salts, enantiomers, stereoisomers, polymorphs or solvates thereof as described in embodiment 17), wherein when X₃ is selected from the group consisting of CR₁, R₁ is trifluoromethoxy.

Embodiment 28): Pertaining to the compound of Formula I or salts, enantiomers, stereoisomers, polymorphs or solvates thereof as described in embodiment 17), wherein when X₃ is selected from the group consisting of CR₁, R₁ is unsubstituted or substituted amino. Optionally, the unsubstituted or substituted amino includes amino, N-methylamino, N-ethylamino, N-phenylamino, N,N-dimethylamino, and N,N-diethylamino etc.

Embodiment 29): Pertaining to the compound of Formula I or salts, enantiomers, stereoisomers, polymorphs or solvates thereof as described in embodiment 17), wherein when X₃ is selected from the group consisting of CR₁, R₁ is unsubstituted or substituted silyl. Optionally, the unsubstituted or substituted silyl is specifically trimethylsilyl, triethylsilyl, tert-butyldimethylsilyl, vinyldimethylsilyl, propyldimethylsilyl, triphenylsilyl, diphenylsilyl, and phenylsilyl etc.

Embodiment 30): Pertaining to the compound of Formula I or salts, enantiomers, stereoisomers, polymorphs or solvates thereof as described in embodiment 17), wherein when X₃ is selected from the group consisting of CR₁, R₁ is unsubstituted or substituted boryl. Optionally, the unsubstituted or substituted boryl is specifically trimethylboryl, triethylboryl, tert-butyldimethylboryl, triphenylboryl, and phenylboryl etc.

Embodiment 31): Pertaining to the compound of Formula I or salts, enantiomers, stereoisomers, polymorphs or solvates thereof as described in embodiment 17), wherein when X₃ is selected from the group consisting of CR₁, R₁ is unsubstituted or substituted alkyl. Optionally, when the R₁ is unsubstituted or substituted linear or branched alkyl, the number of carbon atoms may be for example C₁-C₃₀, C₁-C₂₅, C₁-C₂₀, C₁-C₁₅, C₁-C₁₀, C₁-C₅, and C₁-C₃ etc, preferably linear or branched C₁-C₁₀ alkyl. Further optionally, the linear or branched C₁-C₁₀ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, nonyl, 2,2-dimethylheptyl, 1-ethylpropyl, 1,1-dimethylpropyl, isohexyl, 4-methylhexyl, or 5-methylhexyl etc. Optionally, the linear or branched C₁-C₁₀ alkyl includes but is not limited to methyl, ethyl, and propyl etc. Further, the alkyl is optionally substituted by a substituent optionally selected from the group consisting of e.g., deuterium, halogen, cyano, nitro, hydroxy, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo, or any combination thereof. The substituent is preferably one or more substituents selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl) or any combination thereof.

Embodiment 32): Pertaining to the compound of Formula I or salts, enantiomers, stereoisomers, polymorphs or solvates thereof as described in embodiment 17), wherein when R₁ is unsubstituted or substituted cycloalkyl, it has 3 to 20 carbon atoms (i.e., C₃₋₂₀ cycloalkyl), 3 to 15 carbon atoms (i.e., C₃₋₁₅ cycloalkyl), 3 to 12 carbon atoms (i.e., C₃₋₁₂ cycloalkyl), 3 to 11 carbon atoms (i.e., C₃₋₁₁ cycloalkyl), 3 to 10 carbon atoms (i.e., C₃₋₁₀ cycloalkyl), 3 to 8 carbon atoms (i.e., C₃₋₈ cycloalkyl), 3 to 7 carbon atoms (i.e., C₃₋₇ cycloalkyl), or 3 to 6 carbon atoms (i.e., C₃₋₆ cycloalkyl). Preferably, R₁ is unsubstituted or substituted C₃-C₁₅ cycloalkyl. Optionally, C₃-C₁₅ cycloalkyl includes but is not limited to cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₅₋₂₀ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), adamantanyl, noradamantanyl, bornyl, norbornyl (Also named as bicyclo[2.2.1]heptyl by the IUPAC system) and cubanyl etc. Further optionally, C₃-C₁₅ cycloalkyl includes but is not limited to cyclopropyl, cyclobutyl, and cyclopentyl etc. Wherein, said cycloalkyl is optionally substituted with one or more (e.g., 1-5, 1-4, 1-3, 1-2, or 1) substituents optionally selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, amino, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, acenaphthenyl, oxo or any combination thereof. The substituents are preferably selected from the group consisting of trifluoromethyl, mercapto, hydroxy, amino, halogen, cyano, C₁-C₃ alkyl, C₁-C₃ alkoxy, trifluoromethoxy, heterocyclyl or any combination thereof.

Embodiment 33): Pertaining to the compound of Formula I or salts, enantiomers, stereoisomers, polymorphs or solvates thereof as described in any one of embodiments 1)-17), wherein X₃ is N.

Embodiment 34): Pertaining to the compound of Formula I or salts, enantiomers, stereoisomers, polymorphs or solvates thereof as described in any one of embodiments 1)-17), wherein X₃ is CO.

Embodiment 35): Pertaining to the compound of Formula I or salts, enantiomers, stereoisomers, polymorphs or solvates thereof as described in any one of embodiments 1)-17), wherein when X₃ represents CO, Rₐ₂ is absent and Rₐ₁ is neither hydrogen nor deuterium.

Embodiment 36): Pertaining to the compound of Formula I or salts, enantiomers, stereoisomers, polymorphs or solvates thereof as described in any one of embodiments 1)-17), wherein when Rₐ₂ represents hydrogen or deuterium, X₃ represents CR₁ or N, and Rₐ₁ is neither hydrogen nor deuterium.

Embodiment 37): Pertaining to the compound of Formula I or salts, enantiomers, stereoisomers, polymorphs or solvates thereof as described in any one of embodiments 1)-36), wherein Rₐ₁ is selected from the group consisting of hydrogen, deuterium, unsubstituted or substituted aryl, unsubstituted or substituted heteroaryl, unsubstituted or substituted cycloalkyl, or unsubstituted or substituted heterocyclyl.

Embodiment 38): Pertaining to the compound of Formula I or salts, enantiomers, stereoisomers, polymorphs or solvates thereof as described in embodiment 37), wherein Rₐ₁ is selected from the group consisting of hydrogen, deuterium, unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl; preferably, Rₐ₁ is selected from the group consisting of hydrogen, deuterium, unsubstituted or substituted C₅-C₂₀ aryl, unsubstituted or substituted 5- to 20-membered heteroaryl, unsubstituted or substituted C₃-C₂₀ cycloalkyl, or unsubstituted or substituted 4- to 20-membered heterocyclyl.

Embodiment 39): Pertaining to the compound of Formula I or salts, enantiomers, stereoisomers, polymorphs or solvates thereof as described in embodiment 37), wherein Rₐ₁ is hydrogen.

Embodiment 40): Pertaining to the compound of Formula I or salts, enantiomers, stereoisomers, polymorphs or solvates thereof as described in embodiment 37), wherein Rₐ₁ is deuterium.

Embodiment 41): Pertaining to the compound of Formula I or salts, enantiomers, stereoisomers, polymorphs or solvates thereof as described in embodiment 37), wherein Rₐ₁ is unsubstituted or substituted aryl. Optionally, the unsubstituted or substituted aryl is unsubstituted or substituted C₅-C₃₀ aryl.

Optionally, the unsubstituted or substituted C₅-C₃₀ aryl is unsubstituted or substituted C₅-C₂₅, C₅-C₂₀, C₅—C₁₅ aryl. Optionally, the C₅-C₃₀ aryl may be monocyclic or polycyclic aryl. In some embodiments, monocyclic aryl includes but is not limited to phenyl, biphenyl, terphenyl, quaterphenyl, and quinquephenyl etc. Polycyclic aryl includes but is not limited to naphthyl, anthryl, phenanthryl, pyrenyl, perylenyl, and fluorenyl etc. Fluorenyl is optionally substituted, with examples including 9,9′-dimethylfluorenyl and 9,9′-diphenylfluorenyl etc. Furthermore, two of the substituents may combine with each other to form a spirocyclic structure such as 9,9′-spirobifluorenyl etc. The aryl is optionally substituted aryl. The substituted aryl refers to an aryl group substituted one or more times (e.g., 1-4, 1-3, or 1-2 times) with a substituent. For example, aryl is mono-, di-, or tri-substituted with a substituent optionally selected from the group consisting of e.g., deuterated C₁-C₆ alkyl, deuterated C₃-C₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, unsubstituted or substituted cycloalkyl, alkenyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof (preferably, the substituent is selected from the group consisting of deuterated C₁-C₆ alkyl, deuterated C₃-C₆ cycloalkyl, hydroxy, amino, mercapto, halogen, cyano, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl, and any combination thereof). Further, when Rₐ₁ is substituted aryl and the substituent of aryl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.) (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, or C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, or C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof). Further, when Rₐ₁ is substituted aryl and the substituent of aryl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl and unsubstituted or substituted C₅-C₁₀ aryl etc.) (optionally, the unsubstituted or substituted aryl is selected from the group consisting of biphenyl, terphenyl, naphthyl, anthryl, phenanthryl, fluorenyl, or 9,9′-dimethylfluorenyl etc.; and the unsubstituted or substituted aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof). Further, when Rₐ₁ is substituted aryl and the substituent of aryl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl, or unsubstituted or substituted 5- to 10-membered heteroaryl) (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl; the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof). Further, when Rₐ₁ is substituted aryl and the substituent of aryl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.) (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl, or 5- to 15-membered bridged heterocyclyl, or 7- to 15-membered bridged heterocyclyl, such as azabicyclohexyl, azabicycloheptanyl, diazabicycloheptanyl, azabicyclooctyl, and diazabicyclooctyl), or spiro-heterocyclyl (e.g., 4- to 15-membered spiro-heterocyclyl, or 5- to 15-membered spiro-heterocyclyl, or 7- to 15-membered spiro-heterocyclyl, such as 3-azaspiro[5.5]undecanyl or 7-azaspiro[3.5]nonanyl); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof).

Embodiment 42): Pertaining to the compound of Formula I or salts, enantiomers, stereoisomers, polymorphs or solvates thereof as described in embodiment 37), wherein Rₐ₁ is unsubstituted or substituted heteroaryl. When the Rₐ₁ is unsubstituted or substituted heteroaryl, the unsubstituted or substituted heteroaryl is unsubstituted or substituted 5- to 30-membered heteroaryl (optionally, the unsubstituted or substituted 5- to 30-membered heteroaryl is unsubstituted or substituted 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered heteroaryl).

Optionally, the heteroaryl includes but is not limited to pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, tetrazolyl, pyranyl, thiopyranyl, pyrazinyl, thiazinyl, triazinyl, tetrazinyl, quinolinyl, isoquinolinyl, quinazolinyl, quinoxalinyl, cinnolinyl, naphthyridinyl, acridinyl, xanthenyl, phenanthridinyl, phthalazinyl, triazaindenyl, indolyl, indolinyl, indolizinyl, phthalazinyl, pyrazolopyridyl, pyrazolopyrimidinyl, pyridopyrimidinyl, pyridopyrazinyl, pyrazinopyrazinyl, benzothiazolyl, benzoxazolyl, benzimidazolyl, benzothienyl, benzofuranyl, isobenzofuranyl, dibenzothienyl, dibenzofuranyl, indazolyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, pyrrolopyridinyl, pyrrolothiazolyl, imidazothiazolyl, pyrenyl, dinaphthofuranyl, naphthobenzofuranyl, dinaphthothienyl, or naphthobenzothienyl etc. Wherein the heteroaryl is unsubstituted or substituted. The substituted heteroaryl refers to a heteroaryl substituted one or more times (e.g., 1-4, 1-3, or 1-2 times) with a substituent optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, or any combination thereof (preferably, the substituent is optionally selected from the group consisting of C₁-C₃ alkyl, C₃-C₆ cycloalkyl, hydroxy, amino, mercapto, halogen, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, unsubstituted or substituted cycloalkyl, unsubstituted or substituted aryl, unsubstituted or substituted heteroaryl, unsubstituted or substituted heterocyclyl, or any combination thereof). Further, when Rₐ₁ is substituted heteroaryl and the substituent of heteroaryl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.) (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, or C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged heterocyclyl (e.g., C₃-C₁₅ bridged heterocyclyl, C₅-C₁₅ bridged heterocyclyl, or C₇-C₁₅ bridged heterocyclyl, such as adamantanyl, noradamantanyl, bornyl, bicyclo[2.2.1]heptyl, 2-oxobicyclo[2.2.1]heptyl, or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₁ is substituted heteroaryl and the substituent of heteroaryl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.) (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof. Further, when Rₐ₁ is substituted heteroaryl and the substituent of heteroaryl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl, or unsubstituted or substituted 5- to 10-membered heteroaryl) (optionally, the unsubstituted or substituted heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl; and the unsubstituted or substituted heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when Rₐ₁ is substituted heteroaryl and the substituent of heteroaryl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, or azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 43): Pertaining to the compound of Formula I or salts, enantiomers, stereoisomers, polymorphs or solvates thereof as described in embodiment 37), wherein Rₐ₁ is unsubstituted or substituted cycloalkyl. When the Rₐ₁ is unsubstituted or substituted cycloalkyl, the unsubstituted or substituted cycloalkyl is unsubstituted or substituted C₃-C₂₀, C₃-C₁₅, C₃-C₁₂, C₃-C₁₁, C₃-C₁₀, C₃-C₈, C₃-C₇, C₃-C₆ cycloalkyl etc. Optionally, the cycloalkyl includes but is not limited to cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl) etc. Wherein, the “cycloalkyl” is optionally substituted, and the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, amino, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, unsubstituted or substituted cycloalkyl, deuterated cycloalkyl, alkenyl, unsubstituted or substituted aryl, unsubstituted or substituted heteroaryl, unsubstituted or substituted heterocyclyl, aralkyl, arylalkenyl, alkyl aryl, alkylamino, aralkylamino, heteroarylamino, arylamino, arylphosphino, acenaphthenyl, oxo, or any combination thereof. Further, the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, unsubstituted or substituted cycloalkyl, unsubstituted or substituted aryl, unsubstituted or substituted heteroaryl, unsubstituted or substituted heterocyclyl, or any combination thereof. Further, when Rₐ₁ is substituted cycloalkyl and the substituent of cycloalkyl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.) (optionally, the unsubstituted or substituted cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged heterocyclyl (e.g., C₃-C₁₅ bridged heterocyclyl or C₅-C₁₅ bridged heterocyclyl or C₇-C₁₅ bridged heterocyclyl, such as adamantanyl, noradamantanyl, bornyl, bicyclo[2.2.1]heptyl, 2-oxobicyclo[2.2.1]heptyl or bicyclo[2.2.1]heptentyl); wherein when the unsubstituted or substituted cycloalkyl is substituted cycloalkyl, the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof). Further, when Rₐ₁ is substituted cycloalkyl and the substituent of cycloalkyl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.) (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₁ is substituted cycloalkyl and the substituent of cycloalkyl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl, or unsubstituted or substituted 5- to 10-membered heteroaryl) (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₁ is substituted cycloalkyl and the substituent of cycloalkyl is unsubstituted or substituted heterocyclyl, the heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl, or 5- to 15-membered bridged heterocyclyl, or 7- to 15-membered bridged heterocyclyl, such as azabicyclohexyl, azabicycloheptanyl, diazabicycloheptanyl, and azabicyclooctyl), or spiro-heterocyclyl (e.g., 4- to 15-membered spiro-heterocyclyl, or 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 44): Pertaining to the compound of Formula I or salts, enantiomers, stereoisomers, polymorphs or solvates thereof as described in embodiment 37), wherein Rₐ₁ is unsubstituted or substituted heterocyclyl. When the Rₐ₁ is unsubstituted or substituted heterocyclyl, the unsubstituted or substituted heterocyclyl is unsubstituted or substituted 4- to 30-membered heterocyclyl, 4- to 25-membered heterocyclyl, 4- to 20-membered heterocyclyl, 4- to 15-membered heterocyclyl, 4- to 10-membered heterocyclyl, 10- to 15-membered heterocyclyl, 5- to 15-membered heterocyclyl, 7- to 15-membered heterocyclyl etc. Optionally, the heterocyclyl contains 1-3 heteroatoms, or 1-2 heteroatoms, or 1 heteroatom etc, with each heteroatom independently selected from the group consisting of O, N, and S etc. Further optionally, the heterocyclyl includes but is not limited to azetidinyl, oxetanyl, pyrrolidinyl, imidazolyl, pyrazolidyl, triazolyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl (e.g., 1, 4-diazacycloheptanyl, 4, 5-diazacycloheptanyl, or 1, 3-diazacycloheptanyl etc.), azabicyclohexyl (e.g., 3-azabicyclo[3.1.0]hexyl etc.), azabicycloheptanyl (e.g., 3-azabicyclo[3.1.1]heptanyl, 3-azabicyclo[3.2.0]heptanyl etc.), azabicyclooctyl (e.g., cis-7-azabicyclo[3.3.0]octyl etc.), and spiro-heterocyclyl (7-azaspiro[3.5]nonanyl, 3-azaspiro[5,5]undecanyl etc.). The heterocyclyl may be unsubstituted or substituted as explicitly defined (e.g., mono-, di-, tri-, or poly-substituted) by a substituent optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, oxo and any combination thereof (preferably, the substituent is selected from the group consisting of deuterium, hydroxy, amino, mercapto, nitro, halogen, cyano, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl, and any combination thereof). Further, when Rₐ₁ is substituted heterocyclyl and the substituent of heterocyclyl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl or C₅-C₁₅ bridged cycloalkyl or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₁ is substituted heterocyclyl and the substituent of heterocyclyl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₁ is substituted heterocyclyl and the substituent of heterocyclyl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₁ is substituted heterocyclyl and the substituent of heterocyclyl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.) (optionally, the unsubstituted or substituted heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl); and the unsubstituted or substituted heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof).

Embodiment 45): Pertaining to the compound of Formula I or salts, enantiomers, stereoisomers, polymorphs or solvates thereof as described in any one of embodiments 1)-44), wherein Rₐ2 is selected from the group consisting of hydrogen, deuterium, unsubstituted or substituted aryl, unsubstituted or substituted heteroaryl, unsubstituted or substituted cycloalkyl, or unsubstituted or substituted heterocyclyl, or Rₐ2 is absent; and when X₃ represents CO, Rₐ2 is absent and Rₐ₁ is neither hydrogen nor deuterium, and when Rₐ₂ represents hydrogen or deuterium, X₃ represents CR₁ or N, and Rₐ₁ is neither hydrogen nor deuterium.

Embodiment 46): Pertaining to the compound of Formula I or salts, enantiomers, stereoisomers, polymorphs or solvates thereof as described in embodiment 45), wherein Rₐ₂ is selected from the group consisting of hydrogen, deuterium, unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl, or Rₐ₂ is absent.

Embodiment 47): Pertaining to the compound of Formula I or salts, enantiomers, stereoisomers, polymorphs or solvates thereof as described in embodiment 45) or 46), wherein Rₐ2 is selected from the group consisting of hydrogen, deuterium, unsubstituted or substituted C₅-C₂₀ aryl, unsubstituted or substituted 5- to 20-membered heteroaryl, unsubstituted or substituted C₃-C₂₀ cycloalkyl, or unsubstituted or substituted 4- to 20-membered heterocyclyl, or Rₐ2 is absent.

Embodiment 48): Pertaining to the compound of Formula I or salts, enantiomers, stereoisomers, polymorphs or solvates thereof as described in any one of embodiments 45)-47), wherein Rₐ₂ is selected from the group consisting of hydrogen.

Embodiment 49): Pertaining to the compound of Formula I or salts, enantiomers, stereoisomers, polymorphs or solvates thereof as described in any one of embodiments 45)-47), wherein Rₐ₂ is selected from the group consisting of deuterium.

Embodiment 50): Pertaining to the compound of Formula I or salts, enantiomers, stereoisomers, polymorphs or solvates thereof as described in embodiment 45), wherein Rₐ₂ is selected from the group consisting of unsubstituted or substituted C₅-C₂₀ aryl (e.g., unsubstituted or substituted C₅-C₁₅ aryl, unsubstituted or substituted C₅-C₁₀ aryl etc.). The aryl may be monocyclic or polycyclic aryl. In some embodiments, the monocyclic aryl includes but is not limited to phenyl, biphenyl, terphenyl, quaterphenyl, and quinquephenyl etc. The polycyclic aryl includes but is not limited to naphthyl, anthryl, phenanthryl, pyrenyl, perylenyl, and fluorenyl etc. The fluorenyl may be substituted (e.g., 9,9′-dimethylfluorenyl and 9,9′-diphenylfluorenyl etc.). Preferably, Rₐ₂ is selected from the group consisting of biphenyl, terphenyl, naphthyl, anthryl, phenanthryl, fluorenyl, and 9,9′-dimethylfluorenyl. Furthermore, two of the substituents may combine with each other to form a spirocyclic structure such as 9,9′-spirobifluorenyl etc. The aryl is optionally substituted aryl. The substituted aryl refers to an aryl group substituted one or more times (e.g., 1-4, 1-3, or 1-2 times) with a substituent. For example, aryl is mono-, di-, or tri-substituted with a substituent optionally selected from the group consisting of e.g., deuterated C₁-C₆ alkyl, deuterated C₃-C₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, unsubstituted or substituted cycloalkyl, alkenyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof (preferably, the substituent is selected from the group consisting of deuterated C₁-C₆ alkyl, deuterated C₃-C₆ cycloalkyl, hydroxy, amino, mercapto, halogen, cyano, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl, and any combination thereof). Further, when Rₐ₂ is substituted aryl and the substituent of aryl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.) (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl or C₅-C₁₅ bridged cycloalkyl or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted aryl and the substituent of aryl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.) (optionally, the unsubstituted or substituted aryl is selected from the group consisting of biphenyl, terphenyl, naphthyl, anthryl, phenanthryl, fluorenyl, or 9,9′-dimethylfluorenyl etc.); and the unsubstituted or substituted aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when Rₐ₂ is substituted aryl and the substituent of aryl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl) (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted aryl and the substituent of aryl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.) (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 51): Pertaining to the compound of Formula I or salts, enantiomers, stereoisomers, polymorphs or solvates thereof as described in embodiment 45), wherein Rₐ₂ is selected from the group consisting of unsubstituted or substituted 5- to 25-membered heteroaryl (preferably, unsubstituted or substituted 5- to 20-membered heteroaryl, or unsubstituted or substituted 5- to 15-membered heteroaryl); more preferably, Rₐ₂ is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl. Wherein the heteroaryl may be unsubstituted or substituted. The substituted heteroaryl refers to a heteroaryl substituted one or more times (e.g., 1-4, 1-3, or 1-2 times) with a substituent optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, or any combination thereof (preferably, the substituent is optionally selected from the group consisting of C₁-C₃ alkyl, C₃-C₆ cycloalkyl, hydroxy, amino, mercapto, halogen, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, unsubstituted or substituted cycloalkyl, unsubstituted or substituted aryl, unsubstituted or substituted heteroaryl, unsubstituted or substituted heterocyclyl, or any combination thereof). Further, when Rₐ₁ is substituted heteroaryl and the substituent of heteroaryl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.) (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl or C₅-C₁₅ bridged cycloalkyl or C₇-C₁₅ bridged cycloalkyl, such as adamantanyl, noradamantanyl, bornyl, bicyclo[2.2.1]heptyl, 2-oxobicyclo[2.2.1]heptyl orbicyclo[2.2.1]heptentyl); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted heteroaryl and the substituent of heteroaryl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.) (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted heteroaryl and the substituent of heteroaryl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl) (optionally, the unsubstituted or substituted heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl; and the unsubstituted or substituted heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when Rₐ₂ is substituted heteroaryl and the substituent of heteroaryl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.) (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 52): Pertaining to the compound of Formula I or salts, enantiomers, stereoisomers, polymorphs or solvates thereof as described in embodiment 45), wherein Rₐ2 is selected from the group consisting of unsubstituted or substituted 4- to 20-membered heterocyclyl (e.g., unsubstituted or substituted 4- to 15-membered heterocyclyl); preferably, Rₐ₂ is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl). The heterocyclyl may be unsubstituted or substituted pyrrolidinyl, citly defined (e.g., mono-, di-, tri-, or poly-substituted) by a substituent optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof (preferably, the substituent is selected from the group consisting of deuterium, hydroxy, amino, mercapto, nitro, halogen, cyano, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁-C₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocycly,l and any combination thereof). Further, when Rₐ₂ is substituted heterocyclyl and the substituent of heterocyclyl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl or C₅-C₁₅ bridged cycloalkyl or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted heterocyclyl and the substituent of heterocyclyl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted heterocyclyl and the substituent of heterocyclyl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted heterocyclyl and the substituent of heterocyclyl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.) (optionally, the unsubstituted or substituted heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl); and the unsubstituted or substituted heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof).

Embodiment 53): Pertaining to the compound of Formula I or salts, enantiomers, stereoisomers, polymorphs or solvates thereof as described in embodiment 45), wherein Rₐ₂ is selected from the group consisting of unsubstituted or substituted C₃-C₂₀ cycloalkyl (e.g., unsubstituted or substituted C₃-C₁₅ cycloalkyl); preferably, Rₐ₂ is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl or C₅-C₁₅ bridged cycloalkyl or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl). Wherein the “cycloalkyl” is optionally substituted, and the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, amino, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, unsubstituted or substituted cycloalkyl, deuterated cycloalkyl, alkenyl, unsubstituted or substituted aryl, unsubstituted or substituted heteroaryl, unsubstituted or substituted heterocyclyl, aralkyl, arylalkenyl, alkyl aryl, alkylamino, aralkylamino, heteroarylamino, arylamino, arylphosphino, acenaphthenyl, oxo or any combination thereof. Further, the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, unsubstituted or substituted cycloalkyl, unsubstituted or substituted aryl, unsubstituted or substituted heteroaryl, unsubstituted or substituted heterocyclyl, or any combination thereof. Further, when Rₐ₂ is substituted cycloalkyl and the substituent of cycloalkyl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.) (optionally, the unsubstituted or substituted cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl or C₅-C₁₅ bridged cycloalkyl or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl); and the unsubstituted or substituted cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof). Further, when Rₐ₂ is substituted cycloalkyl and the substituent of cycloalkyl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof. Further, when Rₐ₂ is substituted cycloalkyl and the substituent of cycloalkyl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when Rₐ₂ is substituted cycloalkyl and the substituent of cycloalkyl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆cycloalkyl, amino-substituted C₁-C₆ alkylene, C₁-C₄ alkyl-NHC(O)—, C₁-C₄ alkyl-C(O)NH—, and any combination thereof.

Embodiment 54): Pertaining to the compound of Formula I or salts, enantiomers, stereoisomers, polymorphs or solvates thereof as described in any one of embodiments 45)-53), wherein the substituents of the substituted Rₐ₂ are optionally one or more selected from the group consisting of deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, linear or branched C₁-C₅ alkyl, halogenated C₁-C₅ alkyl, optionally substituted alkyl, optionally substituted aryl, optionally substituted heteroaryl, and optionally substituted heterocyclyl. Further, the optionally substituted alkyl is e.g., optionally substituted C₁-C₃₀ alkyl, optionally substituted C₁-C₂₀ alkyl, optionally substituted C₁-C₁₅ alkyl, and optionally substituted C₁-C₁₀ alkyl etc., wherein the substituents of the optionally substituted alkyl are e.g., selected from the group consisting of C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. The optionally substituted aryl is e.g., optionally substituted C₅-C₃₀ aryl, optionally substituted C₅-C₂₅ aryl, optionally substituted C₅-C₁₅ aryl, and optionally substituted C₅-C₁₀ aryl etc., wherein the substituent of the optionally substituted aryl is e.g., selected from the group consisting of C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. The optionally substituted heteroaryl is e.g., optionally substituted 5- to 30-membered heteroaryl, unsubstituted or substituted 5- to 15-membered, 5- to 12-membered, 5- to 11-membered, 5- to 10-membered, 5- to 9-membered, 5- to 8-membered, 5- to 7-membered, 5- to 6-membered, 6- to 15-membered or 6- to 9-membered heteroaryl, wherein the substituent of the optionally substituted heteroaryl is e.g., selected from the group consisting of C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. The optionally substituted heterocyclyl is optionally substituted 4- to 30-membered heterocyclyl, optionally substituted 4- to 25-membered heterocyclyl, optionally substituted 4- to 20-membered heterocyclyl, optionally substituted 4- to 15-membered heterocyclyl, optionally substituted 4- to 10-membered heterocyclyl, or optionally substituted 10- to 15-membered heterocyclyl, wherein the substituent of the optionally substituted heterocyclyl is e.g., selected from the group consisting of C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 55): Pertaining to the compound of Formula I or salts, enantiomers, stereoisomers, polymorphs or solvates thereof as described in embodiment 45), wherein Rₐ₂ is absent.

Embodiment 56): Pertaining to the compound of Formula I or salts, enantiomers, stereoisomers, polymorphs or solvates thereof as described in embodiment 45), wherein Rₐ₁ and Rₐ2 cannot simultaneously be hydrogen or deuterium.

Embodiment 57): Pertaining to the compound of Formula I or salts, enantiomers, stereoisomers, polymorphs or solvates thereof as described in embodiment 45), wherein when Rₐ2 is absent, X₃ represents CO.

Embodiment 58): Pertaining to the compound of Formula I or salts, enantiomers, stereoisomers, polymorphs or solvates thereof as described in any one of embodiments 1)-53) and 55), wherein Rₐ2 is selected from the group consisting of

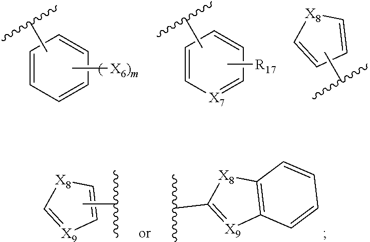

wherein X₆ is selected from the group consisting of deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, halogenated C₁-C₆ alkyl, or linear or branched C₁-C₆ alkyl. Optionally, halogen may be e.g., F, Cl, Br, I. Optionally, C₁-C₆ alkoxy may include, but is not limited to, e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like. Optionally, the halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino, and the like. Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc. Optionally, the linear or branched C₁-C₆ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, and tert-penty letc, and the linear or branched C₁-C₆ alkyl is optionally substituted with one or more substituents optioanlly selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl) or any combination thereof;
m is an integer of 0, 1, 2, 3, 4 or 5, and when m is an integer of 2, 3, 4 or 5, each X₆ is independently identical to or different from each other;
X₇ and X₉ are each independently selected from the group consisting of CH or N;
each Xₛ is independently selected from the group consisting of O, S or NH;
R₁₇ is selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, halogenated C₁-C₅alkyl, or linear or branched C₁-C₅alkyl, wherein the halogen may optionally be e.g., F, Cl, Br, or I. Optionally, the C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like.

Optionally, the halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino, and the like. Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc.

Optionally, the linear or branched C₁-C₅alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, and tert-pentyl etc., and the linear or branched C₁-C₅alkyl is optionally substituted with one or more substituents optioanlly selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl), or any combination thereof.

Embodiment 59): Pertaining to the compound of Formula I or salts, enantiomers, stereoisomers, polymorphs or solvates thereof as described in any one of embodiments 1)-58), wherein Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl.

Embodiment 60): Pertaining to the compound of Formula I or salts, enantiomers, stereoisomers, polymorphs or solvates thereof as described in embodiment 59), wherein Rₐ is selected from the group consisting of deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, unsubstituted or substituted linear or branched C₁-C₅alkyl, unsubstituted or substituted C₃-C₁₅ cycloalkyl, unsubstituted or substituted 4- to 15-membered heterocyclyl, unsubstituted or substituted C₅-C₁₅ aryl, or unsubstituted or substituted 5- to 15-membered heteroaryl.

Embodiment 61): Pertaining to the compound of Formula I or salts, enantiomers, stereoisomers, polymorphs or solvates thereof as described in embodiment 59) or 60), wherein Rₐ is selected from the group consisting of deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, unsubstituted or substituted linear or branched C₁-C₅alkyl, unsubstituted or substituted C₃-C₁₅ cycloalkyl, unsubstituted or substituted 4- to 15-membered heterocyclyl, unsubstituted or substituted C₅-C₁₅ aryl, or unsubstituted or substituted 5- to 15-membered heteroaryl.

Embodiment 62): Pertaining to the compound of Formula I or salts, enantiomers, stereoisomers, polymorphs or solvates thereof as described in embodiment 59), wherein Rₐ is deuterium.

Embodiment 63): Pertaining to the compound of Formula I or salts, enantiomers, stereoisomers, polymorphs or solvates thereof as described in embodiment 59), wherein Rₐ is halogen, which is optionally F, Cl, Br, or I.

Embodiment 64): Pertaining to the compound of Formula I or salts, enantiomers, stereoisomers, polymorphs or solvates thereof as described in embodiment 59), wherein Rₐ is nitro.

Embodiment 65): Pertaining to the compound of Formula I or salts, enantiomers, stereoisomers, polymorphs or solvates thereof as described in embodiment 59), wherein Rₐ is cyano.

Embodiment 66): Pertaining to the compound of Formula I or salts, enantiomers, stereoisomers, polymorphs or solvates thereof as described in embodiment 59), wherein Rₐ is amino.

Embodiment 67): Pertaining to the compound of Formula I or salts, enantiomers, stereoisomers, polymorphs or solvates thereof as described in embodiment 59), wherein Rₐ is hydroxy.

Embodiment 68): Pertaining to the compound of Formula I or salts, enantiomers, stereoisomers, polymorphs or solvates thereof as described in embodiment 59), wherein Rₐ is mercapto.

Embodiment 69): Pertaining to the compound of Formula I or salts, enantiomers, stereoisomers, polymorphs or solvates thereof as described in embodiment 59), wherein Rₐ is alkoxy. Optionally, when the Rₐ is alkoxy, the alkoxy is C₁-C₆ alkoxy. C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like.

Embodiment 70): Pertaining to the compound of Formula I or salts, enantiomers, stereoisomers, polymorphs or solvates thereof as described in embodiment 59), wherein Rₐ is halogenated alkoxy.

Optionally, when the Rₐ is halogenated alkoxy, the halogenated alkoxy is halogenated C₁-C₆ alkoxy; further optionally the halogenated C₁-C₆ alkoxy is trifluoromethoxy etc.

Embodiment 71): Pertaining to the compound of Formula I or salts, enantiomers, stereoisomers, polymorphs or solvates thereof as described in embodiment 59), wherein Rₐ is unsubstituted or substituted amino. Optionally, the unsubstituted or substituted amino includes amino, N-methylamino, N-ethylamino, N-phenylamino, N,N-dimethylamino, and N,N-diethylamino, and the like.

Embodiment 72): Pertaining to the compound of Formula I or salts, enantiomers, stereoisomers, polymorphs or solvates thereof as described in embodiment 59), wherein Rₐ is unsubstituted or substituted silyl. Optionally, the unsubstituted or substituted silyl is specifically trimethylsilyl, triethylsilyl, tert-butyldimethylsilyl, vinyldimethylsilyl, propyldimethylsilyl, triphenylsilyl, diphenylsilyl, and phenylsilyl etc.

Embodiment 73): Pertaining to the compound of Formula I or salts, enantiomers, stereoisomers, polymorphs or solvates thereof as described in embodiment 59), wherein Rₐ is unsubstituted or substituted boryl. Optionally, the unsubstituted or substituted boryl is specifically trimethylboryl, triethylboryl, tert-butyldimethylboryl, triphenylboryl, and phenylboryl etc.

Embodiment 74): Pertaining to the compound of Formula I or salts, enantiomers, stereoisomers, polymorphs or solvates thereof as described in embodiment 59), wherein Rₐ is unsubstituted or substituted linear or branched alkyl. Optionally, the number of carbon atoms may be for example C₁-C₃₀, C₁-C₂₅, C₁-C₂₀, C₁-C₁₅, C₁-C₁₀, C₁-C₅, C₁-C₃ etc, with linear or branched C₁-C₁₀ alkyl being preferred. Further optionally, the linear or branched C₁-C₁₀ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, nonyl, 2,2-dimethylheptyl, 1-ethylpropyl, 1,1-dimethylpropyl, isohexyl, 4-methylhexyl, or 5-methylhexyl etc. Optionally, the linear or branched C₁-C₁₀ alkyl includes but is not limited to methyl, ethyl, and propyl etc. Wherein the alkyl is optionally substituted, and the substituent may be e.g., one or more optionally selected from the group consisting of deuterium, halogen, cyano, nitro, hydroxy, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo, or any combination thereof. The substituent is preferably one or more selected from the group consisting of D (i.e., deuterium), halogen, hydroxy, cyano, C₁₋₃ alkyl, C₁₋₃ alkoxy, trifluoromethyl, heterocyclyl (e.g., 4- to 8-membered heterocyclyl) or any combination thereof.

Embodiment 75): Pertaining to the compound of Formula I or salts, enantiomers, stereoisomers, polymorphs or solvates thereof as described in embodiment 59), wherein Rₐ is unsubstituted or substituted cycloalkyl. Optionally, when the Rₐ is unsubstituted or substituted cycloalkyl, it has 3 to 20 carbon atoms (i.e., C₃₋₂₀ cycloalkyl), 3 to 15 carbon atoms (i.e., C₃₋₁₅ cycloalkyl), 3 to 12 carbon atoms (i.e., C₃₋₁₂ cycloalkyl), 3 to 11 carbon atoms (i.e., C₃₋₁₁ cycloalkyl), 3 to 10 carbon atoms (i.e., C₃₋₁₀ cycloalkyl), 3 to 8 carbon atoms (i.e., C₃₋₈ cycloalkyl), 3 to 7 carbon atoms (i.e., C₃₋₇ cycloalkyl), or 3 to 6 carbon atoms (i.e., C₃₋₆ cycloalkyl). R₁ is preferably unsubstituted or substituted C₃-C₁₅ cycloalkyl. Optionally, C₃-C₁₅ cycloalkyl includes but is not limited to cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl) etc. Further optionally, C₃-C₁₅ cycloalkyl includes but is not limited to cyclopropyl, cyclobutyl, cyclopentyl etc. Wherein the cycloalkyl is optionally substituted with one or more (e.g., 1-5, 1-4, 1-3, 1-2, or 1) substituents optioanlly selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, amino, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, acenaphthenyl, oxo, or any combination thereof. The substituents are preferably one or more selected from the group consisting of trifluoromethyl, mercapto, hydroxy, amino, halogen, cyano, C₁-C₃ alkyl, C₁-C₃ alkoxy, trifluoromethyl, heterocyclyl or any combination thereof.

Embodiment 76): Pertaining to the compound of Formula I or salts, enantiomers, stereoisomers, polymorphs or solvates thereof as described in embodiment 59), wherein Rₐ is unsubstituted or substituted heterocyclyl. Optionally, when the Rₐ is unsubstituted or substituted heterocyclyl, the unsubstituted or substituted heterocyclyl is unsubstituted or substituted 4- to 30-membered heterocyclyl, 4- to 25-membered heterocyclyl, 4- to 20-membered heterocyclyl, 4- to 15-membered heterocyclyl, 4- to 10-membered heterocyclyl, 10- to 15-membered heterocyclyl, 5- to 15-membered heterocyclyl, or 7- to 15-membered heterocyclyl etc. Optionally, the heterocyclyl contains 1-3 heteroatoms, or 1-2 heteroatoms, or 1 heteroatom etc, with each heteroatom independently selected from the group consisting of O, N, and S etc. Further optionally, the heterocyclyl includes but is not limited to azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl (e.g., 1, 4-diazacycloheptanyl, 4, 5-diazacycloheptanyl, or 1, 3-diazacycloheptanyl etc), azabicyclohexyl (e.g., 3-azabicyclo[3.1.0]hexyl etc), azabicycloheptanyl (e.g., 3-azabicyclo[3.1.1]heptanyl, 3-azabicyclo[3.2.0]heptanyl etc), azabicyclooctyl (e.g., cis-7-azabicyclo[3.3.0]octyl etc), and spiro-heterocyclyl (e.g., 4- to 15-membered spiro-heterocyclyl or 5- to 15-membered spiro-heterocyclyl or 7 to 15-membered spiro-heterocyclyl, such as 7-azaspiro[3.5]nonanyl, 3-azaspiro[5,5]undecanyl etc). The heterocyclyl may be unsubstituted or substituted as explicitly defined (e.g., mono-, di-, tri-, or poly-substituted) by a substituent optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃₋₆ cycloalkyl, optionally deuterated C₁-C₆ alkoxy, C₁₋₄ alkyl-NH—, NH₂—C₁₋₆ alkylene, C₁₋₄ alkyl-NHC(O)—, C₁₋₄ alkyl-C(O)NH—, alkenyl, aryl, aralkyl, arylalkenyl, alkylaryl, aralkylamino, heteroarylamino, arylamino, arylphosphino, heteroaryl, acenaphthenyl, and any combination thereof.

Preferably, the substituent is selected from the group consisting of deuterium, hydroxy, amino, mercapto, nitro, halogen, cyano, oxo, optionally deuterated C₁-C₆ alkyl, halogenated C₁-C₆ alkyl, optionally deuterated C₃₋₆ cycloalkyl, optionally deuterated C₁-C₆ alkoxy, C₁₋₄ alkyl-NH—, NH₂—C₁₋₆ alkylene, C₁₋₄ alkyl-NHC(O)—, C₁₋₄ alkyl-C(O)NH—, and any combination thereof.

Embodiment 77): Pertaining to the compound of Formula I or salts, enantiomers, stereoisomers, polymorphs or solvates thereof as described in embodiment 59), wherein Rₐ is unsubstituted or substituted aryl. Optionally, the unsubstituted or substituted aryl is unsubstituted or substituted C₅₋₃₀ aryl. Optionally, the unsubstituted or substituted C₅-30 aryl is unsubstituted or substituted C₅₋₂₅, C₅₋₂₀, or C₅₋₁₅ aryl.

Optionally, the C₅₋₃₀ aryl may be monocyclic or polycyclic aryl. In some embodiments, monocyclic aryl includes but is not limited to phenyl, biphenyl, terphenyl, quaterphenyl, and quinquephenyl etc. Polycyclic aryl includes but is not limited to naphthyl, anthryl, phenanthryl, pyrenyl, perylenyl, and fluorenyl etc.

Fluorenyl is optionally substituted, with examples including 9,9′-dimethylfluorenyl and 9,9′-diphenylfluorenyl etc. Furthermore, two of the substituents may combine with each other to form a spirocyclic structure such as 9,9′-spirobifluorenyl etc. The aryl is optionally substituted aryl. The substituted aryl refers to an aryl group substituted one or more times (e.g., 1-4, 1-3, or 1-2 times) with a substituent. For example, aryl is mono-, di-, or tri-substituted with a substituent optionally selected from the group consisting of e.g., deuterated C₁-C₆ alkyl, deuterated C₃₋₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, cycloalkyl, alkenyl, aryl, aralkyl, arylalkenyl, alkylaryl, aralkylamino, heteroarylamino, arylamino, arylphosphino, heteroaryl, acenaphthenyl, and any combination thereof. Preferably, the substituent is selected from the group consisting of deuterated C₁-C₆ alkyl, deuterated C₃₋₆ cycloalkyl, hydroxy, amino, mercapto, halogen, cyano, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, and any combination thereof.

Embodiment 78): Pertaining to the compound of Formula I or salts, enantiomers, stereoisomers, polymorphs or solvates thereof as described in embodiment 59), wherein Rₐ is unsubstituted or substituted heteroaryl. When the Rₐ is unsubstituted or substituted heteroaryl, the unsubstituted or substituted heteroaryl is unsubstituted or substituted 5- to 30-membered heteroaryl. Optionally, the unsubstituted or substituted 5- to 30-membered heteroaryl is unsubstituted or substituted 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered heteroaryl. Optionally, the heteroaryl includes but is not limited to pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, tetrazolyl, pyranyl, thiopyranyl, pyrazinyl, thiazinyl, triazinyl, tetrazinyl, quinolinyl, isoquinolinyl, quinazolinyl, quinoxalinyl, cinnolinyl, naphthyridinyl, acridinyl, xanthenyl, phenanthridinyl, phthalazinyl, triazaindenyl, indolyl, indolinyl, indolizinyl, phthalazinyl, pyrazolopyridyl, pyrazolopyrimidinyl, pyridopyrimidinyl, pyridopyrazinyl, pyrazinopyrazinyl, benzothiazolyl, benzoxazolyl, benzimidazolyl, benzothienyl, benzofuranyl, isobenzofuranyl, dibenzothienyl, dibenzofuranyl, indazolyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, pyrrolopyridinyl, pyrrolothiazolyl, imidazothiazolyl, pyrenyl, dinaphthofuranyl, naphthobenzofuranyl, dinaphthothienyl, and naphthobenzothienyl etc. Wherein the heteroaryl is unsubstituted or substituted. The substituted heteroaryl refers to a heteroaryl substituted one or more times (e.g., 1-4, 1-3, or 1-2 times) with a substituent optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, aryl, aralkyl, arylalkenyl, alkylaryl, aralkylamino, heteroarylamino, arylamino, arylphosphino, heteroaryl, acenaphthenyl, or any combination thereof. Preferably, the substituent is optionally selected from the group consisting of C₁-C₃ alkyl, C₃-C₆ cycloalkyl, hydroxy, amino, mercapto, halogen, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, or any combination thereof.

Embodiment 79): Pertaining to the compound of Formula I or salts, enantiomers, stereoisomers, polymorphs or solvates thereof as described in any one of embodiments 1)-78), wherein R_{b1}, R_{b2}, R_{b3}, R_{b4}, and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, and unsubstituted or substituted linear or branched alkyl.

Embodiment 80): Pertaining to the compound of Formula I or salts, enantiomers, stereoisomers, polymorphs or solvates thereof as described in any one of embodiments 1)-78), wherein R_{b1}, R_{b2}, R_{b3}, R_{b4}, and R_{b5} are each independently selected from the group consisting of hydrogen, deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, or unsubstituted or substituted linear or branched C₁-C₅alkyl.

Embodiment 81): Pertaining to the compound of Formula I or salts, enantiomers, stereoisomers, polymorphs or solvates thereof as described in any one of embodiments 1)-78), wherein R_{b1}, R_{b2}, R_{b3}, R_{b4}, and R_{b5} are each independently selected from the group consisting of hydrogen, deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, or unsubstituted or substituted linear or branched C₁-C₅alkyl.

Embodiment 82): Pertaining to the compound of Formula I or salts, enantiomers, stereoisomers, polymorphs or solvates thereof as described in any one of embodiments 1)-78), wherein R_{b1}, R_{b2}, R_{b3}, R_{b4}, and R_{b5} are each independently selected from the group consisting of hydrogen.

Embodiment 83): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of deuterium.

Embodiment 84): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of halogen. Optionally, when the R_{b1}, R_{b2}, R_{b3}, R_{b4}, and R_{b5} are each independently selected from the group consisting of halogen, the halogen is F, Cl, Br, or I.

Embodiment 85): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of nitro.

Embodiment 86): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of cyano.

Embodiment 87): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of amino.

Embodiment 88): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of hydroxy.

Embodiment 89): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of mercapto.

Embodiment 90): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of alkoxy. Optionally, when the R_{b1}, R_{b2}, R_{b3}, R_{b4}, and R_{b5} are each independently selected from the group consisting of alkoxy, the alkoxy is C₁-C₆ alkoxy.

Further optionally, the alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like.

Embodiment 91): Pertaining to the compound of Formula I or salts, enantiomers, stereoisomers, polymorphs or solvates thereof as described in embodiment 81), wherein R_{b1}, R_{b2}, R_{b3}, R_{b4}, and R_{b5} are each independently selected from the group consisting of halogenated alkoxy. Optionally, when the R_{b1}, R_{b2}, R_{b3}, R_{b4}, and R_{b5} are each independently selected from the group consisting of halogenated alkoxy, the halogenated alkoxy is halogenated C₁-C₆ alkoxy. Further, the halogenated C₁-C₆ alkoxy is trifluoromethoxy etc.

Embodiment 92): Pertaining to the compound of Formula I or salts, enantiomers, stereoisomers, polymorphs or solvates thereof as described in embodiment 81), wherein the R_{b1}, R_{b2}, R_{b3}, R_{b4}, and R_{b5} are each independently selected from the group consisting of unsubstituted or substituted amino. Optionally, the unsubstituted or substituted amino includes amino, N-methylamino, N-ethylamino, N-phenylamino, N,N-dimethylamino, and N,N-diethylamino, and the like.

Embodiment 93): Pertaining to the compound of Formula I or salts, enantiomers, stereoisomers, polymorphs or solvates thereof as described in embodiment 81), wherein the R_{b1}, R_{b2}, R_{b3}, R_{b4}, and R_{b5} are each independently selected from the group consisting of unsubstituted or substituted silyl. Optionally, the unsubstituted or substituted silyl is specifically trimethylsilyl, triethylsilyl, tert-butyldimethylsilyl, vinyldimethylsilyl, propyldimethylsilyl, triphenylsilyl, diphenylsilyl, and phenylsilyl etc.

Embodiment 94): Pertaining to the compound of Formula I or salts, enantiomers, stereoisomers, polymorphs or solvates thereof as described in embodiment 81), wherein the R_{b1}, R_{b2}, R_{b3}, R_{b4}, and R_{b5} are each independently selected from the group consisting of unsubstituted or substituted boryl. Optionally, the unsubstituted or substituted boryl is specifically trimethylboryl, triethylboryl, tert-butyldimethylboryl, triphenylboryl, and phenylboryl etc.

Embodiment 95): Pertaining to the compound of Formula I or salts, enantiomers, stereoisomers, polymorphs or solvates thereof as described in embodiment 81), wherein the R_{b1}, R_{b2}, R_{b3}, R_{b4}, and R_{b5} are each independently selected from the group consisting of unsubstituted or substituted linear or branched alkyl. Optionally, the number of carbon atoms may be for example C₁-C₃₀, C₁-C₂₅, C₁-C₂₀, C₁-C₁₅, C₁-C₁₀, C₁-C₅ or C₁-C₃ etc, with linear or branched C₁-C₁₀ alkyl being preferred. Further optionally, the linear or branched C₁-C₁₀ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, nonyl, 2,2-dimethylheptyl, 1-ethylpropyl, 1,1-dimethylpropyl, isohexyl, 4-methylhexyl, and 5-methylhexyl etc. Optionally, the linear or branched C₁-C₁₀ alkyl includes but is not limited to methyl, ethyl, and propyl etc. Wherein the alkyl is optionally substituted, and the substituent may be e.g., selected from the group consisting of deuterium, halogen, cyano, nitro, hydroxy, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo or any combination thereof. The substituent is preferably one or more selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl) or any combination thereof.

Embodiment 96): Pertaining to the compound of Formula I or salts, enantiomers, stereoisomers, polymorphs or solvates thereof as described in any one of embodiments 1)-95), wherein n is an integer of 0, 1, 2 or 3, and when n is an integer of 2 or 3, each Rₐ is independently identical to or different from each other. In some embodiments, n is an integer of 0, 1 or 2.

Embodiment 97): Pertaining to the compound of Formula I or salts, enantiomers, stereoisomers, polymorphs or solvates thereof as described in any one of embodiments 1)-96), wherein for better characterization of Rₐ₁, it is further represented by -L₂-L₃ moiety. The chemical structure of said compound is shown in Formula I-1 or Formula I-2:

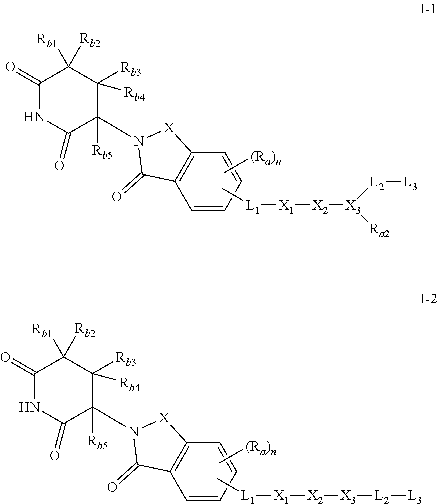

in Formula I-1 or Formula I-2, X, L₁, X₁, X₂, Rₐ, R_{b1}, R_{b2}, R_{b3}, R_{b4}, R_{b5}, and n are as defined in the compounds of Formula I in any one of the preceding embodiments 1)-96);
L₂ is selected from the group consisting of unsubstituted or substituted C₅-C₃₀ arylene, unsubstituted or substituted 5- to 30-membered heteroarylene, unsubstituted or substituted C₃-C₃₀ cycloalkylene, or unsubstituted or substituted 4- to 30-membered heterocyclylene, or L₂ is absent;
L₃ is selected from the group consisting of unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl;
Rₐ₂ is selected from the group consisting of unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl;
wherein in Formula I-1, X₃ is selected from the group consisting of CR₁ or N, and in Formula I-2, X₃ is selected from the group consisting of CR₁, N or CO, wherein R₁ is as defined in the compounds of Formula I in any one of the preceding embodiments 1)-96).

Embodiment 98): Pertaining to the compounds of Formula I-1, Formula I-2 or salts, enantiomers, stereoisomers, polymorphs or solvates thereof as described in embodiment 97), wherein L₂ is selected from the group consisting of unsubstituted or substituted 5- to 20-membered heteroarylene (e.g., unsubstituted or substituted 5- to 15-membered heteroarylene); preferably, the heteroarylene is selected from the group consisting of pyridylene, pyrrolylene, pyrimidinylene, pyridazinylene, furanylene, thienylene, imidazolylene, pyrazolylene, oxazolylene, isoxazolylene, thiazolylene, isothiazolylene, triazolylene, oxadiazolylene, thiadiazolylene, tetrazolylene, pyranylene, thiopyranylene, pyrazinylene, pyridazinylene, thiazinylene, triazinylene, tetrazinylene, quinolinylene, isoquinolinylene, quinazolinylene, quinoxalinylene, cinnolinylene, naphthyridinylene, acridinylene, xanthenylene, phenanthridinylene, phthalazinylene, triazaindenylene, indolylene, indolinylene, indolizinylene, phthalazinylene, pyrazolopyridylene, pyrazolopyrimidinylene, pyridopyrimidinylene, pyridopyrazinylene, pyrazinopyrazinylene, benzothiazolylene, benzoxazolylene, benzimidazolylene, benzothienylene, benzofuranylene, isobenzofuranylene, dibenzothienylene, dibenzofuranylene, indazolylene, carbazolylene, benzocarbazolylene, dibenzocarbazolylene, indolocarbazolylene, indenocarbazolylene, phenazinylene, imidazopyridinylene, phenanthridinylene, phenanthrolinylene, phenothiazinylene, imidazopyridinylene, imidazophenanthridinylene, pyrrolopyridinylene, pyrrolothiazolylene, imidazothiazolylene, benzimidazoquinazolinylene, benzimidazophenanthridinylene, pyrenylene, dinaphthofuranylene, naphthobenzofuranylene, dinaphthothienylene, or naphthobenzothienylene. Wherein the heteroarylene may be unsubstituted or substituted as explicitly defined. The substituted heteroarylene refers to a heteroarylene substituted one or more times (e.g., 1-4, 1-3, or 1-2 times) with a substituent optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, or any combination thereof. Preferably, the substituent is optionally selected from the group consisting of C₁-C₃ alkyl, C₃-C₆ cycloalkyl, hydroxy, amino, mercapto, halogen, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, unsubstituted or substituted cycloalkyl, unsubstituted or substituted aryl, unsubstituted or substituted heteroaryl, unsubstituted or substituted heterocyclyl, or any combination thereof. Further, when L₂ is substituted heteroarylene and the substituent of heteroarylene is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl and unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted heteroarylene and the substituent of heteroarylene is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted heteroarylene and the substituent of heteroarylene is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted heteroarylene and the substituent of heteroarylene is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4 to 15-membered bridged heterocyclyl, 5- to 15-membered bridged heterocyclyl, or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl, 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 99): Pertaining to the compounds of Formula I-1, Formula I-2 or salts, enantiomers, stereoisomers, polymorphs or solvates thereof as described in embodiment 97), wherein L₂ is selected from the group consisting of unsubstituted or substituted C₃-C₂₀ cycloalkylene (e.g., unsubstituted or substituted C₃-C₁₅ cycloalkylene); (preferably, L₂ is selected from the group consisting of cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₃-C₁₅ spiro-cycloalkylene, C₅-C₁₅ spiro-cycloalkylene, or C₇-C₁₅ spiro-cycloalkylene, such as spiro[3.3]heptylene, spiro[2.5]octylene, spiro[3.5]nonylene, spiro[4.4]nonylene, spiro[4.5]decylene, or spiro[5.5]undecylene), or bridged cycloalkylene (e.g., C₃-C₁₅ bridged cycloalkylene, C₅-C₁₅ bridged cycloalkylene, or C₇-C₁₅ bridged cycloalkylene, adamantanylene, noradamantanylene, bornylene, bicyclo[2.2.1]heptylene, 2-oxobicyclo[2.2.1]heptylene, or bicyclo[2.2.1]heptentylene)). Wherein the “cycloalkylene” is optionally substituted, and the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, unsubstituted or substituted cycloalkyl, deuterated cycloalkyl, alkenyl, unsubstituted or substituted aryl, unsubstituted or substituted heteroaryl, unsubstituted or substituted heterocyclyl, aralkyl, arylalkenyl, alkyl aryl, alkylamino, aralkylamino, heteroarylamino, arylamino, arylphosphino, acenaphthenyl, oxo or any combination thereof. Further, the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, unsubstituted or substituted cycloalkyl, unsubstituted or substituted aryl, unsubstituted or substituted heteroaryl, unsubstituted or substituted heterocyclyl, or any combination thereof. Further, when L₂ is substituted cycloalkylene and the substituent of cycloalkylene is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted cycloalkylene and the substituent of cycloalkylene is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted cycloalkylene and the substituent of cycloalkylene is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted cycloalkylene and the substituent of cycloalkylene is unsubstituted or substituted heterocyclyl, the heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl, 5- to 15-membered bridged heterocyclyl, or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl, 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 100): Pertaining to the compounds of Formula I-1, Formula I-2 or salts, enantiomers, stereoisomers, polymorphs or solvates thereof as described in embodiment 97), wherein L₂ is selected from the group consisting of unsubstituted or substituted C₅-C₂₀ arylene (e.g., unsubstituted or substituted C₅-C₁₅ arylene); preferably, L₂ is selected from the group consisting of phenylene, biphenylene, terphenylene, naphthylene, anthrylene, phenanthrylene, fluorenylene, and 9,9′-dimethylfluorenylene. The arylene is optionally substituted arylene. The substituted arylene refers to an arylene group substituted one or more times (e.g., 1-4, 1-3, or 1-2 times) with a substituent. For example, arylene is mono-, di-, or tri-substituted with a substituent optionally selected from the group consisting of e.g., deuterated C₁-C₆ alkyl, deuterated C₃-C₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, unsubstituted or substituted cycloalkyl, alkenyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof. Preferably, the substituent is selected from the group consisting of deuterated C₁-C₆ alkyl, deuterated C₃-C₆ cycloalkyl, hydroxy, amino, mercapto, halogen, cyano, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl, and any combination thereof. Further, when L₂ is substituted arylene and the substituent of arylene is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl and unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted arylene and the substituent of arylene is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, and unsubstituted or substituted C₅-C₁₀ aryl etc.) (optionally, the aryl is selected from the group consisting of biphenyl, terphenyl, naphthyl, anthryl, phenanthryl, fluorenyl, or 9,9′-dimethylfluorenyl etc.; and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when L₂ is substituted arylene and the substituent of arylene is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl and unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof. Further, when L₂ is substituted arylene and the substituent of arylene is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, and unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4 to 15-membered bridged heterocyclyl, 5- to 15-membered bridged heterocyclyl, or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl, 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 101): Pertaining to the compounds of Formula I-1, Formula I-2 or salts, enantiomers, stereoisomers, polymorphs or solvates thereof as described in embodiment 97), wherein L₂ is selected from the group consisting of unsubstituted or substituted 4- to 20-membered heterocyclylene (e.g., unsubstituted or substituted 4- to 15-membered heterocyclylene); preferably, L₂ is selected from the group consisting of azetidinylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, diazacycloheptanylene, diazacyclooctylene, bridged heterocyclylene (e.g., 4- to 15-membered bridged heterocyclylene, 5- to 15-membered bridged heterocyclylene, or 7- to 15-membered bridged heterocyclylene, such as azabicyclohexylene, azabicycloheptanylene, diazabicycloheptanylene, azabicyclooctylene), or spiro-heterocyclylene (e.g., 4- to 15-membered spiro-heterocyclylene, 5- to 15-membered spiro-heterocyclylene, or 7- to 15-membered spiro-heterocyclylene, such as 3-azaspiro[5.5]undecanylene or 7-azaspiro[3.5]nonanylene). The heterocyclylene may be unsubstituted or substituted as explicitly defined (e.g., mono-, di-, tri-, or poly-substituted) by a substituent optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁₋₆ alkylene, C₁-C₄ alkyl-NHC(O)—, C₁-C₄ alkyl-C(O)NH—, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof. Preferably, the substituent is selected from the group consisting of deuterium, hydroxy, amino, mercapto, nitro, halogen, cyano, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁₋₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl, and any combination thereof. Further, when L₂ is substituted heterocyclylene and the substituent of heterocyclylene is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, and unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted heterocyclylene and the substituent of heterocyclylene is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₂ is substituted heterocyclylene and the substituent of heterocyclylene is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, ditriazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. When L₂ is substituted heterocyclylene and the substituent of heterocyclylene is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4 to 15-membered bridged heterocyclyl, 5- to 15-membered bridged heterocyclyl, or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl, 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 102): Pertaining to the compounds of Formula I-1, Formula I-2 or salts, enantiomers, stereoisomers, polymorphs or solvates thereof as described in embodiment 97), wherein L₂ is absent.

Embodiment 103): Pertaining to the compounds of Formula I-1, Formula I-2 or salts, enantiomers, stereoisomers, polymorphs or solvates thereof as described in any one of embodiments 97)-102), wherein L₂ is selected from the group consisting of

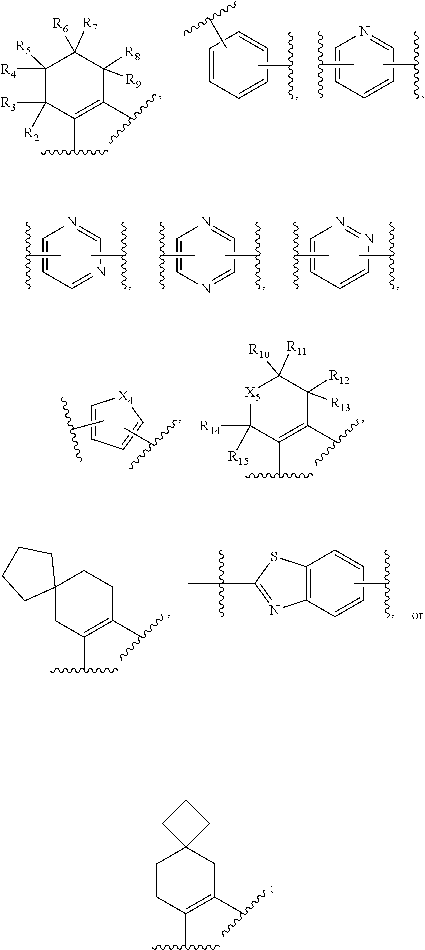

wherein X₄ is selected from the group consisting of O, S, or NH;
X₅ is selected from the group consisting of O, S, or NR₁₆, where R₁₆ represents hydrogen or C₁-C₃ alkyl;
R₂, R₃, R₄, R₅, R₆, R₇, R₈, R₉, R₁₀, R₁₁, R₁₂, R₁₃, R₁₄, and R₁₅ are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, halogenated C₁-C₅ alkyl, or linear or branched C₁-C₅ alkyl. Optionally, halogen may be e.g., F, Cl, Br, and I. Optionally, C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like. Optionally, halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino, and the like. Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc. Optionally, the linear or branched C₁-C₅alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, and tert-pentyl etc., and the linear or branched C₁-C₅ alkyl is optionally substituted with one or more substituents optionally selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl) or any combination thereof.

Embodiment 104): Pertaining to the compounds of Formula I-1, Formula I-2 or salts, enantiomers, stereoisomers, polymorphs or solvates thereof as described in any one of embodiments 97)-103), wherein L₃ is selected from the group consisting of unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl.

Embodiment 105): Pertaining to the compounds of Formula I-1, Formula I-2 or salts, enantiomers, stereoisomers, polymorphs or solvates thereof as described in embodiment 104), wherein L₃ is selected from the group consisting of unsubstituted or substituted C₅-C₂₀ aryl (e.g., unsubstituted or substituted C₅-C₁₅ aryl, unsubstituted or substituted C₅-C₁₀ aryl etc.). preferably, L₃ is selected from the group consisting of biphenyl, terphenyl, naphthyl, anthryl, phenanthryl, fluorenyl, and 9,9′-dimethylfluorenyl. The aryl may be monocyclic or polycyclic aryl. In some embodiments, the monocyclic aryl includes but is not limited to phenyl, biphenyl, terphenyl, quaterphenyl, and quinquephenyl etc. The polycyclic aryl includes but is not limited to naphthyl, anthryl, phenanthryl, pyrenyl, perylenyl, and fluorenyl etc. The fluorenyl may be substituted (e.g., 9,9′-dimethylfluorenyl and 9,9′-diphenylfluorenyl etc.). Preferably, L₃ is selected from the group consisting of biphenyl, terphenyl, naphthyl, anthryl, phenanthryl, fluorenyl, 9,9′-dimethylfluorenyl. Furthermore, two of the substituents may combine with each other to form a spirocyclic structure such as 9,9′-spirobifluorenyl etc. The aryl is optionally substituted aryl. The substituted aryl refers to an aryl group substituted one or more times (e.g., 1-4, 1-3, or 1-2 times) with a substituent. For example, aryl is mono-, di-, or tri-substituted with a substituent optionally selected from the group consisting of e.g., deuterated C₁-C₆ alkyl, deuterated C₃-C₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, unsubstituted or substituted cycloalkyl, alkenyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof (preferably, the substituent is selected from the group consisting of deuterated C₁-C₆ alkyl, deuterated C₃-C₆ cycloalkyl, hydroxy, amino, mercapto, halogen, cyano, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl, and any combination thereof). Further, when L₃ is substituted aryl and the substituent of aryl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH— and any combination thereof. Further, when L₃ is substituted aryl and the substituent of aryl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, unsubstituted or substituted C₅-C₁₀ aryl etc.) (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when L₃ is substituted aryl and the substituent of aryl is unsubstituted or substituted heteroaryl, the unsubstituted or substituted heteroaryl is selected from the group consisting of unsubstituted or substituted 5- to 25-membered heteroaryl (e.g., unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted aryl and the substituent of aryl is unsubstituted or substituted heterocyclyl, and the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4 to 15-membered bridged heterocyclyl, 5- to 15-membered bridged heterocyclyl, or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl, 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 106): Pertaining to the compounds of Formula I-1, Formula I-2 or salts, enantiomers, stereoisomers, polymorphs or solvates thereof as described in embodiment 104), wherein L₃ is selected from the group consisting of unsubstituted or substituted 5- to 25-membered heteroaryl (preferably, unsubstituted or substituted 5- to 20-membered heteroaryl, or unsubstituted or substituted 5- to 15-membered heteroaryl); more preferably, L₃ is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl. Wherein the heteroaryl may be unsubstituted or substituted. The substituted heteroaryl refers to a heteroaryl substituted one or more times (e.g., 1-4, 1-3, or 1-2 times) with a substituent optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, or any combination thereof (preferably, the substituent is optionally selected from the group consisting of C₁-C₃ alkyl, C₃-C₆ cycloalkyl, hydroxy, amino, mercapto, halogen, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, unsubstituted or substituted cycloalkyl, unsubstituted or substituted aryl, unsubstituted or substituted heterocyclyl, unsubstituted or substituted heteroaryl, unsubstituted or substituted heterocyclyl or any combination thereof). Further, when L₃ is substituted heteroaryl and the substituent of heteroaryl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆cycloalkyl, amino-substituted C₁-C₆ alkylene, C₁-C₄ alkyl-NHC(O)—, C₁-C₄ alkyl-C(O)NH— and any combination thereof. Further, when L₃ is substituted heteroaryl and the substituent of heteroaryl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted heteroaryl and the substituent of heteroaryl is unsubstituted or substituted heteroaryl, the unsubstituted or substituted heteroaryl is selected from the group consisting of unsubstituted or substituted 5- to 25-membered heteroaryl (preferably, unsubstituted or substituted 5- to 20-membered heteroaryl, unsubstituted or substituted 5- to 15-membered heteroaryl or unsubstituted or substituted 5- to 10-membered heteroaryl); (optionally, the unsubstituted or substituted heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the unsubstituted or substituted heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when L₃ is substituted heteroaryl and the substituent of heteroaryl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, azacyclopentyl, azacyclohexyl, azacycloheptanyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, diazacycloheptanyl, diazacyclooctyl, bridged heterocyclyl (e.g., 4- to 15-membered bridged heterocyclyl, 5- to 15-membered bridged heterocyclyl, or 7- to 15-membered bridged heterocyclyl, such as azabicyclohexyl, azabicycloheptanyl, azabicycloheptanyl, azabicyclooctyl), or spiro-heterocyclyl (e.g., 4- to 15-membered spiro-heterocyclyl, 5- to 15-membered spiro-heterocyclyl, or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 107): Pertaining to the compounds of Formula I-1, Formula I-2 or salts, enantiomers, stereoisomers, polymorphs or solvates thereof as described in embodiment 104), wherein L₃ is selected from the group consisting of unsubstituted or substituted 4- to 20-membered heterocyclyl (e.g., unsubstituted or substituted 4- to 15-membered heterocyclyl); (preferably, L₃ is selected from the group consisting of azetidinyl, azacyclopentyl, azacyclohexyl, azacycloheptanyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, diazacycloheptanyl, diazacyclooctyl, bridged heterocyclyl (e.g., 4- to 15-membered bridged heterocyclyl, such as azabicyclohexyl, azabicycloheptanyl, azabicycloheptanyl, azabicyclooctyl), or spiro-heterocyclyl (e.g., 4- to 15-membered spiro-heterocyclyl, such as 3-azaspiro[5.5]undecanyl or 7-azaspiro[3.5]nonanyl)). The heterocyclyl may be unsubstituted or substituted as explicitly defined (e.g., mono-, di-, tri-, or poly-substituted) by a substituent optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁₋₆ alkylene, C₁-C₄ alkyl-NHC(O)—, C₁-C₄ alkyl-C(O)NH—, alkenyl, unsubstituted or substituted cycloalkyl, unsubstituted or substituted aryl, aralkyl, arylalkenyl, alkylaryl, aralkylamino, heteroarylamino, arylamino, arylphosphino, unsubstituted or substituted heteroaryl, unsubstituted or substituted heterocyclyl, acenaphthenyl, and any combination thereof (preferably, the substituent is selected from the group consisting of deuterium, hydroxy, amino, mercapto, nitro, halogen, cyano, oxo, optionally deuterated C₁-C₆ alkyl, halogenated C₁-C₆ alkyl, optionally deuterated C₃-C₆ cycloalkyl, optionally deuterated C₁-C₆ alkoxy, C₁-C₄ alkyl-NH—, NH₂—C₁₋₆ alkylene, C₁-C₄ alkyl-NHC(O)—, C₁-C₄ alkyl-C(O)NH—, unsubstituted or substituted cycloalkyl, unsubstituted or substituted aryl, unsubstituted or substituted heteroaryl, unsubstituted or substituted heterocyclyl and any combination thereof). Further, when L₃ is substituted heterocyclyl and the substituent of heterocyclyl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, unsubstituted or substituted C₃-C₆ cycloalkyl etc.); (optionally, the cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted heterocyclyl and the substituent of heterocyclyl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted heterocyclyl and the substituent of heterocyclyl is unsubstituted or substituted heteroaryl, the unsubstituted or substituted heteroaryl is selected from the group consisting of unsubstituted or substituted 5- to 25-membered heteroaryl (preferably, unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted heterocyclyl and the substituent of heterocyclyl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.) (optionally, the unsubstituted or substituted heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4 to 15-membered bridged heterocyclyl, or 5- to 15-membered bridged heterocyclyl, or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl, or 5- to 15-membered spiro-heterocyclyl, or 7- to 15-membered spiro-heterocyclyl, such as 3-azaspiro[5.5]undecanyl or 7-azaspiro[3.5]nonanyl)); and the unsubstituted or substituted heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof).

Embodiment 108): Pertaining to the compounds of Formula I-1, Formula I-2 or salts, enantiomers, stereoisomers, polymorphs or solvates thereof as described in embodiment 104), wherein L₃ is selected from the group consisting of unsubstituted or substituted C₃-C₂₀ cycloalkyl (e.g., unsubstituted or substituted C₃-C₁₅ cycloalkyl). Preferably, L₃ is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl). Wherein, the “cycloalkyl” is optionally substituted, and the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, unsubstituted or substituted cycloalkyl, deuterated cycloalkyl, alkenyl, unsubstituted or substituted aryl, unsubstituted or substituted heteroaryl, unsubstituted or substituted heterocyclyl, aralkyl, arylalkenyl, alkyl aryl, alkylamino, aralkylamino, heteroarylamino, arylamino, arylphosphino, acenaphthenyl, oxo or any combination thereof. Further, the substituents are preferably one or more (e.g., 1-5, 1-4, 1-3, 1-2, or 1) selected from the group consisting of trifluoromethyl, mercapto, hydroxy, amino, halogen, cyano, C₁-C₃ alkyl, C₁-C₃ alkoxy, trifluoromethyl, unsubstituted or substituted cycloalkyl, unsubstituted or substituted aryl, unsubstituted or substituted heteroaryl, unsubstituted or substituted heterocyclyl or any combination thereof. Further, when L₃ is substituted cycloalkyl and the substituent of cycloalkyl is unsubstituted or substituted cycloalkyl, the unsubstituted or substituted cycloalkyl is selected from the group consisting of unsubstituted or substituted C₃-C₂₀ cycloalkyl (e.g., unsubstituted or substituted C₃-C₁₅ cycloalkyl, unsubstituted or substituted C₃-C₁₀ cycloalkyl, unsubstituted or substituted C₃-C₆ cycloalkyl etc.) (optionally, the unsubstituted or substituted cycloalkyl is selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl, C₅-C₁₅ spiro-cycloalkyl, or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₃-C₁₅ bridged cycloalkyl, C₅-C₁₅ bridged cycloalkyl, or C₇-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl)); and the unsubstituted or substituted cycloalkyl is optionally substituted cycloalkyl, and the number of substituents is preferably one or more (e.g., 1-5, 1-4, 1-3, 1-2, or 1), and said substituents are selected from the group consisting of e.g., C₁-C₆ alkyl, C₁-C₆ alkoxy, C₃-C₆ cycloalkyl, hydroxy, amino, mercapto, halogen (e.g., fluorine, chlorine, bromine, and iodine etc.), cyano, oxo, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof). Further, when L₃ is substituted cycloalkyl and the substituent of cycloalkyl is unsubstituted or substituted aryl, the unsubstituted or substituted aryl is selected from the group consisting of unsubstituted or substituted C₅-C₂₀ aryl (e.g., unsubstituted or substituted C₅-C₁₅ aryl, unsubstituted or substituted C₅-C₁₀ aryl etc.); (optionally, the aryl is selected from the group consisting of biphenyl, terphenyl, naphthyl, anthryl, phenanthryl, fluorenyl, or 9,9′-dimethylfluorenyl etc.); and the aryl is optionally substituted aryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted cycloalkyl and the substituent of cycloalkyl is unsubstituted or substituted heteroaryl, the unsubstituted or substituted heteroaryl is selected from the group consisting of unsubstituted or substituted 5- to 25-membered heteroaryl (preferably, unsubstituted or substituted 5- to 20-membered heteroaryl, unsubstituted or substituted 5- to 15-membered heteroaryl or unsubstituted or substituted 5- to 10-membered heteroaryl); (optionally, the heteroaryl is selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, dithiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, naphthyridinyl, acridinyl, xanthenyl, phenanthridinyl, phthalazinyl, triazaindenyl, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl); and the heteroaryl is optionally substituted heteroaryl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof. Further, when L₃ is substituted cycloalkyl and the substituent of cycloalkyl is unsubstituted or substituted heterocyclyl, the unsubstituted or substituted heterocyclyl is selected from the group consisting of unsubstituted or substituted 4- to 20-membered heterocyclyl (e.g., unsubstituted or substituted 4- to 15-membered heterocyclyl, unsubstituted or substituted 4- to 10-membered heterocyclyl, or unsubstituted or substituted 4- to 8-membered heterocyclyl etc.); (optionally, the heterocyclyl is selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl, diazacyclooctyl, bridged heterocyclyl (e.g., 4- to 15-membered bridged heterocyclyl or 5- to 15-membered bridged heterocyclyl or 7- to 15-membered bridged heterocyclyl, such as azabicyclohexyl, azabicycloheptanyl, diazabicycloheptanyl, azabicyclooctyl), or spiro-heterocyclyl (e.g., 4- to 15-membered spiro-heterocyclyl or 5- to 15-membered spiro-heterocyclyl or 7- to 15-membered spiro-heterocyclyl, such as 3-azaspiro[5.5]undecanyl or 7-azaspiro[3.5]nonanyl)); and the heterocyclyl is optionally substituted heterocyclyl, and the number of substituents is preferably one or more (e.g., 1-5, 1-4, 1-3, 1-2, or 1), and said substituents are selected from the group consisting of e.g., C₁-C₆ alkyl, C₃-C₆ cycloalkyl, hydroxy, amino, mercapto, halogen (e.g., fluorine, chlorine, bromine, and iodine etc.), cyano, oxo, C₁-C₆ alkoxy, deuterated C₁-C₆ alkoxy, C₁-C₄ alkylamino, halogenated C₁-C₆ alkyl, halogenated C₃-C₆ cycloalkyl, amino-substituted C₁-C₆ alkylene, C₁-C₄ alkyl-NHC(O)—, C₁-C₄ alkyl-C(O)NH—, and any combination thereof.

Embodiment 109): Pertaining to the compounds of Formula I-1, Formula I-2 or salts, enantiomers, stereoisomers, polymorphs or solvates thereof as described in embodiment 104), wherein the substituent of the substituted L₃ is optionally one or more selected from the group consisting of deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, linear or branched C₁-C₅ alkyl, or halogenated C₁-C₅alkyl.

Embodiment 110): Pertaining to the compounds of Formula I-1, Formula I-2 or salts, enantiomers, stereoisomers, polymorphs or solvates thereof as described in any one of embodiments 104)-109), wherein L₃ is selected from the group consisting of:

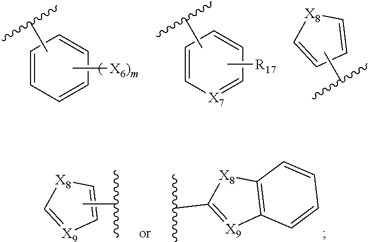

wherein X₆ is selected from the group consisting of deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, halogenated C₁-C₆ alkyl or linear or branched C₁-C₆ alkyl. Optionally, the halogen may be e.g., F, Cl, Br, and I. Optionally, C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like. Optionally, the halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino, and the like. Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc. Optionally, the linear or branched C₁-C₆ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl etc., and the linear or branched C₁-C₆ alkyl is optionally substituted by one or more optionally selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl) or any combination thereof;
m is an integer of 0, 1, 2, 3, 4 or 5, and when m is an integer of 2, 3, 4 or 5, each X₆ is independently identical to or different from each other;
X₇ and X₉ are each independently selected from the group consisting of CH or N;
each X₈ is independently selected from the group consisting of O, S or NH;
R₁₇ is selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, halogenated C₁-C₅alkyl or linear or branched C₁-C₅alkyl, wherein the halogen may optionally be e.g., F, Cl, Br, and I. Optionally, the C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, 2-ethylbutoxy, and the like. Optionally, the halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino, and the like. Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc. Optionally, the linear or branched C₁-C₅alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl etc., and the linear or branched C₁-C₅alkyl is optionally substituted by one or more optionally selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl), or any combination thereof.

Embodiment 111): Pertaining to the compounds of Formula I-1, Formula I-2 or salts, enantiomers, stereoisomers, polymorphs or solvates thereof as described in any one of embodiments 97)-110), wherein the further selection of Rₐ₂ is as described in Formula I. Rₐ₂ may be the same as or different from L₃; and Rₐ₂ must be present.

Especially preferably the compounds of the present disclosure and their salts (especially pharmaceutically acceptable salts, such as hydrochloride, etc.), enantiomers, stereoisomers, polymorphs, or solvates thereof in Table 1 below are provided: Table 1. The compounds of the present disclosure

TABLE 1
The compounds of the present disclosure

| Compound | | |
|---|---|---|
| No. | Structure of the compounds | The compounds′ name |
| 1 GT- 03464 | 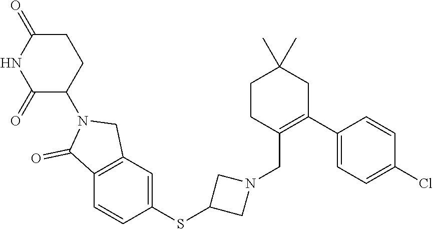 [SMILES: CC1(C)CCC(CN2CC(Sc3ccc4c(c3)CN(C3CCC(=O)NC3=O)C4=O)C2)=C(c2ccc(Cl)cc2)C1] | 3-(5-((1-((4′-chloro-5,5- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 2 | 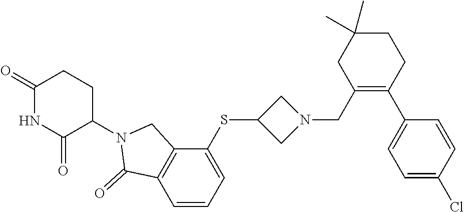 [SMILES: CC1(C)CCC(c2ccc(Cl)cc2)=C(CN2CC(Sc3cccc4c3CN(C3CCC(=O)NC3=O)C4=O)C2)C1] | 3-(4-((1-((4′-chloro-4,4- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 3 GT- 06757 | 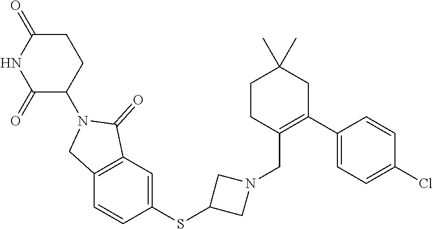 [SMILES: CC1(C)CCC(CN2CC(Sc3ccc4c(c3)C(=O)N(C3CCC(=O)NC3=O)C4)C2)=C(c2ccc(Cl)cc2)C1] | 3-(6-((1-((4′-chloro-5,5- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 4 | 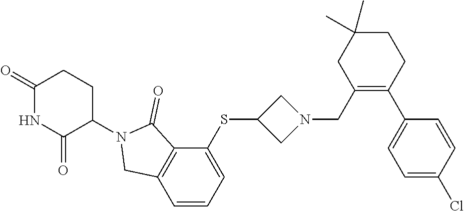 [SMILES: CC1(C)CCC(c2ccc(Cl)cc2)=C(CN2CC(Sc3cccc4c3C(=O)N(C3CCC(=O)NC3=O)C4)C2)C1] | 3-(7-((1-((4′-chloro-4,4- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 5 GT- 04245 | 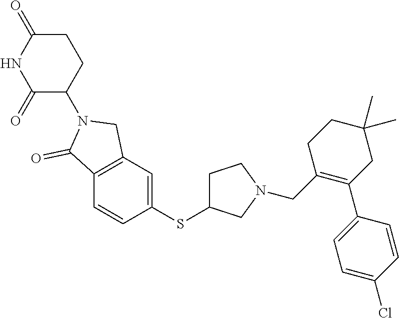 [SMILES: CC1(C)CCC(CN2CCC(Sc3ccc4c(c3)CN(C3CCC(=O)NC3=O)C4=O)C2)=C(c2ccc(Cl)cc2)C1] | 3-(5-((1-((4′-chloro-5,5- dimethyl-3,4,5,6- tetrahydro-[1,1′-biphenyl]- 2-yl)methyl)pyrrolidin-3- yl)thio)-1-oxoisoindolin-2- yl)piperidine-2,6-dione |
| 6 GT- 03462 | 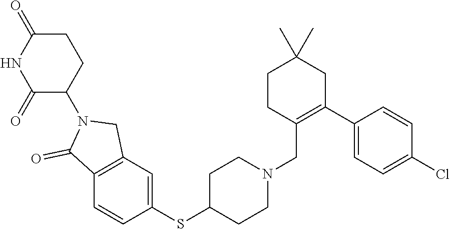 [SMILES: CC1(C)CCC(CN2CCC(Sc3ccc4c(c3)CN(C3CCC(=O)NC3=O)C4=O)CC2)=C(c2ccc(Cl)cc2)C1] | 3-(5-((1-((4′-chloro-5,5- dimethyl-3,4,5,6- tetrahydro-[1,1′-biphenyl]- 2-yl)methyl)piperidin-4- yl)thio)-1-oxoisoindolin-2- yl)piperidine-2,6-dione |
| 7 GT- 06641 | 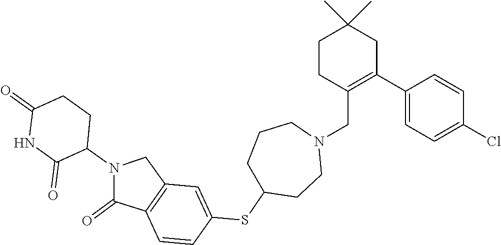 [SMILES: CC1(C)CCC(CN2CCCC(Sc3ccc4c(c3)CN(C3CCC(=O)NC3=O)C4=O)CC2)=C(c2ccc(Cl)cc2)C1] | 3-(5-((1-((4′-chloro-5,5- dimethyl-3,4,5,6- tetrahydro-[1,1′-biphenyl]- 2-yl)methyl)azepan-4- yl)thio)-1-oxoisoindolin-2- yl)piperidine-2,6-dione |
| 8 | 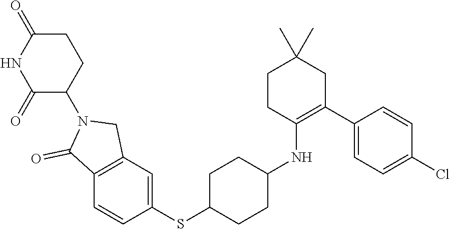 [SMILES: CC1(C)CCC(NC2CCC(Sc3ccc4c(c3)CN(C3CCC(=O)NC3=O)C4=O)CC2)=C(c2ccc(Cl)cc2)C1] | 3-(5-((4-((4′-chloro-5,5- dimethyl-3,4,5,6- tetrahydro-[1,1′-biphenyl]- 2- yl)amino)cyclohexyl)thio)- 1-oxoisoindolin-2- yl)piperidine-2,6-dione |
| 9 GT- 06813 | 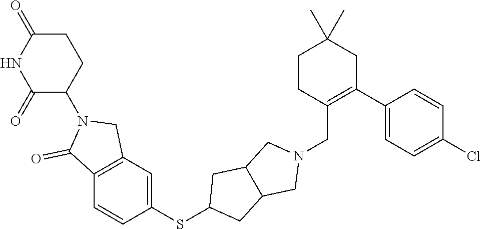 [SMILES: CC1(C)CCC(CN2CC3CC(Sc4ccc5c(c4)CN(C4CCC(=O)NC4=O)C5=O)CC3C2)=C(c2ccc(Cl)cc2)C1] | 3-(5-((2-((4′-chloro-5,5- dimethyl-3,4,5,6- tetrahydro-[1,1′-biphenyl]- 2- yl)methyl)octahydro- cyclopenta[c]pyrrol- 5-yl)thio)-1- oxoisoindolin-2- yl)piperidine-2,6-dione |
| 10 | 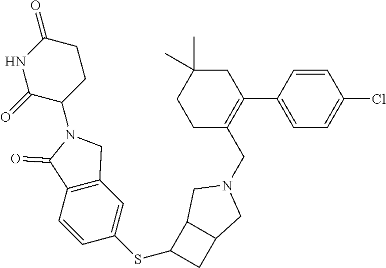 [SMILES: CC1(C)CCC(CN2CC3CC(Sc4ccc5c(c4)CN(C4CCC(=O)NC4=O)C5=O)C3C2)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azabicyclo[3.2.0]heptan-6- yl)thio)-1-oxoisoindolin-2- yl)piperidine-2,6-dione |
| 11 | 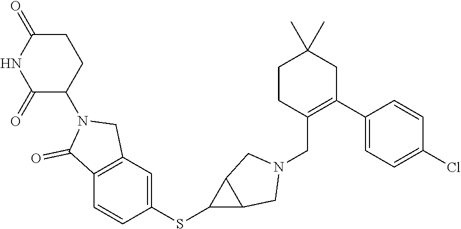 [SMILES: CC1(C)CCC(CN2CC3C(C2)C3Sc2ccc3c(c2)CN(C2CCC(=O)NC2=O)C3=O)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azabicyclo[3.1.0]hexan-6- yl)thio)-1-oxoisoindolin-2- yl)piperidine-2,6-dione |
| 12 GT- 06873 | 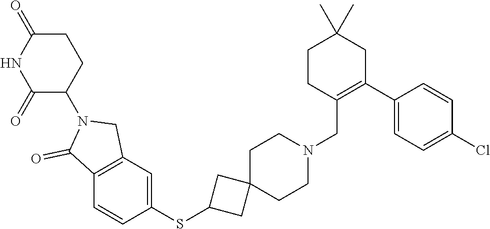 [SMILES: CC1(C)CCC(CN2CCC3(CC2)CC(Sc2ccc4c(c2)CN(C2CCC(=O)NC2=O)C4=O)C3)=C(c2ccc(Cl)cc2)C1] | 3-(5-((7-((4′-chloro-5,5- dimethyl-3,4,5,6- tetrahydro-[1,1′-biphenyl]- 2-yl)methyl)-7- azaspiro[3.5]nonan-2- yl)thio)-1-oxoisoindolin-2- yl)piperidine-2,6-dione |
| 13 GT- 06874 | 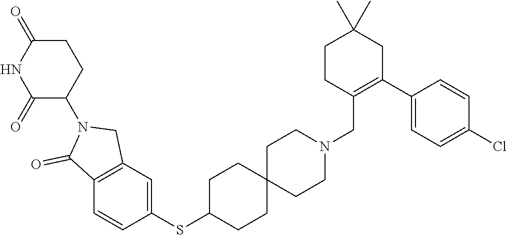 [SMILES: CC1(C)CCC(CN2CCC3(CCC(Sc4ccc5c(c4)CN(C4CCC(=O)NC4=O)C5=O)CC3)CC2)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azaspiro[5.5]undecan-9- yl)thio)-1-oxoisoindolin-2- yl)piperidine-2,6-dione |
| 14 | 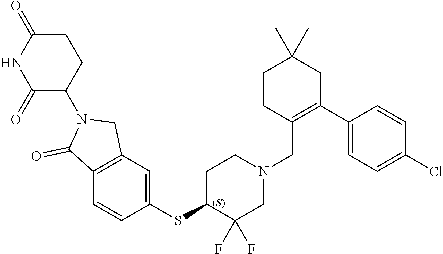 | 3-(5-(((S)-1-((4′-chloro-5,5- |
| 15 | 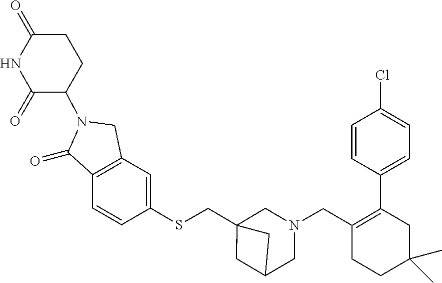 [SMILES: CC1(C)CCC(CN2CC3CC(CSc4ccc5c(c4)CN(C4CCC(=O)NC4=O)C5=O)(C3)C2)=C(c2ccc(Cl)cc2)C1] | 3-(5-(((3-((4′-chloro-5,5- dimethyl-3,4,5,6- tetrahydro-[1,1′-biphenyl]- 2-yl)methyl)-3- azabicyclo[3.1.1 ]heptan-1- yl)methyl)thio)-1- oxoisoindolin-2- yl)piperidine-2,6-dione |
| 16 | 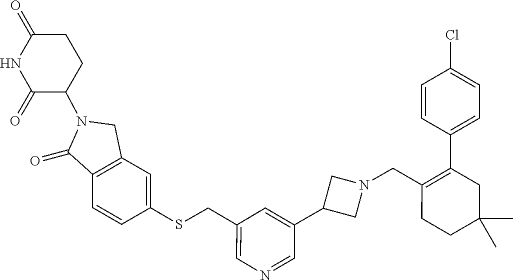 [SMILES: CC1(C)CCC(CN2CC(c3cncc(CSc4ccc5c(c4)CN(C4CCC(=O)NC4=O)C5=O)c3)C2)=C(c2ccc(Cl)cc2)C1] | 3-(5-(((5-(1-((4′-chloro-5,5- dimethyl-3,4,5,6- tetrahydro-[1,1′-biphenyl]- 2-yl)methyl)azetidin-3- yl)pyridin-3- yl)methyl)thio)-1- oxoisoindolin-2- yl)piperidine-2,6-dione |
| 17 GT- 06137 | 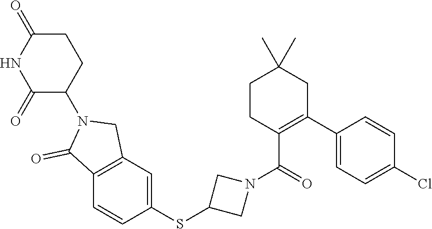 [SMILES: CC1(C)CCC(C(=O)N2CC(Sc3ccc4c(c3)CN(C3CCC(=O)NC3=O)C4=O)C2)=C(c2ccc(Cl)cc2)C1] | 3-(5-((1-(4′-chloro-5,5- dimethyl-3,4,5,6- tetrahydro-[1,1′-biphenyl]- 2-carbonyl)azetidin-3- yl)thio)-1-oxoisoindolin-2- yl)piperidine-2,6-dione |
| 18 GT- 06139 | 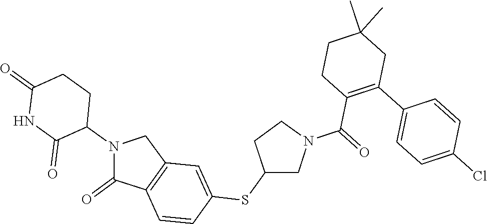 [SMILES: CC1(C)CCC(C(=O)N2CCC(Sc3ccc4c(c3)CN(C3CCC(=O)NC3=O)C4=O)C2)=C(c2ccc(Cl)cc2)C1] | 3-(5-((1-(4′-chloro-5,5- dimethyl-3,4,5,6- tetrahydro-[1,1′-biphenyl]- 2-carbonyl)pyrrolidin-3- yl)thio)-1-oxoisoindolin-2- yl)piperidine-2,6-dione |
| 19 GT- 06140 | 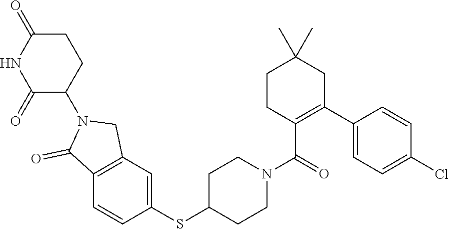 [SMILES: CC1(C)CCC(C(=O)N2CCC(Sc3ccc4c(c3)CN(C3CCC(=O)NC3=O)C4=O)CC2)=C(c2ccc(Cl)cc2)C1] | 3-(5-((1-(4′-chloro-5,5- dimethyl-3,4,5,6- tetrahydro-[1,1′-biphenyl]- 2-carbonyl)piperidin-4- yl)thio)-1-oxoisoindolin-2- yl)piperidine-2,6-dione |
| 20 GT- 06642 | 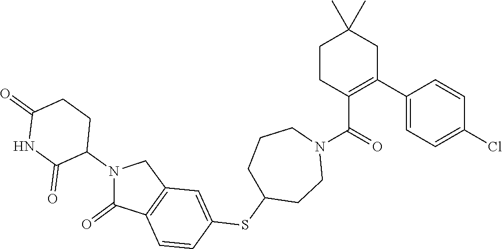 [SMILES: CC1(C)CCC(C(=O)N2CCCC(Sc3ccc4c(c3)CN(C3CCC(=O)NC3=O)C4=O)CC2)=C(c2ccc(Cl)cc2)C1] | 3-(5-((1-(4′-chloro-5,5- dimethyl-3,4,5,6- tetrahydro-[1,1′-biphenyl]- 2-carbonyl)azepan-4- yl)thio)-1-oxoisoindolin-2- yl)piperidine-2,6-dione |
| 21 | 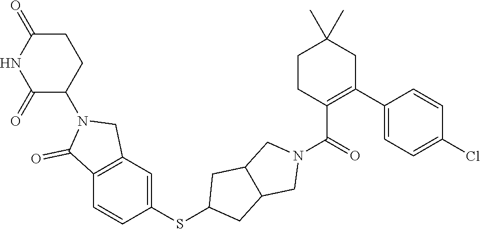 [SMILES: CC1(C)CCC(C(=O)N2CC3CC(Sc4ccc5c(c4)CN(C4CCC(=O)NC4=O)C5=O)CC3C2)=C(c2ccc(Cl)cc2)C1] | 3-(5-((2-(4′-chloro-5,5- dimethyl-3,4,5,6- tetrahydro-[1,1′-biphenyl]- 2- carbonyl)octahydro- cyclopenta[c]pyrrol- 5-yl)thio)-1- oxoisoindolin-2- yl)piperidine-2,6-dione |
| 22 | 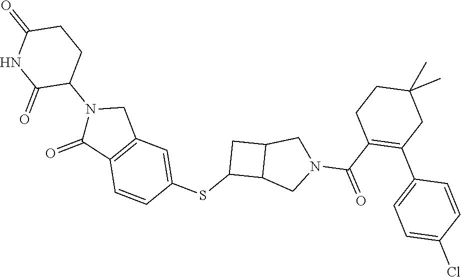 [SMILES: CC1(C)CCC(C(=O)N2CC3CC(Sc4ccc5c(c4)CN(C4CCC(=O)NC4=O)C5=O)C3C2)=C(c2ccc(Cl)cc2)C1] | 3-(5-((3-(4′-chloro-5,5- dimethyl-3,4,5,6- tetrahydro-[1,1′-biphenyl]- 2-carbonyl)-3- azabicyclo[3.2.0]heptan-6- yl)thio)-1-oxoisoindolin-2- yl)piperidine-2,6-dione |
| 23 | 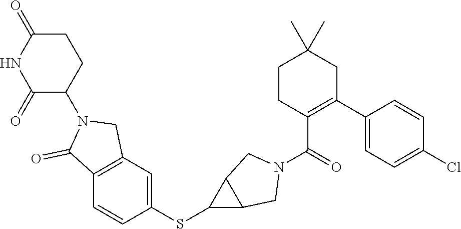 [SMILES: CC1(C)CCC(C(=O)N2CC3C(C2)C3Sc2ccc3c(c2)CN(C2CCC(=O)NC2=O)C3=O)=C(c2ccc(Cl)cc2)C1] | 3-(5-((3-(4′-chloro-5,5- dimethyl-3,4,5,6- tetrahydro-[1,1′-biphenyl]- 2-carbonyl)-3- azabicyclo[3.1.0]hexan-6- yl)thio)-1-oxoisoindolin-2- yl)piperidine-2,6-dione |
| 24 GT- 06918 | 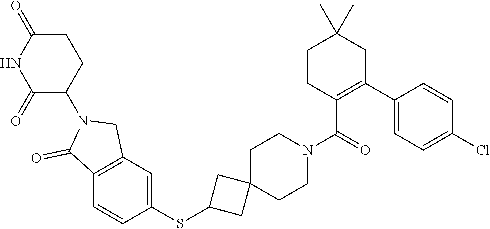 [SMILES: CC1(C)CCC(C(=O)N2CCC3(CC2)CC(Sc2ccc4c(c2)CN(C2CCC(=O)NC2=O)C4=O)C3)=C(c2ccc(Cl)cc2)C1] | 3-(5-((7-(4′-chloro-5,5- dimethyl-3,4,5,6- tetrahydro-[1,1′-biphenyl]- 2-carbonyl)-7- azaspiro[3.5]nonan-2- yl)thio)-1-oxoisoindolin-2- yl)piperidine-2,6-dione |
| 25 GT- 07016 | 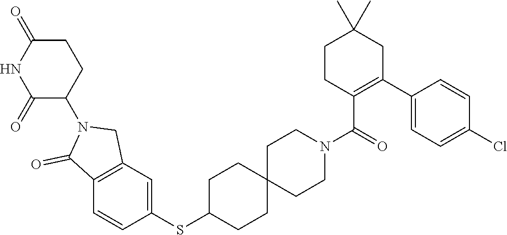 [SMILES: CC1(C)CCC(C(=O)N2CCC3(CCC(Sc4ccc5c(c4)CN(C4CCC(=O)NC4=O)C5=O)CC3)CC2)=C(c2ccc(Cl)cc2)C1] | 3-(5-((3-(4′-chloro-5,5- dimethyl-3,4,5,6- tetrahydro-[1,1′-biphenyl]- 2-carbonyl)-3- azaspiro[5.5]undecan-9- yl)thio)-1-oxoisoindolin-2- yl)piperidine-2,6-dione |
| 26 | 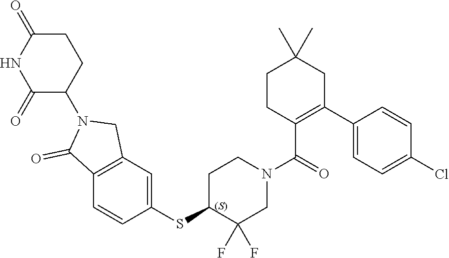 | 3-(5-(((S)-1-(4′-chloro-5,5- dimethyl-3,4,5,6- tetrahydro-[1,1′-biphenyl]- 2-carbonyl)-3,3- difluoropiperidin-4-yl)thio)- 1-oxoisoindolin-2- yl)piperidine-2,6-dione |
| 27 | 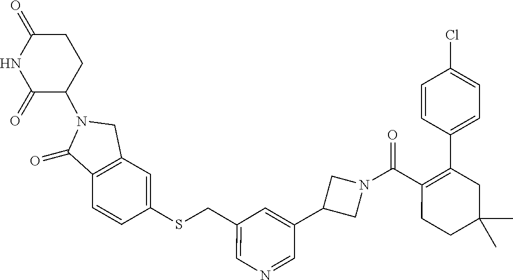 [SMILES: CC1(C)CCC(C(=O)N2CC(c3cncc(CSc4ccc5c(c4)CN(C4CCC(=O)NC4=O)C5=O)c3)C2)=C(c2ccc(Cl)cc2)C1] | 3-(5-(((5-(1-(4′-chloro-5,5- dimethyl-3,4,5,6- tetrahydro-[1,1′-biphenyl]- 2-carbonyl)azetidin-3- yl)pyridin-3- yl)methyl)thio)-1- oxoisoindolin-2- yl)piperidine-2,6-dione |
| 28 GT- 03360 | 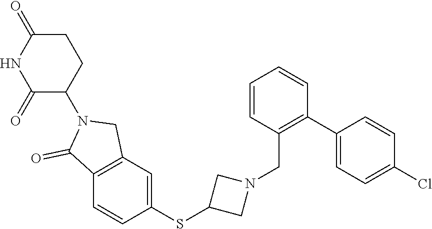 [SMILES: O=C1CCC(N2Cc3cc(SC4CN(Cc5ccccc5-c5ccc(Cl)cc5)C4)ccc3C2=O)C(=O)N1] | 3-(5-((1-((4′-chloro-[1,1′- biphenyl]-2- yl)methyl)azetidin-3- yl)thio)-1-oxoisoindolin-2- yl)piperidine-2,6-dione |
| 29 GT- 04195 | 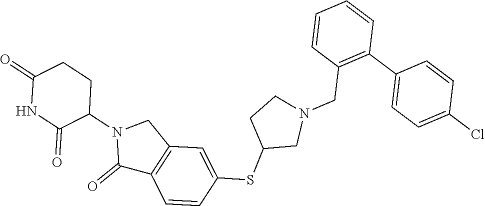 [SMILES: O=C1CCC(N2Cc3cc(SC4CCN(Cc5ccccc5-c5ccc(Cl)cc5)C4)ccc3C2=O)C(=O)N1] | 3-(5-((1-((4′-chloro-[1,1′- biphenyl]-2- yl)methyl)pyrrolidin-3- yl)thio)-1-oxoisoindolin-2- yl)piperidine-2,6-dione |
| 30 GT- 03357 | 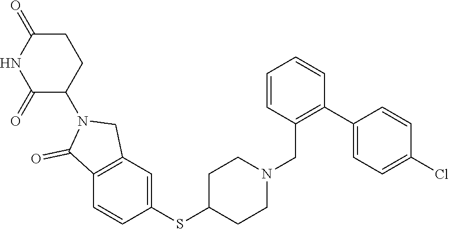 [SMILES: O=C1CCC(N2Cc3cc(SC4CCN(Cc5ccccc5-c5ccc(Cl)cc5)CC4)ccc3C2=O)C(=O)N1] | 3-(5-((1-((4′-chloro-[1,1′- biphenyl]-2- yl)methyl)piperidin-4- yl)thio)-1-oxoisoindolin-2- yl)piperidine-2,6-dione |
| 31 GT- 06643 | 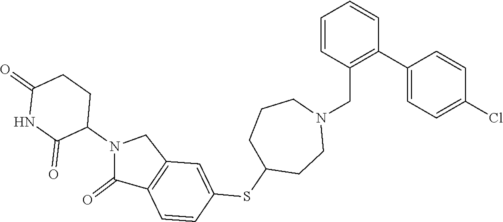 [SMILES: O=C1CCC(N2Cc3cc(SC4CCCN(Cc5ccccc5-c5ccc(Cl)cc5)CC4)ccc3C2=O)C(=O)N1] | 3-(5-((1-((4′-chloro-[1,1′- biphenyl]-2- yl)methyl)azepan-4- yl)thio)-1-oxoisoindolin-2- yl)piperidine-2,6-dione |
| 32 GT- 06914 | 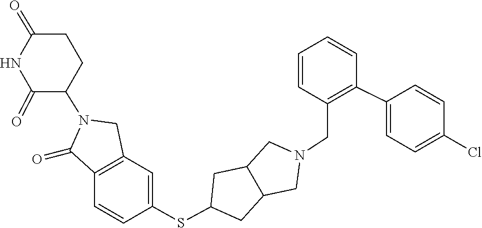 [SMILES: O=C1CCC(N2Cc3cc(SC4CC5CN(Cc6ccccc6-c6ccc(Cl)cc6)CC5C4)ccc3C2=O)C(=O)N1] | 3-(5-((2-((4′-chloro-[1,1′- biphenyl]-2- yl)methyl) octahydrocyclopenta[c] pyrrol-5-yl)thio)-1- oxoisoindolin-2- yl)piperidine-2,6-dione |
| 33 | 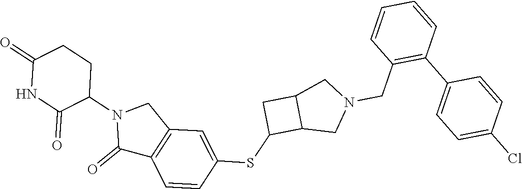 [SMILES: O=C1CCC(N2Cc3cc(SC4CC5CN(Cc6ccccc6-c6ccc(Cl)cc6)CC54)ccc3C2=O)C(=O)N1] | 3-(5-((3-((4′-chloro-[1,1′- biphenyl]-2-yl)methyl)-3- azabicyclo[3.2.0]heptan-6- yl)thio)-1-oxoisoindolin-2- yl)piperidine-2,6-dione |
| 34 | 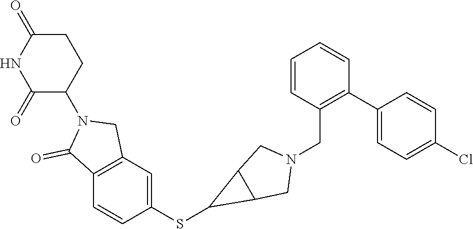 [SMILES: O=C1CCC(N2Cc3cc(SC4C5CN(Cc6ccccc6-c6ccc(Cl)cc6)CC54)ccc3C2=O)C(=O)N1] | 3-(5-((3-((4′-chloro-[1,1′- biphenyl]-2-yl)methyl)-3- azabicyclo[3.1.0]hexan-6- yl)thio)-1-oxoisoindolin-2- yl)piperidine-2,6-dione |
| 35 | 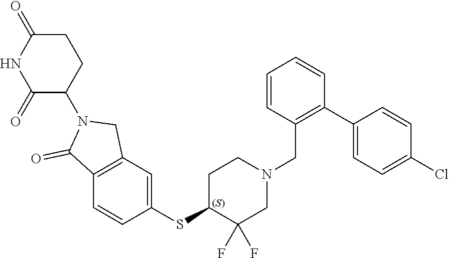 | 3-(5-(((S)-1-((4′-chloro- [1,1′-biphenyl]-2- yl)methyl)-3,3- difluoropiperidin-4-yl)thio)- 1-oxoisoindolin-2- yl)piperidine-2,6-dione |
| 36 GT- 06515 | 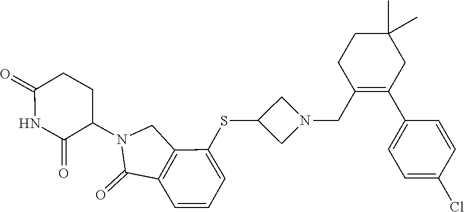 [SMILES: CC1(C)CCC(CN2CC(Sc3cccc4c3CN(C3CCC(=O)NC3=O)C4=O)C2)=C(c2ccc(Cl)cc2)C1] | 3-(4-((1-((4′-chloro-5,5- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 37 GT 03892 | 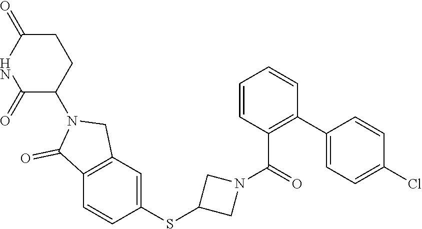 [SMILES: O=C1CCC(N2Cc3cc(SC4CN(C(=O)c5ccccc5-c5ccc(Cl)cc5)C4)ccc3C2=O)C(=O)N1] | 3-(5-((1-(4′-chloro-[1,1′- biphenyl]-2- carbonyl)azetidin-3- yl)thio)-1-oxoisoindolin-2- yl)piperidine-2,6-dione |
| 38 GT- 06181 | 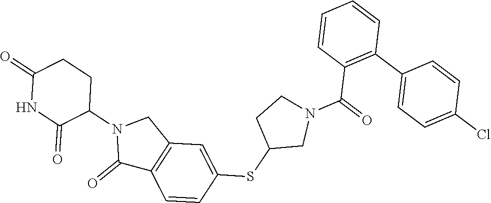 [SMILES: O=C1CCC(N2Cc3cc(SC4CCN(C(=O)c5ccccc5-c5ccc(Cl)cc5)C4)ccc3C2=O)C(=O)N1] | 3-(5-((1-(4′-chloro-[1,1′- biphenyl]-2- carbonyl)pyrrolidin-3- yl)thio)-1-oxoisoindolin-2- yl)piperidine-2,6-dione |
| 39 GT- 06182 | 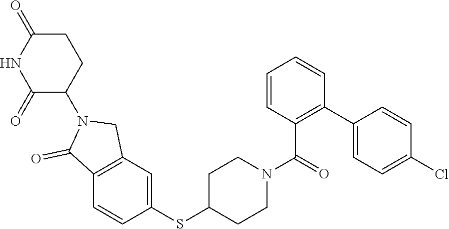 [SMILES: O=C1CCC(N2Cc3cc(SC4CCN(C(=O)c5ccccc5-c5ccc(Cl)cc5)CC4)ccc3C2=O)C(=O)N1] | 3-(5-((1-(4′-chloro-[1,1′- biphenyl]-2- carbonyl)piperidin-4- yl)thio)-1-oxoisoindolin-2- yl)piperidine-2,6-dione |
| 40 GT- 06644 | 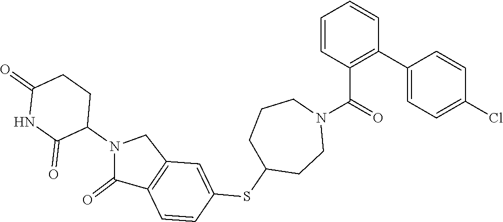 [SMILES: O=C1CCC(N2Cc3cc(SC4CCCN(C(=O)c5ccccc5-c5ccc(Cl)cc5)CC4)ccc3C2=O)C(=O)N1] | 3-(5-((1-(4′-chloro-[1,1′- biphenyl]-2- carbonyl)azepan-4-yl)thio)- 1-oxoisoindolin-2- yl)piperidine-2,6-dione |
| 41 | 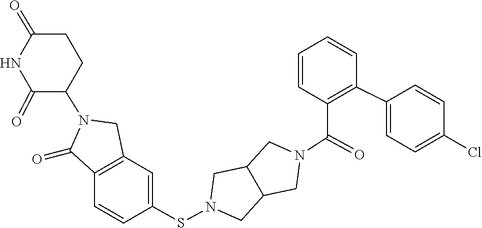 [SMILES: O=C1CCC(N2Cc3cc(SN4CC5CN(C(=O)c6ccccc6-c6ccc(Cl)cc6)CC5C4)ccc3C2=O)C(=O)N1] | 3-(5-((2-(4′-chloro-[1,1′- biphenyl]-2- carbonyl) octahydrocyclopenta [c]pyrrol-5-yl)thio)-1- oxoisoindolin-2- yl)piperidine-2,6-dione |
| 42 | 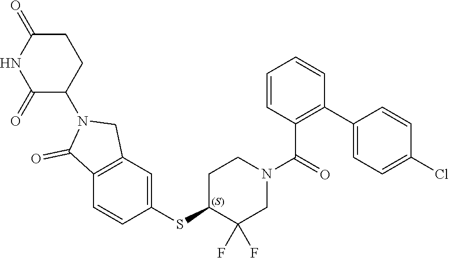 | 3-(5-(((S)-1-(4′-chloro-[1,1′- biphenyl]-2-carbonyl)-3,3- difluoropiperidin-4-yl)thio)- 1-oxoisoindolin-2- yl)piperidine-2,6-dione |
| 43 GT- 06225 | 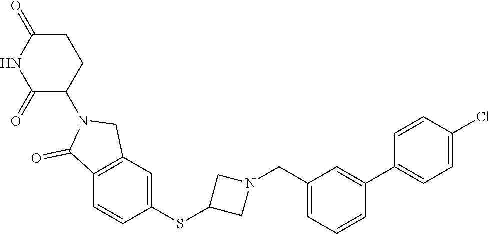 [SMILES: O=C1CCC(N2Cc3cc(SC4CN(Cc5cccc(-c6ccc(Cl)cc6)c5)C4)ccc3C2=O)C(=O)N1] | 3-(5-((1-((4′-chloro-[1,1′- biphenyl]-3- yl)methyl)azetidin-3- yl)thio)-1-oxoisoindolin-2- yl)piperidine-2,6-dione |
| 44 GT- 06226 | 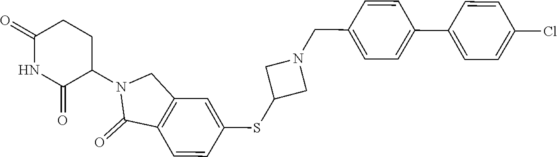 [SMILES: O=C1CCC(N2Cc3cc(SC4CN(Cc5ccc(-c6ccc(Cl)cc6)cc5)C4)ccc3C2=O)C(=O)N1] | 3-(5-((1-((4′-chloro-[1,1′- biphenyl]-4- yl)methyl)azetidin-3- yl)thio)-1-oxoisoindolin-2- yl)piperidine-2,6-dione |
| 45 GT 06350 | 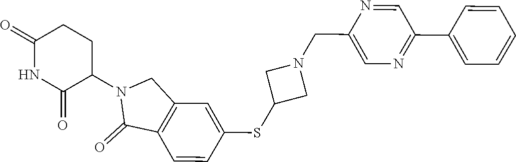 [SMILES: O=C1CCC(N2Cc3cc(SC4CN(Cc5cnc(-c6ccccc6)cn5)C4)ccc3C2=O)C(=O)N1] | 3-(1-oxo-5-((1-((5- phenylpyrazin-2- yl)methyl)azetidin-3- yl)thio)isoindolin-2- yl)piperidine-2,6-dione |
| 46 GT- 06808 | 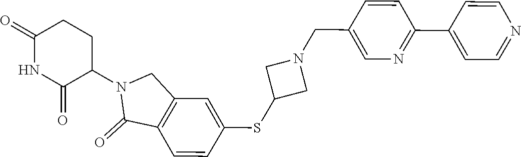 [SMILES: O=C1CCC(N2Cc3cc(SC4CN(Cc5ccc(-c6ccncc6)nc5)C4)ccc3C2=O)C(=O)N1] | 3-(5-((1-([2,4′-bipyridin]-5- ylmethyl)azetidin-3- yl)thio)-1-oxoisoindolin-2- yl)piperidine-2,6-dione |
| 47 GT- 06517 | 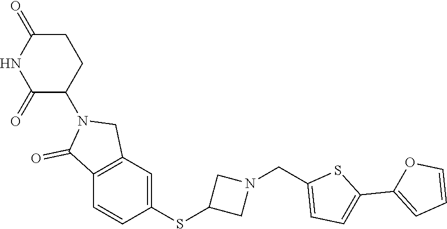 [SMILES: O=C1CCC(N2Cc3cc(SC4CN(Cc5ccc(-c6ccco6)s5)C4)ccc3C2=O)C(=O)N1] | 3-(5-((1-((5-(furan-2- yl)thiophen-2- yl)methyl)azetidin-3- yl)thio)-1-oxoisoindolin-2- yl)piperidine-2,6-dione |
| 48 GT- 03457 | 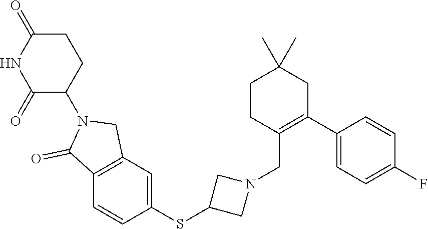 [SMILES: CC1(C)CCC(CN2CC(Sc3ccc4c(c3)CN(C3CCC(=O)NC3=O)C4=O)C2)=C(c2ccc(F)cc2)C1] | 3-(5-((1-((4′-fluoro-5,5- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 49 GT- 06301 | 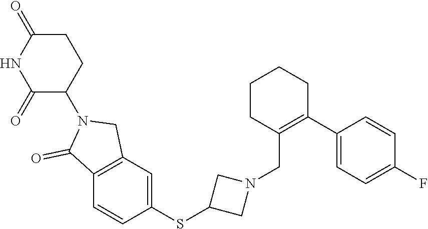 [SMILES: O=C1CCC(N2Cc3cc(SC4CN(CC5=C(c6ccc(F)cc6)CCCC5)C4)ccc3C2=O)C(=O)N1] | 3-(5-((1-((4′-fluoro-3,4,5,6- tetrahydro-[1,1′-biphenyl]- 2-yl)methyl)azetidin-3- yl)thio)-1-oxoisoindolin-2- yl)piperidine-2,6-dione |
| 50 GT- 06351 | 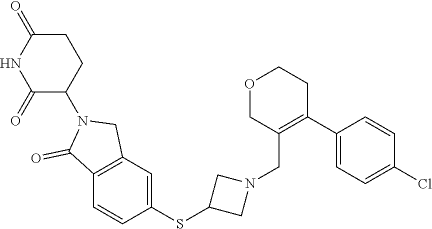 [SMILES: O=C1CCC(N2Cc3cc(SC4CN(CC5=C(c6ccc(Cl)cc6)CCOC5)C4)ccc3C2=O)C(=O)N1] | 3-(5-((1-((4-(4- chlorophenyl)-5,6-dihydro- 2H-pyran-3- yl)methyl)azetidin-3- yl)thio)-1-oxoisoindolin-2- yl)piperidine-2,6-dione |
| 51 GT- 06513 | 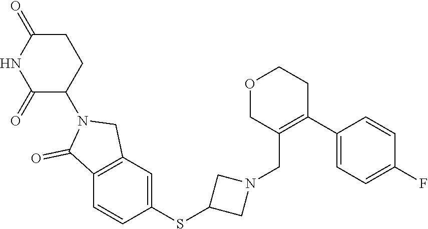 [SMILES: O=C1CCC(N2Cc3cc(SC4CN(CC5=C(c6ccc(F)cc6)CCOC5)C4)ccc3C2=O)C(=O)N1] | 3-(5-((1-((4-(4- fluorophenyl)-5,6-dihydro- 2H-pyran-3- yl)methyl)azetidin-3- yl)thio)-1-oxoisoindolin-2- yl)piperidine-2,6-dione |
| 52 GT 06516 | 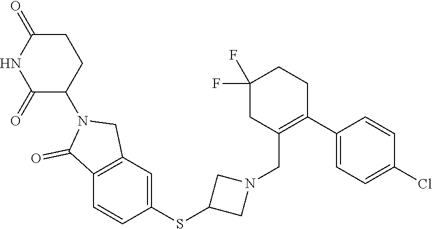 [SMILES: O=C1CCC(N2Cc3cc(SC4CN(CC5=C(c6ccc(Cl)cc6)CCC(F)(F)C5)C4)ccc3C2=O)C(=O)N1] | 3-(5-((1-((4′-chloro-4,4- difluoro-3,4,5,6-tetrahydro- [1,1′-biphenyl]-2- yl)methyl)azetidin-3- yl)thio)-1-oxoisoindolin-2- yl)piperidine-2,6-dione |
| 53 GT 07064 | 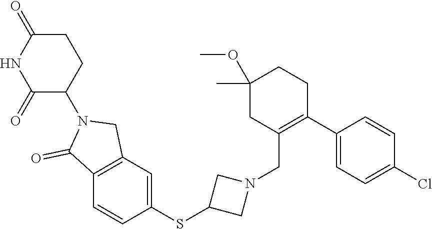 [SMILES: COC1(C)CCC(c2ccc(Cl)cc2)=C(CN2CC(Sc3ccc4c(c3)CN(C3CCC(=O)NC3=O)C4=O)C2)C1] | 3-(5-((1-((4′-chloro-4- methoxy-4-methyl-3,4,5,6- tetrahydro-[1,1′-biphenyl]- 2-yl)methyl)azetidin-3- yl)thio)-1-oxoisoindolin-2- yl)piperidine-2,6-dione |
| 54 GT- 06514 | 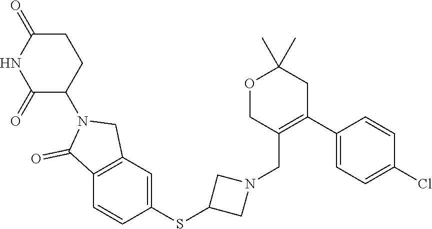 [SMILES: CC1(C)CC(c2ccc(Cl)cc2)=C(CN2CC(Sc3ccc4c(c3)CN(C3CCC(=O)NC3=O)C4=O)C2)CO1] | 3-(5-((1-((4-(4- chlorophenyl)-6,6- dimethyl-5,6-dihydro-2H- pyran-3-yl)methyl)azetidin- 3-yl)thio)-1-oxoisoindolin- 2-yl)piperidine-2,6-dione |
| 55 | 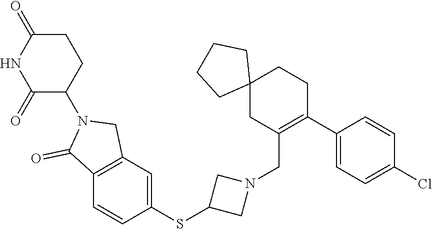 [SMILES: O=C1CCC(N2Cc3cc(SC4CN(CC5=C(c6ccc(Cl)cc6)CCC6(CCCC6)C5)C4)ccc3C2=O)C(=O)N1] | 3-(5-((1-((8-(4- chlorophenyl)spiro[4.5]dec- 7-en-7-yl)methyl)azetidin- 3-yl)thio)-1-oxoisoindolin- 2-yl)piperidine-2,6-dione |
| 56 | 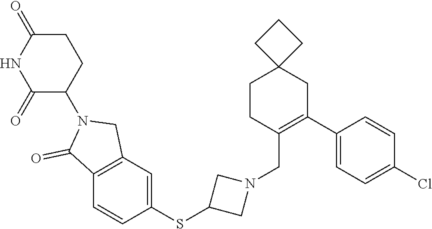 [SMILES: O=C1CCC(N2Cc3cc(SC4CN(CC5=C(c6ccc(Cl)cc6)CC6(CCC6)CC5)C4)ccc3C2=O)C(=O)N1] | 3-(5-((1-((6-(4- chlorophenyl)spiro[3.5]non- 6-en-7-yl)methyl)azetidin- 3-yl)thio)-1-oxoisoindolin- 2-yl)piperidine-2,6-dione |
| 57 GT- 06138 | 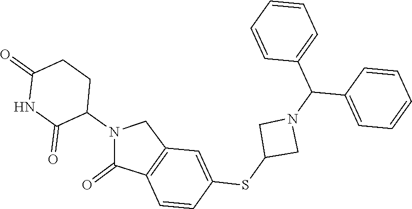 [SMILES: O=C1CCC(N2Cc3cc(SC4CN(C(c5ccccc5)c5ccccc5)C4)ccc3C2=O)C(=O)N1] | 3-(5-((1- benzhydrylazetidin-3- yl)thio)-1-oxoisoindolin-2- yl)piperidine-2,6-dione |
| 58 GT- 06196 | 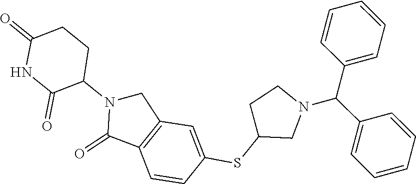 [SMILES: O=C1CCC(N2Cc3cc(SC4CCN(C(c5ccccc5)c5ccccc5)C4)ccc3C2=O)C(=O)N1] | 3-(5-((1- benzhydrylpyrrolidin-3- yl)thio)-1-oxoisoindolin-2- yl)piperidine-2,6-dione |
| 59 GT- 06184 | 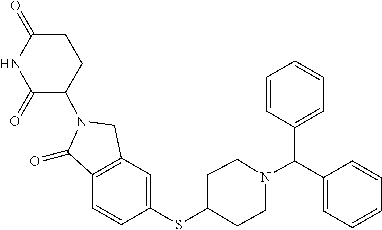 [SMILES: O=C1CCC(N2Cc3cc(SC4CCN(C(c5ccccc5)c5ccccc5)CC4)ccc3C2=O)C(=O)N1] | 3-(5-((1- benzhydrylpiperidin-4- yl)thio)-1-oxoisoindolin-2- yl)piperidine-2,6-dione |
| 60 GT- 06645 | 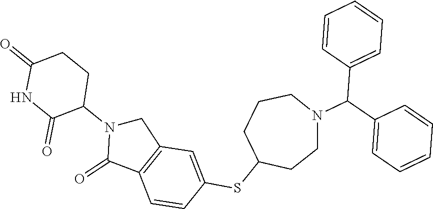 [SMILES: O=C1CCC(N2Cc3cc(SC4CCCN(C(c5ccccc5)c5ccccc5)CC4)ccc3C2=O)C(=O)N1] | 3-(5-((1-benzhydrylazepan- 4-yl)thio)-1-oxoisoindolin- 2-yl)piperidine-2,6-dione |
| 61 | 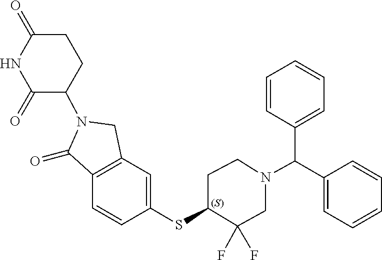 | 3-(5-(((S)-1-benzhydryl- 3,3-difluoropiperidin-4- yl)thio)-1-oxoisoindolin-2- yl)piperidine-2,6-dione |
| 62 GT- 06920 | 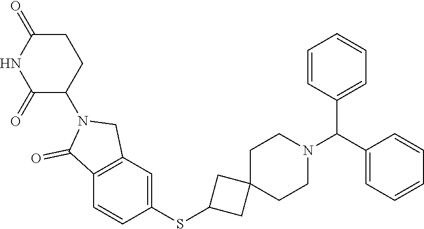 [SMILES: O=C1CCC(N2Cc3cc(SC4CC5(CCN(C(c6ccccc6)c6ccccc6)CC5)C4)ccc3C2=O)C(=O)N1] | 3-(5-((7-benzhydryl-7- azaspiro[3.5]nonan-2- yl)thio)-1-oxoisoindolin-2- yl)piperidine-2,6-dione |
| 63 GT- 06958 | 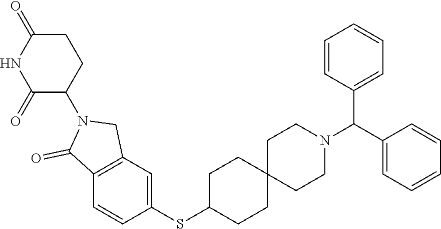 [SMILES: O=C1CCC(N2Cc3cc(SC4CCC5(CC4)CCN(C(c4ccccc4)c4ccccc4)CC5)ccc3C2=O)C(=O)N1] | 3-(5-((3-benzhydryl-3- azaspiro[5.5]undecan-9- yl)thio)-1-oxoisoindolin-2- yl)piperidine-2,6-dione |
| 64 GT- 06922 | 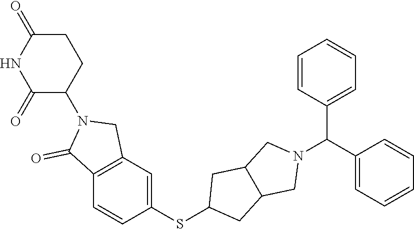 [SMILES: O=C1CCC(N2Cc3cc(SC4CC5CN(C(c6ccccc6)c6ccccc6)CC5C4)ccc3C2=O)C(=O)N1] | 3-(5-((2- benzhydryloctahydro- cyclopenta[c] pyrrol-5-yl)thio)-1- oxoisoindolin-2- yl)piperidine-2,6-dione |
| 65 | 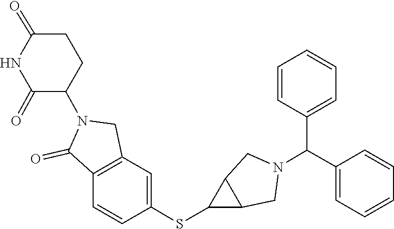 [SMILES: O=C1CCC(N2Cc3cc(SC4C5CN(C(c6ccccc6)c6ccccc6)CC54)ccc3C2=O)C(=O)N1] | 3-(5-((3-benzhydryl-3- azabicyclo[3.1.0]hexan-6- yl)thio)-1-oxoisoindolin-2- yl)piperidine-2,6-dione |
| 66 | 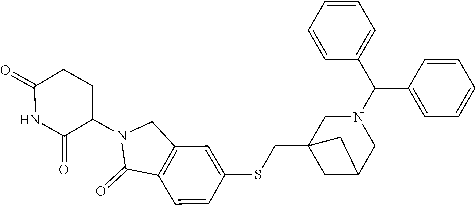 [SMILES: O=C1CCC(N2Cc3cc(SCC45CC(CN(C(c6ccccc6)c6ccccc6)C4)C5)ccc3C2=O)C(=O)N1] | 3-(5-(((3-benzhydryl-3- azabicyclo[3.1.1]heptan-1- yl)methyl)thio)-1- oxoisoindolin-2- yl)piperidine-2,6-dione |
| 67 GT- 06758 | 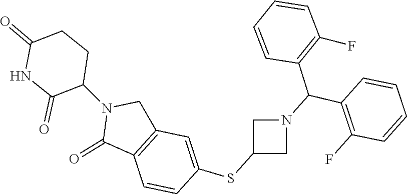 [SMILES: O=C1CCC(N2Cc3cc(SC4CN(C(c5ccccc5F)c5ccccc5F)C4)ccc3C2=O)C(=O)N1] | 3-(5-((1-(bis(2- fluorophenyl)methyl) azetidin-3-yl)thio)-1- oxoisoindolin-2- yl)piperidine-2,6-dione |
| 68 GT- 06349 | 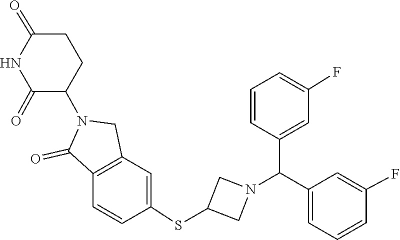 [SMILES: O=C1CCC(N2Cc3cc(SC4CN(C(c5cccc(F)c5)c5cccc(F)c5)C4)ccc3C2=O)C(=O)N1] | 3-(5-((1-(bis(3- fluorophenyl)methyl) azetidin-3-yl)thio)-1- oxoisoindolin-2- yl)piperidine-2,6-dione |
| 69 GT- 06503 | 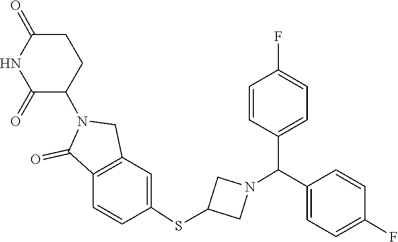 [SMILES: O=C1CCC(N2Cc3cc(SC4CN(C(c5ccc(F)cc5)c5ccc(F)cc5)C4)ccc3C2=O)C(=O)N1] | 3-(5-((1-(bis(4- fluorophenyl)methyl) azetidin-3-yl)thio)-1- oxoisoindolin-2- yl)piperidine-2,6-dione |
| 70 GT 06504 | 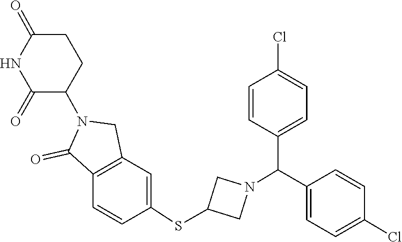 [SMILES: O=C1CCC(N2Cc3cc(SC4CN(C(c5ccc(Cl)cc5)c5ccc(Cl)cc5)C4)ccc3C2=O)C(=O)N1] | 3-(5-((1-(bis(4- chlorophenyl)methyl) azetidin-3-yl)thio)-1- oxoisoindolin-2- yl)piperidine-2,6-dione |
| 71 | 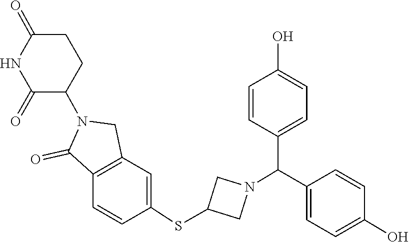 [SMILES: O=C1CCC(N2Cc3cc(SC4CN(C(c5ccc(O)cc5)c5ccc(O)cc5)C4)ccc3C2=O)C(=O)N1] | 3-(5-((1-(bis(4- hydroxyphenyl)methyl) azetidin-3-yl)thio)-1- oxoisoindolin-2- yl)piperidine-2,6-dione |
| 72 GT 06923 | 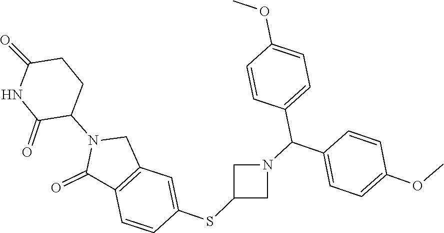 [SMILES: COc1ccc(C(c2ccc(OC)cc2)N2CC(Sc3ccc4c(c3)CN(C3CCC(=O)NC3=O)C4=O)C2)cc1] | 3-(5-((1-(bis(4- methoxyphenyl)methyl) azetidin-3-yl)thio)-1- oxoisoindolin-2- yl)piperidine-2,6-dione |
| 73 | 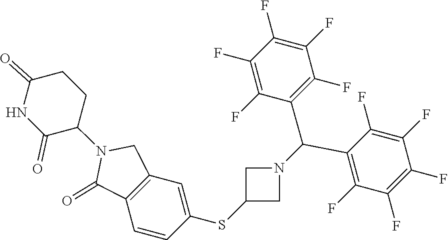 [SMILES: O=C1CCC(N2Cc3cc(SC4CN(C(c5c(F)c(F)c(F)c(F)c5F)c5c(F)c(F)c(F)c(F)c5F)C4)ccc3C2=O)C(=O)N1] | 3-(5-((1- (bis(perfluorophenyl) methyl)azetidin- 3-yl)thio)-1- oxoisoindolin-2- yl)piperidine-2,6-dione |
| 74 GT- 06518 | 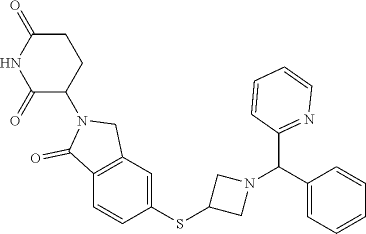 [SMILES: O=C1CCC(N2Cc3cc(SC4CN(C(c5ccccc5)c5ccccn5)C4)ccc3C2=O)C(=O)N1] | 3-(1-oxo-5-((1- (phenyl(pyridin-2- yl)methyl)azetidin-3- yl)thio)isoindolin-2- yl)piperidine-2,6-dione |
| 75 GT- 06807 | 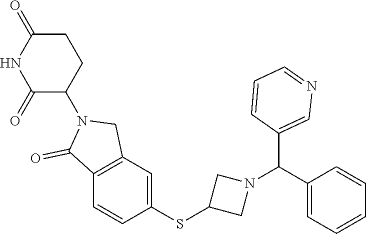 [SMILES: O=C1CCC(N2Cc3cc(SC4CN(C(c5ccccc5)c5cccnc5)C4)ccc3C2=O)C(=O)N1] | 3-(1-oxo-5-((1- (phenyl(pyridin-3- yl)methyl)azetidin-3- yl)thio)isoindolin-2- yl)piperidine-2,6-dione |
| 76 | 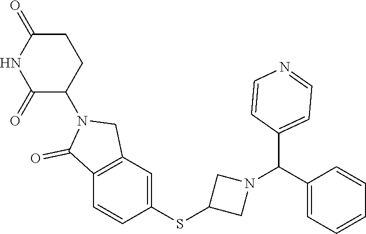 [SMILES: O=C1CCC(N2Cc3cc(SC4CN(C(c5ccccc5)c5ccncc5)C4)ccc3C2=O)C(=O)N1] | 3-(1-oxo-5-((1- (phenyl(pyridin-4- yl)methyl)azetidin-3- yl)thio)isoindolin-2- yl)piperidine-2,6-dione |
| 77 GT- 06811 | 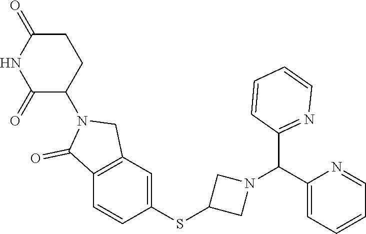 [SMILES: O=C1CCC(N2Cc3cc(SC4CN(C(c5ccccn5)c5ccccn5)C4)ccc3C2=O)C(=O)N1] | 3-(5-((1-(di(pyridin-2- yl)methyl)azetidin-3- yl)thio)-1-oxoisoindolin-2- yl)piperidine-2,6-dione |
| 78 | 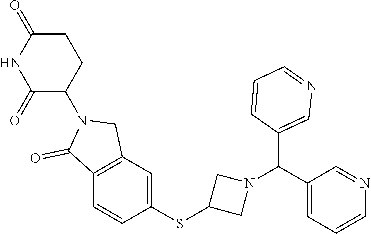 [SMILES: O=C1CCC(N2Cc3cc(SC4CN(C(c5cccnc5)c5cccnc5)C4)ccc3C2=O)C(=O)N1] | 3-(5-((1-(di(pyridin-3- yl)methyl)azetidin-3- yl)thio)-1-oxoisoindolin-2- yl)piperidine-2,6-dione |
| 79 | 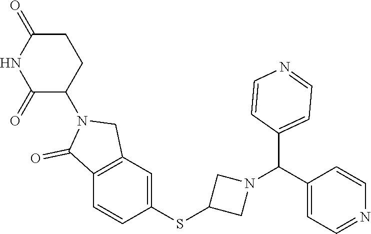 [SMILES: O=C1CCC(N2Cc3cc(SC4CN(C(c5ccncc5)c5ccncc5)C4)ccc3C2=O)C(=O)N1] | 3-(5-((1-(di(pyridin-4- yl)methyl)azetidin-3- yl)thio)-1-oxoisoindolin-2- yl)piperidine-2,6-dione |
| 80 | 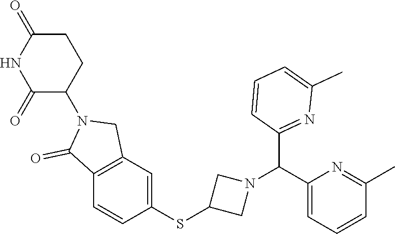 [SMILES: Cc1cccc(C(c2cccc(C)n2)N2CC(Sc3ccc4c(c3)CN(C3CCC(=O)NC3=O)C4=O)C2)n1] | 3-(5-((1-(bis(6- methylpyridin-2- yl)methyl)azetidin-3- yl)thio)-1-oxoisoindolin-2- yl)piperidine-2,6-dione |
| 81 | 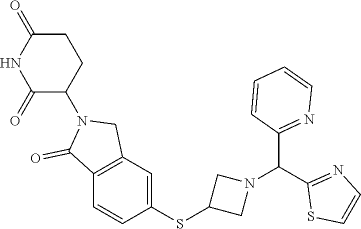 [SMILES: O=C1CCC(N2Cc3cc(SC4CN(C(c5ccccn5)c5nccs5)C4)ccc3C2=O)C(=O)N1] | 3-(1-oxo-5-((1-(pyridin-2- yl(thiazol-2- yl)methyl)azetidin-3- yl)thio)isoindolin-2- yl)piperidine-2,6-dione |
| 82 | 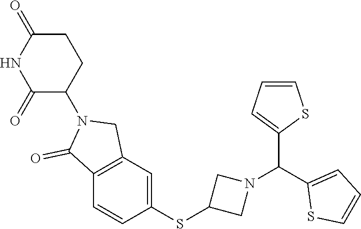 [SMILES: O=C1CCC(N2Cc3cc(SC4CN(C(c5cccs5)c5cccs5)C4)ccc3C2=O)C(=O)N1] | 3-(5-((1-(di(thiophen-2- yl)methyl)azetidin-3- yl)thio)-1-oxoisoindolin-2- yl)piperidine-2,6-dione |
| 83 | 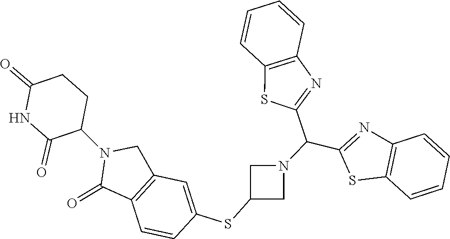 [SMILES: O=C1CCC(N2Cc3cc(SC4CN(C(c5nc6ccccc6s5)c5nc6ccccc6s5)C4)ccc3C2=O)C(=O)N1] | 3-(5-((1- (bis(benzo[d]thiazol-2- yl)methyl)azetidin-3- yl)thio)-1-oxoisoindolin-2- yl)piperidine-2,6-dione |
| 84 GT 03369 | 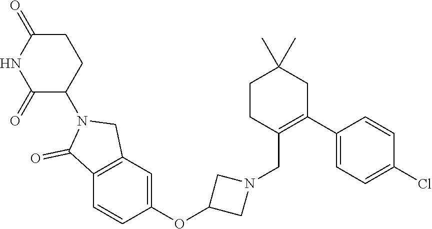 [SMILES: CC1(C)CCC(CN2CC(Oc3ccc4c(c3)CN(C3CCC(=O)NC3=O)C4=O)C2)=C(c2ccc(Cl)cc2)C1] | 3-(5-((1-((4′-chloro-5,5- dimethyl-3,4,5,6- tetrahydro-[1,1′-biphenyl]- 2-yl)methyl)azetidin-3- yl)oxy)-1-oxoisoindolin-2- yl)piperidine-2,6-dione |
| 85 | 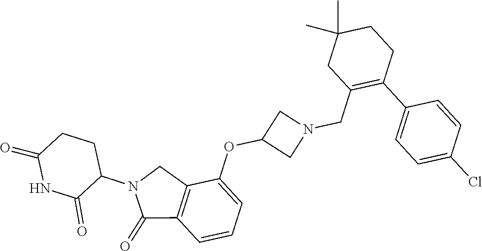 [SMILES: CC1(C)CCC(c2ccc(Cl)cc2)=C(CN2CC(Oc3cccc4c3CN(C3CCC(=O)NC3=O)C4=O)C2)C1] | 3-(4-((1-((4′-chloro-4,4- dimethyl-3,4,5,6- tetrahydro-[1,1′-biphenyl]- 2-yl)methyl)azetidin-3- yl)oxy)-1-oxoisoindolin-2- yl)piperidine-2,6-dione |
| 86 GT- 06872 | 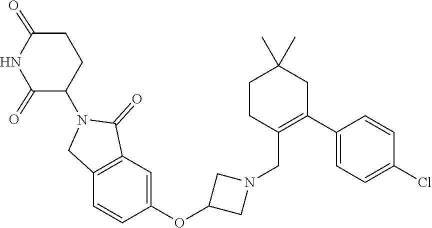 [SMILES: CC1(C)CCC(CN2CC(Oc3ccc4c(c3)C(=O)N(C3CCC(=O)NC3=O)C4)C2)=C(c2ccc(Cl)cc2)C1] | 3-(6-((1-((4′-chloro-5,5- dimethyl-3,4,5,6- tetrahydro-[1,1′-biphenyl]- 2-yl)methyl)azetidin-3- yl)oxy)-1-oxoisoindolin-2- yl)piperidine-2,6-dione |
| 87 | 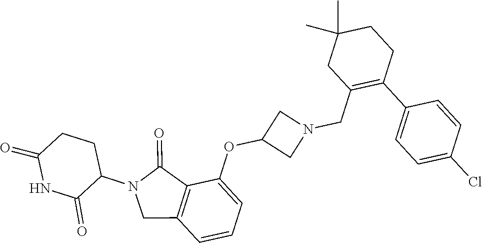 [SMILES: CC1(C)CCC(c2ccc(Cl)cc2)=C(CN2CC(Oc3cccc4c3C(=O)N(C3CCC(=O)NC3=O)C4)C2)C1] | 3-(7-((1-((4′-chloro-4,4- dimethyl-3,4,5,6- tetrahydro-[1,1′-biphenyl]- 2-yl)methyl)azetidin-3- yl)oxy)-1-oxoisoindolin-2- yl)piperidine-2,6-dione |
| 88 GT- 06815 | 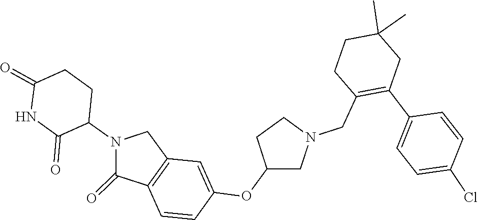 [SMILES: CC1(C)CCC(CN2CCC(Oc3ccc4c(c3)CN(C3CCC(=O)NC3=O)C4=O)C2)=C(c2ccc(Cl)cc2)C1] | 3-(5-((1-((4′-chloro-5,5- dimethyl-3,4,5,6- tetrahydro-[1,1′-biphenyl]- 2-yl)methyl)pyrrolidin-3- yl)oxy)-1-oxoisoindolin-2- yl)piperidine-2,6-dione |
| 89 GT- 07061 | 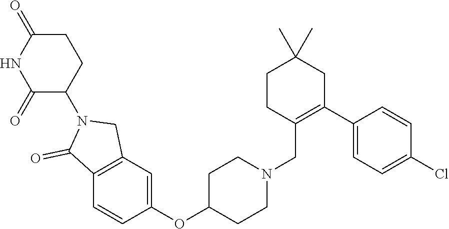 [SMILES: CC1(C)CCC(CN2CCC(Oc3ccc4c(c3)CN(C3CCC(=O)NC3=O)C4=O)CC2)=C(c2ccc(Cl)cc2)C1] | 3-(5-((1-((4′-chloro-5,5- dimethyl-3,4,5,6- tetrahydro-[1,1′-biphenyl]- 2-yl)methyl)piperidin-4- yl)oxy)-1-oxoisoindolin-2- yl)piperidine-2,6-dione |
| 90 GT- 06929 | 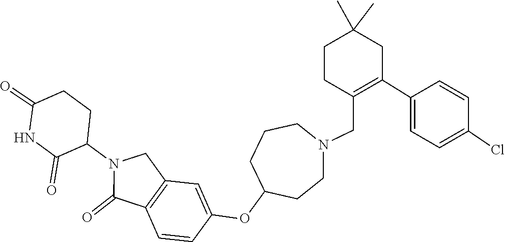 [SMILES: CC1(C)CCC(CN2CCCC(Oc3ccc4c(c3)CN(C3CCC(=O)NC3=O)C4=O)CC2)=C(c2ccc(Cl)cc2)C1] | 3-(5-((1-((4′-chloro-5,5- dimethyl-3,4,5,6- tetrahydro-[1,1′-biphenyl]- 2-yl)methyl)azepan-4- yl)oxy)-1-oxoisoindolin-2- yl)piperidine-2,6-dione |
| 91 | 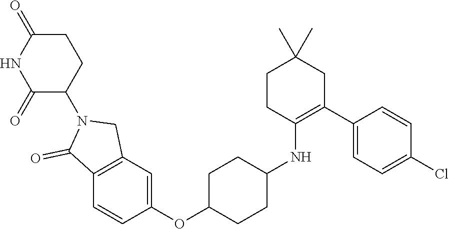 [SMILES: CC1(C)CCC(NC2CCC(Oc3ccc4c(c3)CN(C3CCC(=O)NC3=O)C4=O)CC2)=C(c2ccc(Cl)cc2)C1] | 3-(5-((4-((4′-chloro-5,5- dimethyl-3,4,5,6- tetrahydro-[1,1′-biphenyl]- 2- yl)amino)cyclohexyl)oxy)- 1-oxoisoindolin-2- yl)piperidine-2,6-dione |
| 92 | 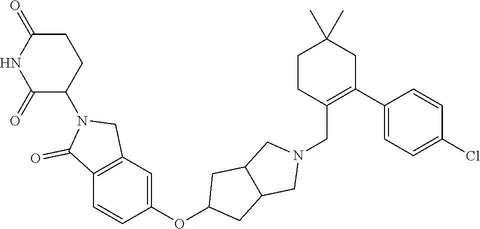 [SMILES: CC1(C)CCC(CN2CC3CC(Oc4ccc5c(c4)CN(C4CCC(=O)NC4=O)C5=O)CC3C2)=C(c2ccc(Cl)cc2)C1] | 3-(5-((2-((4′-chloro-5,5- dimethyl-3,4,5,6- tetrahydro-[1,1′-biphenyl]- 2- yl)methyl)octahydrocyclope nta[c]pyrrol-5-yl)oxy)-1- oxoisoindolin-2- yl)piperidine-2,6-dione |
| 93 | 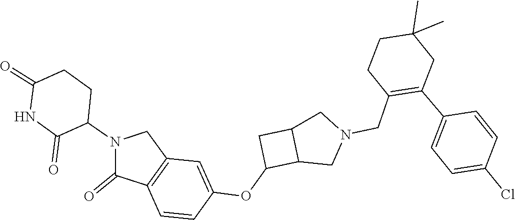 [SMILES: CC1(C)CCC(CN2CC3CC(Oc4ccc5c(c4)CN(C4CCC(=O)NC4=O)C5=O)C3C2)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azabicyclo[3.2.0]heptan-6- yl)oxy)-1-oxoisoindolin-2- yl)piperidine-2,6-dione |
| 94 | 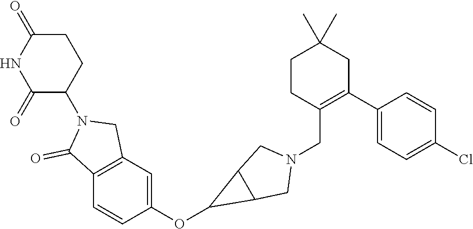 [SMILES: CC1(C)CCC(CN2CC3C(C2)C3Oc2ccc3c(c2)CN(C2CCC(=O)NC2=O)C3=O)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azabicyclo[3.1.0]hexan-6- yl)oxy)-1-oxoisoindolin-2- yl)piperidine-2,6-dione |
| 95 GT- 06933 | 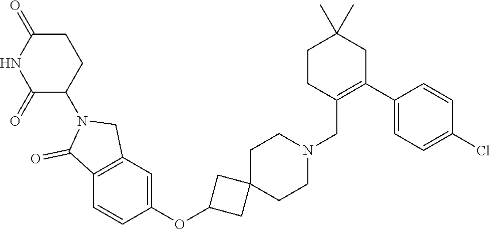 [SMILES: CC1(C)CCC(CN2CCC3(CC2)CC(Oc2ccc4c(c2)CN(C2CCC(=O)NC2=O)C4=O)C3)=C(c2ccc(Cl)cc2)C1] | 3-(5-((7-((4′-chloro-5,5- dimethyl-3,4,5,6- tetrahydro-[1,1′-biphenyl]- 2-yl)methyl)-7- azaspiro[3.5]nonan-2- yl)oxy)-1-oxoisoindolin-2- yl)piperidine-2,6-dione |
| 96 | 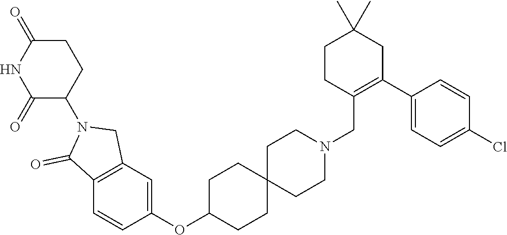 [SMILES: CC1(C)CCC(CN2CCC3(CCC(Oc4ccc5c(c4)CN(C4CCC(=O)NC4=O)C5=O)CC3)CC2)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azaspiro[5.5]undecan-9- yl)oxy)-1-oxoisoindolin-2- yl)piperidine-2,6-dione |
| 97 | 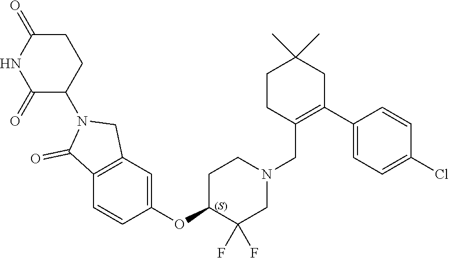 | 3-(5-(((S)-1-((4′-chloro-5,5- dimethyl-3,4,5,6- tetrahydro-[1,1′-biphenyl]- 2-yl)methyl)-3,3- difluoropiperidin-4-yl)oxy)- 1-oxoisoindolin-2- yl)piperidine-2,6-dione |
| 98 | 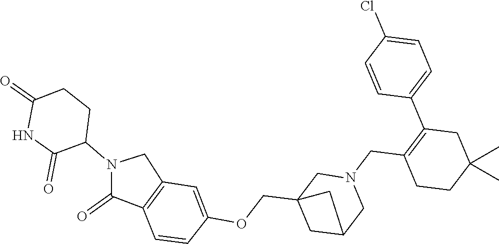 [SMILES: CC1(C)CCC(CN2CC3CC(COc4ccc5c(c4)CN(C4CCC(=O)NC4=O)C5=O)(C3)C2)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azabicyclo[3.1.1]heptan-1- yl)methoxy)-1- oxoisoindolin-2- yl)piperidine-2,6-dione |
| 99 | 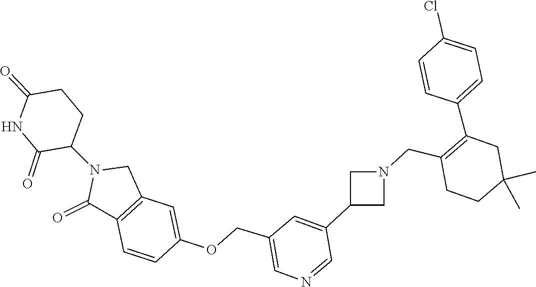 [SMILES: CC1(C)CCC(CN2CC(c3cncc(COc4ccc5c(c4)CN(C4CCC(=O)NC4=O)C5=O)c3)C2)=C(c2ccc(Cl)cc2)C1] | 3-(5-((5-(1-((4′-chloro-5,5- dimethyl-3,4,5,6- tetrahydro-[1,1′-biphenyl]- 2-yl)methyl)azetidin-3- yl)pyridin-3-yl)methoxy)-1- oxoisoindolin-2- yl)piperidine-2,6-dione |
| 100 GT- 06180 | 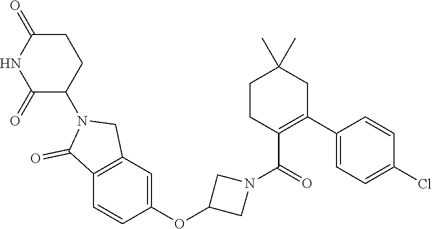 [SMILES: CC1(C)CCC(C(=O)N2CC(Oc3ccc4c(c3)CN(C3CCC(=O)NC3=O)C4=O)C2)=C(c2ccc(Cl)cc2)C1] | 3-(5-((1-(4′-chloro-5,5- dimethyl-3,4,5,6- tetrahydro-[1,1′-biphenyl]- 2-carbonyl)azetidin-3- yl)oxy)-1-oxoisoindolin-2- yl)piperidine-2,6-dione |
| 101 GT- 06816 | 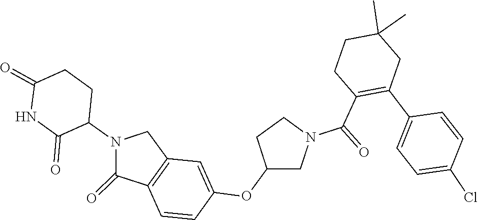 [SMILES: CC1(C)CCC(C(=O)N2CCC(Oc3ccc4c(c3)CN(C3CCC(=O)NC3=O)C4=O)C2)=C(c2ccc(Cl)cc2)C1] | 3-(5-((1-(4′-chloro-5,5- dimethyl-3,4,5,6- tetrahydro-[1,1′-biphenyl]- 2-carbonyl)pyrrolidin-3- yl)oxy)-1-oxoisoindolin-2- yl)piperidine-2,6-dione |
| 102 GT- 06192 | 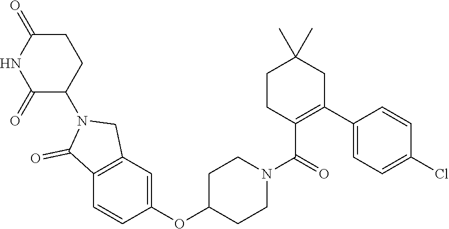 [SMILES: CC1(C)CCC(C(=O)N2CCC(Oc3ccc4c(c3)CN(C3CCC(=O)NC3=O)C4=O)CC2)=C(c2ccc(Cl)cc2)C1] | 3-(5-((1-(4′-chloro-5,5- dimethyl-3,4,5,6- tetrahydro-[1,1′-biphenyl]- 2-carbonyl)piperidin-4- yl)oxy)-1-oxoisoindolin-2- yl)piperidine-2,6-dione |
| 103 GT- 07018 | 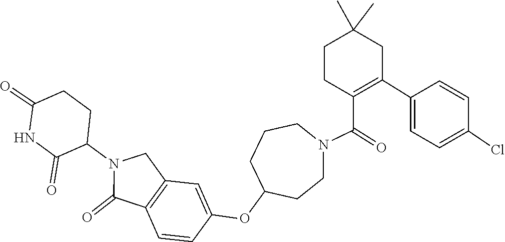 [SMILES: CC1(C)CCC(C(=O)N2CCCC(Oc3ccc4c(c3)CN(C3CCC(=O)NC3=O)C4=O)CC2)=C(c2ccc(Cl)cc2)C1] | 3-(5-((1-(4′-chloro-5,5- dimethyl-3,4,5,6- tetrahydro-[1,1′-biphenyl]- 2-carbonyl)azepan-4- yl)oxy)-1-oxoisoindolin-2- yl)piperidine-2,6-dione |
| 104 | 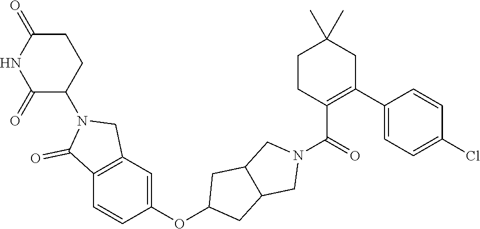 [SMILES: CC1(C)CCC(C(=O)N2CC3CC(Oc4ccc5c(c4)CN(C4CCC(=O)NC4=O)C5=O)CC3C2)=C(c2ccc(Cl)cc2)C1] | 3-(5-((2-(4′-chloro-5,5- dimethyl-3,4,5,6- tetrahydro-[1,1′-biphenyl]- 2-carbonyl) octahydrocyclopenta [c]pyrrol-5-yl)oxy)-1- oxoisoindolin-2- yl)piperidine-2,6-dione |
| 105 | 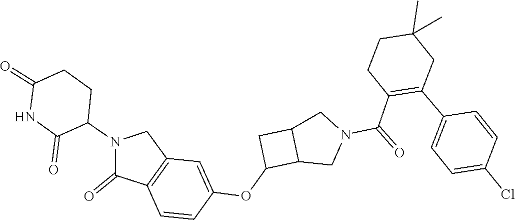 [SMILES: CC1(C)CCC(C(=O)N2CC3CC(Oc4ccc5c(c4)CN(C4CCC(=O)NC4=O)C5=O)C3C2)=C(c2ccc(Cl)cc2)C1] | 3-(5-((3-(4′-chloro-5,5- dimethyl-3,4,5,6- tetrahydro-[1,1′-biphenyl]- 2-carbonyl)-3- azabicyclo[3.2.0]heptan-6- yl)oxy)-1-oxoisoindolin-2- yl)piperidine-2,6-dione |
| 106 | 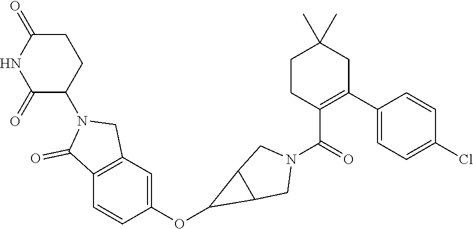 [SMILES: CC1(C)CCC(C(=O)N2CC3C(C2)C3Oc2ccc3c(c2)CN(C2CCC(=O)NC2=O)C3=O)=C(c2ccc(Cl)cc2)C1] | 3-(5-((3-(4′-chloro-5,5- dimethyl-3,4,5,6- tetrahydro-[1,1′-biphenyl]- 2-carbonyl)-3- azabicyclo[3.1.0]hexan-6- yl)oxy)-1-oxoisoindolin-2- yl)piperidine-2,6-dione |
| 107 GT- 07020 | 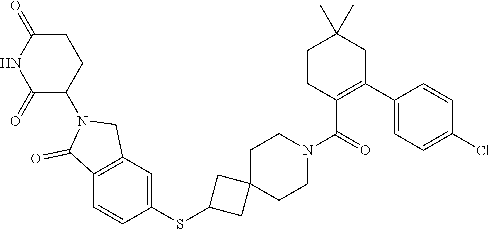 [SMILES: CC1(C)CCC(C(=O)N2CCC3(CC2)CC(Sc2ccc4c(c2)CN(C2CCC(=O)NC2=O)C4=O)C3)=C(c2ccc(Cl)cc2)C1] | 3-(5-((7-(4′-chloro-5,5- dimethyl-3,4,5,6- tetrahydro-[1,1′-biphenyl]- 2-carbonyl)-7- azaspiro[3.5]nonan-2- yl)oxy)-1-oxoisoindolin-2- yl)piperidine-2,6-dione |
| 108 | 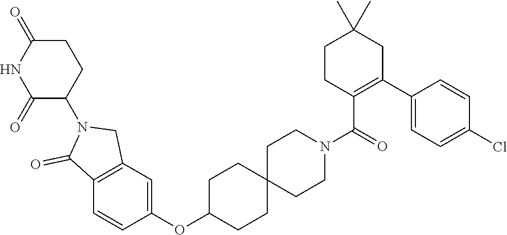 [SMILES: CC1(C)CCC(C(=O)N2CCC3(CCC(Oc4ccc5c(c4)CN(C4CCC(=O)NC4=O)C5=O)CC3)CC2)=C(c2ccc(Cl)cc2)C1] | 3-(5-((3-(4′-chloro-5,5- dimethyl-3,4,5,6- tetrahydro-[1,1′-biphenyl]- 2-carbonyl)-3- azaspiro[5.5]undecan-9- yl)oxy)-1-oxoisoindolin-2- yl)piperidine-2,6-dione |
| 109 | 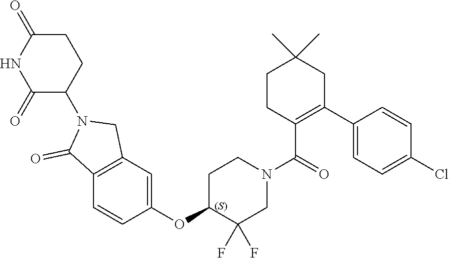 | 3-(5-(((S)-1-(4′-chloro-5,5- dimethyl-3,4,5,6- tetrahydro-[1,1′-biphenyl]- 2-carbonyl)-3,3- difluoropiperidin-4-yl)oxy)- 1-oxoisoindolin-2- yl)piperidine-2,6-dione |
| 110 | 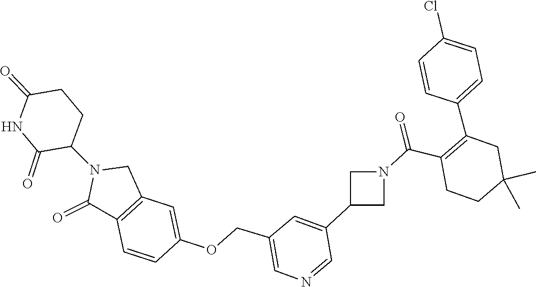 [SMILES: CC1(C)CCC(C(=O)N2CC(c3cncc(COc4ccc5c(c4)CN(C4CCC(=O)NC4=O)C5=O)c3)C2)=C(c2ccc(Cl)cc2)C1] | 3-(5-((5-(1-(4′-chloro-5,5- dimethyl-3,4,5,6- tetrahydro-[1,1′-biphenyl]- 2-carbonyl)azetidin-3- yl)pyridin-3-yl)methoxy)-1- oxoisoindolin-2- yl)piperidine-2,6-dione |
| 111 GT- 03363 | 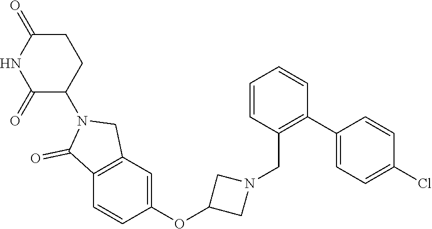 [SMILES: O=C1CCC(N2Cc3cc(OC4CN(Cc5ccccc5-c5ccc(Cl)cc5)C4)ccc3C2=O)C(=O)N1] | 3-(5-((1-((4′-chloro-[1,1′- biphenyl]-2- yl)methyl)azetidin-3- yl)oxy)-1-oxoisoindolin-2- yl)piperidine-2,6-dione |
| 112 GT- 06817 | 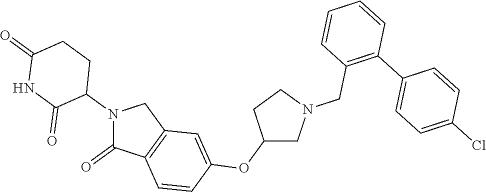 [SMILES: O=C1CCC(N2Cc3cc(OC4CCN(Cc5ccccc5-c5ccc(Cl)cc5)C4)ccc3C2=O)C(=O)N1] | 3-(5-((1-((4′-chloro-[1,1′- biphenyl]-2- yl)methyl)pyrrolidin-3- yl)oxy)-1-oxoisoindolin-2- yl)piperidine-2,6-dione |
| 113 GT- 06193 | 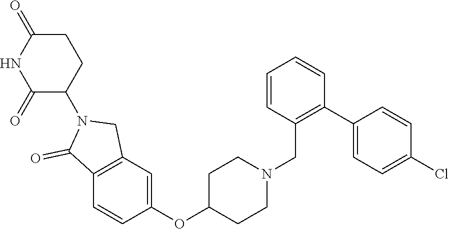 [SMILES: O=C1CCC(N2Cc3cc(OC4CCN(Cc5ccccc5-c5ccc(Cl)cc5)CC4)ccc3C2=O)C(=O)N1] | 3-(5-((1-((4′-chloro-[1,1′- biphenyl]-2- yl)methyl)piperidin-4- yl)oxy)-1-oxoisoindolin-2- yl)piperidine-2,6-dione |
| 114 GT- 06934 | 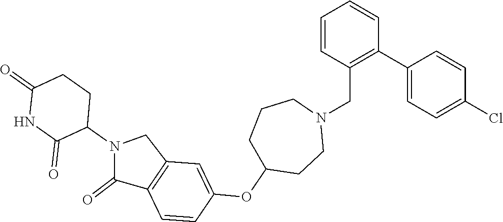 [SMILES: O=C1CCC(N2Cc3cc(OC4CCCN(Cc5ccccc5-c5ccc(Cl)cc5)CC4)ccc3C2=O)C(=O)N1] | 3-(5-((1-((4′-chloro-[1,1′- biphenyl]-2- yl)methyl)azepan-4- yl)oxy)-1-oxoisoindolin-2- yl)piperidine-2,6-dione |
| 115 GT- 06953 | 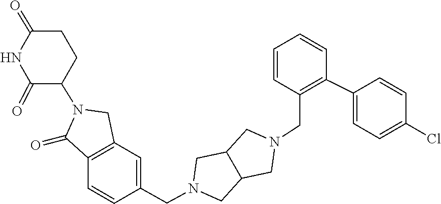 [SMILES: O=C1CCC(N2Cc3cc(CN4CC5CN(Cc6ccccc6-c6ccc(Cl)cc6)CC5C4)ccc3C2=O)C(=O)N1] | 3-(5-((2-((4′-chloro-[1,1′- biphenyl]-2-yl)methyl) octahydrocyclopenta [c]pyrrol-5-yl)oxy)-1- oxoisoindolin-2- yl)piperidine-2,6-dione |
| 116 | 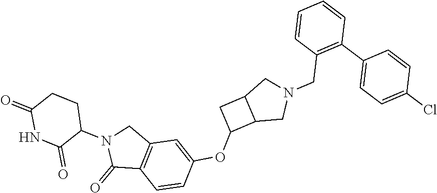 [SMILES: O=C1CCC(N2Cc3cc(OC4CC5CN(Cc6ccccc6-c6ccc(Cl)cc6)CC54)ccc3C2=O)C(=O)N1] | 3-(5-((3-((4′-chloro-[1,1′- biphenyl]-2-yl)methyl)-3- azabicyclo[3.2.0]heptan-6- yl)oxy)-1-oxoisoindolin-2- yl)piperidine-2,6-dione |
| 117 | 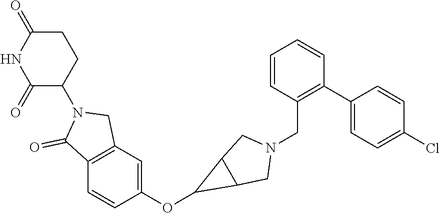 [SMILES: O=C1CCC(N2Cc3cc(OC4C5CN(Cc6ccccc6-c6ccc(Cl)cc6)CC54)ccc3C2=O)C(=O)N1] | 3-(5-((3-((4′-chloro-[1,1′- biphenyl]-2-yl)methyl)-3- azabicyclo[3.1.0]hexan-6- yl)oxy)-1-oxoisoindolin-2- yl)piperidine-2,6-dione |
| 118 | 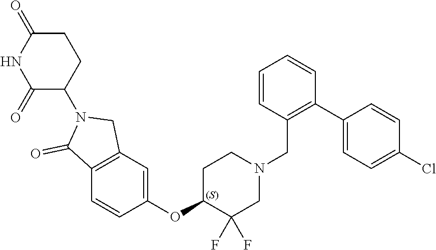 | 3-(5-(((S)-1-((4′-chloro- [1,1′-biphenyl]-2- yl)methyl)-3,3- difluoropiperidin-4-yl)oxy)- 1-oxoisoindolin-2- yl)piperidine-2,6-dione |
| 119 GT- 06183 | 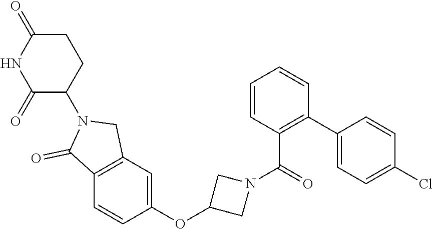 [SMILES: O=C1CCC(N2Cc3cc(OC4CN(C(=O)c5ccccc5-c5ccc(Cl)cc5)C4)ccc3C2=O)C(=O)N1] | 3-(5-((1-(4′-chloro-[1,1′- biphenyl]-2- carbonyl)azetidin-3- yl)oxy)-1-oxoisoindolin-2- yl)piperidine-2,6-dione |
| 120 GT- 06818 | 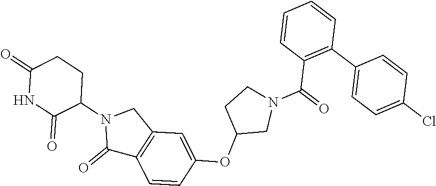 [SMILES: O=C1CCC(N2Cc3cc(OC4CCN(C(=O)c5ccccc5-c5ccc(Cl)cc5)C4)ccc3C2=O)C(=O)N1] | 3-(5-((1-(4′-chloro-[1,1′- biphenyl]-2- carbonyl)pyrrolidin-3- yl)oxy)-1-oxoisoindolin-2- yl)piperidine-2,6-dione |
| 121 GT- 06204 | 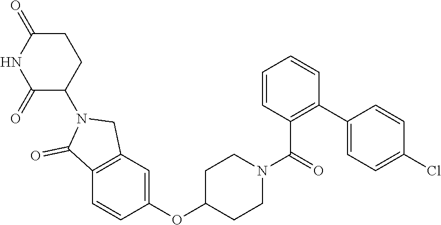 [SMILES: O=C1CCC(N2Cc3cc(OC4CCN(C(=O)c5ccccc5-c5ccc(Cl)cc5)CC4)ccc3C2=O)C(=O)N1] | 3-(5-((1-(4′-chloro-[1,1′- biphenyl]-2- carbonyl)piperidin-4- yl)oxy)-1-oxoisoindolin-2- yl)piperidine-2,6-dione |
| 122 GT- 06935 | 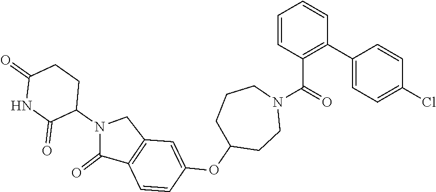 [SMILES: O=C1CCC(N2Cc3cc(OC4CCCN(C(=O)c5ccccc5-c5ccc(Cl)cc5)CC4)ccc3C2=O)C(=O)N1] | 3-(5-((1-(4′-chloro-[1,1′- biphenyl]-2- carbonyl)azepan-4-yl)oxy)- 1-oxoisoindolin-2- yl)piperidine-2,6-dione |
| 123 GT- 06954 | 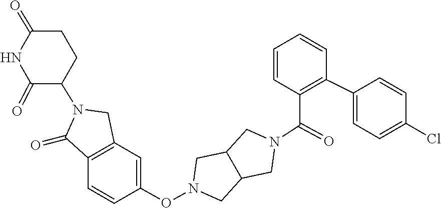 [SMILES: O=C1CCC(N2Cc3cc(ON4CC5CN(C(=O)c6ccccc6-c6ccc(Cl)cc6)CC5C4)ccc3C2=O)C(=O)N1] | 3-(5-((2-(4′-chloro-[1,1′- biphenyl]-2- carbonyl)octahydro- cyclopenta[c]pyrrol- 5-yl)oxy)-1- oxoisoindolin-2- yl)piperidine-2,6-dione |
| 124 | 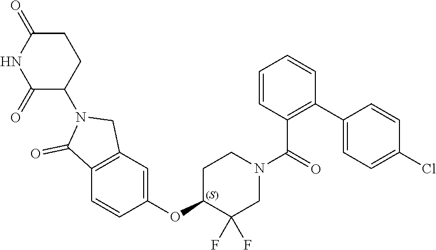 | 3-(5-(((S)-1-(4′-chloro-[1,1′- biphenyl]-2-carbonyl)-3,3- difluoropiperidin-4-yl)oxy)- 1-oxoisoindolin-2- yl)piperidine-2,6-dione |
| 125 GT- 06227 | 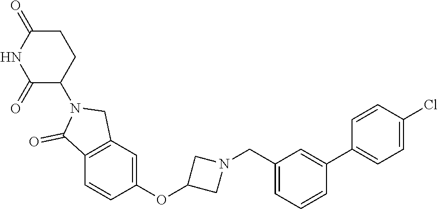 [SMILES: O=C1CCC(N2Cc3cc(OC4CN(Cc5cccc(-c6ccc(Cl)cc6)c5)C4)ccc3C2=O)C(=O)N1] | 3-(5-((1-((4′-chloro-[1,1′- biphenyl]-3- yl)methyl)azetidin-3- yl)oxy)-1-oxoisoindolin-2- yl)piperidine-2,6-dione |
| 126 GT- 06230 | 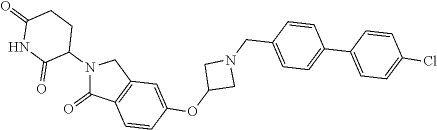 [SMILES: O=C1CCC(N2Cc3cc(OC4CN(Cc5ccc(-c6ccc(Cl)cc6)cc5)C4)ccc3C2=O)C(=O)N1] | 3-(5-((1-((4′-chloro-[1,1′- biphenyl]-4- yl)methyl)azetidin-3- yl)oxy)-1-oxoisoindolin-2- yl)piperidine-2,6-dione |
| 127 GT- 06809 | 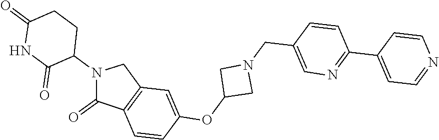 [SMILES: O=C1CCC(N2Cc3cc(OC4CN(Cc5ccc(-c6ccncc6)nc5)C4)ccc3C2=O)C(=O)N1] | 3-(5-((1-([2,4′-bipyridin]-5- ylmethyl)azetidin-3- yl)oxy)-1-oxoisoindolin-2- yl)piperidine-2,6-dione |
| 128 GT- 06353 | 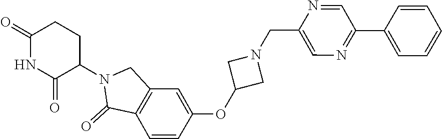 [SMILES: O=C1CCC(N2Cc3cc(OC4CN(Cc5cnc(-c6ccccc6)cn5)C4)ccc3C2=O)C(=O)N1] | 3-(1-oxo-5-((1-((5- phenylpyrazin-2- yl)methyl)azetidin-3- yl)oxy)isoindolin-2- yl)piperidine-2,6-dione |
| 129 GT- 06562 | 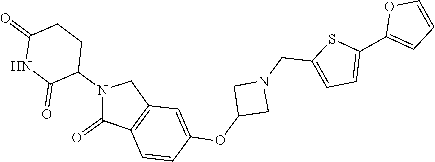 [SMILES: O=C1CCC(N2Cc3cc(OC4CN(Cc5ccc(-c6ccco6)s5)C4)ccc3C2=O)C(=O)N1] | 3-(5-((1-((5-(furan-2- yl)thiophen-2- yl)methyl)azetidin-3- yl)oxy)-1-oxoisoindolin-2- yl)piperidine-2,6-dione |
| 130 GT- 03459 | 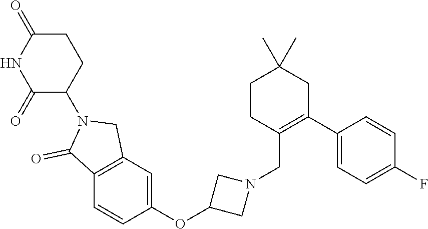 [SMILES: CC1(C)CCC(CN2CC(Oc3ccc4c(c3)CN(C3CCC(=O)NC3=O)C4=O)C2)=C(c2ccc(F)cc2)C1] | 3-(5-((1-((4′-fluoro-5,5- dimethyl-3,4,5,6- tetrahydro-[1,1′-biphenyl]- 2-yl)methyl)azetidin-3- yl)oxy)-1-oxoisoindolin-2- yl)piperidine-2,6-dione |
| 131 GT- 07062 | 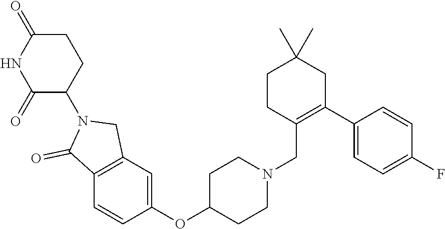 [SMILES: CC1(C)CCC(CN2CCC(Oc3ccc4c(c3)CN(C3CCC(=O)NC3=O)C4=O)CC2)=C(c2ccc(F)cc2)C1] | 3-(5-((1-((4′-fluoro-5,5- dimethyl-3,4,5,6- tetrahydro-[1,1′-biphenyl]- 2-yl)methyl)piperidin-4- yl)oxy)-1-oxoisoindolin-2- yl)piperidine-2,6-dione |
| 132 | 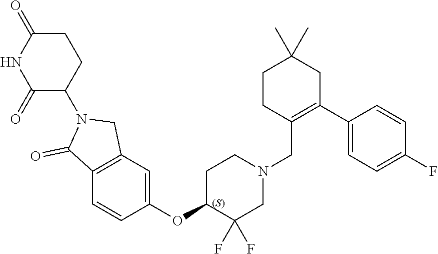 | 3-(5-(((S)-3,3-difluoro-1- ((4′-fluoro-5,5-dimethyl- 3,4,5,6-tetrahydro-[1,1′- biphenyl]-2- yl)methyl)piperidin-4- yl)oxy)-1-oxoisoindolin-2- yl)piperidine-2,6-dione |
| 133 GT- 06302 | 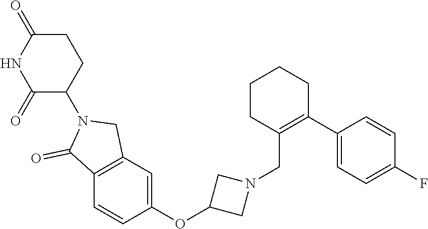 [SMILES: O=C1CCC(N2Cc3cc(OC4CN(CC5=C(c6ccc(F)cc6)CCCC5)C4)ccc3C2=O)C(=O)N1] | 3-(5-((1-((4′-fluoro-3,4,5,6- tetrahydro-[1,1′-biphenyl]- 2-yl)methyl)azetidin-3- yl)oxy)-1-oxoisoindolin-2- yl)piperidine-2,6-dione |
| 134 GT- 06354 | 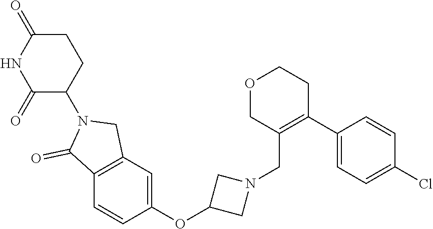 [SMILES: O=C1CCC(N2Cc3cc(OC4CN(CC5=C(c6ccc(Cl)cc6)CCOC5)C4)ccc3C2=O)C(=O)N1] | 3-(5-((1-((4-(4- chlorophenyl)-5,6-dihydro- 2H-pyran-3- yl)methyl)azetidin-3- yl)oxy)-1-oxoisoindolin-2- yl)piperidine-2,6-dione |
| 135 GT- 06560 | 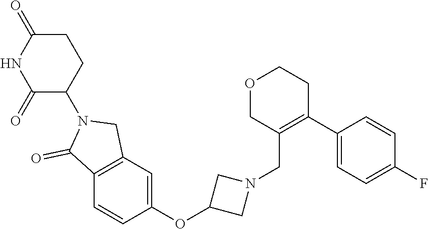 [SMILES: O=C1CCC(N2Cc3cc(OC4CN(CC5=C(c6ccc(F)cc6)CCOC5)C4)ccc3C2=O)C(=O)N1] | 3-(5-((1-((4-(4- fluorophenyl)-5,6-dihydro- 2H-pyran-3- yl)methyl)azetidin-3- yl)oxy)-1-oxoisoindolin-2- yl)piperidine-2,6-dione |
| 136 GT- 06563 | 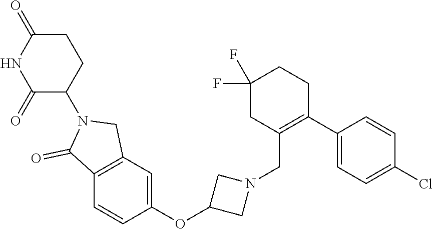 [SMILES: O=C1CCC(N2Cc3cc(OC4CN(CC5=C(c6ccc(Cl)cc6)CCC(F)(F)C5)C4)ccc3C2=O)C(=O)N1] | 3-(5-((1-((4′-chloro-4,4- difluoro-3,4,5,6-tetrahydro- [1,1′-biphenyl]-2- yl)methyl)azetidin-3- yl)oxy)-1-oxoisoindolin-2- yl)piperidine-2,6-dione |
| 137 GT- 07065 | 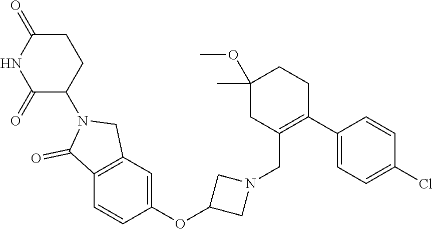 [SMILES: COC1(C)CCC(c2ccc(Cl)cc2)=C(CN2CC(Oc3ccc4c(c3)CN(C3CCC(=O)NC3=O)C4=O)C2)C1] | 3-(5-((1-((4′-chloro-4- methoxy-4-methyl-3,4,5,6- tetrahydro-[1,1′-biphenyl]- 2-yl)methyl)azetidin-3- yl)oxy)-1-oxoisoindolin-2- yl)piperidine-2,6-dione |
| 138 GT- 06561 | 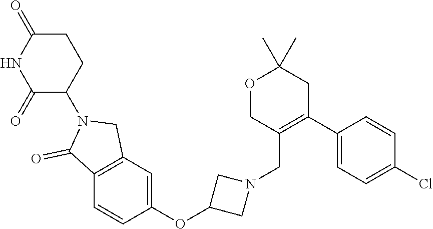 [SMILES: CC1(C)CC(c2ccc(Cl)cc2)=C(CN2CC(Oc3ccc4c(c3)CN(C3CCC(=O)NC3=O)C4=O)C2)CO1] | 3-(5-((1-((4-(4- chlorophenyl)-6,6- dimethyl-5,6-dihydro-2H- pyran-3-yl)methyl)azetidin- 3-yl)oxy)-1-oxoisoindolin- 2-yl)piperidine-2,6-dione |
| 139 | 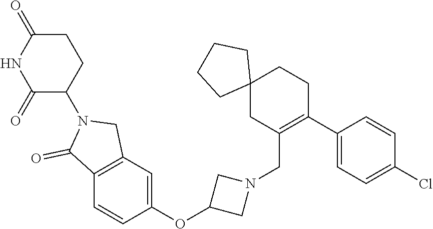 [SMILES: O=C1CCC(N2Cc3cc(OC4CN(CC5=C(c6ccc(Cl)cc6)CCC6(CCCC6)C5)C4)ccc3C2=O)C(=O)N1] | 3-(5-((1-((8-(4- chlorophenyl)spiro[4.5]dec- 7-en-7-yl)methyl)azetidin- 3-yl)oxy)-1-oxoisoindolin- 2-yl)piperidine-2,6-dione |
| 140 | 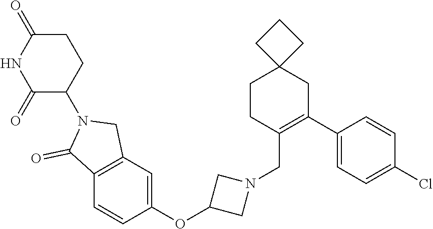 [SMILES: O=C1CCC(N2Cc3cc(OC4CN(CC5=C(c6ccc(Cl)cc6)CC6(CCC6)CC5)C4)ccc3C2=O)C(=O)N1] | 3-(5-((1-((6-(4- chlorophenyl)spiro[3.5]non- 6-en-7-yl)methyl)azetidin- 3-yl)oxy)-1-oxoisoindolin- 2-yl)piperidine-2,6-dione |
| 141 GT- 06185 | 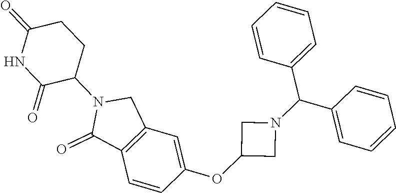 [SMILES: O=C1CCC(N2Cc3cc(OC4CN(C(c5ccccc5)c5ccccc5)C4)ccc3C2=O)C(=O)N1] | 3-(5-((1- benzhydrylazetidin-3- yl)oxy)-1-oxoisoindolin-2- yl)piperidine-2,6-dione |
| 142 GT- 06819 | 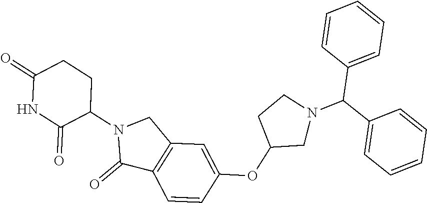 [SMILES: O=C1CCC(N2Cc3cc(OC4CCN(C(c5ccccc5)c5ccccc5)C4)ccc3C2=O)C(=O)N1] | 3-(5-((1- benzhydrylpyrrolidin-3- yl)oxy)-1-oxoisoindolin-2- yl)piperidine-2,6-dione |
| 143 GT- 06195 | 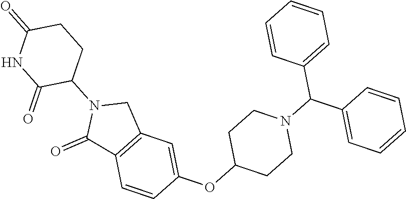 [SMILES: O=C1CCC(N2Cc3cc(OC4CCN(C(c5ccccc5)c5ccccc5)CC4)ccc3C2=O)C(=O)N1] | 3-(5-((1- benzhydrylpiperidin-4- yl)oxy)-1-oxoisoindolin-2- yl)piperidine-2,6-dione |
| 144 GT- 06928 | 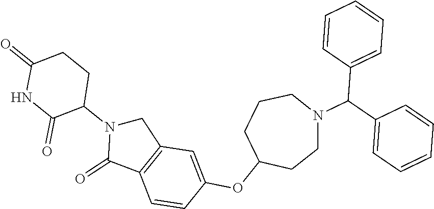 [SMILES: O=C1CCC(N2Cc3cc(OC4CCCN(C(c5ccccc5)c5ccccc5)CC4)ccc3C2=O)C(=O)N1] | 3-(5-((1-benzhydrylazepan- 4-yl)oxy)-1-oxoisoindolin- 2-yl)piperidine-2,6-dione |
| 145 GT- 06956 | 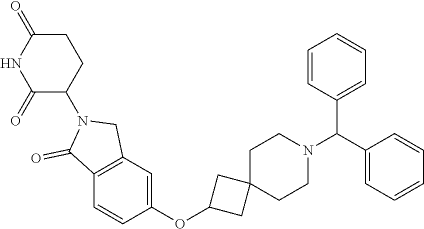 [SMILES: O=C1CCC(N2Cc3cc(OC4CC5(CCN(C(c6ccccc6)c6ccccc6)CC5)C4)ccc3C2=O)C(=O)N1] | 3-(5-((7-benzhydryl-7- azaspiro[3.5]nonan-2- yl)oxy)-1-oxoisoindolin-2- yl)piperidine-2,6-dione |
| 146 | 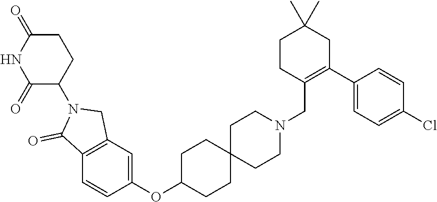 [SMILES: CC1(C)CCC(CN2CCC3(CCC(Oc4ccc5c(c4)CN(C4CCC(=O)NC4=O)C5=O)CC3)CC2)=C(c2ccc(Cl)cc2)C1] | 3-(5-((3-((4′-chloro-5,5- dimethyl-3,4,5,6- tetrahydro-[1,1′-biphenyl]- 2-yl)methyl)-3- azaspiro[5.5]undecan-9- yl)oxy)-1-oxoisoindolin-2- yl)piperidine-2,6-dione |
| 147 GT- 06955 | 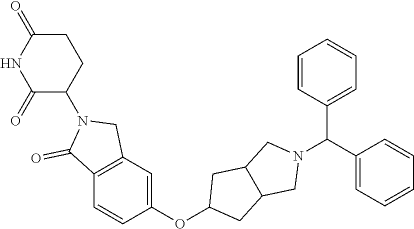 [SMILES: O=C1CCC(N2Cc3cc(OC4CC5CN(C(c6ccccc6)c6ccccc6)CC5C4)ccc3C2=O)C(=O)N1] | 3-(5-((2- benzhydryloctahydro- cyclopenta[c]pyrrol- 5-yl)oxy)-1- oxoisoindolin-2- yl)piperidine-2,6-dione |
| 148 | 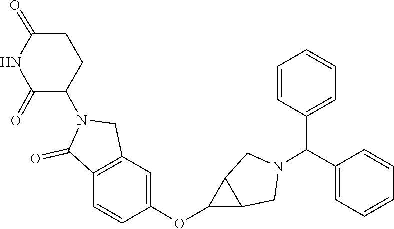 [SMILES: O=C1CCC(N2Cc3cc(OC4C5CN(C(c6ccccc6)c6ccccc6)CC54)ccc3C2=O)C(=O)N1] | 3-(5-((3-benzhydryl-3- azabicyclo[3.1.0]hexan-6- yl)oxy)-1-oxoisoindolin-2- yl)piperidine-2,6-dione |
| 149 | 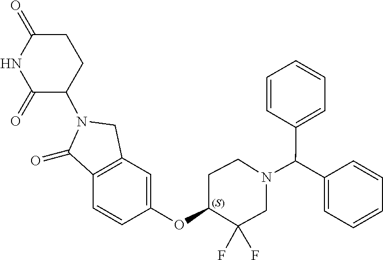 | 3-(5-(((S)-1-benzhydryl- 3,3-difluoropiperidin-4- yl)oxy)-1-oxoisoindolin-2- yl)piperidine-2,6-dione |
| 150 | 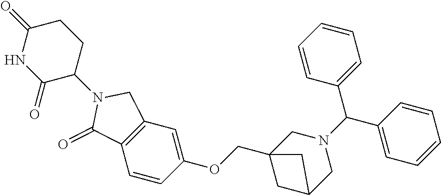 [SMILES: O=C1CCC(N2Cc3cc(OCC45CC(CN(C(c6ccccc6)c6ccccc6)C4)C5)ccc3C2=O)C(=O)N1] | 3-(5-((3-benzhydryl-3- azabicyclo[3.1.1 ]heptan-1- yl)methoxy)-1- oxoisoindolin-2- yl)piperidine-2,6-dione |
| 151 GT- 06759 | 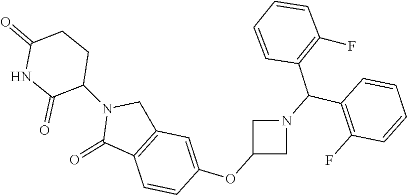 [SMILES: O=C1CCC(N2Cc3cc(OC4CN(C(c5ccccc5F)c5ccccc5F)C4)ccc3C2=O)C(=O)N1] | 3-(5-((1-(bis(2- fluorophenyl)methyl) azetidin-3-yl)oxy)-1- oxoisoindolin-2- yl)piperidine-2,6-dione |
| 152 GT- 06352 | 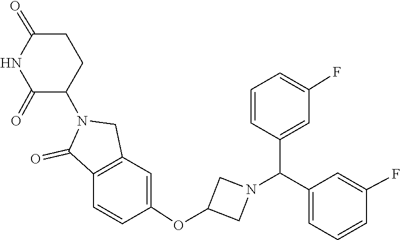 [SMILES: O=C1CCC(N2Cc3cc(OC4CN(C(c5cccc(F)c5)c5cccc(F)c5)C4)ccc3C2=O)C(=O)N1] | 3-(5-((1-(bis(3- fluorophenyl)methyl) azetidin-3-yl)oxy)-1- oxoisoindolin-2- yl)piperidine-2,6-dione |
| 153 GT- 06519 | 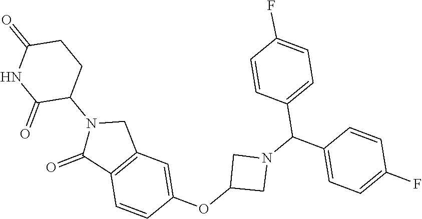 [SMILES: O=C1CCC(N2Cc3cc(OC4CN(C(c5ccc(F)cc5)c5ccc(F)cc5)C4)ccc3C2=O)C(=O)N1] | 3-(5-((1-(bis(4- fluorophenyl)methyl) azetidin-3-yl)oxy)-1- oxoisoindolin-2- yl)piperidine-2,6-dione |
| 154 GT- 06520 | 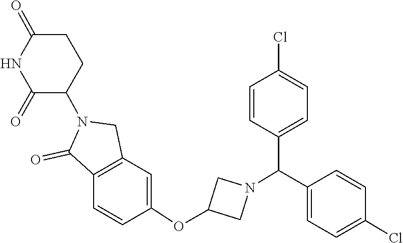 [SMILES: O=C1CCC(N2Cc3cc(OC4CN(C(c5ccc(Cl)cc5)c5ccc(Cl)cc5)C4)ccc3C2=O)C(=O)N1] | 3-(5-((1-(bis(4- chlorophenyl)methyl) azetidin-3-yl)oxy)-1- oxoisoindolin-2- yl)piperidine-2,6-dione |
| 155 | 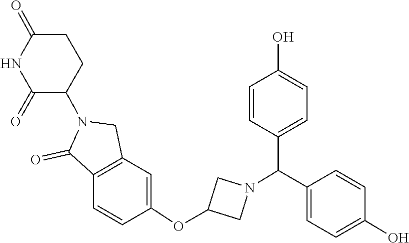 [SMILES: O=C1CCC(N2Cc3cc(OC4CN(C(c5ccc(O)cc5)c5ccc(O)cc5)C4)ccc3C2=O)C(=O)N1] | 3-(5-((1-(bis(4- hydroxyphenyl)methyl) azetidin-3-yl)oxy)-1- oxoisoindolin-2- yl)piperidine-2,6-dione |
| 156 GT- 06959 | 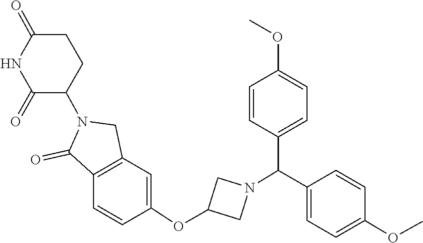 [SMILES: COc1ccc(C(c2ccc(OC)cc2)N2CC(Oc3ccc4c(c3)CN(C3CCC(=O)NC3=O)C4=O)C2)cc1] | 3-(5-((1-(bis(4- methoxyphenyl)methyl) azetidin-3-yl)oxy)-1- oxoisoindolin-2- yl)piperidine-2,6-dione |
| 157 | 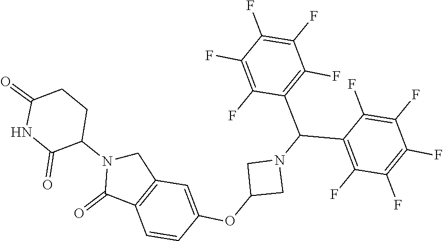 [SMILES: O=C1CCC(N2Cc3cc(OC4CN(C(c5c(F)c(F)c(F)c(F)c5F)c5c(F)c(F)c(F)c(F)c5F)C4)ccc3C2=O)C(=O)N1] | 3-(5-((1- (bis(perfluorophenyl) methyl)azetidin-3- yl)oxy)-1- oxoisoindolin-2- yl)piperidine-2,6-dione |
| 158 GT- 06559 | 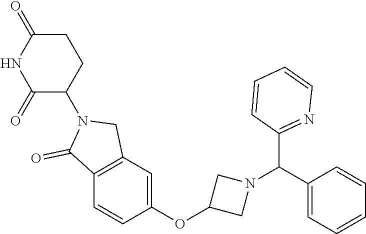 [SMILES: O=C1CCC(N2Cc3cc(OC4CN(C(c5ccccc5)c5ccccn5)C4)ccc3C2=O)C(=O)N1] | 3-(1-oxo-5-((1- (phenyl(pyridin-2- yl)methyl)azetidin-3- yl)oxy)isoindolin-2- yl)piperidine-2,6-dione |
| 159 GT- 06760 | 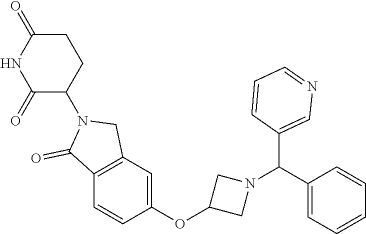 [SMILES: O=C1CCC(N2Cc3cc(OC4CN(C(c5ccccc5)c5cccnc5)C4)ccc3C2=O)C(=O)N1] | 3-(1-oxo-5-((1- (phenyl(pyridin-3- yl)methyl)azetidin-3- yl)oxy)isoindolin-2- yl)piperidine-2,6-dione |
| 160 | 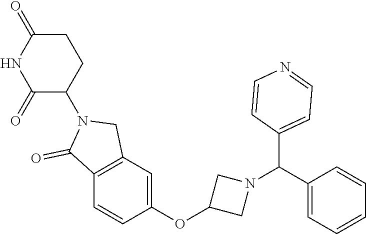 [SMILES: O=C1CCC(N2Cc3cc(OC4CN(C(c5ccccc5)c5ccncc5)C4)ccc3C2=O)C(=O)N1] | 3-(1-oxo-5-((1- (phenyl(pyridin-4- yl)methyl)azetidin-3- yl)oxy)isoindolin-2- yl)piperidine-2,6-dione |
| 161 GT- 06814 | 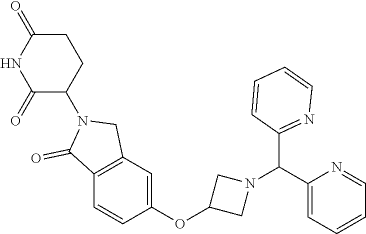 [SMILES: O=C1CCC(N2Cc3cc(OC4CN(C(c5ccccn5)c5ccccn5)C4)ccc3C2=O)C(=O)N1] | 3-(5-((1-(di(pyridin-2- yl)methyl)azetidin-3- yl)oxy)-1-oxoisoindolin-2- yl)piperidine-2,6-dione |
| 162 | 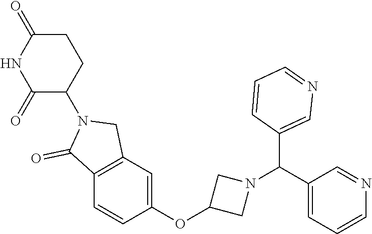 [SMILES: O=C1CCC(N2Cc3cc(OC4CN(C(c5cccnc5)c5cccnc5)C4)ccc3C2=O)C(=O)N1] | 3-(5-((1-(di(pyridin-3- yl)methyl)azetidin-3- yl)oxy)-1-oxoisoindolin-2- yl)piperidine-2,6-dione |
| 163 | 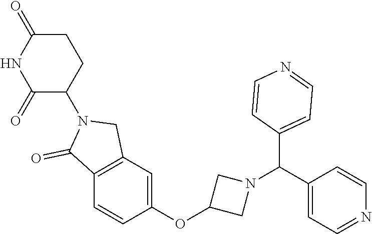 [SMILES: O=C1CCC(N2Cc3cc(OC4CN(C(c5ccncc5)c5ccncc5)C4)ccc3C2=O)C(=O)N1] | 3-(5-((1-(di(pyridin-4- yl)methyl)azetidin-3- yl)oxy)-1-oxoisoindolin-2- yl)piperidine-2,6-dione |
| 164 | 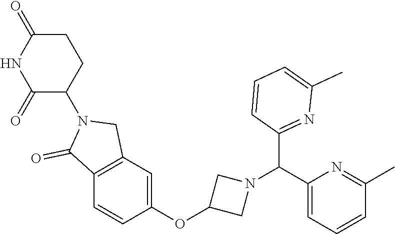 [SMILES: Cc1cccc(C(c2cccc(C)n2)N2CC(Oc3ccc4c(c3)CN(C3CCC(=O)NC3=O)C4=O)C2)n1] | 3-(5-((1-(bis(6- methylpyridin-2- yl)methyl)azetidin-3- yl)oxy)-1-oxoisoindolin-2- yl)piperidine-2,6-dione |
| 165 | 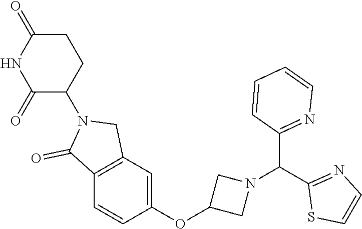 [SMILES: O=C1CCC(N2Cc3cc(OC4CN(C(c5ccccn5)c5nccs5)C4)ccc3C2=O)C(=O)N1] | 3-(1-oxo-5-((1-(pyridin-2- yl(thiazol-2- yl)methyl)azetidin-3- yl)oxy)isoindolin-2- yl)piperidine-2,6-dione |
| 166 | 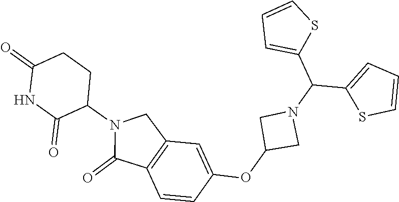 [SMILES: O=C1CCC(N2Cc3cc(OC4CN(C(c5cccs5)c5cccs5)C4)ccc3C2=O)C(=O)N1] | 3-(5-((1-(di(thiophen-2- yl)methyl)azetidin-3- yl)oxy)-1-oxoisoindolin-2- yl)piperidine-2,6-dione |
| 167 | 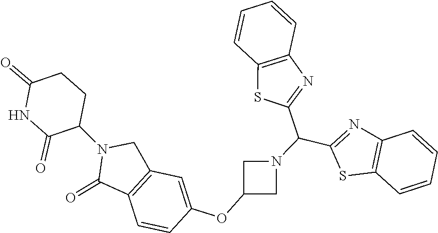 [SMILES: O=C1CCC(N2Cc3cc(OC4CN(C(c5nc6ccccc6s5)c5nc6ccccc6s5)C4)ccc3C2=O)C(=O)N1] | 3-(5-((1- (bis(benzo[d]thiazol-2- yl)methyl)azetidin-3- yl)oxy)-1-oxoisoindolin-2- yl)piperidine-2,6-dione |
| 168 GT- 06812 | 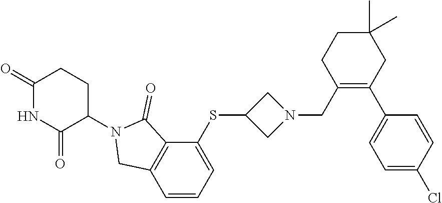 [SMILES: CC1(C)CCC(CN2CC(Sc3cccc4c3C(=O)N(C3CCC(=O)NC3=O)C4)C2)=C(c2ccc(Cl)cc2)C1] | 3-(7-((1-((4′-chloro-5,5- dimethyl-3,4,5,6- tetrahydro-[1,1′-biphenyl]- 2-yl)methyl)azetidin-3- yl)thio)-1-oxoisoindolin-2- yl)piperidine-2,6-dione |
| 169 GT- 06960 | 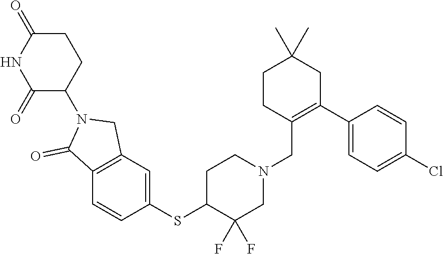 [SMILES: CC1(C)CCC(CN2CCC(Sc3ccc4c(c3)CN(C3CCC(=O)NC3=O)C4=O)C(F)(F)C2)=C(c2ccc(Cl)cc2)C1] | 3-(5-((1-((4′-chloro-5,5- dimethyl-3,4,5,6- tetrahydro-[1,1′-biphenyl]- 2-yl)methyl)-3,3- difluoropiperidin-4-yl)thio)- 1-oxoisoindolin-2- yl)piperidine-2,6-dione |
| 170 GT- 07022 | 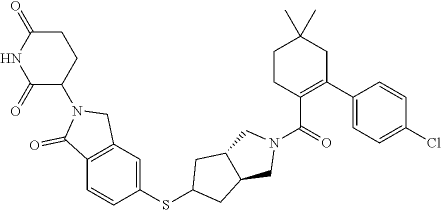 [SMILES: CC1(C)CCC(C(=O)N2C[C@H]3CC(Sc4ccc5c(c4)CN(C4CCC(=O)NC4=O)C5=O)C[C@@H]3C2)=C(c2ccc(Cl)cc2)C1] | 3-(5-(((3aS,6aS)-2-(4′- chloro-5,5-dimethyl- 3,4,5,6-tetrahydro-[1,1′- biphenyl]-2- carbonyl)octahydro- cyclopenta[c]pyrrol-5- yl)thio)-1- oxoisoindolin-2- yl)piperidine-2,6-dione |
| 171 GT- 07015 | 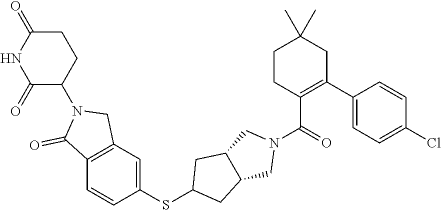 [SMILES: CC1(C)CCC(C(=O)N2C[C@H]3CC(Sc4ccc5c(c4)CN(C4CCC(=O)NC4=O)C5=O)C[C@H]3C2)=C(c2ccc(Cl)cc2)C1] | 3-(5-(((3aR,6aS)-2-(4′- chloro-5,5-dimethyl- 3,4,5,6-tetrahydro-[1,1′- biphenyl]-2- carbonyl)octahydro- cyclopenta[c]pyrrol- 5-yl)thio)-1- oxoisoindolin-2- yl)piperidine-2,6-dione |
| 172 GT- 07017 | 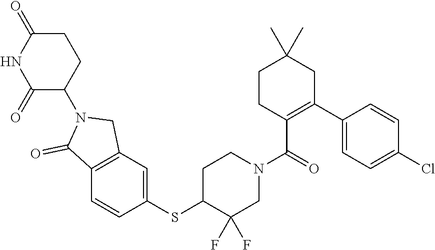 [SMILES: CC1(C)CCC(C(=O)N2CCC(Sc3ccc4c(c3)CN(C3CCC(=O)NC3=O)C4=O)C(F)(F)C2)=C(c2ccc(Cl)cc2)C1] | 3-(5-((1-(4′-chloro-5,5- dimethyl-3,4,5,6- tetrahydro-[1,1′-biphenyl]- 2-carbonyl)-3,3- difluoropiperidin-4-yl)thio)- 1-oxoisoindolin-2- yl)piperidine-2,6-dione |
| 173 GT- 06924 | 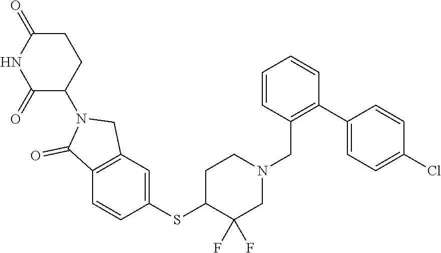 [SMILES: O=C1CCC(N2Cc3cc(SC4CCN(Cc5ccccc5-c5ccc(Cl)cc5)CC4(F)F)ccc3C2=O)C(=O)N1] | 3-(5-((1-((4′-chloro-[1,1′- biphenyl]-2-yl)methyl)-3,3- difluoropiperidin-4-yl)thio)- 1-oxoisoindolin-2- yl)piperidine-2,6-dione |
| 174 GT- 07023 | 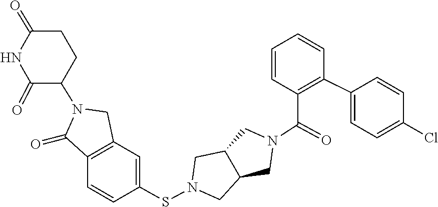 [SMILES: O=C1CCC(N2Cc3cc(SN4C[C@@H]5CN(C(=O)c6ccccc6-c6ccc(Cl)cc6)C[C@H]5C4)ccc3C2=O)C(=O)N1] | 3-(5-(((3aS,6aS)-2-(4′- chloro-[1,1′-biphenyl]-2- carbonyl)octahydro- cyclopenta[c]pyrrol- 5-yl)thio)-1- oxoisoindolin-2- yl)piperidine-2,6-dione |
| 175 GT- 07024 | 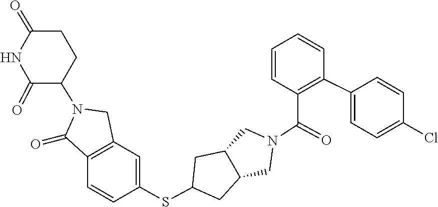 [SMILES: O=C1CCC(N2Cc3cc(SC4C[C@@H]5CN(C(=O)c6ccccc6-c6ccc(Cl)cc6)C[C@@H]5C4)ccc3C2=O)C(=O)N1] | 3-(5-(((3aR,6aS)-2-(4′- chloro-[1,1′-biphenyl]-2- carbonyl)octahydro- cyclopenta[c]pyrrol-5- yl)thio)-1- oxoisoindolin-2- yl)piperidine-2,6-dione |
| 176 GT- 06925 | 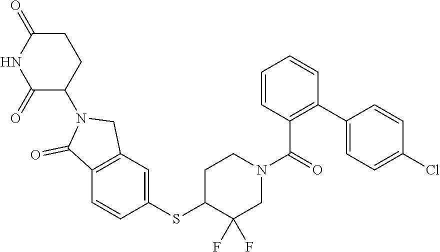 [SMILES: O=C1CCC(N2Cc3cc(SC4CCN(C(=O)c5ccccc5-c5ccc(Cl)cc5)CC4(F)F)ccc3C2=O)C(=O)N1] | 3-(5-((1-(4′-chloro-[1,1′- biphenyl]-2-carbonyl)-3,3- difluoropiperidin-4-yl)thio)- 1-oxoisoindolin-2- yl)piperidine-2,6-dione |
| 177 GT- 06926 | 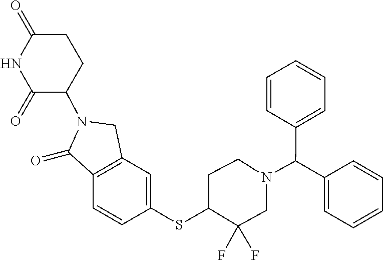 [SMILES: O=C1CCC(N2Cc3cc(SC4CCN(C(c5ccccc5)c5ccccc5)CC4(F)F)ccc3C2=O)C(=O)N1] | 3-(5-((1-benzhydryl-3,3- difluoropiperidin-4-yl)thio)- 1-oxoisoindolin-2- yl)piperidine-2,6-dione |
| 178 GT- 06952 | 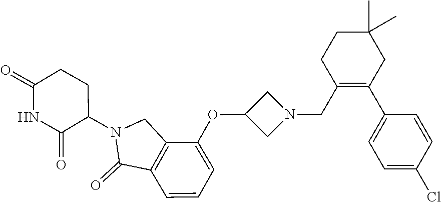 [SMILES: CC1(C)CCC(CN2CC(Oc3cccc4c3CN(C3CCC(=O)NC3=O)C4=O)C2)=C(c2ccc(Cl)cc2)C1] | 3-(4-((1-((4′-chloro-5,5- dimethyl-3,4,5,6- tetrahydro-[1,1′-biphenyl]- 2-yl)methyl)azetidin-3- yl)oxy)-1-oxoisoindolin-2- yl)piperidine-2,6-dione |
| 179 GT- 07063 | 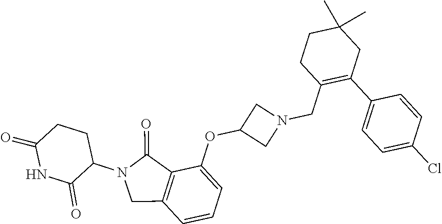 [SMILES: CC1(C)CCC(CN2CC(Oc3cccc4c3C(=O)N(C3CCC(=O)NC3=O)C4)C2)=C(c2ccc(Cl)cc2)C1] | 3-(7-((1-((4′-chloro-5,5- dimethyl-3,4,5,6- tetrahydro-[1,1′-biphenyl]- 2-yl)methyl))zetidine-3- yl)oxy)-1-oxoisoindolin-2- yl)piperidine-2,6-dione |
| 180 GT- 06930 | 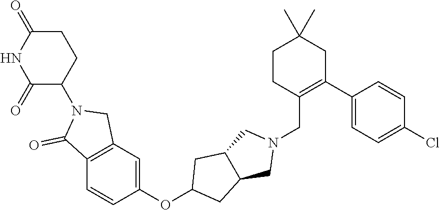 [SMILES: CC1(C)CCC(CN2C[C@H]3CC(Oc4ccc5c(c4)CN(C4CCC(=O)NC4=O)C5=O)C[C@@H]3C2)=C(c2ccc(Cl)cc2)C1] | 3-(5-(((3aS,6aS)-2-((4′- chloro-5,5-dimethyl- 3,4,5,6-tetrahydro-[1,1′- biphenyl]-2- yl)methyl)octahydro- cyclopenta[c]pyrrol- 5-yl)oxy)-1- oxoisoindolin-2- yl)piperidine-2,6-dione |
| 181 GT- 06931 | 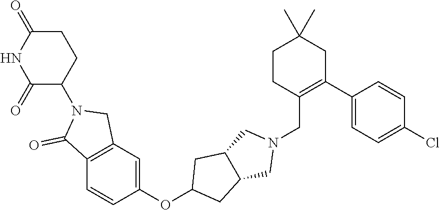 [SMILES: CC1(C)CCC(CN2C[C@H]3CC(Oc4ccc5c(c4)CN(C4CCC(=O)NC4=O)C5=O)C[C@H]3C2)=C(c2ccc(Cl)cc2)C1] | 3-(5-(((3aR,6aS)-2-((4′- chloro-5,5-dimethyl- 3,4,5,6-tetrahydro-[1,1′- biphenyl]-2- yl)methyl)octahydro- cyclopenta[c]pyrrol- 5-yl)oxy)-1- oxoisoindolin-2- yl)piperidine-2,6-dione |
| 182 GT- 07025 | 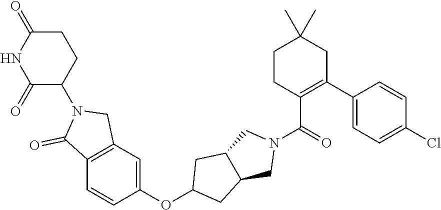 [SMILES: CC1(C)CCC(C(=O)N2C[C@H]3CC(Oc4ccc5c(c4)CN(C4CCC(=O)NC4=O)C5=O)C[C@@H]3C2)=C(c2ccc(Cl)cc2)C1] | 3-(5-(((3aS,6aS)-2-(4′- chloro-5,5-dimethyl- 3,4,5,6-tetrahydro-[1,1′- biphenyl]-2- carbonyl)octahydro- cyclopenta[c]pyrrol- 5-yl)oxy)-1- oxoisoindolin-2- yl)piperidine-2,6-dione |
| 183 GT- 07019 | 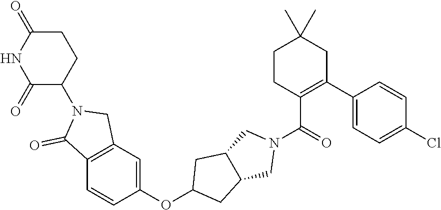 [SMILES: CC1(C)CCC(C(=O)N2C[C@H]3CC(Oc4ccc5c(c4)CN(C4CCC(=O)NC4=O)C5=O)C[C@H]3C2)=C(c2ccc(Cl)cc2)C1] | 3-(5-(((3aR,6aS)-2-(4′- chloro-5,5-dimethyl- 3,4,5,6-tetrahydro-[1,1′- biphenyl]-2- carbonyl)octahydro- cyclopenta[c]pyrrol- 5-yl)oxy)-1- oxoisoindolin-2- yl)piperidine-2,6-dione |
| 184 GT- 06228 | 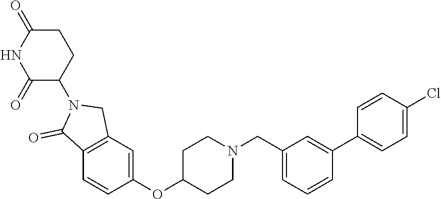 [SMILES: O=C1CCC(N2Cc3cc(OC4CCN(Cc5cccc(-c6ccc(Cl)cc6)c5)CC4)ccc3C2=O)C(=O)N1] | 3-(5-((1-((4′-chloro-[1,1′- biphenyl]-3- yl)methyl)piperidin-4- yl)oxy)-1-oxoisoindolin-2- yl)piperidine-2,6-dione |

Specifically, the aforementioned structure may be further substituted by one or more substituents selected from the group consisting of: deuterium, halogen, cyano, nitro, hydroxy, carbonyl, ester group, imide group, phosphine oxide group, alkoxy, aryloxy, alkylthio, arylthio, alkylsulfonyl, arylsulfonyl, silyl, boryl, alkyl, cycloalkyl, alkenyl, aryl, aralkyl, arylalkenyl, alkylaryl, alkylamino, aralkylamino, heteroarylamino, arylamino, arylphosphino, and heteroaryl etc.

In another aspect, the present disclosure provides an embodiment A1): the compound of Formula A:

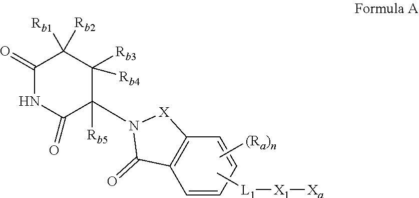

or a salt thereof (including pharmaceutically acceptable salts), enantiomers, stereoisomers, polymorphs or solvates thereof,
wherein X is selected from the group consisting of CO or CH₂;
L₁ is selected from the group consisting of S or O;
X₁ is selected from the group consisting of unsubstituted or substituted linear or branched alkylene, or X₁ is absent;
Xₐ represents the structure of Formula A1:

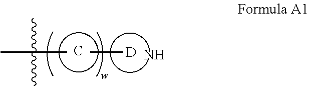

wherein ring C is selected from the group consisting of unsubstituted or substituted arylene, unsubstituted or substituted heteroarylene, unsubstituted or substituted heterocyclylene, or unsubstituted or substituted cycloalkylene, and w represents an integer of 0 or 1; and
ring D represents unsubstituted or substituted nitrogen-containing heterocyclyl;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl;
R_{b1}, R_{b2}, R_{b3}, R_{b4} and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl; and
n is an integer of 0, 1, 2 or 3, and when n is an integer of 2 or 3, each Rₐ is independently identical to or different from each other.

The present disclosure provides a compound of Formula A or a salt thereof (including pharmaceutically acceptable salts), stereoisomers (including enantiomers), polymorphs, or solvates thereof, which exhibit CRBN E3 ubiquitin ligase binding activity to facilitate substrate protein recruitment, thereby demonstrating immunomodulatory, anti-inflammatory, and anti-tumor activities or favorable pharmacokinetic properties, and which may serve as intermediates for preparing the compounds of Formula I or salts, stereoisomers (including enantiomers), polymorphs, or solvates thereof of the present disclosure.

Embodiment A2): Pertaining to the compound of Formula A or salts, enantiomers, stereoisomers, polymorphs or solvates thereof as described in embodiment A1), wherein X in Formula A is CO.

Embodiment A3): Pertaining to the compound of Formula A or salts, enantiomers, stereoisomers, polymorphs or solvates thereof as described in embodiment A1), wherein X in Formula is CH₂.

Embodiment A4): Pertaining to the compound of Formula A or salts, enantiomers, stereoisomers, polymorphs or solvates thereof as described in embodiment A1), wherein L₁ in Formula A is S.

Embodiment A5): Pertaining to the compound of Formula A or salts, enantiomers, stereoisomers, polymorphs or solvates thereof as described in embodiment A1), wherein L₁ in Formula A is O.

Embodiment A6): Pertaining to the compound of Formula A or salts, enantiomers, stereoisomers, polymorphs or solvates thereof as described in any one of embodiments A1)-A5), wherein X₁ is unsubstituted or substituted linear or branched alkylene. When X₁ is unsubstituted or substituted linear or branched alkylene, the X₁ represents unsubstituted or substituted linear or branched C₁-C₅ alkylene, or unsubstituted or substituted linear or branched C₁-C₃ alkylene etc.

Embodiment A7): Pertaining to the compound of Formula A or salts, enantiomers, stereoisomers, polymorphs or solvates thereof as described in any one of embodiments A1)-A6), wherein when X₁ is unsubstituted or substituted linear or branched C₁-C₅alkylene, the linear or branched C₁-C₅alkylene may be methylene, ethylene, propylene, n-propylene, isopropylene, butylene, n-butylene, isobutylene, tert-butylene, sec-butylene, 1-methyl-butylene, 1-ethyl-butylene, pentylene, n-pentylene, isopentylene, neopentylene, or tert-pentylene etc.

Embodiment A8): Pertaining to the compound of Formula A or salts, enantiomers, stereoisomers, polymorphs or solvates thereof as described in any one of embodiments A1)-A7), wherein when X₁ is unsubstituted or substituted linear or branched C₁-C₃ alkylene, the linear or branched alkylene may be e.g., methylene, ethylene, propylene, or isopropylene.

Embodiment A9): Pertaining to the compound of Formula A or salts, enantiomers, stereoisomers, polymorphs or solvates thereof as described in any one of embodiments A1)-A8), wherein the X₁ is selected from the group consisting of methylene.

Embodiment A10): Pertaining to the compound of Formula A or salts, enantiomers, stereoisomers, polymorphs or solvates thereof as described in any one of embodiments A1)-A8), wherein the substituents of the substituted X₁ is optionally selected from the group consisting of deuterium, halogen, cyano, nitro, hydroxy, mercapto, carbonyl, ester group, imide group, amino, phosphine oxide group, alkoxy, halogenated alkoxy, aryloxy, alkylthio, arylthio, alkylsulfonyl, arylsulfonyl, silyl, boryl, alkyl, cycloalkyl, alkenyl, aryl, aralkyl, arylalkenyl, alkyl aryl, alkylamino, aralkylamino, heteroaryl amino, aryl amino, arylphosphino, heteroaryl, and acenaphthenyl etc., or any combination thereof. Optionally, the substituents of the substituted X₁ may be e.g., one or more substituents selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl), nitro, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo or any combination thereof. Further optionally, the substituents of the substituted X₁ may be e.g., selected from the group consisting of deuterium, halogen, nitro, mercapto, hydroxy, cyano, C₁-C₃ alkyl, C₁-C₃ alkoxy, halogenated C₁-C₃ alkoxy, halogenated C₁-C₃ alkyl, amino, silyl, boryl, or any combination thereof. Further optionally, the substituents of the substituted X₁ may be e.g., selected from the group consisting of deuterium, F, Cl, methoxy, methyl, hydroxy, or any combination thereof.

Embodiment A11): Pertaining to the compound of Formula A or salts, enantiomers, stereoisomers, polymorphs or solvates thereof as described in any one of embodiments A1)-A5), wherein X₁ is absent.

Embodiment A12): Pertaining to the compound of Formula A or salts, enantiomers, stereoisomers, polymorphs or solvates thereof as described in any one of embodiments A1)-A11), wherein Xₐ represents the structure of Formula A1:

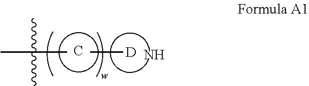

wherein ring C is selected from the group consisting of unsubstituted or substituted C₅-C₃₀ arylene, unsubstituted or substituted 5- to 30-membered heteroarylene, unsubstituted or substituted 4- to 30-membered heterocyclylene, or unsubstituted or substituted C₃-C₃₀ cycloalkylene; preferably, ring C is selected from the group consisting of unsubstituted or substituted C₅-C₂₀ arylene, unsubstituted or substituted 5- to 20-membered heteroarylene, unsubstituted or substituted 4- to 20-membered heterocyclylene, or unsubstituted or substituted C₃-C₂₀ cycloalkylene;
w represents an integer of 0 or 1; and
ring D represents unsubstituted or substituted 4- to 30-membered nitrogen-containing heterocyclyl; preferably, ring D represents unsubstituted or substituted 4- to 20-membered nitrogen-containing heterocyclyl.

Embodiment A13): Pertaining to the compound of Formula A or salts, enantiomers, stereoisomers, polymorphs or solvates thereof as described in any one of embodiments A1)-A12), wherein Xₐ represents the structure of Formula A1:

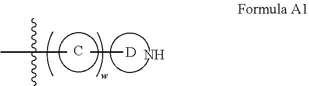

wherein ring C is selected from the group consisting of unsubstituted or substituted C₅-C₁₅ arylene, unsubstituted or substituted 5- to 15-membered heteroarylene, unsubstituted or substituted 4- to 15-membered heterocyclylene, or unsubstituted or substituted C₃-C₁₅ cycloalkylene, wherein the substituents of the substituted ring C are optionally one or more substituents selected from the group consisting of halogen, mercapto, hydroxy, amino, cyano, C₁-C₆ alkyl, C₁-C₆ alkoxy, halogenated C₁-C₆ alkyl, or any combination thereof;
w represents an integer of 0 or 1; and
ring D represents unsubstituted or substituted 4- to 15-membered nitrogen-containing heterocyclyl.

Embodiment A14): Pertaining to the compound of Formula A or salts, enantiomers, stereoisomers, polymorphs or solvates thereof as described in any one of embodiments A1)-A13), wherein the ring C is selected from the group consisting of the following groups: azetidinylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, azacyclooctylene, diazacycloheptanylene, diazacyclooctylene, bridged heterocyclylene (e.g., 5- to 15-membered bridged heterocyclylene, such as azabicyclohexylene, azabicycloheptanylene or azabicyclooctylene), or spiro-heterocyclylene (e.g., 5- to 15-membered spiro-heterocyclylene, such as 3-azaspiro[5.5]undecanylene or 7-azaspiro[3.5]nonanylene); or
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene; or
cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₅-C₁₅ spiro-cycloalkylene, such as spiro[3.3]heptylene, spiro[2.5]octylene, spiro[3.5]nonylene, spiro[4.4]nonylene, spiro[4.5]decylene, or spiro[5.5]undecylene), or bridged cycloalkylene (e.g., C₅-C₁₅ bridged cycloalkylene, adamantanylene, noradamantanylene, bornylene, bicyclo[2.2.1]heptylene, 2-oxobicyclo[2.2.1]heptylene, or bicyclo[2.2.1]heptentylene); or
phenylene or naphthylene;
wherein ring C is optionally substituted by one or more substituents selected from the group consisting of deuterium, halogen, nitro, mercapto, hydroxy, cyano, C₁-C₆ alkyl, C₁-C₆ alkoxy, halogenated C₁-C₆ alkoxy, halogenated C₁-C₆ alkyl, unsubstituted or substituted amino, unsubstituted or substituted silyl, unsubstituted or substituted boryl, or any combination thereof.

Embodiment A15): Pertaining to the compound of Formula A or salts, enantiomers, stereoisomers, polymorphs or solvates thereof as described in any one of embodiments A1)-A14), wherein the ring D is selected from the group consisting of the following groups:
azetidinyl, pyrrolidinyl, imidazolidinyl, pyrazolidyl, oxazolidinyl, thiazolidinyl, piperidinyl, piperazinyl, morpholinyl, thiomorpholinyl, azacycloheptanyl, azacyclooctyl, diazacycloheptanyl, diazacyclooctyl, nitrogen-containing bridged heterocyclyl (e.g., 5- to 15-membered bridged heterocyclyl, such as azabicyclohexyl, azabicycloheptanyl or azabicyclooctyl), or nitrogen-containing spiro-heterocyclyl (e.g., 5- to 15-membered spiro-heterocyclyl, such as 3-azaspiro[5.5]undecanyl or 7-azaspiro[3.5]nonanyl);
wherein the ring D is optionally substituted by one or more substituents selected from the group consisting of deuterium, halogen, nitro, mercapto, hydroxy, cyano, oxo, C₁-C₆ alkyl, C₁-C₆ alkoxy, halogenated C₁-C₆ alkoxy, halogenated C₁-C₆ alkyl, unsubstituted or substituted amino, unsubstituted or substituted silyl, unsubstituted or substituted boryl, or any combination thereof.

Embodiment A16): Pertaining to the compound of Formula A or salts, enantiomers, stereoisomers, polymorphs or solvates thereof as described in any one of embodiments A1)-A15), wherein Xₐ is selected from the group consisting of:

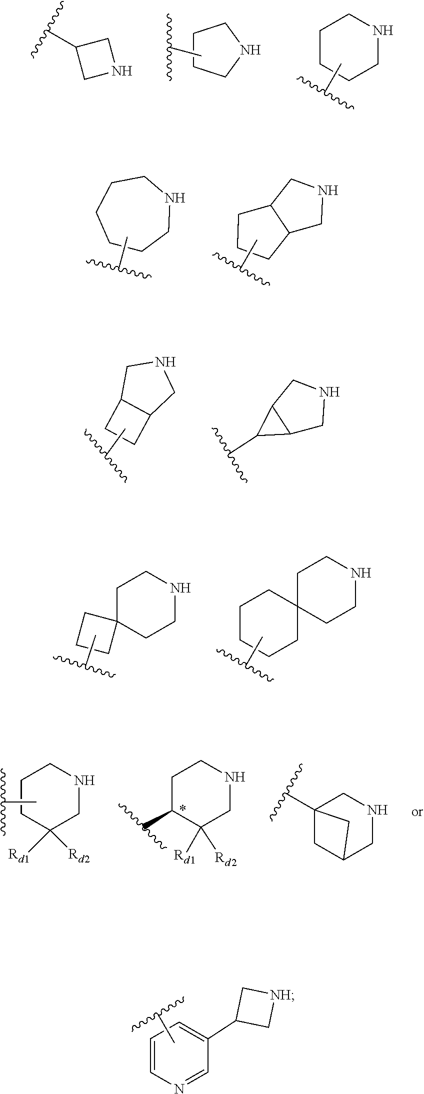

wherein R_{d1} and Rd₂ are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, and unsubstituted or substituted linear or branched C₁-C₅alkyl; and R_{d1} and Rd₂ are not hydrogen at the same time.

Embodiment A17): Pertaining to the compound of Formula A or salts, enantiomers, stereoisomers, polymorphs or solvates thereof as described in embodiment A16), wherein R_{d1} and R_{d2} are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, and unsubstituted or substituted linear or branched C₁-C₅alkyl; and R_{d1} and R_{d2} are not hydrogen at the same time. In specific embodiments, optionally, the halogen may be e.g., F, Cl, Br or I. Optionally, the C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, and 2-ethylbutoxy etc. Optionally, the halogenated C₁-C₆ alkoxy may be e.g., trifluoromethoxy etc. Optionally, the unsubstituted or substituted amino includes amino, N-methylamino, N-ethylamino, N-phenylamino, N,N-dimethylamino, and N,N-diethylamino etc. Optionally, the unsubstituted or substituted silyl may be e.g., trimethylsilyl, triethylsilyl, tert-butyldimethylsilyl, vinyldimethylsilyl, propyldimethylsilyl, triphenylsilyl, diphenylsilyl, and phenylsilyl etc. Optionally, the unsubstituted or substituted boryl may include e.g., trimethylboryl, triethylboryl, tert-butyldimethylboryl, triphenylboryl, and phenylboryl etc. Optionally, the linear or branched C₁-C₅ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, and tert-pentyl etc. Wherein said linear or branched C₁-C₅alkyl is optionally substituted with one or more substituents optionally selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl), or any combination thereof.

Embodiment A18): Pertaining to the compound of Formula A or salts, enantiomers, stereoisomers, polymorphs or solvates thereof as described in any one of embodiments A1)-A17), wherein Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl.

Embodiment A19): Pertaining to the compound of Formula A or salts, enantiomers, stereoisomers, polymorphs or solvates thereof as described in embodiment A18), wherein Rₐ is selected from the group consisting of deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, unsubstituted or substituted linear or branched C₁-C₅alkyl, unsubstituted or substituted C₃-C₁₅ cycloalkyl, unsubstituted or substituted 4- to 15-membered heterocyclyl, unsubstituted or substituted C₅-C₁₅ aryl, or unsubstituted or substituted 5- to 15-membered heteroaryl.

Embodiment A20): Pertaining to the compound of Formula A or salts, enantiomers, stereoisomers, polymorphs or solvates thereof as described in embodiment A18) or A19), wherein Rₐ is selected from the group consisting of deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, unsubstituted or substituted linear or branched C₁-C₅ alkyl, unsubstituted or substituted C₃-C₁₅ cycloalkyl, unsubstituted or substituted 4- to 15-membered heterocyclyl, unsubstituted or substituted C₅-C₁₅ aryl, or unsubstituted or substituted 5- to 15-membered heteroaryl.

Embodiment A21): Pertaining to the compound of Formula A or salts, enantiomers, stereoisomers, polymorphs or solvates thereof as described in embodiment A20), wherein Rₐ is deuterium.

Embodiment A22): Pertaining to the compound of Formula A or salts, enantiomers, stereoisomers, polymorphs or solvates thereof as described in embodiment A20), wherein Rₐ is halogen, and the halogen is optionally F, Cl, Br, or I.

Embodiment A23): Pertaining to the compound of Formula A or salts, enantiomers, stereoisomers, polymorphs or solvates thereof as described in embodiment A20), wherein Rₐ is nitro.

Embodiment A24): Pertaining to the compound of Formula A or salts, enantiomers, stereoisomers, polymorphs or solvates thereof as described in embodiment A20), wherein Rₐ is cyano.

Embodiment A25): Pertaining to the compound of Formula A or salts, enantiomers, stereoisomers, polymorphs or solvates thereof as described in embodiment A20), wherein Rₐ is amino.

Embodiment A26): Pertaining to the compound of Formula A or salts, enantiomers, stereoisomers, polymorphs or solvates thereof as described in embodiment A20), wherein Rₐ is hydroxy.

Embodiment A27): Pertaining to the compound of Formula A or salts, enantiomers, stereoisomers, polymorphs or solvates thereof as described in embodiment A20), wherein Rₐ is mercapto.

Embodiment A28): Pertaining to the compound of Formula A or salts, enantiomers, stereoisomers, polymorphs or solvates thereof as described in embodiment A20), wherein Rₐ is alkoxy. Optionally, when Rₐ is alkoxy, the alkoxy is C₁-C₆ alkoxy. C₁-C₆ alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, and 2-ethylbutoxy etc.

Embodiment A29): Pertaining to the compound of Formula A or salts, enantiomers, stereoisomers, polymorphs or solvates thereof as described in embodiment A20), wherein Rₐ is halogenated alkoxy.

Optionally, when Rₐ is halogenated alkoxy, the halogenated alkoxy is halogenated C₁-C₆ alkoxy; Further optionally, the halogenated C₁-C₆ alkoxy is trifluoromethoxy etc.

Embodiment A30): Pertaining to the compound of Formula A or salts, enantiomers, stereoisomers, polymorphs or solvates thereof as described in embodiment A20), wherein Rₐ is unsubstituted or substituted amino. Optionally, the unsubstituted or substituted amino includes amino, N-methylamino, N-ethylamino, N-phenylamino, N,N-dimethylamino, and N,N-diethylamino etc.

Embodiment A31): Pertaining to the compound of Formula A or salts, enantiomers, stereoisomers, polymorphs or solvates thereof as described in embodiment A20), wherein Rₐ is unsubstituted or substituted silyl. Optionally, the unsubstituted or substituted silyl is specifically trimethylsilyl, triethylsilyl, tert-butyldimethylsilyl, vinyldimethylsilyl, propyldimethylsilyl, triphenylsilyl, diphenylsilyl, and phenylsilyl etc.

Embodiment A32): Pertaining to the compound of Formula A or salts, enantiomers, stereoisomers, polymorphs or solvates thereof as described in embodiment A20), wherein Rₐ is unsubstituted or substituted boryl. Optionally, the unsubstituted or substituted boryl is specifically trimethylboryl, triethylboryl, tert-butyldimethylboryl, triphenylboryl, and phenylboryl etc.

Embodiment A33): Pertaining to the compound of Formula A or salts, enantiomers, stereoisomers, polymorphs or solvates thereof as described in embodiment A20), wherein Rₐ is unsubstituted or substituted linear or branched alkyl, optionally the number of carbon atoms may be for example C₁-C₃₀, C₁-C₂₅, C₁-C₂₀, C₁-C₁₅, C₁-C₁₀, C₁-C₅, and C₁-C₃ etc, preferably linear or branched C₁-C₁₀ alkyl. Further optionally, the linear or branched C₁-C₁₀ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, nonyl, 2,2-dimethylheptyl, 1-ethylpropyl, 1,1-dimethylpropyl, isohexyl, 4-methylhexyl, or 5-methylhexyl etc. Optionally, the linear or branched C₁-C₁₀ alkyl includes but is not limited to methyl, ethyl, and propyl etc. Wherein the alkyl is optionally substituted by one or more substituents optionally selected from the group consisting of deuterium, halogen, cyano, nitro, hydroxy, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo, or any combination thereof. The substituent are preferably one or more selected from the group consisting of D (i.e., deuterium), halogen, hydroxy, cyano, C₁₋₃ alkyl, C₁₋₃ alkoxy, trifluoromethyl, heterocyclyl (e.g., 4- to 8-membered heterocyclyl) or any combination thereof. Embodiment A34): Pertaining to the compound of Formula A or salts, enantiomers, stereoisomers, polymorphs or solvates thereof as described in embodiment A20), wherein Rₐ is unsubstituted or substituted cycloalkyl. Optionally, when the Rₐ is unsubstituted or substituted cycloalkyl, it has 3 to 20 carbon atoms (i.e., C₃₋₂₀ cycloalkyl), 3 to 15 carbon atoms (i.e., C₃₋₁₅ cycloalkyl), 3 to 12 carbon atoms (i.e., C₃₋₁₂ cycloalkyl), 3 to 11 carbon atoms (i.e., C₃₋₁₁ cycloalkyl), 3 to 10 carbon atoms (i.e., C₃₋₁₀ cycloalkyl), 3 to 8 carbon atoms (i.e., C₃₋₈ cycloalkyl), 3 to 7 carbon atoms (i.e., C₃₋₇ cycloalkyl), or 3 to 6 carbon atoms (i.e., C₃₋₆ cycloalkyl). Preferably, R₁ is unsubstituted or substituted C₃-C₁₅ cycloalkyl.

Optionally, C₃-C₁₅ cycloalkyl includes but is not limited to cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₃-C₁₅ spiro-cycloalkyl or C₅-C₁₅ spiro-cycloalkyl or C₇-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl) etc. Further optionally, C₃-C₁₅ cycloalkyl includes but is not limited to cyclopropyl, cyclobutyl, and cyclopentyl etc. Wherein said cycloalkyl is optionally substituted with one or more (e.g., 1-5, 1-4, 1-3, 1-2, or 1) substituents optionally selected from the group consisting of trifluoromethyl, mercapto, hydroxy, amino, halogen, cyano, C₁-C₃ alkyl, C₁-C₃ alkoxy, trifluoromethyl, heterocyclyl, deuterium, nitro, carbonyl, ester group, imide group, amino, phosphine oxide group, deuterated alkoxy, trifluoromethoxy, aryloxy, alkylthio, arylthio, alkylsulfonyl, arylsulfonyl, silyl, boryl, alkyl, deuterated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, acenaphthenyl, oxo or any combination thereof. The substituents are preferably one or more selected from the group consisting of trifluoromethyl, mercapto, hydroxy, amino, halogen, cyano, C₁-C₃ alkyl, C₁-C₃ alkoxy, trifluoromethyl, heterocyclyl, or any combination thereof.

Embodiment A35): Pertaining to the compound of Formula A or salts, enantiomers, stereoisomers, polymorphs or solvates thereof as described in embodiment A20), wherein Rₐ is unsubstituted or substituted heterocyclyl. Optionally, when the Rₐ is unsubstituted or substituted heterocyclyl, the unsubstituted or substituted heterocyclyl is unsubstituted or substituted 4- to 30-membered heterocyclyl, 4- to 25-membered heterocyclyl, 4- to 20-membered heterocyclyl, 4- to 15-membered heterocyclyl, 4- to 10-membered heterocyclyl, 10- to 15-membered heterocyclyl, 5- to 15-membered heterocyclyl, or 7- to 15-membered heterocyclyl etc. Optionally, the heterocyclyl contains 1-3 heteroatoms, or 1-2 heteroatoms, or 1 heteroatom etc, with each heteroatom independently selected from the group consisting of O, N, and S etc. Further optionally, the heterocyclyl includes but is not limited to azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, diazacycloheptanyl (e.g., 1, 4-diazacycloheptanyl, 4, 5-diazacycloheptanyl, or 1, 3-diazacycloheptanyl etc.), azabicyclohexyl (e.g., 3-azabicyclo[3.1.0]hexyl etc.), azabicycloheptanyl (e.g., 3-azabicyclo[3.1.1]heptanyl, 3-azabicyclo[3.2.0]heptanyl etc), azabicyclooctyl (e.g., cis-7-azabicyclo[3.3.0]octyl etc.), and spiro-heterocyclyl (e.g., 4- to 15-membered spiro-heterocyclyl, or 5- to 15-membered spiro-heterocyclyl, or 7 to 15-membered spiro-heterocyclyl, such as 7-azaspiro[3.5]nonanyl, 3-azaspiro[5,5]undecanyl etc.). The heterocyclyl may be unsubstituted or substituted as explicitly defined (e.g., mono-, di-, tri-, or poly-substituted) by a substituent optionally selected from the group consisting of deuterium, hydroxy, amino, mercapto, nitro, halogen, cyano, carbonyl, ester group, imide group, phosphine oxide group, trifluoromethoxy, oxo, optionally deuterated C₁-C₆ alkyl, halogenated C₁-C₆ alkyl, optionally deuterated C₃₋₆ cycloalkyl, optionally deuterated C₁-C₆ alkoxy, C₁₋₄ alkyl-NH—, NH₂—C₁₋₆ alkylene, C₁₋₄ alkyl-NHC(O)—, C₁₋₄ alkyl-C(O)NH—, alkenyl, aryl, aralkyl, arylalkenyl, alkylaryl, aralkylamino, heteroarylamino, arylamino, arylphosphino, heteroaryl, acenaphthenyl, and any combination thereof (preferably, the substituent is selected from the group consisting of deuterium, hydroxy, amino, mercapto, nitro, halogen, cyano, oxo, optionally deuterated C₁-C₆ alkyl, halogenated C₁-C₆ alkyl, optionally deuterated C₃₋₆ cycloalkyl, optionally deuterated C₁-C₆ alkoxy, C₁₋₄ alkyl-NH—, NH₂—C₁₋₆ alkylene, C₁₋₄ alkyl-NHC(O)—, C₁₋₄ alkyl-C(O)NH—, and any combination thereof).

Embodiment A36): Pertaining to the compound of Formula A or salts, enantiomers, stereoisomers, polymorphs or solvates thereof as described in embodiment A20), wherein Rₐ is unsubstituted or substituted aryl. Optionally, the unsubstituted or substituted aryl is unsubstituted or substituted C₅₋₃₀ aryl. Optionally, the unsubstituted or substituted C₅₋₃₀ aryl is unsubstituted or substituted C₅₋₂₅, C₅₋₂₀, or C₅₋₁₅ aryl. Optionally, C₅₋₃₀ aryl may be monocyclic or polycyclic aryl. In some embodiments, the monocyclic aryl includes but is not limited to phenyl, biphenyl, terphenyl, quaterphenyl, and quinquephenyl etc. Polycyclic aryl includes but is not limited to naphthyl, anthryl, phenanthryl, pyrenyl, perylenyl, and fluorenyl etc. The fluorenyl may be substituted (e.g., 9,9′-dimethylfluorenyl and 9,9′-diphenylfluorenyl etc.). Furthermore, two of the substituents may combine with each other to form a spirocyclic structure such as 9,9′-spirobifluorenyl etc. The aryl is optionally substituted aryl. The substituted aryl refers to an aryl group substituted one or more times (e.g., 1-4, 1-3, or 1-2 times) with a substituent. For example, aryl is mono-, di-, or tri-substituted with a substituent optionally selected from the group consisting of e.g., deuterated C₁-C₆ alkyl, deuterated C₃₋₆ cycloalkyl, deuterium, hydroxy, amino, mercapto, halogen, cyano, nitro, carbonyl, ester group, imide group, phosphine oxide group, trifluoromethoxy, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, alkyl, cycloalkyl, alkenyl, aryl, aralkyl, arylalkenyl, alkylaryl, aralkylamino, heteroarylamino, arylamino, arylphosphino, heteroaryl, acenaphthenyl, and any combination thereof. Preferably, the substituent is selected from the group consisting of deuterated C₁-C₆ alkyl, deuterated C₃₋₆ cycloalkyl, hydroxy, amino, mercapto, halogen, cyano, deuterated C₁-C₆ alkoxy, C₁-C₄ alkylamino, halogenated C₁-C₆ alkyl, amino-substituted C₁-C₆ alkylene, C₁-C₄ alkyl-NHC(O)—, C₁-C₄ alkyl-C(O)NH—, and any combination thereof.

Embodiment A37): Pertaining to the compound of Formula A or salts, enantiomers, stereoisomers, polymorphs or solvates thereof as described in embodiment A20), wherein Rₐ is unsubstituted or substituted heteroaryl. When the Rₐ is unsubstituted or substituted heteroaryl, the unsubstituted or substituted heteroaryl is unsubstituted or substituted 5- to 30-membered heteroaryl. Optionally, the unsubstituted or substituted 5- to 30-membered heteroaryl is unsubstituted or substituted 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered heteroaryl. Optionally, the heteroaryl includes but is not limited to pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, tetrazolyl, pyranyl, thiopyranyl, pyrazinyl, thiazinyl, triazinyl, tetrazinyl, quinolinyl, isoquinolinyl, quinazolinyl, quinoxalinyl, cinnolinyl, naphthyridinyl, acridinyl, xanthenyl, III phenanthridinyl, phthalazinyl, triazaindenyl, indolyl, indolinyl, indolizinyl, phthalazinyl, pyrazolopyridyl, pyrazolopyrimidinyl, pyridopyrimidinyl, pyridopyrazinyl, pyrazinopyrazinyl, benzothiazolyl, benzoxazolyl, benzimidazolyl, benzothienyl, benzofuranyl, isobenzofuranyl, dibenzothienyl, dibenzofuranyl, indazolyl, carbazolyl, benzocarbazolyl, dibenzocarbazolyl, indolocarbazolyl, indenocarbazolyl, phenazinyl, imidazopyridinyl, phenanthridinyl, phenanthrolinyl, phenothiazinyl, imidazopyridinyl, imidazophenanthridinyl, benzimidazoquinazolinyl, benzimidazophenanthridinyl, pyrrolopyridinyl, pyrrolothiazolyl, imidazothiazolyl, pyrenyl, dinaphthofuranyl, naphthobenzofuranyl, dinaphthothienyl, and naphthobenzothienyl etc. Wherein the heteroaryl is unsubstituted or substituted. The substituted heteroaryl refers to a heteroaryl substituted one or more times (e.g., 1-4, 1-3, or 1-2 times) with a substituent optionally selected from the group consisting of C₁-C₃ alkyl, C₃-C₆ cycloalkyl, hydroxy, amino, mercapto, halogen, deuterium, nitro, carbonyl, ester group, imide group, phosphine oxide group, trifluoromethoxy, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, alkenyl, aryl, aralkyl, arylalkenyl, alkylaryl, aralkylamino, heteroarylamino, arylamino, arylphosphino, heteroaryl, acenaphthenyl, or any combination thereof. Preferably, the substituent is optionally selected from the group consisting of C₁-C₃ alkyl, C₃-C₆ cycloalkyl, hydroxy, amino, mercapto, halogen, C₁-C₃ alkoxy, C₁-C₃ alkylamino, halogenated C₁-C₃ alkyl, amino-substituted C₁-C₃ alkylene, C₁-C₃ alkyl-NHC(O)—, C₁-C₃ alkyl-C(O)NH—, cyano, or any combination thereof.

Embodiment A38): Pertaining to the compound of Formula A or salts, enantiomers, stereoisomers, polymorphs or solvates thereof as described in any one of embodiments A1)-A37), wherein R_{b1}, R_{b2}, R_{b3}, R_{b4}, and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl.

Embodiment A39): Pertaining to the compound of Formula A or salts, enantiomers, stereoisomers, polymorphs or solvates thereof as described in any one of embodiments A1)-A37), wherein R_{b1}, R_{b2}, R_{b3}, R_{b4}, and R_{b5} are each independently selected from the group consisting of hydrogen, deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, or unsubstituted or substituted linear or branched C₁-C₅alkyl.

Embodiment A40): Pertaining to the compound of Formula A or salts, enantiomers, stereoisomers, polymorphs or solvates thereof as described in any one of embodiments A1)-A37), wherein R_{b1}, R_{b2}, R_{b3}, R_{b4}, and R_{b5} are each independently selected from the group consisting of hydrogen, deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, or unsubstituted or substituted linear or branched C₁-C₅alkyl.

Embodiment A41): Pertaining to the compound of Formula A or salts, enantiomers, stereoisomers, polymorphs or solvates thereof as described in any one of embodiments A1)-A37), wherein R_{b1}, R_{b2}, R_{b3}, R_{b4}, and R_{b5} are each independently selected from the group consisting of hydrogen.

Embodiment A42): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of deuterium.

Embodiment A43): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of halogen. Optionally, when the R_{b1}, R_{b2}, R_{b3}, R_{b4}, and R_{b5} are each independently selected from the group consisting of halogen, the halogen is F, Cl, Br, or I.

Embodiment A44): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of nitro.

Embodiment A45): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of cyano.

Embodiment A46): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of amino.

Embodiment A47): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of hydroxy.

Embodiment A48): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of mercapto.

Embodiment A49): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of alkoxy. Optionally, when the R_{b1}, R_{b2}, R_{b3}, R_{b4}, and R_{b5} are each independently selected from the group consisting of alkoxy, the alkoxy is C₁-C₆ alkoxy.

Further optionally, the alkoxy may include but is not limited to e.g., methoxy, ethoxy, n-propoxy, isopropoxy, n-butoxy, isobutoxy, tert-butoxy, sec-butoxy, n-pentoxy, neopentoxy, isopentoxy, n-hexoxy, 3,3-dimethylbutoxy, and 2-ethylbutoxy etc.

Embodiment A50): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of halogenated alkoxy. Optionally, when the R_{b1}, R_{b2}, R_{b3}, R_{b4}, and R_{b5} are each independently selected from the group consisting of halogenated alkoxy, the halogenated alkoxy is halogenated C₁-C₆ alkoxy. Further the halogenated C₁-C₆ alkoxy is trifluoromethoxy etc.

Embodiment A51): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of unsubstituted or substituted amino. Optionally, the unsubstituted or substituted amino includes amino, N-methylamino, N-ethylamino, N-phenylamino, N,N-dimethylamino, and N,N-diethylamino etc.

Embodiment A52): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of unsubstituted or substituted silyl. Optionally, the unsubstituted or substituted silyl is specifically trimethylsilyl, triethylsilyl, tert-butyldimethylsilyl, vinyldimethylsilyl, propyldimethylsilyl, triphenylsilyl, diphenylsilyl, and phenylsilyl etc.

Embodiment A53): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of unsubstituted or substituted boryl. Optionally, the unsubstituted or substituted boryl is specifically trimethylboryl, triethylboryl, tert-butyldimethylboryl, triphenylboryl, and phenylboryl etc.

Embodiment A54): Pertaining to the compound of Formula A or salts, enantiomers, stereoisomers, polymorphs or solvates thereof as described in embodiment A38), wherein R_{b1}, R_{b2}, R_{b3}, R_{b4}, and R_{b5} are each independently selected from the group consisting of unsubstituted or substituted linear or branched alkyl. Optionally, the number of carbon atoms may be for example C₁-C₃₀, C₁-C₂₅, C₁-C₂₀, C₁-C₁₅, C₁-C₁₀, C₁-C₅ or C₁-C₃ etc, with linear or branched C₁-C₁₀ alkyl being preferred. Further optionally, linear or branched C₁-C₁₀ alkyl includes but is not limited to methyl, ethyl, propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, nonyl, 2,2-dimethylheptyl, 1-ethylpropyl, 1,1-dimethylpropyl, isohexyl, 4-methylhexyl, and 5-methylhexyl etc. Optionally, the linear or branched C₁-C₁₀ alkyl includes but is not limited to methyl, ethyl, and propyl etc. Wherein the alkyl is optionally substituted, and the substituent may be e.g., selected from the group consisting of deuterium, halogen, cyano, nitro, hydroxy, mercapto, carbonyl, ester group, imide group, amino, phosphine oxide group, alkoxy, deuterated alkoxy, trifluoromethoxy, aryloxy, alkylthio, arylthio, alkylsulfonyl, arylsulfonyl, silyl, boryl, alkyl, deuterated alkyl, halogenated alkyl, amino-substituted alkylene, alkyl-NHC(O)—, alkyl-C(O)NH—, cycloalkyl, deuterated cycloalkyl, alkenyl, aryl, aralkyl, arylalkenyl, alkyl aryl, alkylamino, aralkylamino, heteroarylamino, arylamino, arylphosphino, heteroaryl, acenaphthenyl, oxo or any combination thereof (preferably one or more selected from the group consisting of D (i.e., deuterium), halogen, hydroxy, cyano, C₁-C₃ alkyl, C₁-C₃ alkoxy, trifluoromethyl, heterocyclyl (e.g., 4- to 8-membered heterocyclyl), or any combination thereof).

Embodiment A55): Pertaining to the compound of Formula A or salts, enantiomers, stereoisomers, polymorphs or solvates thereof as described in any one of embodiment A1)-A54), wherein n is an integer of 0, 1, 2 or 3, and when n is an integer of 2 or 3, each Rₐ is independently identical to or different from each other. In some embodiments, n is an integer of 0, 1 or 2.

Especially preferably the compounds of the present disclosure and their salts (especially pharmaceutically acceptable salts, such as hydrochloride, etc.), enantiomers, stereoisomers, polymorphs, or solvates thereof in Table 2 below are provided:

TABLE 2
The compounds of the present disclosure

| Compound | | |
|---|---|---|
| No. | Structure of the compounds | The compounds′ name |
| GT- 03613 | 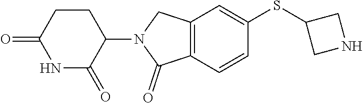 [SMILES: O=C1CCC(N2Cc3cc(SC4CNC4)ccc3C2=O)C(=O)N1] | 3-(5-(azetidin-3-ylthio)-1- oxoisoindolin-2-yl)piperidine- 2,6-dione |
| GT- 04309 | 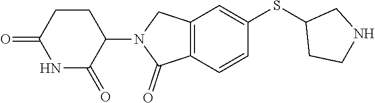 [SMILES: O=C1CCC(N2Cc3cc(SC4CCNC4)ccc3C2=O)C(=O)N1] | 3-(1-oxo-5-(pyrrolidin-3- ylthio)isoindolin-2- yl)piperidine-2,6-dione |
| GT- 03616 | 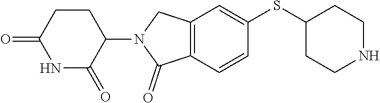 [SMILES: O=C1CCC(N2Cc3cc(SC4CCNCC4)ccc3C2=O)C(=O)N1] | 3-(1-oxo-5-(piperidin-4- ylthio)isoindolin-2- yl)piperidine-2,6-dione |
| GT- 06995 | 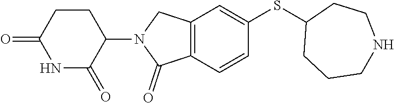 [SMILES: O=C1CCC(N2Cc3cc(SC4CCCNCC4)ccc3C2=O)C(=O)N1] | 3-(5-(azepan-4-ylthio)-1- oxoisoindolin-2-yl)piperidine- 2,6-dione |
| GT- 06992 | 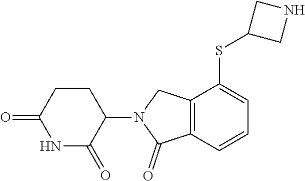 [SMILES: O=C1CCC(N2Cc3c(SC4CNC4)cccc3C2=O)C(=O)N1] | 3-(4-(azetidin-3-ylthio)-1- oxoisoindolin-2-yl)piperidine- 2,6-dione |
| GT- 06993 | 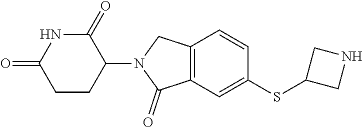 [SMILES: O=C1CCC(N2Cc3ccc(SC4CNC4)cc3C2=O)C(=O)N1] | 3-(6-(azetidin-3-ylthio)-1- oxoisoindolin-2-yl)piperidine- 2,6-dione |
| GT- 06994 | 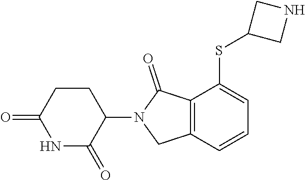 [SMILES: O=C1CCC(N2Cc3cccc(SC4CNC4)c3C2=O)C(=O)N1] | 3-(7-(azetidin-3-ylthio)-1- oxoisoindolin-2-yl)piperidine- 2,6-dione |
| GT- 06997 | 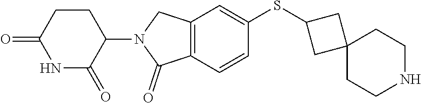 [SMILES: O=C1CCC(N2Cc3cc(SC4CC5(CCNCC5)C4)ccc3C2=O)C(=O)N1] | 3-(5-((7-azaspiro[3.5]nonan-2- yl)thio)-1-oxoisoindolin-2- yl)piperidine-2,6-dione |
| GT- 06998 | 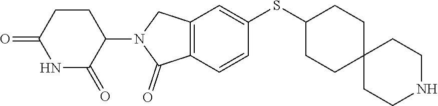 [SMILES: O=C1CCC(N2Cc3cc(SC4CCC5(CCNCC5)CC4)ccc3C2=O)C(=O)N1] | 3-(5-((3-azaspiro[5.5]undecan- 9-yl)thio)-1-oxoisoindolin-2- yl)piperidine-2,6-dione |
| GT- 06996 | 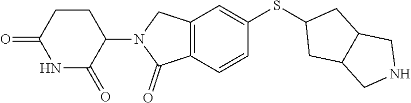 [SMILES: O=C1CCC(N2Cc3cc(SC4CC5CNCC5C4)ccc3C2=O)C(=O)N1] | 3-(5- ((octahydrocyclopenta[c]pyrrol- 5-yl)thio)-1-oxoisoindolin-2- yl)piperidine-2,6-dione |
| GT- 06999 | 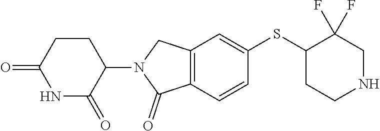 [SMILES: O=C1CCC(N2Cc3cc(SC4CCNCC4(F)F)ccc3C2=O)C(=O)N1] | 3-(5-((3,3-difluoropiperidin-4- yl)thio)-1-oxoisoindolin-2- yl)piperidine-2,6-dione |
| | 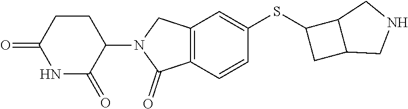 [SMILES: O=C1CCC(N2Cc3cc(SC4CC5CNCC54)ccc3C2=O)C(=O)N1] | 3-(5-((3- azabicyclo[3.2.0]heptan-6- yl)thio)-1-oxoisoindolin-2- yl)piperidine-2,6-dione |
| | 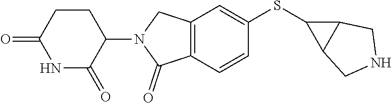 [SMILES: O=C1CCC(N2Cc3cc(SC4C5CNCC54)ccc3C2=O)C(=O)N1] | 3-(5-((3- azabicyclo[3.1.0]hexan-6- yl)thio)-1-oxoisoindolin-2- yl)piperidine-2,6-dione |
| | 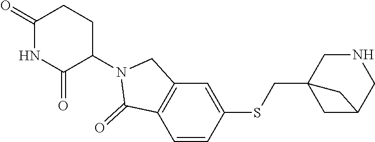 [SMILES: O=C1CCC(N2Cc3cc(SCC45CNCC(C4)C5)ccc3C2=O)C(=O)N1] | 3-(5-(((3- azabicyclo[3.1.1]heptan-1- yl)methyl)thio)-1-oxoisoindolin- 2-yl)piperidine-2,6-dione |
| | 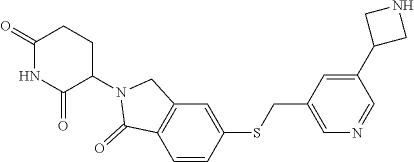 [SMILES: O=C1CCC(N2Cc3cc(SCc4cncc(C5CNC5)c4)ccc3C2=O)C(=O)N1] | 3-(5-(((5-(azetidin-3-yl)pyridin- 3-yl)methyl)thio)-1- oxoisoindolin-2-yl)piperidine- 2,6-dione |
| GT- 03614 | 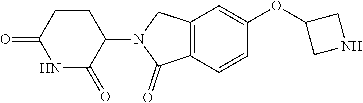 [SMILES: O=C1CCC(N2Cc3cc(OC4CNC4)ccc3C2=O)C(=O)N1] | 3-(5-(azetidin-3-yloxy)-1- oxoisoindolin-2-yl)piperidine- 2,6-dione |
| GT- 07002 | 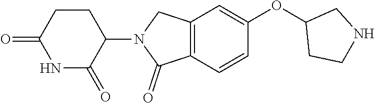 [SMILES: O=C1CCC(N2Cc3cc(OC4CCNC4)ccc3C2=O)C(=O)N1] | 3-(1-oxo-5-(pyrrolidin-3- yloxy)isoindolin-2- yl)piperidine-2,6-dione |
| GT- 03612 | 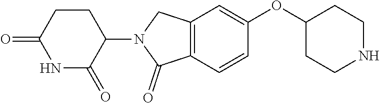 [SMILES: O=C1CCC(N2Cc3cc(OC4CCNCC4)ccc3C2=O)C(=O)N1] | 3-(1-oxo-5-(piperidin-4- yloxy)isoindolin-2- yl)piperidine-2,6-dione |
| GT- 07003 | 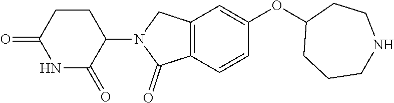 [SMILES: O=C1CCC(N2Cc3cc(OC4CCCNCC4)ccc3C2=O)C(=O)N1] | 3-(5-(azepan-4-yloxy)-1- oxoisoindolin-2-yl)piperidine- 2,6-dione |
| GT- 07000 | 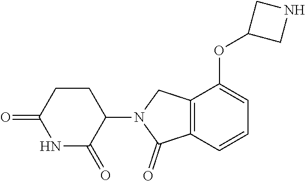 [SMILES: O=C1CCC(N2Cc3c(OC4CNC4)cccc3C2=O)C(=O)N1] | 3-(4-(azetidin-3-yloxy)-1- oxoisoindolin-2-yl)piperidine- 2,6-dione |
| GT- 07001 | 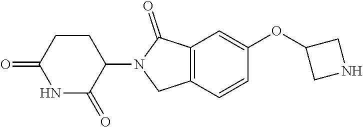 [SMILES: O=C1CCC(N2Cc3ccc(OC4CNC4)cc3C2=O)C(=O)N1] | 3-(6-(azetidin-3-yloxy)-1- oxoisoindolin-2-yl)piperidine- 2,6-dione |
| GT- 07052 | 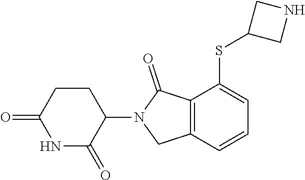 [SMILES: O=C1CCC(N2Cc3cccc(SC4CNC4)c3C2=O)C(=O)N1] | 3-(7-(azetidin-3-yloxy)-1- oxoisoindolin-2-yl)piperidine- 2,6-dione |
| GT- 07005 | 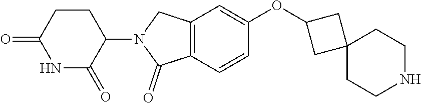 [SMILES: O=C1CCC(N2Cc3cc(OC4CC5(CCNCC5)C4)ccc3C2=O)C(=O)N1] | 3-(5-((7-azaspiro[3.5]nonan-2- yl)oxy)-1-oxoisoindolin-2- yl)piperidine-2,6-dione |
| GT- 07004 | 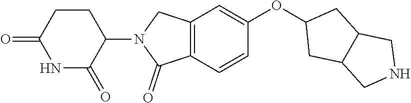 [SMILES: O=C1CCC(N2Cc3cc(OC4CC5CNCC5C4)ccc3C2=O)C(=O)N1] | 3-(5- ((octahydrocyclopenta[c]pyrrol- 5-yl)oxy)-1-oxoisoindolin-2- yl)piperidine-2,6-dione |
| | 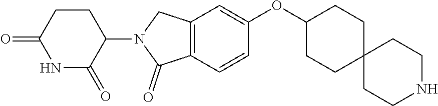 [SMILES: O=C1CCC(N2Cc3cc(OC4CCC5(CCNCC5)CC4)ccc3C2=O)C(=O)N1] | 3-(5-((3-azaspiro[5.5]undecan- 9-yl)oxy)-1-oxoisoindolin-2- yl)piperidine-2,6-dione |
| | 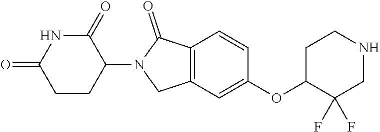 [SMILES: O=C1CCC(N2Cc3cc(OC4CCNCC4(F)F)ccc3C2=O)C(=O)N1] | 3-(5-((3,3-difluoropiperidin-4- yl)oxy)-1-oxoisoindolin-2- yl)piperidine-2,6-dione |
| | 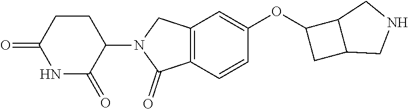 [SMILES: O=C1CCC(N2Cc3cc(OC4CC5CNCC54)ccc3C2=O)C(=O)N1] | 3-(5-((3- azabicyclo[3.2.0]heptan-6- yl)oxy)-1-oxoisoindolin-2- yl)piperidine-2,6-dione |
| | 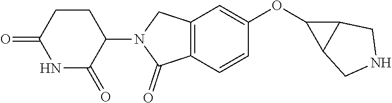 [SMILES: O=C1CCC(N2Cc3cc(OC4C5CNCC54)ccc3C2=O)C(=O)N1] | 3-(5-((3- azabicyclo[3.1.0]hexan-6- yl)oxy)-1-oxoisoindolin-2- yl)piperidine-2,6-dione |
| | 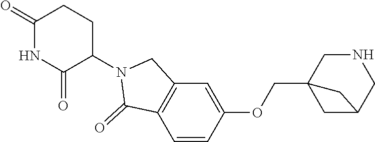 [SMILES: O=C1CCC(N2Cc3cc(OCC45CNCC(C4)C5)ccc3C2=O)C(=O)N1] | 3-(5-((3- azabicyclo[3.1.1 ]heptan-1- yl)methoxy)-1-oxoisoindolin-2- yl)piperidine-2,6-dione |
| | 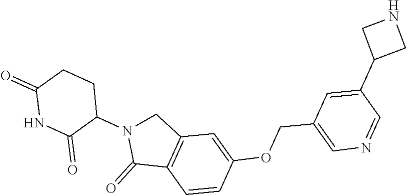 [SMILES: O=C1CCC(N2Cc3cc(OCc4cncc(C5CNC5)c4)ccc3C2=O)C(=O)N1] | 3-(5-((5-(azetidin-3-yl)pyridin- 3-yl)methoxy)-1-oxoisoindolin- 2-yl)piperidine-2,6-dione |

In a further aspect, the present disclosure provides the compounds of Formula I, Formula A, or salts, enantiomers, stereoisomers, polymorphs or solvates thereof of the present disclosure, for use as a medicament.

### Pharmaceutically Compositions

In a further aspect, the present disclosure provides a pharmaceutical composition comprising the compounds of the present disclosure (the compounds of Formula I or Formula A) or salts, enantiomers, stereoisomers, polymorphs or solvates thereof, and at least one pharmaceutically acceptable salt, and at least one pharmaceutically acceptable carrier or excipient.

The composition may comprise one or more pharmaceutically acceptable carriers, which generally refer to carriers used for the administration of therapeutic agents and do not themselves induce the production of antibodies harmful to the individual receiving the composition and exhibit no excessive toxicity upon administration. These carriers are well-known to those skilled in the art, e.g., relevant information on pharmaceutically acceptable carriers disclosed in Remington's Pharmaceutical Sciences (Mack Pub. Co., N. J, 1991). Specifically, the carrier may comprise, but is not limited to, one or more selected from the group consisting of saline, buffers, glucose, water, glycerol, ethanol, adjuvants, and combinations thereof. Each carrier must be “acceptable” in the sense of being compatible with the other ingredients of the formulation (including the compounds useful in the present disclosure) and not injurious to the patient.

The composition may comprise one or more pharmaceutically acceptable excipients, which refer to a pharmaceutically acceptable material, such as a filler, stabilizer, dispersant, suspending agent, diluent, vehicle, and thickener, solvent, or encapsulating material, with which the useful compounds according to the present disclosure are carried or transported into or administered to a patient so that they may perform their intended function. Generally, such constructs are carried or transported from one organ or part of the body to another organ or part of the body. The excipient is compatible with the other ingredients of the formulation, including the compounds useful in the present disclosure, and is not harmful to the patient, and the excipient must be “acceptable”. Some examples of materials that may be used as pharmaceutically acceptable excipients include sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; vehicles such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; polyethylene oxide, polyvinylpyrrolidone, polyacrylamide, poloxamer, and other common non-toxic compatible substances used in pharmaceutical formulations.

The pharmaceutical composition of the present disclosure comprising, as an active ingredient, the compounds of the present disclosure (the compounds of Formula I or Formula A) or a pharmaceutically acceptable salt thereof can be formulated into any suitable formulations such as sprays, patches, tablets (such as conventional tablets, dispersible tablets, orally disintegrating tablets), capsules (such as soft capsules, hard capsules, enteric-coated capsules), dragees, troches, powders, granules, powder injections, suppositories, or liquid formulations (such as suspensions (e.g., aqueous or oily suspensions), solutions, emulsions, or syrups), or conventional injection dosage forms such as injectable solutions (e.g., sterile injectable solutions formulated according to methods known in the art using water, Ringer's solution, or isotonic sodium chloride solution or the like as a vehicle or solvent) or lyophilized injectable formulation and the like, depending upon a suitable route of administration (including, but not limited to, nasal administration, inhalation administration, topical administration, oral administration, oral mucosal administration, rectal administration, intrapleural administration, intraperitoneal administration, vaginal administration, intramuscular administration, subcutaneous administration, transdermal administration, epidural administration, intrathecal administration, and intravenous administration). Those skilled in the art can also formulate the compounds of the present disclosure (the compounds of Formula I or Formula A) into conventional, dispersible, chewable, orally disintegrating or rapidly dissolving formulations as needed.

The compounds of the present disclosure (the compounds of Formula I or Formula A), as an active ingredient, is contained in the pharmaceutically acceptable carrier or diluent in an amount sufficient to deliver to a subject a therapeutically effective amount for the indication to be treated, without causing serious toxic effects in the subject treated. A dosage of the active compound for all diseases or disorders mentioned herein ranges, for example, from about 5 ng/kg to 500 mg/kg, from about 10 ng/kg to 300 mg/kg per day, such as from 0.1 to 100 mg/kg, or from 0.5 to about 25 mg per kilogram body weight of the subject per day.

The compounds of the present disclosure (the compounds of Formula I or Formula A) or pharmaceutically acceptable salts thereof can be conveniently administered in any suitable unit dosage form. Suitable unit dosage form specifications include, but are not limited to, less than 1 mg, 1 mg to 3000 mg, 5 mg to 1000 mg, for example, 5 to 500 mg, 25 to 250 mg of active ingredient per unit dosage form.

The pharmaceutical composition provided in the present disclosure further comprises at least one additional therapeutic agent for treating or preventing diseases associated with cereblon protein. In one embodiment, the disease or disorder associated with cereblon protein is selected from the group consisting of: tumor, infectious disease, inflammatory disease, immune system diseases, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes. Specifically, the disease or disorder associated with cereblon protein is selected from the group consisting of: myeloma (e.g., multiple myeloma, plasma cell myeloma, smoldering myeloma, smoldering multiple myeloma); myelofibrosis; bone marrow disease; myelodysplastic syndrome (MDS); previously treated myelodysplastic syndrome; transplantation-related cancer; neutropenia; leukemia (e.g., acute myeloid leukemia, chronic myelogenous leukemia, B-cell chronic lymphocytic leukemia, leukemia-associated anemia, acute myeloid leukemia (AML), monocytic leukemia, acute lymphoblastic leukemia (ALL), acute B-cell lymphoblastic leukemia, acute T-cell lymphoblastic leukemia, chronic lymphocytic leukemia, lymphoma cell leukemia, T-cell lymphoblastic leukemia, myelomonocytic leukemia); lymphoma (e.g., diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, follicular lymphoma (FL), Burkitt lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma); thyroid cancer; melanoma; lung cancer (e.g., lung adenocarcinoma, lung squamous cell carcinoma, non-small cell lung cancer, and small cell lung cancer); inflammatory myofibroblastoma; colorectal cancer; intestinal cancer; brain glioma; astroblastoma; neuroglioma; peripheral neuroepithelioma; ovarian cancer; bronchial cancer; prostate cancer; breast cancer (e.g., triple negative breast cancer, sporadic breast cancer, and patients with Cowden syndrome); pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma (e.g., rhabdomyosarcoma, various lipogenic tumors, Ewing's sarcoma/primitive neuroectodermal tumors (Ewing/PNETs), and leiomyosarcoma); urothelial carcinoma; basal cell carcinoma; oral squamous cell carcinoma; cholangiocarcinoma; bone cancer; cervical cancer; skin cancer; bone tumors; brain tumors; lung or mesothelium tumor; colonic tumors; ureteral tumors; Richter syndrome (RS); sepsis syndrome; autoimmune diseases (e.g., rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, atopic dermatitis, vitiligo, experimental autoimmune encephalomyelitis, and Arthus reaction); keratoconjunctivitis; inflammatory diseases (e.g., Crohn's disease and ulcerative colitis, pneumonia, osteoarthritis, synovitis, systemic inflammatory response syndrome, airway inflammation, bronchitis, rheumatoid spondylitis, gouty arthritis, eczema, psoriasis, dermatitis, uveitis, conjunctivitis, acute respiratory distress syndrome, bacteremia, endotoxemia, aphthous ulcer, gingivitis, pancreatitis, regional ileitis, atrophic gastritis, peritonitis, pepticulcer, or irritable bowel syndrome); cerebral malaria; infectious diseases (e.g., viral pneumonia, Acquired immunodeficiency syndrome (AIDS), COVID-19 novel coronavirus infection, gram-negative bacteria infection, gram-positive bacteria infection, tuberculosis); septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.

### Kits/Packaged Products

The present disclosure further provides a kit comprising the pharmaceutical composition, which comprises a pharmaceutically acceptable carrier or excipient, and the compounds of Formula I as defined in any one of embodiments 1)-111) or the compounds of Formula A as defined in any one of embodiments A1)-A55); instructions for use describing how to administer the pharmaceutical composition in combination with chemotherapy, radiotherapy, and/or targeted therapy for the prevention or treatment of cancer.

The present disclosure provides kits comprising the compounds, pharmaceutical compositions, or other agents described herein, which may be used to perform the methods described herein. In some embodiments, the kits contain at least one purified compound in one or more containers. In some embodiments, the kits include all components necessary and/or sufficient to perform detection assays, including all controls, instructions for conducting the assays, and any necessary software for analyzing and presenting results. The kits may also include labels or indications describing the kit components or a set of instructions for their use in the ligand detection methods of the present disclosure. Such instructions may be associated with the package insert and/or packaging of the kit or its components. Those skilled in the art will readily recognize that the compounds, pharmaceutical compositions, or other agents of the present disclosure can be easily incorporated into established kit formats well-known in the field. Such kits may further include, for example, other compounds and/or compositions, one or more devices for administering said compounds and/or compositions, and written instructions in a form prescribed by governmental agencies regulating the manufacture, use, or sale of pharmaceutical or biological products.

### Uses and Therapeutic Methods

In a further aspect, the present disclosure provides use of the compounds or salts, enantiomers, stereoisomers, polymorphs or solvates thereof of the present disclosure, or the pharmaceutical composition of the present disclosure for the manufacture of a medicament for the treatment of a disease or disorder associated with cereblon protein.

Specifically, in the use provided by the present disclosure, the disease or disorder associated with cereblon protein is selected from the group consisting of: tumor, infectious disease, inflammatory disease, immune system diseases, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes.

More specifically, in the use provided by the present disclosure, the disease or disorder associated with cereblon protein is selected from the group consisting of: myeloma (e.g., multiple myeloma, plasma cell myeloma, smoldering myeloma, smoldering multiple myeloma); myelofibrosis; bone marrow disease; myelodysplastic syndrome (MDS); previously treated myelodysplastic syndrome; transplantation-related cancer; neutropenia; leukemia (e.g., acute myeloid leukemia, chronic myelogenous leukemia, B-cell chronic lymphocytic leukemia, leukemia-associated anemia, acute myeloid leukemia (AML), monocytic leukemia, acute lymphoblastic leukemia (ALL), acute B-cell lymphoblastic leukemia, acute T-cell lymphoblastic leukemia, chronic lymphocytic leukemia, lymphoma cell leukemia, T-cell lymphoblastic leukemia, myelomonocytic leukemia); lymphoma (e.g., diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, follicular lymphoma (FL), Burkitt lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma); thyroid cancer; melanoma; lung cancer (e.g., lung adenocarcinoma, lung squamous cell carcinoma, non-small cell lung cancer, and small cell lung cancer); inflammatory myofibroblastoma; colorectal cancer; intestinal cancer; brain glioma; astroblastoma; neuroglioma; peripheral neuroepithelioma; ovarian cancer; bronchial cancer; prostate cancer; breast cancer (e.g., triple negative breast cancer, sporadic breast cancer, and patients with Cowden syndrome); pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma (e.g., rhabdomyosarcoma, various lipogenic tumors, Ewing's sarcoma/primitive neuroectodermal tumors (Ewing/PNETs), and leiomyosarcoma); urothelial carcinoma; basal cell carcinoma; oral squamous cell carcinoma; cholangiocarcinoma; bone cancer; cervical cancer; skin cancer; bone tumors; brain tumors; lung or mesothelium tumor; colonic tumors; ureteral tumors; Richter syndrome (RS); sepsis syndrome; autoimmune diseases (e.g., rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, atopic dermatitis, vitiligo, experimental autoimmune encephalomyelitis, and Arthus reaction); keratoconjunctivitis; inflammatory diseases (e.g., Crohn's disease and ulcerative colitis, pneumonia, osteoarthritis, synovitis, systemic inflammatory response syndrome, airway inflammation, bronchitis, rheumatoid spondylitis, gouty arthritis, eczema, psoriasis, dermatitis, uveitis, conjunctivitis, acute respiratory distress syndrome, bacteremia, endotoxemia, aphthous ulcer, gingivitis, pancreatitis, regional ileitis, atrophic gastritis, peritonitis, pepticulcer, or irritable bowel syndrome); cerebral malaria; infectious diseases (e.g., viral pneumonia, Acquired immunodeficiency syndrome (AIDS), COVID-19 novel coronavirus infection, gram-negative bacteria infection, gram-positive bacteria infection, tuberculosis); septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.

In a further aspect, the present disclosure provides a method for treating or preventing a disease or disorder associated with cereblon protein, comprising administering to a subject a therapeutically effective amount of the compounds or salts, enantiomers, stereoisomers, polymorphs or solvates thereof of the present disclosure, or the pharmaceutical composition of the present disclosure.

Specifically, in the therapeutic method provided by the present disclosure, the disease or disorder associated with cereblon protein is selected from the group consisting of: tumor, infectious disease, inflammatory disease, immune system diseases, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes.

More specifically, in the method provided by the present disclosure, the disease or disorder associated with cereblon protein is selected from the group consisting of: myeloma (e.g., multiple myeloma, plasma cell myeloma, smoldering myeloma, smoldering multiple myeloma); myelofibrosis; bone marrow disease; myelodysplastic syndrome (MDS); previously treated myelodysplastic syndrome; transplantation-related cancer; neutropenia; leukemia (e.g., acute myeloid leukemia, chronic myelogenous leukemia, B-cell chronic lymphocytic leukemia, leukemia-associated anemia, acute myeloid leukemia (AML), monocytic leukemia, acute lymphoblastic leukemia (ALL), acute B-cell lymphoblastic leukemia, acute T-cell lymphoblastic leukemia, chronic lymphocytic leukemia, lymphoma cell leukemia, T-cell lymphoblastic leukemia, myelomonocytic leukemia); lymphoma (e.g., diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, follicular lymphoma (FL), Burkitt lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma); thyroid cancer; melanoma; lung cancer (e.g., lung adenocarcinoma, lung squamous cell carcinoma, non-small cell lung cancer, and small cell lung cancer); inflammatory myofibroblastoma; colorectal cancer; intestinal cancer; brain glioma; astroblastoma; neuroglioma; peripheral neuroepithelioma; ovarian cancer; bronchial cancer; prostate cancer; breast cancer (e.g., triple negative breast cancer, sporadic breast cancer, and patients with Cowden syndrome); pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma (e.g., rhabdomyosarcoma, various lipogenic tumors, Ewing's sarcoma/primitive neuroectodermal tumors (Ewing/PNETs), and leiomyosarcoma); urothelial carcinoma; basal cell carcinoma; oral squamous cell carcinoma; cholangiocarcinoma; bone cancer; cervical cancer; skin cancer; bone tumors; brain tumors; lung or mesothelium tumor; colonic tumors; ureteral tumors; Richter syndrome (RS); sepsis syndrome; autoimmune diseases (e.g., rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, atopic dermatitis, vitiligo, experimental autoimmune encephalomyelitis, and Arthus reaction); keratoconjunctivitis; inflammatory diseases (e.g., Crohn's disease and ulcerative colitis, pneumonia, osteoarthritis, synovitis, systemic inflammatory response syndrome, airway inflammation, bronchitis, rheumatoid spondylitis, gouty arthritis, eczema, psoriasis, dermatitis, uveitis, conjunctivitis, acute respiratory distress syndrome, bacteremia, endotoxemia, aphthous ulcer, gingivitis, pancreatitis, regional ileitis, atrophic gastritis, peritonitis, pepticulcer, or irritable bowel syndrome); cerebral malaria; infectious diseases (e.g., viral pneumonia, Acquired immunodeficiency syndrome (AIDS), COVID-19 novel coronavirus infection, gram-negative bacteria infection, gram-positive bacteria infection, tuberculosis); septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.

In the method for treating or preventing a disease or disorder associated with cereblon protein, the compounds of Formula I or Formula A or salts, stereoisomers, polymorphs or solvates thereof, or the pharmaceutical composition of the present disclosure is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

In another aspect, the present disclosure provides use of the compound of Formula A or salts, enantiomers, stereoisomers, polymorphs or solvates thereof of the present disclosure for the manufacture of a Cereblon protein (CRBN)-binding agent.

In another aspect, the present disclosure provides use of the compound of Formula A or salts, enantiomers, stereoisomers, polymorphs or solvates thereof of the present disclosure for the preparation of the compound of Formula I or salts, enantiomers, polymorphs or solvates thereof of the present disclosure.

### Examples

In the following description, numerous specific details are set forth in order to provide a thorough understanding of the present disclosure. The present disclosure may be practiced without some or all of these specific details. In other cases, well-known process operations have not been described in detail in order not to unnecessarily obscure the present disclosure. Although the present disclosure will be described in conjunction with specific embodiments, it should be understood that this is not intended to limit the present disclosure to these embodiments.

The following abbreviations are used throughout the specification and examples:
AcOH acetic acid
AlCl₃ aluminum chloride
dioxane 1,4-dioxane
DMF N,N-dimethylformamide
DCM dichloromethane
DIEA or DIPEA N,N-diisopropylethylamine
DMSO dimethyl sulfoxide
EDCI 1-(3-dimethylaminopropyl)-3-ethylcarbodiimide hydrochloride
ESI electrospray ionization
EtOAc ethyl acetate
eq or equiv equivalent
HATU 2-(7-Azabenzotriazol-1-yl)-N,N,N′,N′-tetramethyluronium hexafluorophosphate
¹H NMR proton nuclear magnetic resonance
HOAt 1-hydroxy-7-azabenzotriazole
KOAc potassium acetate
LC/MS liquid chromatography-mass spectrometry
TEA triethylamine
OMs methanesulfonyloxy
ONs 4-nitrobenzenesulfonyl
OTs 4-methylbenzenesulfonyloxy
Pd(OAc)2 palladium(II) acetate
TLC thin-layer chromatography
Toluene toluene
   Xantphos 4,5-bis(diphenylphosphino)-9,9-dimethylxanthene

The preparation methods for the compounds of Formula I and Formula A of the present disclosure are described in greater detail below. However, these specific methods do not limit the invention in any way. The compounds of the present invention may also be conveniently prepared by optionally combining various synthetic methods described in this specification or known in the art. Such combinations can be readily accomplished by those skilled in the art to which this invention pertains.

All temperatures are reported in degrees Celsius (° C.). Compounds were characterized by ¹H-NMR (400 MHz, Bruker Advance 400 spectrometer). Chemical shifts (6) are reported in parts per million (ppm) relative to the solvent used. Multiplicity is reported as: s=singlet, d=doublet, t=triplet, q=quartet, p=pentet, hex=hextet, hep=heptet, m=multiplet, br. =broad; coupling constants (J) are reported in Hertz (Hz). Alternatively, compounds were characterized by LC/MS using either: (1) a Sciex API 2000 system with Agilent 1100 binary pump, DAD and ELSD detectors, or (2) an Agilent 6125B quadrupole MS system with Agilent 1260 quaternary pump, DAD and ELSD detectors.

Solvents and reagents are processed as follows:
the solvents used in the reactions such as N,N-dimethylformamide, ethyl acetate, anhydrous toluene, dichloromethane, methanol, ethanol, and hydrochloric acid were all purchased from Sinopharm Group; Other reaction reagents and drugs can be purchased from commercial suppliers and used without special instructions.

General synthetic methods of the compounds of Formula I

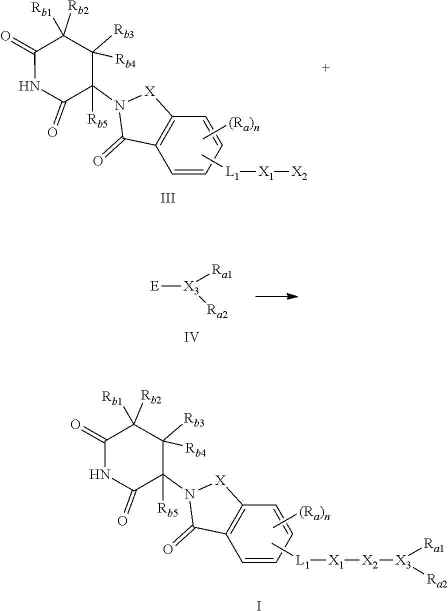

Wherein (1) E-X₃ represents CO, Rₐ₂ represents hydrogen; or (2) E-X₃ represents halogen-CH, MsO—CH, TsO—CH, or NsO—CH; or (3) E-X₃ represents CO, Rₐ₂ represents hydroxy.

Synthetic method I:

In one embodiment of the present application, when E-X₃ represents CO and Rₐ₂ represents hydrogen, the synthesis of the compounds of Formula I comprises:

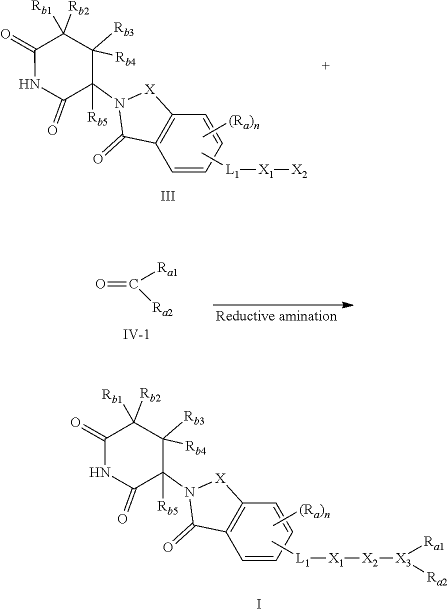

In synthetic method I, (Rₐ)n, X, L₁, X₁, R_{b1}, R_{b2}, R_{b3}, R_{b4}, R_{b5}, Rₐ₁, and Rₐ₂ are as defined in the compounds of Formula (I) and its various sub-embodiments of the present disclosure. In the compound of Formula (III), X₂ represents optionally substituted nitrogen-containing heterocyclyl, optionally substituted aryl-NH₂, optionally substituted heteroaryl-NH₂, or optionally substituted cycloalkyl-NH₂.

Depending on the target compound, modifications and adjustments can be made to the substrates, reaction conditions (including stoichiometry, temperature, time, etc.), and workup procedures in synthetic method I using techniques and methods well-known to those skilled in the art to obtain the desired target compound. The reductive amination reaction in synthetic method I may employ conventional techniques and methods familiar to those skilled in the art. For example, the reductive amination may be conducted in the presence of sodium triacetoxyborohydride and 1,2-dichloroethane, or sodium cyanoborohydride and 1,2-dichloroethane, at temperatures ranging from room temperature to 80° C. (e.g., 40° C.-60° C., 40° C.-50° C., or 50° C.-60° C.). The molar ratio of the compound of Formula III to the compound of Formula IV-1 may be, for instance, 1:1.1-2, 1:1.1-1.5, 1:1.1-1.2, or 1:1.2-1.3 etc.

General Synthetic Procedure: To a solution of the amine substrate (Formula III) (1.0 equiv.) and the aldehyde substrate (Formula IV-1) (1.2 eq.) in 1,2-dichloroethane was added acetic acid (1 drop). The reaction solution was stirred at 50° C. for 2 hours, followed by the addition of sodium triacetoxyborohydride (2.0 eq.) to the solution. The reaction solution was stirred for another 17 hours. After monitoring the reaction via LCMS and confirming its completion, the reaction solution was filtered. The filtrate was subjected to HPLC for separation and purification to afford the target compound.

### Synthetic Method II:

When E-X₃ represents halogen-CH, MsO-CH, TsO-CH, or NsO-CH, the synthesis of the compounds of Formula I comprises:

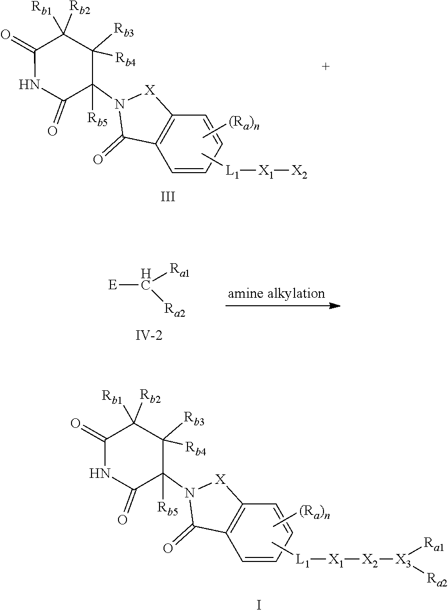

Wherein E is Cl, Br, I, OMs, OTs, or ONs.

In synthetic method II, (Rₐ)n, X, L₁, X₁, R_{b1}, R_{b2}, R_{b3}, R_{b4}, R_{b5}, Rₐ₁, and Rₐ₂ are as defined in the compounds of Formula (I) and its various sub-embodiments of the present disclosure. In the compound of Formula (III), X₂ represents optionally substituted nitrogen-containing heterocyclyl, optionally substituted aryl-NH₂, optionally substituted heteroaryl-NH₂, or optionally substituted cycloalkyl-NH₂.

The amine alkylation reaction in synthetic method II may employ conventional techniques and methods familiar to those skilled in the art. For example, the amine alkylation may be conducted in the presence of either DIEA and sodium iodide, or triethylamine and sodium iodide at temperatures ranging from room temperature to 80° C. (typically 40° C.-60° C., 40° C.-50° C., or 50° C.-60° C.), with the molar ratio of the compound of Formula III to the compound of Formula IV-2 being 1:1.1-2, 1:1.1-1.5, 1:1.1-1.2, or 1:1.2-1.3, etc.

General Synthetic Procedure: To a solution of the amine substrate (Formula III) (1.0 equiv) and the bromide substrate (Formula IV-2) (1.2 equiv) in N,N-dimethylformamide was added triethylamine (2.0 equiv) and sodium iodide (1.0 equiv). The reaction solution was stirred at 50° C. for 2 hours. After monitoring the reaction via LCMS and confirming its completion, the reaction solution was filtered. The filtrate was subjected to HPLC for separation and purification to afford the target compound.

### Synthetic Method III:

When E-X₃ represents CO and Rₐ₂ represents hydroxy, the synthesis of the compounds of Formula I comprises:

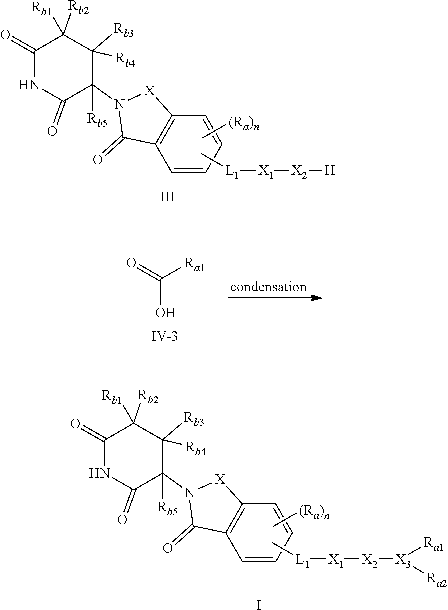

In synthetic method III, (Rₐ)n, X, L₁, X₁, R_{b1}, R_{b2}, R_{b3}, R_{b4}, R_{b5}, Rₐ₁, and Rₐ₂ are as defined in the compounds of Formula (I) and its various sub-embodiments of the present disclosure. In the compound of Formula (III), X₂ represents optionally substituted nitrogen-containing heterocyclyl, optionally substituted aryl-NH₂, optionally substituted heteroaryl-NH₂, or optionally substituted cycloalkyl-NH₂.

The amide condensation reaction in synthetic method III may employ conventional techniques and methods familiar to those skilled in the art. For example, the amide condensation may be conducted in the presence of either HATU, DIEA and DMF, or HOAt, EDCI, TEA and DCM at temperatures ranging from room temperature to 80° C. (e.g., from room temperature to 30° C., 40° C.-60° C., 40° C.-50° C., or 50° C.-60° C.), with the molar ratio of the compound of Formula III to the compound of Formula IV-3 being 1:1.1-2, 1:1.1-1.5, 1:1.1-1.2, or 1:1.2-1.3, etc.

General Synthetic Procedure: To a solution of the amine substrate (1.0 equiv) and the carboxylic acid substrate (1.2 equiv) in N,N-dimethylformamide were added HATU (1.5 equiv) and triethylamine (3.0 equiv). The reaction solution was stirred at 50° C. for 2 hours. After monitoring the reaction via LCMS and confirming its completion, the reaction solution was filtered. The filtrate was subjected to HPLC for separation and purification to afford the target compound.

General synthetic method A of the compounds of Formula A

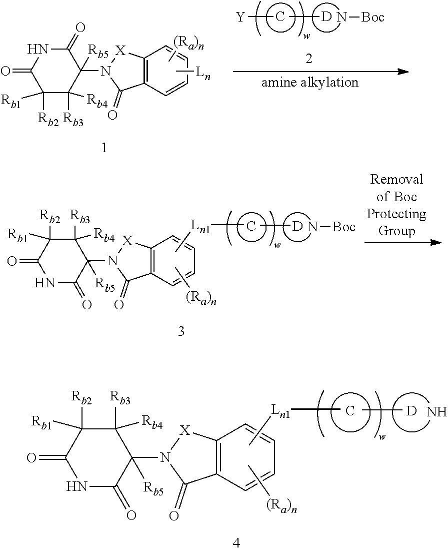

Wherein L. is OH or SH, Y is Cl, Br, I, OMs, OTs, or ONs etc, and Lₙ₁ is O or S.

In synthetic method A, (Rₐ)n, X, ring C, w, R_{b1}, R_{b2}, R_{b3}, R_{b4} and R_{b5} are as defined in the compounds of Formula A and its various sub-embodiments of the present disclosure. The nitrogen-containing heterocycle D in Reaction Substrate 2 of step 1 may be an optionally substituted heterocyclic group including, but not limited to, azetidine, pyrrolidine, imidazolidine, pyrazolidine, piperidine, piperazine, azacycloheptane, azacyclooctane, 7-azaspiro[3.5]nonane, 3-azaspiro[5.5]undecane, octahydrocyclopenta[c]pyrrole, 3-azabicyclo[3.2.0]heptane, 3-azabicyclo[3.1.0]hexane, and 3-azabicyclo[3.1.1]heptane etc.

The amine alkylation reaction in synthetic method A may employ conventional techniques and methods familiar to those skilled in the art. For example, the amine alkylation may be conducted in the presence of potassium carbonate and DMF, or DIEA and sodium iodide, or triethylamine and sodium iodide at temperatures ranging from room temperature to 95° C. (e.g., 40° C.-60° C., 40° C.-50° C., 50° C.-60° C., 60° C.-80° C., or 60° C.-90° C.). The molar ratio of the reaction substrate 1 to the reaction substrate 2 in step 1 may be, for instance, 1:1.1-2, 1:1.1-1.5, 1:1.1-1.2, or 1:1.2-1.3 etc.

General Synthetic Procedure: Step 1: to a solution of the reaction substrate 1 (1.0 equiv) and reaction substrate 2 (1.2 equiv) in N,N-dimethylformamide was added potassium carbonate (2.0 equiv). The reaction flask was purged with N₂ three times to remove air, and the reaction mixture was heated to 80° C. with stirring for 16 hours. After monitoring the reaction via TLC and confirming its completion, the reaction mixture was cooled to room temperature, diluted with water and extracted with dichloromethane. The combined organic phases were dried over anhydrous sodium sulfate, filtered, and the filtrate was concentrated under reduced pressure. The crude residue was purified by silica gel column chromatography to afford Boc-protected product 3.

Step 2: To a solution of the Boc-protected product 3 from Step 1 in dichloromethane was added a solution of HCl in ethyl acetate (2.0 equiv). The reaction mixture was stirred at room temperature for 1 hour. After monitoring the reaction via LCMS and confirming its completion, the reaction mixture was filtered to afford the target product 4 as a solid.

### Example A1: Preparation of 3-(5-(azetidin-3-yloxy)-1-oxoisoindolin-2-yl)piperidine-2,6-dione (GT-03614)

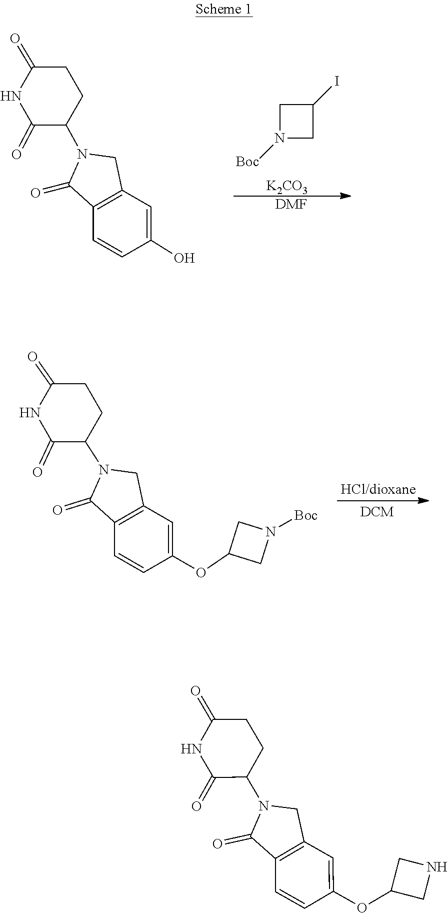

Step 1): To a solution of 3-(5-hydroxy-1-oxoisoindolin-2-yl)piperidine-2,6-dione (7.0 g, 26.9 mmol) in N,N-dimethylformamide (100 mL) were successively added tert-butyl 3-iodoazetidine-1-carboxylate (7.6 g, 26.9 mmol) and potassium carbonate (7.4 g, 53.8 mmol). The reaction mixture was stirred at 90° C. for 18 hours. After monitoring the reaction via TLC and confirming its completion, the reaction mixture was cooled to room temperature and concentrated under reduced pressure. The residue was washed with water (100 mL) and extracted with ethyl acetate (100 mL×3). The combined organic phases were washed with saturated brine (100 mL), filtered, and the filtrate was concentrated under reduced pressure. The residue was purified by column chromatography to afford tert-butyl 3-((2-(2,6-dioxopiperidin-3-yl)-1-oxoisoindolin-5-yl)oxy)azetidine-1-carboxylate (2.0 g, yield: 18%, white solid).

¹H NMR (400 MHz, DMSO) δ 10.96 (s, 1H), 7.65 (d, J=8.4 Hz, 1H), 7.04 (s, 1H), 6.98 (d, J=8.4, Hz, 1H), 5.12-5.03 (m, 2H), 4.43-4.24 (m, 4H), 3.82 (d, J=9.2 Hz, 2H), 2.96-2.85 (m, 1H), 2.61-2.57 (m, 1H), 2.41-2.36 (m, 1H), 2.03-1.93 (m, 1H), 1.39 (s, 9H). LC/MS (ESI) m/z: calcd for C₂₁H₂₅N₃NaO₆⁺ [M+Na]⁺, 438.16; found, 438.2.

Step 2): To a solution of tert-butyl 3-((2-(2,6-dioxopiperidin-3-yl)-1-oxoisoindolin-5-yl)oxy)azetidine-1-carboxylate (2.0 g, 4.8 mmol) in dichloromethane (20 mL) was added hydrochloric acid/1,4-dioxane solution (80 mL, 3 M). The reaction mixture was stirred at room temperature for 1 hour. After monitoring the reaction via TLC and confirming its completion, the reaction mixture was filtered. The filter cake was washed with ethyl acetate (10 mL×2) to afford 3-(5-(azetidin-3-yloxy)-1-oxoisoindolin-2-yl)piperidine-2,6-dione (GT-03614) (1.6 g, yield: 92%, white solid). ¹H NMR (400 MHz, DMSO) δ 10.97 (s, 1H), 9.32 (brs, 1H), 9.30 (brs, 1H), 7.67 (d, J=8.4 Hz, 1H), 7.10 (s, 1H), 7.02 (d, J=8.4, 1H), 5.20-5.16 (m, 1H), 5.10-5.06 (m, 1H), 4.48-4.46 (m, 2H), 4.34 (dd, J=48.6, 17.2 Hz, 2H), 4.01 (brs, 2H), 2.97-2.85 (m, 1H), 2.62-2.60 (m, 1H), 2.39-2.37 (m, 1H), 2.03-1.95 (m, 1H), 1.24 (s, 1H). LC/MS (ESI) m/z: calcd for C₁₆H₁₈N₃O₄⁺ [M+H]⁺, 316.13; found, 316.2.

### Example A2: Preparation of 3-(5-(azetidin-3-ylthio)-1-oxoisoindolin-2-yl)piperidine-2,6-dione (GT-03613)

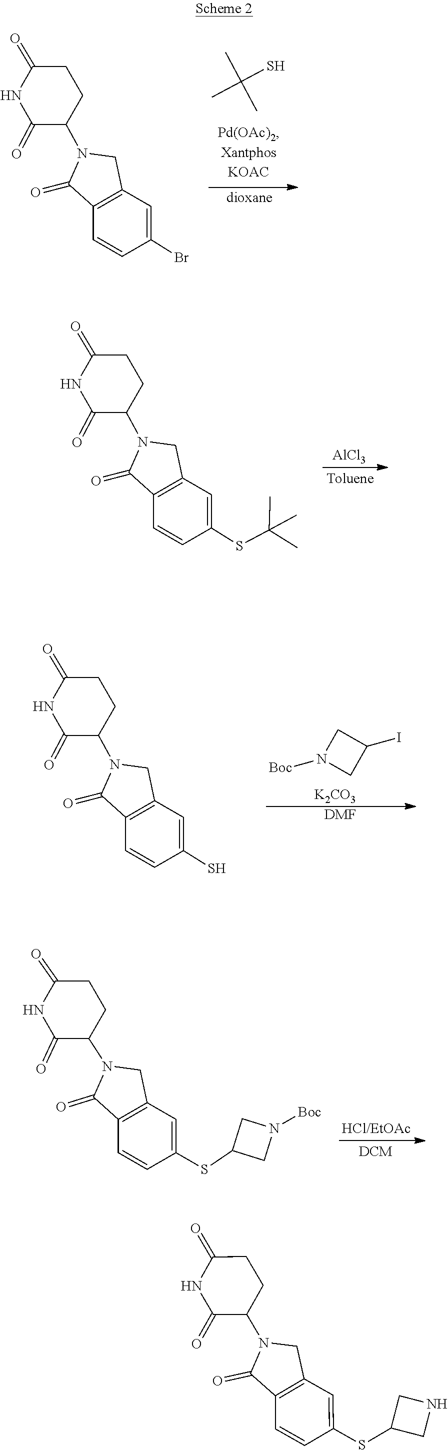

A mixture of 3-(5-bromo-1-oxoisoindolin-2-yl)piperidine-2,6-dione (10.0 g, 30.9 mmol), Xantphos (0.54 g, 0.93 mmol), palladium acetate (0.21 g, 0.93 mmol), potassium acetate (6.07 g, 61.9 mmol), and tert-butylthiol (5.2 mL, 46.4 mmol) in 1,4-dioxane (120 mL) was degassed with nitrogen three times and heated to 90° C. for 18 hours. After monitoring the reaction via LCMS and confirming its completion, the reaction mixture was cooled to room temperature and concentrated under reduced pressure. The residue was washed with water (50 mL) and extracted with dichloromethane (100 mL×3).

The combined organic phases were dried over anhydrous sodium sulfate, filtered, and the filtrate was concentrated under reduced pressure. The residue was triturated with ethanol (40 mL) and filtered to afford 3-(5-(tert-butylthio)-1-oxoisoindolin-2-yl)piperidine-2,6-dione (9.4 g, 91%, brown solid). LC/MS (ESI) m/z: calcd for C₁₇H₂₁N₂O₃S⁺ [M+H]⁺, 333.13; found, 333.1.

To a solution of 3-(5-(tert-butylthio)-1-oxoisoindolin-2-yl)piperidine-2,6-dione (9.4 g, 28.3 mmol) in anhydrous toluene (120 mL) was added aluminum chloride (11.3 g, 84.8 mmol) portionwise.

The reaction mixture was heated to 35° C. for 18 hours. After monitoring the reaction via LCMS and confirming its completion, the reaction mixture was cooled to room temperature, quenched with 20% aqueous citric acid (50 mL), and stirred for 2 hours. The resulting precipitate was filtered and washed with ethyl acetate (50 mL×2) to give 3-(5-mercapto-1-oxoisoindolin-2-yl)piperidine-2,6-dione (7.2 g, 92%, brown solid). LC/MS (ESI) m/z: calcd for C₁₃H₁₃N₂O₃S⁺ [M+H]⁺, 277.06; found, 277.0.

To a solution of 3-(5-mercapto-1-oxoisoindolin-2-yl)piperidine-2,6-dione (3.3 g, 11.9 mmol) in N,N-dimethylformamide (33 mL) were successively added tert-butyl 3-iodoazetidine-1-carboxylate (3.38 g, 11.9 mmol) and potassium carbonate (4.13 g, 29.8 mmol). The reaction mixture was heated to 80° C. for 18 hours. After monitoring the reaction via LCMS and confirming its completion, the reaction mixture was diluted with ethyl acetate (200 mL) and washed with water (50 mL×3). The organic phases were dried over anhydrous sodium sulfate, filtered, and the filtrate was concentrated under reduced pressure to give tert-butyl 3-((2-(2,6-dioxopiperidin-3-yl)-1-oxoisoindolin-5-yl)thio)azetidine-1-carboxylate (4.5 g, yield: 87%, yellow solid). LC/MS (ESI) m/z: calcd for C₂₁H₂₅N₃NaO₅S⁺ [M+Na]⁺, 454.14; found, 454.1.

To a solution of tert-butyl 3-((2-(2,6-dioxopiperidin-3-yl)-1-oxoisoindolin-5-yl)thio)azetidine-1-carboxylate (8.8 g, 20.4 mmol) in dichloromethane (60 mL) was added HCl/ethyl acetate solution (88 mL, 3M). The reaction mixture was stirred to room temperature for 3 hours. After monitoring the reaction via TLC and confirming its completion, the reaction mixture was filtered. The filter cake was washed with ethyl acetate (10 mL×2) to afford 3-(5-(azetidin-3-ylthio)-1-oxoisoindolin-2-yl)piperidine-2,6-dione (GT-03613) (5 g, yield: 67%, yellow solid). ¹H NMR (400 MHz, DMSO) δ 10.99 (s, 1H), 9.27 (brs, 2H), 7.69 (d, J=8.0 Hz, 1H), 7.52 (s, 1H), 7.40 (d, J=8.0 Hz, 1H), 5.11 (dd, J=13.2, 5.2 Hz, 1H), 4.68-4.29 (m, 5H), 3.88 (s, 2H), 2.92-2.89 (m, 1H), 2.62-2.59 (m, 1H), 2.45-2.33 (m, 1H), 2.01-1.98 (m, 1H).

LC/MS (ESI) m/z: calcd for C₁₆H₁₈N₃O₃S⁺ [M+H]⁺, 332.11; found, 332.0.

### Example A3: Preparation of 3-(1-oxo-5-(pyrrolidin-3-ylthio)isoindolin-2-yl)piperidine-2,6-dione (GT-04309)

The target compound (GT-04309) was prepared as a yellow solid (3.7 g, yield: 99%) according to the method described in Scheme 2. ¹H NMR (400 MHz, DMSO) δ 10.99 (s, 1H), 9.18 (brs, 2H), 7.69 (d, J=8.0 Hz, 1H), 7.65 (s, 1H), 7.51 (d, J=8.0 Hz, 1H), 5.13-5.08 (m, 1H), 4.39 (dd, J=48.6, 17.2 Hz, 2H), 4.26-4.12 (m, 1H), 3.71-3.63 (m, 1H), 3.28 (s, 1H), 3.12-3.10 (m, 1H), 2.95-2.83 (m, 1H), 2.61-2.59 (m, 1H), 2.46-2.25 (m, 2H), 2.04-1.82 (m, 2H), 1.28-1.23 (m, 1H). LC/MS (ESI) m/z: calcd for C₁₇H₁₉N₃O₃S⁺ [M+H]⁺, 346.12; found, 346.0.

### Example A4: Preparation of 3-(1-oxo-5-(piperidin-4-ylthio)isoindolin-2-yl)piperidine-2,6-dione (GT-03616)

The target compound (GT-03616) was prepared as a white solid (2.7 g, yield: 51%) according to the method described in Scheme 2. ¹H NMR (400 MHz, DMSO) δ 11.00 (s, 1H), 8.67 (s, 1H), 8.59 (s, 1H), 7.73-7.63 (m, 2H), 7.52 (dd, J=8.0 Hz, 1H), 5.11 (dd, J=13.2, 5.2 Hz, 1H), 4.38 (dd, J=48.6, 17.2 Hz, 2H), 3.75-3.61 (m, 1H), 3.33-3.30 (m, 2H), 3.06-2.85 (m, 3H), 2.62-2.59 (m, 1H), 2.42-2.39 (m, 1H), 2.12-2.09 (m, 2H), 2.04-1.95 (m, 1H), 1.74-1.59 (m, 2H). LC/MS (ESI) m/z: calcd for C₁₈H₂₂N₃O₃S⁺ [M+H]⁺, 360.14; found, 360.1.

### Example A5: Preparation of 3-(5-(azepan-4-ylthio)-1-oxoisoindolin-2-yl)piperidine-2,6-dione (GT-06995)

The target compound (GT-06995) was prepared as a white solid (2.0 g, yield: 86%) according to the method described in synthetic method A. ¹H NMR (400 MHz, MeOD-d4) δ 7.75 (d, J=8.0 Hz, 1H), 7.64 (s, 1H), 7.53 (d, J=8.0 Hz, 1H), 5.14 (dd, J=13.3, 5.2 Hz, 1H), 4.60-4.41 (m, 2H), 3.79 (tt, J=8.7, 4.3 Hz, 1H), 3.42 (ddd, J=14.0, 7.6, 2.0 Hz, 1H), 3.32-3.20 (m, 3H), 3.00-2.73 (m, 2H), 2.51 (qd, J=13.2, 4.8 Hz, 1H), 2.41-2.16 (m, 3H), 2.15-1.88 (m, 3H), 1.86-1.73 (m, 1H). LCMS (ESI) calcd for C₁₉H₂₄N₃O₃S⁺ [M+H]⁺: 374.15, found, 374.1.

### Example A6: Preparation of 3-(4-(azetidin-3-ylthio)-1-oxoisoindolin-2-yl)piperidine-2,6-dione (GT-06992)

The target compound (GT-06992) was prepared as a white solid (4.5 g, yield: 76%) according to the method described in synthetic method A. ¹H NMR (400 MHz, DMSO-d6) δ 11.01 (s, 1H), 9.08 (s, 2H), 7.69 (dd, J=6.2, 2.2 Hz, 1H), 7.62-7.52 (m, 2H), 5.15 (dd, J=13.3, 5.1 Hz, 1H), 4.56-4.44 (m, 3H), 4.40 (s, 1H), 4.27 (d, J=17.5 Hz, 1H), 3.95-3.85 (m, 2H), 3.00-2.87 (m, 1H), 2.60 (d, J=17.6 Hz, 1H), 2.45 (dd, J=13.1, 4.4 Hz, 1H), 2.01 (dd, J=11.0, 5.9 Hz, 1H). LCMS (ESI) calcd for C₁₆H₁₈N₃O₃S⁺ [M+H]⁺: 332.11, found, 332.1.

### Example A7: Preparation of 3-(6-(azetidin-3-ylthio)-1-oxoisoindolin-2-yl)piperidine-2,6-dione (GT-06993)

The target compound (GT-06993) was prepared as a white solid (2.5 g, yield: 70%) according to the method described in synthetic method A. ¹H NMR (400 MHz, MeOD-d4) δ 7.76 (s, 1H), 7.66 (dd, J=8.0, 1.5 Hz, 1H), 7.61 (d, J=8.0 Hz, 1H), 5.16 (dd, J=13.3, 5.2 Hz, 1H), 4.58-4.40 (m, 5H), 4.02-3.97 (m, 2H), 2.92-2.86 (m, 1H), 2.82-2.78 (m, 1H), 2.56-2.44 (m, 1H), 2.21-2.15 (m, 1H). LCMS (ESI) calcd for C₁₆H₁₈N₃O₃S⁺ [M+H]⁺: 332.11, found, 332.1.

### Example A8: Preparation of 3-(7-(azetidin-3-ylthio)-1-oxoisoindolin-2-yl)piperidine-2,6-dione (GT-06994)

The target compound (GT-06994) was prepared as a white solid (3.1 g, yield: 78%) according to the method described in synthetic method A. ¹H NMR (400 MHz, MeOD) δ 7.58 (t, J=7.7 Hz, 1H), 7.49-7.38 (m, 1H), 7.15 (d, J=7.7 Hz, 1H), 5.10 (dd, J=13.3, 5.2 Hz, 1H), 4.70-4.60 (m, 2H), 4.58-4.53 (m, 1H), 4.49-4.43 (m, 2H), 4.00 (dd, J=11.8, 5.9 Hz, 2H), 2.97-2.85 (m, 1H), 2.82-2.76 (m, 1H), 2.54-2.46 (m, 1H), 2.20-2.14 (m, 1H). LCMS (ESI) calcd for C₁₆H₁₈N₃O₃S⁺ [M+H]⁺: 332.11, found, 332.1.

### Example A9: Preparation of 3-(5-((7-azaspiro[3.5]nonan-2-yl)thio)-1-oxoisoindolin-2-yl)piperidine-2,6-dione (GT-06997)

The target compound (GT-06997) was prepared as a white solid (480 mg, yield: 69%) according to the method described in synthetic method A. ¹H NMR (400 MHz, D₂O) δ 7.59 (d, J=8.0 Hz, 1H), 7.26-7.24 (m, 2H), 5.04 (dd, J=13.2, 5.2 Hz, 1H), 4.45-4.32 (m, 2H), 4.05-3.85 (m, 1H), 3.14-3.06 (m, 2H), 3.06-2.98 (m, 2H), 2.86-2.82 (m, 2H), 2.52-2.35 (m, 3H), 2.23-2.11 (m, 1H), 1.89-1.84 (m, 4H), 1.80-1.73 (m, 2H). LCMS (ESI) calcd for C₂H₂₆N₃O₃S⁺ [M+H]⁺: 400.17, found, 400.1.

### Example A10: Preparation of 3-(5-((3-azaspiro[5.5]undecan-9-yl)thio)-1-oxoisoindolin-2-yl)piperidine-2,6-dione (GT-06998)

The target compound (GT-06998) was prepared as a white solid (350 mg, yield: 90%) according to the method described in synthetic method A. ¹H NMR (400 MHz, MeOD-d4) δ 7.70 (d, J=8.0 Hz, 1H), 7.56 (s, 1H), 7.49 (dd, J=8.0, 1.4 Hz, 1H), 5.14 (dd, J=13.3, 5.2 Hz, 1H), 4.53-4.39 (m, 2H), 3.49-3.38 (m, 1H), 3.19-3.11 (m, 4H), 2.91-2.86 (m, 1H), 2.81-2.76 (m, 1H), 2.50-2.46 (m, 1H), 2.22-2.13 (m, 1H), 1.99-1.91 (m, 2H), 1.85-1.71 (m, 4H), 1.65-1.54 (m, 4H), 1.47-1.37 (m, 2H).

LCMS (ESI) calcd for C₂₃H₃₀N₃O₃S⁺ [M+H]⁺: 428.20, found, 428.2.

Example All: Preparation of 3-(5-((octahydrocyclopenta[c]pyrrol-5-yl)thio)-1-oxoisoindolin-2-yl)piperidine-2,6-dione (GT-06996)

The target compound (GT-06996) was prepared as a white solid (960 mg, yield: 88%) according to the method described in synthetic method A. ¹H NMR (400 MHz, DMSO-d6) δ 10.99 (s, 1H), 9.37 (s, 2H), 7.78-7.53 (m, 2H), 7.52-7.34 (m, 1H), 5.10 (dd, J=13.2, 4.9 Hz, 1H), 4.49 (d, J=17.5 Hz, 1H), 4.30 (d, J=17.5 Hz, 1H), 4.14-3.64 (m, 1H), 3.31-3.26 (m, 1H), 3.24-3.04 (m, 3H), 3.02-2.82 (m, 4H), 2.60 (d, J=16.5 Hz, 1H), 2.43-2.32 (m, 2H), 2.18-2.04 (m, 1H), 2.02-1.94 (m, 1H), 1.84-1.73 (m, 1H), 1.58-1.47 (m, 1H). LCMS (ESI) calcd for C₂₀H₂₄N₃O₃S⁺ [M+H]⁺: 386.15, found, 386.1.

### Example A12: Preparation of 3-(5-((3,3-difluoropiperidin-4-yl)thio)-1-oxoisoindolin-2-yl)piperidine-2,6-dione (GT-06999)

The target compound (GT-06999) was prepared as a white solid (1.65 g, yield: 80%) according to the method described in synthetic method A. ¹H NMR (400 MHz, DMSO-d6) δ 11.00 (s, 1H), 10.36 (s, 1H), 9.46 (s, 1H), 7.80-7.67 (m, 2H), 7.61 (d, J=8.0 Hz, 1H), 5.12 (dd, J=13.3, 4.9 Hz, 1H), 4.46 (d, J=17.5 Hz, 1H), 4.33 (d, J=17.5 Hz, 1H), 4.31-4.18 (m, 1H), 3.86-3.81 (m, 1H), 3.64-3.59 (m, 1H), 3.29-3.26 (m, 1H), 3.12 (t, J=12.1 Hz, 1H), 2.97-2.84 (m, 1H), 2.61 (d, J=16.8 Hz, 1H), 2.46-2.31 (m, 2H), 2.02-1.94 (m, 2H). LCMS (ESI) calcd for C₁₈H₂₀F₂N₃O₃S⁺ [M+H]⁺: 396.12, found, 396.1.

### Example A13: Preparation of 3-(1-oxo-5-(pyrrolidin-3-yloxy)isoindolin-2-yl)piperidine-2,6-dione (GT-07002)

The target compound (GT-07002) was prepared as a white solid (1.0 g, yield: 86%) according to the method described in synthetic method A. ¹H NMR (400 MHz, DMSO-d6) δ 10.98 (s, 1H), 9.84 (s, 1H), 9.61 (s, 1H), 7.66 (d, J=8.4 Hz, 1H), 7.23 (s, 1H), 7.09 (dd, J=8.4, 1.9 Hz, 1H), 5.29-5.22 (m, 1H), 5.08 (dd, J=13.3, 5.0 Hz, 1H), 4.41 (d, J=17.3 Hz, 1H), 4.29 (d, J=17.3 Hz, 1H), 3.55-3.49 (m, 1H), 3.38-3.34 (m, 2H), 3.30-3.17 (m, 1H), 2.96-2.87 (m, 1H), 2.60 (d, J=17.1 Hz, 1H), 2.42-2.37 (m, 1H), 2.30-2.11 (m, 2H), 2.01-1.97 (m, 1H). LCMS (ESI) calcd for C₁₇H₂₀N₃O₄⁺ [M+H]⁺: 330.14, found, 330.1.

### Example A14: Preparation of 3-(1-oxo-5-(piperidin-4-yloxy)isoindolin-2-yl)piperidine-2,6-dione (GT-03612)

The target compound (GT-03612) was prepared as a white solid (2.3 g, yield: 98%) according to the method described in synthetic method A. ¹H NMR (400 MHz, DMSO) δ 10.96 (s, 1H), 9.15 (s, 2H), 7.65 (d, J=8.4 Hz, 1H), 7.25 (s, 1H), 7.13 (dd, J=8.4, 1.9 Hz, 1H), 5.07 (dd, J=13.3, 5.0 Hz, 1H), 4.84-4.75 (m, 1H), 4.40 (d, J=17.2 Hz, 1H), 4.27 (d, J=17.2 Hz, 1H), 3.22 (s, 2H), 3.07 (d, J=6.0 Hz, 2H), 2.91 (ddd, J=13.5, 12.5, 5.4 Hz, 1H), 2.60 (d, J=16.9 Hz, 1H), 2.39 (qd, J=13.2, 4.3 Hz, 1H), 2.15 (s, 2H), 2.03-1.95 (m, 1H), 1.90 (d, J=11.9 Hz, 2H). LCMS (ESI) calcd for C₁₈H₂₂N₃O₄⁺[M+H]⁺: 344.16, found, 344.1.

### Example A15: Preparation of 3-(5-(azepan-4-yloxy)-1-oxoisoindolin-2-yl)piperidine-2,6-dione (GT-07003)

The target compound (GT-07003) was prepared as a white solid (440 mg, yield: 91%) according to the method described in synthetic method A. ¹H NMR (400 MHz, DMSO-d6) δ 10.97 (s, 1H), 8.76 (s, 2H), 7.64 (d, J=8.4 Hz, 1H), 7.20 (s, 1H), 7.07 (d, J=8.5 Hz, 1H), 5.08 (dd, J=13.3, 5.0 Hz, 1H), 4.87-4.78 (m, 1H), 4.39 (d, J=17.1 Hz, 1H), 4.27 (d, J=17.2 Hz, 1H), 3.30-3.24 (m, 1H), 3.21-3.04 (m, 3H), 2.99-2.83 (m, 1H), 2.60 (d, J=17.1 Hz, 1H), 2.43-2.34 (m, 1H), 2.27-2.14 (m, 1H), 2.13-1.83 (m, 5H), 1.78-1.71 (m, 1H). LCMS (ESI) calcd for C₁₉H₂₄N₃O₄′ [M+H]⁺: 358.18, found, 358.1.

### Example A16: Preparation of 3-(4-(azetidin-3-yloxy)-1-oxoisoindolin-2-yl)piperidine-2,6-dione (GT-07000)

The target compound (GT-07000) was prepared as a white solid (4.2 g, yield: 95%) according to the method described in synthetic method A. ¹H NMR (400 MHz, MeOD) δ 7.59-7.40 (m, 2H), 6.99-6.97 (m, 1H), 5.37-5.27 (m, 1H), 5.17 (dd, J=13.3, 5.1 Hz, 1H), 4.64-4.59 (m, 2H), 4.56-4.44 (m, 2H), 4.23-4.19 (m, 2H), 2.97-2.88 (m, 1H), 2.83-2.77 (m, 1H), 2.56-2.47 (m, 1H), 2.23-2.17 (m, 1H).LCMS (ESI) calcd for C₁₆H₁₈N₃O₄⁺ [M+H]⁺: 316.13, found, 316.1.

### Example A17: Preparation of 3-(6-(azetidin-3-yloxy)-1-oxoisoindolin-2-yl)piperidine-2,6-dione (GT-07001)

The target compound (GT-07001) was prepared as a white solid (1.1 g, yield: 93%) according to the method described in synthetic method A. ¹H NMR (400 MHz, DMSO-d6) δ 10.99 (s, 1H), 9.23 (s, 1H), 9.06 (s, 1H), 7.58 (d, J=8.2 Hz, 1H), 7.20 (dd, J=8.3, 2.5 Hz, 1H), 7.11 (d, J=2.4 Hz, 1H), 5.28-5.17 (m, 1H), 5.11 (dd, J=13.3, 5.1 Hz, 1H), 4.55-4.45 (m, 2H), 4.44-4.35 (m, 1H), 4.26 (d, J=17.1 Hz, 1H), 4.05-4.01 (m, 2H), 2.96-2.87 (m, 1H), 2.60 (d, J=17.2 Hz, 1H), 2.45-2.34 (m, 1H), 2.03-1.97 (m, 1H). LCMS (ESI) calcd for C₁₆H₁₈N₃O₄⁺ [M+H]⁺: 316.13, found, 316.1.

### Example A18: Preparation of 3-(7-(azetidin-3-yloxy)-1-oxoisoindolin-2-yl)piperidine-2,6-dione (GT-07052)

The target compound (GT-07052) was prepared as a white solid (95 mg, yield: 100%) according to the method described in synthetic method A. ¹H NMR (400 MHz, DMSO-d6) δ 10.98 (s, 1H), 9.07 (s, 1H), 8.89 (s, 1H), 7.56 (t, J=7.9 Hz, 1H), 7.25 (d, J=7.5 Hz, 1H), 6.84 (d, J=8.2 Hz, 1H), 5.28-5.12 (m, 1H), 5.01 (dd, J=13.2, 5.0 Hz, 1H), 4.57-4.37 (m, 3H), 4.30 (d, J=17.5 Hz, 1H), 4.07 (s, 2H), 2.95-2.84 (m, 1H), 2.68-2.59 (m, 1H), 2.42-2.34 (m, 1H), 2.02-1.96 (m, 1H). LCMS (ESI) calcd for C₁₆H₁₈N₃O₄⁺ [M+H]⁺: 316.13, found, 316.1.

### Example A19: Preparation of 3-(5-((7-azaspiro[3.5]nonan-2-yl)oxy)-1-oxoisoindolin-2-yl)piperidine-2,6-dione (GT-07005)

The target compound (GT-07005) was prepared as a white solid (2.1 g, yield: 95%) according to the method described in synthetic method A. ¹H NMR (400 MHz, DMSO-d6) δ 10.97 (s, 1H), 8.97 (s, 2H), 7.62 (d, J=8.4 Hz, 1H), 7.06 (s, 1H), 6.97 (dd, J=8.4, 1.9 Hz, 1H), 5.07 (dd, J=13.3, 5.0 Hz, 1H), 4.90-4.79 (m, 1H), 4.38 (d, J=17.3 Hz, 1H), 4.26 (d, J=17.2 Hz, 1H), 3.07-3.00 (m, 2H), 2.98-2.92 (m, 2H), 2.87 (d, J=5.3 Hz, 1H), 2.64-2.52 (m, 2H), 2.50-2.47 (m, 1H), 2.39-2.33 (m, 1H), 2.03-1.95 (m, 1H), 1.93-1.85 (m, 2H), 1.83-1.80 (m, 2H), 1.79-1.71 (m, 2H). LCMS (ESI) calcd for C₁₉H₂₆N₃O₄⁺ [M+H]⁺: 384.19, found, 384.2.

### Example A20: Preparation of 3-(5-((octahydrocyclopenta[c]pyrrol-5-yl)oxy)-1-oxoisoindolin-2-yl)piperidine-2,6-dione (GT-07004)

The target compound (GT-07004) was prepared as a white solid (1.5 g, yield: 86%) according to the method described in synthetic method A. ¹H NMR (400 MHz, DMSO) δ 11.03 (s, 1H), 9.50 (s, 1H), 9.39 (s, 1H), 7.71-7.66 (m, 1H), 7.29-7.26 (m, 1H), 7.16-7.09 (m, 1H), 5.19-5.12 (m, 1H), 5.08-5.06 (m, 1H), 4.45 (d, J=17.2 Hz, 1H), 4.32 (d, J=17.2 Hz, 1H), 3.49-3.22 (m, 2H), 3.16-2.87 (m, 5H), 2.65 (d, J=16.9 Hz, 1H), 2.47-2.43 (m, 1H), 2.25-2.22 (m, 1H), 2.14-2.01 (m, 2H), 1.94-1.83 (m, 1H). LCMS (ESI) calcd for C₂₀H₂₄N₃O₄⁺ [M+H]⁺: 370.18, found, 370.1.

### Example 1: Preparation of 3-(5-((1-((4′-chloro-5,5-dimethyl-3,4,5,6-tetrahydro-[1,1′-biphenyl]-2-yl)methyl)azetidin-3-yl)thio)-1-oxoisoindolin-2-yl)piperidine-2,6-dione (GT-03464; compound 1)

The synthesis was performed according to synthetic method I using compound (GT-03613) as the starting material.

The target compound (GT-03464) was prepared as a white solid (10 mg, yield: 20%). ¹H NMR (400 MHz, MeOD) δ 7.66 (d, J=8.0 Hz, 1H), 7.33-7.28 (m, 3H), 7.24-7.15 (m, 1H), 7.05-6.99 (m, 2H), 5.05 (dd, J=13.2, 5.2 Hz, 2H), 4.59-4.44 (m, 4H), 4.41-4.30 (m, 4H), 3.74 (s, 2H), 2.95-2.75 (m, 2H), 2.73-2.67 (m, 1H), 2.40-2.36 (m, 1H), 2.17-2.02 (m, 5H), 1.49-1.43 (m 2H), 0.92 (s, 6H).

LC/MS (ESI) m/z: calcd for C₃₁H₃₅ClN₃O₃S⁺ [M+H]⁺, 564.21; found, 564.2.

### Example 2: Preparation of 3-(5-((1-((4′-chloro-5,5-dimethyl-3,4,5,6-tetrahydro-[1,1′-biphenyl]-2-yl)methyl)pyrrolidin-3-yl)thio)-1-oxoisoindolin-2-yl)piperidine-2,6-dione (GT-04245; compound 5)

The synthesis was performed according to synthetic method I using compound (GT-04309) as the starting material.

The target compound (GT-04245) was prepared as a white solid (12 mg, yield: 18%). ¹H NMR (400 MHz, MeOD) δ 7.67 (d, J=8.0 Hz, 1H), 7.53-7.33 (m, 2H), 7.29 (d, J=8.4 Hz, 2H), 7.00 (d, J=8.4 Hz, 2H), 5.06 (dd, J=13.2, 5.2 Hz, 1H), 4.46-4.33 (m, 2H), 4.15-3.90 (m, 1H), 3.85-3.77 (m, 1H), 3.72-3.65 (m, 1H), 3.56-3.48 (m, 1H), 3.37-3.28 (m, 1H), 3.14-3.08 (m, 1H), 2.87-2.77 (m, 2H), 2.74-2.63 (m, 1H), 2.50-2.29 (m, 2H), 2.22-2.15 (m, 2H), 2.09-2.03 (m, 3H), 1.96-1.79 (m, 1H), 1.47 (t, J=6.0 Hz, 2H), 0.92 (s, 6H). LC/MS (ESI) m/z: calcd for C₃₂H₃₇ClN₃O₃S⁺ [M+H]⁺, 578.22; found, 578.3.

### Example 3: Preparation of 3-(5-((1-((4′-chloro-5,5-dimethyl-3,4,5,6-tetrahydro-[1,1′-biphenyl]-2-yl)methyl)piperidin-4-yl)thio)-1-oxoisoindolin-2-yl)piperidine-2,6-dione (GT-03462; compound 6)

The synthesis was performed according to synthetic method I using compound (GT-03616) as the starting material.

The target compound (GT-03462) was prepared as a white solid (6 mg, yield: 16%). ¹H NMR (400 MHz, MeOD) δ 7.65 (d, J=8.0 Hz, 1H), 7.51 (brs, 1H), 7.46-7.41 (m, 1H), 7.35-7.29 (m, 2H), 7.06-6.99 (s, 2H), 5.05 (dd, J=13.2, 5.2 Hz, 1H), 4.60-4.50 (m, 2H), 4.45-4.30 (m, 2H), 3.53-3.46 (m, 2H), 3.39-3.31 (m, 3H), 2.89-2.74 (m, 1H), 2.74-2.54 (m, 2H), 2.47-2.28 (m, 1H), 2.26-1.96 (m, 7H), 1.78-1.67 (m, 1H), 1.53-1.42 (m, 2H), 0.92 (s, 6H). LC/MS (ESI) m/z: calcd for C₃₃H₃₉ClN₃O₃S⁺ [M+H]⁺, 592.24; found, 592.3.

### Example 4: Preparation of 3-(5-((1-((4′-chloro-[1,1′-biphenyl]-2-yl)methyl)azetidin-3-yl)thio)-1-oxoisoindolin-2-yl)piperidine-2,6-dione (GT-03360; compound 28)

The synthesis was performed according to synthetic method II using compound (GT-03613) as the starting material.

The target compound (GT-03360) was prepared as a white solid (20 mg, yield: 47%). ¹H NMR (400 MHz, MeOD) δ 7.64 (d, J=8.0 Hz, 1H), 7.44-7.40 (m, 4H), 7.30 (d, J=12.8 Hz, 2H), 7.24-7.22 (m, 3H), 7.21 (d, J=8.4 Hz, 1H), 5.07-5.03 (m, 1H), 4.41-4.36 (m, 5H), 4.28-4.17 (m, 2H), 3.76-3.70 (m, 2H), 2.86-2.76 (m, 1H), 2.71-2.66 (m, 1H), 2.41-2.37 (m, 1H), 2.10-2.05 (m, 1H). LC/MS (ESI) m/z: calcd for C₂₉H₂₇ClN₃O₃S⁺ [M+H]⁺, 532.15; found, 532.1.

### Example 5: Preparation of 3-(5-((1-((4′-chloro-[1,1′-biphenyl]-2-yl)methyl)pyrrolidin-3-yl)thio)-1-oxoisoindolin-2-yl)piperidine-2,6-dione (GT-04195; compound 29)

The synthesis was performed according to synthetic method II using compound (GT-04309) as the starting material.

The target compound (GT-04195) was prepared as a white solid (27 mg, yield: 45%). ¹H NMR (400 MHz, MeOD) δ 7.79 (d, J=8.0 Hz, 1H), 7.72-7.69 (m, 1H), 7.62-7.55 (m, 2H), 7.53-7.48 (m, 3H), 7.44-7.41 (m, 2H), 7.34 (d, J=8.4 Hz, 2H), 5.18 (dd, J=13.2, 5.2 Hz, 1H), 4.57-4.47 (m, 4H), 4.20-4.04 (m, 1H), 3.64-3.44 (m, 1H), 3.10-2.86 (m, 2H), 2.84-2.82 (m, 1H), 2.54-2.49 (m, 2H), 2.22-2.20 (m, 1H), 2.02-1.91 (m, 1H), 1.46-1.37 (m, 3H). LC/MS (ESI) m/z: calcd for C₃₀H₂₉ClN₃O₃S+[M+H]⁺, 546.16; found, 546.2.

### Example 6: Preparation of 3-(5-((1-((4′-chloro-[1,1′-biphenyl]-2-yl)methyl)piperidin-4-yl)thio)-1-oxoisoindolin-2-yl)piperidine-2,6-dione (GT-03357; compound 30)

The synthesis was performed according to synthetic method II using compound (GT-03616) as the starting material.

The target compound (GT-03357) was prepared as a white solid (18 mg, yield: 51%). ¹H NMR (400 MHz, MeOD) δ 7.78-7.73 (m, 2H), 7.65-7.48 (m, 6H), 7.47-7.33 (m, 3H), 5.16 (dd, J=13.2, 5.2 Hz, 1H), 4.54-4.41 (m, 2H), 4.39 (s, 1H), 3.51-3.42 (m, 1H), 3.39-3.35 (m, 2H), 3.29-3.01 (m, 1H), 3.00-2.86 (m, 1H), 2.83-2.77 (m, 2H), 2.59-2.40 (m, 1H), 2.23-2.11 (m, 3H), 1.82-1.73 (m, 2H), 1.41-1.38 (m, 1H).LC/MS (ESI) m/z: calcd for C₃₁H₃₁ClN₃O₃S⁺ [M+H]⁺, 560.18; found, 560.2.

### Example 7: Preparation of 3-(5-((1-(4′-chloro-[1,1′-biphenyl]-2-carbonyl)azetidin-3-yl)thio)-1-oxoisoindolin-2-yl)piperidine-2,6-dione (GT-03892; compound 37)

The synthesis was performed according to synthetic method III using compound (GT-03613) as the starting material.

The target compound (GT-03892) was prepared as a white solid (15 mg, yield: 20%). ¹H NMR (400 MHz, MeOD) δ 7.73-7.71 (m, 1H), 7.59-7.54 (m, 1H), 7.52-7.43 (m, 7H), 7.33 (s, 1H), 7.29-7.24 (m, 1H), 5.17-5.10 (m, 1H), 4.53-4.43 (m, 3H), 4.22-4.13 (m, 2H), 3.83-3.77 (m, 1H), 3.04-2.86 (m, 2H), 2.84-2.79 (m, 1H), 2.55-2.44 (m, 1H), 2.23-2.13 (m, 1H). LC/MS (ESI) m/z: calcd for C₂₉H₂₅ClN₃O₄S⁺ [M+H]⁺, 546.12; found, 546.1.

### Example 8: Preparation of 3-(5-((1-((4′-fluoro-5,5-dimethyl-3,4,5,6-tetrahydro-[1,1′-biphenyl]-2-yl)methyl)azetidin-3-yl)thio)-1-oxoisoindolin-2-yl)piperidine-2,6-dione (GT-03457; compound 48)

The synthesis was performed according to synthetic method I using compound GT-03613 as the starting material.

The target compound (GT-03457) was prepared as a white solid (10 mg, yield: 20%). ¹H NMR (400 MHz, MeOD) δ 7.77 (d, J=8.0 Hz, 1H), 7.42 (s, 1H), 7.33 (s, 1H), 7.15 (d, J=11.2 Hz, 4H), 5.14 (dd, J=13.2, 5.2 Hz, 1H), 4.66-4.55 (m, 5H), 4.50 (d, J=6.0 Hz, 3H), 4.41-4.31 (m, 1H), 3.92-3.82 (m, 2H), 2.99-2.89 (m, 1H), 2.85-2.75 (m, 1H), 2.56-2.48 (m, 1H), 2.32-2.07 (m, 5H), 1.56 (t, J=6.4 Hz, 2H), 1.02 (s, 6H). LC/MS (ESI) m/z: calcd for C₃₁H₃₅FN₃O₃S⁺ [M+H]⁺, 548.24; found, 548.3.

### Example 9: Preparation of 3-(5-((1-((4′-chloro-5,5-dimethyl-3,4,5,6-tetrahydro-[1,1′-biphenyl]-2-yl)methyl)azetidin-3-yl)oxy)-1-oxoisoindolin-2-yl)piperidine-2,6-dione (GT-03369; compound 84)

The synthesis was performed according to synthetic method I using compound GT-03614 as the starting material.

The target compound (GT-03369) was prepared as a white solid (20 mg, yield: 40%). ¹H NMR (400 MHz, MeOD) δ 7.65 (d, J=8.4 Hz, 1H), 7.39-7.31 (m, 1H), 7.26-7.16 (m, 1H), 7.08-6.97 (m, 2H), 6.92-6.85 (m, 1H), 6.77-6.63 (m, 1H), 5.05-5.01 (m, 1H), 4.98-4.95 (m, 1H), 4.67-4.57 (m, 1H), 4.42-4.32 (m, 2H), 4.22 (brs, 1H), 3.91-3.66 (m, 3H), 2.84-2.76 (m, 1H), 2.70-2.66 (m, 1H), 2.41-2.36 (m, 1H), 2.14-2.09 (m, 2H), 2.03 (brs, 3H), 1.46 (t, J=6.4 Hz, 2H), 0.92 (s, 6H). LC/MS (ESI) m/z: calcd for C₃₁H₃₅ClN₃O₄⁺ [M+H]⁺, 548.23; found, 548.2.

### Example 10: Preparation of 3-(5-((1-((4′-chloro-[1,1′-biphenyl]-2-yl)methyl)azetidin-3-yl)oxy)-1-oxoisoindolin-2-yl)piperidine-2,6-dione (GT-03363; compound 111)

The synthesis was performed according to synthetic method II using compound (GT-03614) as the starting material.

The target compound (GT-03363) was prepared as a white solid (20 mg, yield: 45%). ¹H NMR (400 MHz, MeOD) δ 7.63 (d, J=8.4 Hz, 1H), 7.53-7.47 (m, 1H), 7.46-7.42 (m, 2H), 7.36 (d, J=8.0 Hz, 2H), 7.30-7.27 (m, 1H), 7.25-7.23 (m, 2H), 6.81 (brs, 1H), 6.77 (d, J=8.2 Hz, 1H), 5.05-5.00 (m, 1H), 4.98-4.94 (m, 1H), 4.50 (brs, 2H), 4.45 (brs, 2H), 4.35 (d, J=5.8 Hz, 2H), 3.91 (brs, 2H), 2.85-2.77 (m, 1H), 2.71-2.65 (m, 1H), 2.43-2.32 (m, 1H), 2.09-2.03 (m, 1H). LC/MS (ESI) m/z: calcd for C₂₉H₂₇ClN₃O₄⁺ [M+H]⁺, 516.17; found, 516.2.

### Example 11: Preparation of 3-(5-((1-((4′-fluoro-5,5-dimethyl-3,4,5,6-tetrahydro-[1,1′-biphenyl]-2-yl)methyl)azetidin-3-yl)oxy)-1-oxoisoindolin-2-yl)piperidine-2,6-dione (GT-03459; compound 130)

The synthesis was performed according to synthetic method I using compound (GT-03614) as the starting material.

The target compound (GT-03459) was prepared as a white solid (10 mg, yield: 20%). ¹H NMR (400 MHz, MeOD) δ 7.76 (d, J=8.4 Hz, 1H), 7.19-7.15 (m, 2H), 7.12-6.99 (m, 2H), 6.98-6.75 (m, 2H), 5.14 (dd, J=13.2, 5.2 Hz, 1H), 5.10-5.06 (m, 1H), 4.70-4.58 (m, 2H), 4.53-4.40 (m, 2H), 4.18-3.75 (m, 2H), 3.94 (s, 2H), 2.99-2.87 (m, 1H), 2.86-2.75 (m, 1H), 2.59-2.41 (m, 1H), 2.32-2.17 (m, 3H), 2.16 (s, 2H), 1.57 (t, J=6.4 Hz, 2H), 1.03 (s, 6H). LC/MS (ESI) m/z: calcd for C₃₁H₃₅FN₃O₄⁺ [M+H]⁺, 532.26; found, 532.3.

### Example 12: Preparation of 3-(5-((1-((4-(4-chlorophenyl)-5,6-dihydro-2H-pyran-3-yl)methyl)azetidin-3-yl)oxy)-1-oxoisoindolin-2-yl)piperidine-2,6-dione (GT-06354; compound 134)

The synthesis was performed according to synthetic method I using compound (GT-03614) as the starting material.

The target compound (GT-06354) was prepared as a white solid (12 mg, yield: 19%). ¹H NMR (400 MHz, DMSO-d6) δ 10.98 (s, 1H), 7.69-7.63 (m, 1H), 7.59-7.35 (m, 3H), 7.31-7.27 (m, 2H), 6.99-6.92 (m, 1H), 5.09 (dd, J=13.2, 5.0 Hz, 1H), 4.99-4.93 (m, 1H), 4.71-4.58 (m, 1H), 4.40 (d, J=17.4 Hz, 1H), 4.32-4.18 (m, 4H), 4.07-3.97 (m, 1H), 3.96-3.69 (m, 4H), 3.66-3.51 (m, 1H), 2.99-2.84 (m, 1H), 2.60 (d, J=17.1 Hz, 1H), 2.55-2.52 (m, 1H), 2.45-2.32 (m, 2H), 2.05-1.93 (m, 1H).

LCMS (ESI) calcd for C₂₈H₂₉ClN₃₀₅⁺ [M+H]⁺: 522.19, found, 522.2.

### Example 13: Preparation of 3-(4-((1-((4′-chloro-5,5-dimethyl-3,4,5,6-tetrahydro-[1,1′-biphenyl]-2-yl)methyl)azetidin-3-yl)thio)-1-oxoisoindolin-2-yl)piperidine-2,6-dione (GT-06515)

The target compound (GT-07004) was prepared as a white solid (1.5 g, yield: 86%) according to the method described in synthetic method I (GT-06515) (white solid, 16 mg, yield: 24%). ¹H NMR (400 MHz, DMSO-d6) δ 11.04 (s, 1H), 10.86 (s, 1H), 7.66 (d, J=7.1 Hz, 1H), 7.61-7.49 (m, 1H), 7.46-7.40 (m, 3H), 7.21-7.13 (m, 2H), 5.15 (dd, J=15.9, 10.6 Hz, 1H), 4.69-4.53 (m, 1H), 4.42-4.06 (m, 4H), 3.75 (s, 1H), 3.69-3.61 (m, 1H), 2.95-2.89 (m, 1H), 2.68-2.64 (m, 1H), 2.55-2.53 (m, 2H), 2.46-2.42 (m, 1H), 2.27-2.11 (m, 2H), 2.10-1.95 (m, 3H), 1.53-1.36 (m, 2H), 0.96 (d, J=7.3 Hz, 6H).

LCMS (ESI) calcd for C₃₁H₃₅ClN₃O₃S⁺ [M+H]⁺: 564.21, found, 564.3.

### Example 14: Preparation of 3-(6-((1-((4′-chloro-5,5-dimethyl-3,4,5,6-tetrahydro-[1,1′-biphenyl]-2-yl)methyl)azetidin-3-yl)thio)-1-oxoisoindolin-2-yl)piperidine-2,6-dione (GT-06757)

The target compound (GT-06757) was prepared as a white solid (14 mg, yield: 17%) according to the method described in synthetic method I. ¹H NMR (400 MHz, DMSO-d6) δ 10.93 (s, 1H), 7.54 (d, J=7.8 Hz, 1H), 7.48 (s, 1H), 7.39 (d, J=7.9 Hz, 3H), 7.13-7.09 (m, 2H), 5.05 (dd, J=13.3, 5.1 Hz, 1H), 4.56-4.47 (m, 1H), 4.39 (d, J=17.6 Hz, 1H), 4.28-4.17 (m, 3H), 4.07-4.00 (m 1H), 3.80-3.54 (m, 3H), 2.94-2.78 (m, 1H), 2.56-2.51 (m, 1H), 2.40-2.26 (m, 1H), 2.12-2.04 (m, 2H), 1.95 (s, 3H), 1.35 (t, J=6.1 Hz, 2H), 0.87 (s, 6H). LCMS (ESI) calcd for C₃₁H₃₅ClN₃O₃S⁺ [M+H]⁺: 564.21, found, 564.3.

### Example 15: Preparation of 3-(7-((1-((4′-chloro-5,5-dimethyl-3,4,5,6-tetrahydro-[1,1′-biphenyl]-2-yl)methyl)azetidin-3-yl)thio)-1-oxoisoindolin-2-yl)piperidine-2,6-dione (GT-06812)

The target compound (GT-06812) was prepared as a white solid (13 mg, yield: 20%) according to the method described in synthetic method I. ¹H NMR (400 MHz, DMSO-d6) δ 11.00 (s, 1H), 7.55 (t, J=7.6 Hz, 1H), 7.49-7.44 (m, 1H), 7.42-7.39 (m, 1H), 7.35 (d, J=8.0 Hz, 1H), 7.21 (d, J=8.1 Hz, 1H), 7.14 (d, J=7.8 Hz, 1H), 7.01 (d, J=8.1 Hz, 1H), 5.08-5.03 (m, 1H), 4.76-4.68 (m, 1H), 4.53-4.21 (m, 4H), 4.14-4.08 (m, 1H), 3.86-3.74 (m, 2H), 3.00-2.82 (m, 1H), 2.61 (d, J=17.6 Hz, 1H), 2.56-2.51 (m, 1H), 2.45-2.32 (m, 1H), 2.18-2.12 (m, 2H), 2.05-1.98 (m, 3H), 1.44-1.42 (m, 2H), 0.95 (s, 6H). LCMS (ESI) calcd for C₃₁H₃₅ClN₃O₃S⁺ [M+H]⁺: 564.21, found, 564.3.

### Example 16: Preparation of 3-(5-((1-((4′-chloro-5,5-dimethyl-3,4,5,6-tetrahydro-[1,1′-biphenyl]-2-yl)methyl)azepan-4-yl)thio)-1-oxoisoindolin-2-yl)piperidine-2,6-dione (GT-06641)

The target compound (GT-06641) was prepared as a white solid (41 mg, yield: 52%) according to the method described in synthetic method I. ¹H NMR (400 MHz, DMSO-d6) δ 11.00 (s, 1H), 7.69-7.66 (m, 1H), 7.57 (d, J=6.7 Hz, 1H), 7.47-7.41 (m, 3H), 7.14 (dd, J=8.4, 3.0 Hz, 2H), 5.11 (d, J=13.3 Hz, 1H), 4.52-4.39 (m, 1H), 4.39-4.25 (m, 1H), 3.78-3.65 (m, 1H), 3.58-3.51 (m, 2H), 3.27-3.18 (m, 1H), 3.10-2.81 (m, 3H), 2.61 (d, J=16.7 Hz, 1H), 2.44-2.15 (m, 3H), 2.14-1.94 (m, 6H), 1.92-1.69 (m, 2H), 1.68-1.37 (m, 4H), 0.96 (s, 3H), 0.95 (s, 3H). LCMS (ESI) calcd for C₃₄H₄₁ClN₃O₃S+[M+H]⁺: 606.26, found, 606.3.

### Example 17: Preparation of 3-(5-((2-((4′-chloro-5,5-dimethyl-3,4,5,6-tetrahydro-[1,1′-biphenyl]-2-yl)methyl)octahydrocyclopenta[c]pyrrol-5-yl)thio)-1-oxoisoindolin-2-yl)piperidine-2,6-dione (GT-06813)

The target compound (GT-06813) was prepared as a white solid (20 mg, yield: 32%) according to the method described in synthetic method I. ¹H NMR (400 MHz, DMSO-d6) δ 10.99 (s, 1H), 7.68-7.62 (m, 1H), 7.59-7.50 (m, 1H), 7.47-7.42 (m, 2H), 7.41-7.36 (m, 1H), 7.19-7.13 (m, 2H), 5.10 (dd, J=13.2, 5.1 Hz, 1H), 4.44 (d, J=17.6 Hz, 1H), 4.31 (d, J=17.6 Hz, 1H), 3.99-3.90 (m, 1H), 3.74-3.51 (m, 4H), 3.31-3.20 (m, 2H), 3.19-3.02 (m, 1H), 3.01-2.84 (m, 3H), 2.81-2.67 (m, 1H), 2.61 (d, J=16.3 Hz, 1H), 2.39 (d, J=12.9 Hz, 1H), 2.35-2.22 (m, 4H), 2.09-1.90 (m, 4H), 1.71-1.57 (m, 1H), 0.97 (s, 6H). LCMS (ESI) calcd for C₃₅H₄₁ClN₃O₃S⁺ [M+H]⁺: 618.26, found, 618.3.

### Example 18: Preparation of 3-(5-((7-((4′-chloro-5,5-dimethyl-3,4,5,6-tetrahydro-[1,1′-biphenyl]-2-yl)methyl)-7-azaspiro[3.5]nonan-2-yl)thio)-1-oxoisoindolin-2-yl)piperidine-2,6-dione (GT-06873)

The target compound (GT-06873) was prepared as a white solid (14 mg, yield: 22%) according to the method described in synthetic method I. ¹H NMR (400 MHz, DMSO-d6) δ 10.91 (s, 1H), 9.26 (s, 1H), 7.55 (d, J=8.0 Hz, 1H), 7.37 (d, J=8.3 Hz, 2H), 7.32 (s, 1H), 7.21 (d, J=8.3 Hz, 1H), 7.06 (d, J=8.3 Hz, 2H), 5.02 (dd, J=13.3, 5.1 Hz, 1H), 4.36-4.17 (m, 2H), 4.04-3.90 (m, 1H), 3.48-3.41 (m, 2H), 3.12-3.02 (m, 2H), 2.92-2.76 (m, 1H), 2.58-2.51 (m, 2H), 2.39-2.27 (m, 3H), 2.25-2.14 (m, 2H), 1.96 (brs, 2H), 1.95-1.88 (m, 2H), 1.88-1.72 (m, 4H), 1.71-1.59 (m, 2H), 1.41-1.38 (m, 2H), 0.89 (s, 6H). LCMS (ESI) calcd for C₃₆H₄₃ClN₃O₃S⁺ [M+H]⁺: 632.27, found, 632.3.

### Example 19: Preparation of 3-(5-((3-((4′-chloro-5,5-dimethyl-3,4,5,6-tetrahydro-[1,1′-biphenyl]-2-yl)methyl)-3-azaspiro[5.5]undecan-9-yl)thio)-1-oxoisoindolin-2-yl)piperidine-2,6-dione (GT-06874)

The target compound (GT-06874) was prepared as a white solid (19 mg, yield: 30%) according to the method described in synthetic method I. ¹H NMR (400 MHz, DMSO-d6) δ 10.92 (s, 1H), 9.18 (s, 1H), 7.57 (d, J=8.0 Hz, 1H), 7.50 (s, 1H), 7.37 (d, J=8.1 Hz, 3H), 7.06 (d, J=8.3 Hz, 2H), 5.03 (dd, J=13.3, 5.1 Hz, 1H), 4.35 (d, J=17.5 Hz, 1H), 4.23 (d, J=17.4 Hz, 1H), 3.59-3.43 (m, 2H), 3.02-2.99 (m, 2H), 2.92-2.77 (m, 1H), 2.66-2.55 (m, 2H), 2.53 (d, J=16.8 Hz, 1H), 2.31-2.27 (m, 1H), 2.20 (brs, 2H), 2.06-1.86 (m, 3H), 1.85-1.59 (m, 5H), 1.52-1.40 (m, 1H), 1.46-1.19 (m, 8H), 1.09-1.04 (m, 1H), 0.89 (s, 6H). LCMS (ESI) calcd for C₃₈H₄₇ClN₃O₃S⁺ [M+H]⁺: 660.30, found, 660.3.

### Example 20: Preparation of 3-(5-((1-((4′-chloro-5,5-dimethyl-3,4,5,6-tetrahydro-[1,1′-biphenyl]-2-yl)methyl)-3,3-difluoropiperidin-4-yl)thio)-1-oxoisoindolin-2-yl)piperidine-2,6-dione (GT-06960)

The target compound (GT-06960) was prepared as a white solid (9 mg, yield: 12%) according to the method described in synthetic method I. ¹H NMR (400 MHz, DMSO-d6) δ 10.92 (s, 1H), 7.60-7.59 (m, 2H), 7.47 (d, J=8.1 Hz, 1H), 7.34 (d, J=8.1 Hz, 2H), 7.04 (d, J=8.1 Hz, 2H), 5.03 (dd, J=13.3, 5.0 Hz, 1H), 4.36 (d, J=17.4 Hz, 1H), 4.23 (d, J=17.5 Hz, 1H), 3.95-3.83 (m, 1H), 3.27-3.19 (m, 4H), 2.91-2.77 (m, 2H), 2.53 (d, J=17.2 Hz, 1H), 2.37-2.29 (m, 2H), 2.12 (brs, 2H), 2.06-1.86 (m, 4H), 1.82-1.54 (m, 1H), 1.37 (t, J=6.1 Hz, 2H), 0.89 (s, 3H), 0.88 (s, 3H). LCMS (ESI) calcd for C₃₃H₃₇ClF₂N₃O₃S⁺ [M+H]⁺: 628.22, found, 628.3.

### Example 21: Preparation of 3-(5-((1-(4′-chloro-5,5-dimethyl-3,4,5,6-tetrahydro-[1,1′-biphenyl]-2-carbonyl)azetidin-3-yl)thio)-1-oxoisoindolin-2-yl)piperidine-2,6-dione (GT-06137)

The target compound (GT-06137) was prepared as a white solid (39 mg, yield: 47%) according to the method described in synthetic method III. ¹H NMR (400 MHz, DMSO-d6) δ 10.99 (s, 1H), 7.63 (d, J=8.0 Hz, 1H), 7.40 (dd, J=8.4, 1.3 Hz, 2H), 7.33 (s, 1H), 7.25-7.21 (m, 3H), 5.10 (dd, J=13.1, 4.7 Hz, 1H), 4.42 (d, J=17.5 Hz, 1H), 4.36-4.27 (m, 1H), 4.27-4.04 (m, 3H), 3.44 (dd, J=10.2, 3.6 Hz, 1H), 3.23 (dd, J=8.5, 3.8 Hz, 1H), 3.00-2.81 (m, 1H), 2.60 (d, J=17.0 Hz, 1H), 2.39 (dd, J=13.2, 4.4 Hz, 1H), 2.25-2.16 (m, 3H), 2.12-1.93 (m, 2H), 1.40 (t, J=6.3 Hz, 2H), 0.96 (s, 3H), 0.95 (s, 3H).

LCMS (ESI) calcd for C₃₁H₃₃ClN₃O₄S⁺ [M+H]⁺: 578.19, found, 578.2.

### Example 22: Preparation of 3-(5-((1-(4′-chloro-5,5-dimethyl-3,4,5,6-tetrahydro-[1,1′-biphenyl]-2-carbonyl)pyrrolidin-3-yl)thio)-1-oxoisoindolin-2-yl)piperidine-2,6-dione (GT-06139)

The target compound (GT-06139) was prepared as a white solid (28 mg, yield: 34%) according to the method described in synthetic method III. ¹H NMR (400 MHz, DMSO-d6) δ 11.00 (s, 1H), 7.67-7.64 (m, 1H), 7.54-7.43 (m, 1H), 7.41-7.31 (m, 3H), 7.26 (d, J=8.5 Hz, 1H), 7.21 (d, J=7.9 Hz, 1H), 5.19-5.04 (m, 1H), 4.51-4.26 (m, 2H), 4.00-3.89 (m, 1H), 3.18-3.08 (m, 3H), 2.98-2.88 (m, 2H), 2.61 (d, J=16.8 Hz, 1H), 2.44-2.37 (m, 1H), 2.34-2.21 (m, 2H), 2.16-1.98 (m, 4H), 1.72-1.59 (m, 1H), 1.47-1.42 (m, 1H), 1.40-1.27 (m, 1H), 0.99 (d, J=4.0 Hz, 6H). LCMS (ESI) calcd for C₃₂H₃₅ClN₃O₄S⁺ [M+H]⁺: 592.20, found, 592.2.

### Example 23: Preparation of 3-(5-((1-(4′-chloro-5,5-dimethyl-3,4,5,6-tetrahydro-[1,1′-biphenyl]-2-carbonyl)piperidin-4-yl)thio)-1-oxoisoindolin-2-yl)piperidine-2,6-dione (GT-06140)

The target compound (GT-06140) was prepared as a white solid (38 mg, yield: 47%) according to the method described in synthetic method III. ¹H NMR (400 MHz, DMSO-d6) δ 10.99 (s, 1H), 7.63 (d, J=8.0 Hz, 1H), 7.52 (s, 1H), 7.48-7.35 (m, 3H), 7.30 (d, J=8.1 Hz, 1H), 7.22 (d, J=7.8 Hz, 1H), 5.10 (dd, J=13.1, 4.9 Hz, 1H), 4.42 (d, J=17.5 Hz, 1H), 4.29 (d, J=17.3 Hz, 1H), 4.19-4.04 (m, 1H), 3.07-2.85 (m, 2H), 2.70-2.59 (m, 4H), 2.47-2.24 (m, 4H), 2.09-1.81 (m, 5H), 1.64-1.51 (m, 1H), 1.48-1.42 (m, 2H), 1.00 (s, 3H), 0.97 (s, 3H). LCMS (ESI) calcd for C₃₃H₃₇ClN₃O₄S⁺ [M+H]⁺: 606.22, found, 606.2.

### Example 24: Preparation of 3-(5-((1-(4′-chloro-5,5-dimethyl-3,4,5,6-tetrahydro-[1,1′-biphenyl]-2-carbonyl)azepan-4-yl)thio)-1-oxoisoindolin-2-yl)piperidine-2,6-dione (GT-06642)

The target compound (GT-06642) was prepared as a white solid (28 mg, yield: 35%) according to the method described in synthetic method III. ¹H NMR (400 MHz, DMSO-d6) δ 10.74 (s, 1H), 7.46-7.35 (m, 1H), 7.26-7.21 (m, 1H), 7.17-7.04 (m, 5H), 4.85 (d, J=12.7 Hz, 1H), 4.18 (d, J=16.3 Hz, 1H), 4.05 (d, J=17.7 Hz, 1H), 3.09-2.98 (m, 4H), 2.78-2.56 (m, 2H), 2.35 (d, J=17.7 Hz, 1H), 2.23-2.02 (m, 4H), 2.03-1.81 (m, 2H), 1.80-1.71 (m, 1H), 1.68-1.60 (m, 2H), 1.32-1.12 (m, 5H), 0.73 (s, 3H), 0.71 (s, 3H). LCMS (ESI) calcd for C₃₄H₃₉ClN₃O₄S⁺ [M+H]⁺: 606.23, found, 606.2.

### Example 25: Preparation of 3-(5-(((3aS,6aS)-2-(4′-chloro-5,5-dimethyl-3,4,5,6-tetrahydro-[1,1′-biphenyl]-2-carbonyl)octahydrocyclopenta[c]pyrrol-5-yl)thio)-1-oxoisoindolin-2-yl)piperidine-2,6-dione (GT-07022)

The target compound (GT-07022) was prepared as a white solid (20 mg, yield: 31%) according to the method described in synthetic method III. ¹H NMR (400 MHz, DMSO-d6) δ 10.91 (s, 1H), 7.55 (d, J=8.0 Hz, 1H), 7.42 (s, 1H), 7.35-7.26 (m, 3H), 7.22 (d, J=8.2 Hz, 2H), 5.02 (dd, J=13.3, 4.9 Hz, 1H), 4.34 (d, J=17.4 Hz, 1H), 4.22 (d, J=17.2 Hz, 1H), 3.62-3.44 (m, 1H), 3.17-3.04 (m, 3H), 2.84-2.79 (m, 3H), 2.55-2.43 (m, 2H), 2.38-2.26 (m, 3H), 2.23-2.10 (m, 5H), 2.00-1.81 (m, 2H), 1.39-1.30 (m, 2H), 0.91 (s, 3H), 0.90 (s, 3H). LCMS (ESI) calcd for C₃₅H₃₉ClN₃O₄S⁺ [M+H]⁺: 632.23, found, 632.2.

### Example 26: Preparation of 3-(5-(((3aR,6aS)-2-(4′-chloro-5,5-dimethyl-3,4,5,6-tetrahydro-[1,1′-biphenyl]-2-carbonyl)octahydrocyclopenta[c]pyrrol-5-yl)thio)-1-oxoisoindolin-2-yl)piperidine-2,6-dione (GT-07015)

The target compound (GT-07015) was prepared as a white solid (14 mg, yield: 22%) according to the method described in synthetic method III. ¹H NMR (400 MHz, DMSO-d6) δ 10.92 (s, 1H), 7.58 (d, J=7.9 Hz, 1H), 7.38 (s, 1H), 7.31 (d, J=8.4 Hz, 2H), 7.27-7.21 (m, 3H), 5.04 (dd, J=13.2, 4.8 Hz, 1H), 4.37 (d, J=17.0 Hz, 1H), 4.25 (d, J=17.6 Hz, 1H), 3.56-3.41 (m, 1H), 3.04-2.96 (m, 1H), 2.88-2.81 (m, 1H), 2.77-2.63 (m, 1H), 2.59-2.49 (m, 4H), 2.35-2.30 (m, 1H), 2.29-2.03 (m, 4H), 1.97-1.89 (m, 2H), 1.70-1.44 (m, 3H), 1.36 (t, J=5.9 Hz, 3H), 0.92 (s, 3H), 0.91 (s, 3H). LCMS (ESI) calcd for C₃₅H₃₉ClN₃O₄S⁺ [M+H]⁺: 632.23, found, 632.2.

### Example 27: Preparation of 3-(5-((7-(4′-chloro-5,5-dimethyl-3,4,5,6-tetrahydro-[1,1′-biphenyl]-2-carbonyl)-7-azaspiro[3.5]nonan-2-yl)thio)-1-oxoisoindolin-2-yl)piperidine-2,6-dione (GT-06918)

The target compound (GT-06918) was prepared as a white solid (27 mg, yield: 51%) according to the method described in synthetic method III. ¹H NMR (400 MHz, DMSO-d6) δ 10.97 (s, 1H), 7.61 (d, J=8.0 Hz, 1H), 7.40-7.34 (m, 3H), 7.28-7.24 (m, 3H), 5.09 (dd, J=13.2, 4.9 Hz, 1H), 4.41 (d, J=17.2 Hz, 1H), 4.28 (d, J=17.4 Hz, 1H), 4.09-3.90 (m, 1H), 3.47-4.34 (m, 1H), 3.22-2.85 (m, 4H), 2.60 (d, J=16.6 Hz, 1H), 2.47-2.32 (m, 4H), 2.30-2.23 (m, 1H), 2.20-1.84 (m, 4H), 1.74-1.65 (m, 1H), 1.61-1.50 (m, 1H), 1.50-1.34 (m, 3H), 1.34-1.06 (m, 2H), 0.97 (s, 6H). LCMS (ESI) calcd for C₃₆H₄₁ClN₃O₄S⁺ [M+H]⁺: 646.25, found, 646.3.

### Example 28: Preparation of 3-(5-((3-(4′-chloro-5,5-dimethyl-3,4,5,6-tetrahydro-[1,1′-biphenyl]-2-carbonyl)-3-azaspiro[5.5]undecan-9-yl)thio)-1-oxoisoindolin-2-yl)piperidine-2,6-dione (GT-07016)

The target compound (GT-07016) was prepared as a white solid (17 mg, yield: 32%) according to the method described in synthetic method III. ¹H NMR (400 MHz, DMSO-d6) δ 10.91 (s, 1H), 7.55 (d, J=8.0 Hz, 1H), 7.48 (s, 1H), 7.38-7.32 (m, 1H), 7.30 (d, J=8.6 Hz, 2H), 7.20 (d, J=8.6 Hz, 2H), 5.02 (dd, J=13.3, 5.1 Hz, 1H), 4.35 (d, J=17.4 Hz, 1H), 4.22 (d, J=17.4 Hz, 1H), 3.54-3.49 (m, 1H), 3.35-3.29 (m, 2H), 3.07-2.76 (m, 4H), 2.53 (d, J=16.0 Hz, 1H), 2.40-2.21 (m, 3H), 2.02-1.82 (m, 3H), 1.74-1.61 (m, 3H), 1.43-1.32 (m, 4H), 1.17-0.98 (m, 4H), 0.92 (s, 3H), 0.90 (s, 3H), 0.88-0.84 (m, 1H), 0.78-0.72 (m, 1H). LCMS (ESI) calcd for C₃₈H₄₅ClN₃O₄S⁺ [M+H]⁺: 674.28, found, 674.3.

### Example 29: Preparation of 3-(5-((1-(4′-chloro-5,5-dimethyl-3,4,5,6-tetrahydro-[1,1′-biphenyl]-2-carbonyl)-3,3-difluoropiperidin-4-yl)thio)-1-oxoisoindolin-2-yl)piperidine-2,6-dione (GT-07017)

The target compound (GT-07017) was prepared as a white solid (26 mg, yield: 41%) according to the method described in synthetic method III. ¹H NMR (400 MHz, DMSO-d6) δ 10.93 (s, 1H), 7.59-7.55 (m, 2H), 7.45-7.41 (m, 1H), 7.33-7.27 (m, 2H), 7.23-7.09 (m, 2H), 5.03 (dd, J=13.3, 5.0 Hz, 1H), 4.35 (d, J=17.4 Hz, 1H), 4.22 (d, J=17.4 Hz, 1H), 4.16-3.94 (m, 1H), 3.94-3.69 (m, 1H), 3.61-3.40 (m, 1H), 3.08-2.97 (m, 1H), 2.92-2.77 (m, 1H), 2.52 (d, J=16.6 Hz, 1H), 2.42-2.21 (m, 4H), 2.12-1.75 (m, 4H), 1.53-1.26 (m, 3H), 0.93 (s, 6H), 0.90 (s, 3H). LCMS (ESI) calcd for C₃₃H₃₅ClF₂N₃O₄S⁺ [M+H]⁺: 642.20, found, 642.3.

### Example 30: Preparation of 3-(5-((1-((4′-chloro-[1,1′-biphenyl]-2-yl)methyl)azepan-4-yl)thio)-1-oxoisoindolin-2-yl)piperidine-2,6-dione (GT-06643)

The target compound (GT-06643) was prepared as a white solid (45 mg, yield: 60%) according to the method described in synthetic method I. ¹H NMR (400 MHz, DMSO-d6) δ 11.00 (s, 1H), 8.05-8.03 (m, 1H), 7.66 (d, J=7.9 Hz, 1H), 7.57-7.52 (m, 5H), 7.47-7.35 (m, 3H), 7.35-7.27 (m, 1H), 5.11 (dd, J=13.2, 4.9 Hz, 1H), 4.46-4.26 (m, 4H), 3.83-3.58 (m, 1H), 3.24-3.07 (m, 1H), 2.96-2.87 (m, 3H), 2.61 (d, J=16.9 Hz, 1H), 2.45-2.38 (m, 1H), 2.11-1.99 (m, 4H), 1.86-1.74 (m, 2H), 1.70-1.39 (m, 2H). LCMS (ESI) calcd for C₃₂H₃₃ClN₃O₃S⁺ [M+H]⁺: 574.19, found, 574.2.

### Example 31: Preparation of 3-(5-((2-((4′-chloro-[1,1′-biphenyl]-2-yl)methyl)octahydrocyclopenta[c]pyrrol-5-yl)thio)-1-oxoisoindolin-2-yl)piperidine-2,6-dione (GT-06914)

The target compound (GT-06914) was prepared as a white solid (30 mg, yield: 51%) according to the method described in synthetic method I. ¹H NMR (400 MHz, DMSO-d6) δ 10.92 (s, 1H), 8.00-7.84 (m, 1H), 7.60-7.51 (m, 1H), 7.52-7.39 (m, 5H), 7.39-7.29 (m, 3H), 7.28-7.20 (m, 1H), 5.05-5.01 (m, 1H), 4.46-4.15 (m, 4H), 3.51-3.48 (m, 1H), 3.44-3.40 (m, 1H), 3.10-2.85 (m, 1H), 2.96-2.74 (m, 3H), 2.73-2.58 (m, 2H), 2.53 (d, J=17.4 Hz, 1H), 2.39-2.26 (m, 1H), 2.25-2.13 (m, 1H), 2.03-1.82 (m, 2H), 1.78-1.45 (m, 1H), 1.41-1.35 (m, 1H). LCMS (ESI) calcd for C₃₃H₃₃ClN₃O₃S⁺[M+H]⁺: 586.19, found, 586.3.

### Example 32: Preparation of 3-(5-((1-((4′-chloro-[1,1′-biphenyl]-2-yl)methyl)-3,3-difluoropiperidin-4-yl)thio)-1-oxoisoindolin-2-yl)piperidine-2,6-dione (GT-06924)

The target compound (GT-06924) was prepared as a white solid (5 mg, yield: 7%) according to the method described in synthetic method I. ¹H NMR (400 MHz, DMSO-d6) δ 10.90 (s, 1H), 7.58 (d, J=7.9 Hz, 2H), 7.50-7.40 (m, 6H), 7.35-7.28 (m, 3H), 5.02 (dd, J=13.1, 4.8 Hz, 1H), 4.70-4.48 (m, 1H), 4.35 (d, J=16.9 Hz, 1H), 4.22 (d, J=17.3 Hz, 1H), 4.13-4.02 (m, 1H), 3.18-3.03 (m, 4H), 2.88-2.79 (m, 1H), 2.73-2.57 (m, 1H), 2.52 (d, J=17.3 Hz, 1H), 2.39-2.22 (m, 1H), 1.98-1.84 (m, 1H), 1.78-1.66 (m, 1H), 1.58-1.45 (m, 1H). LCMS (ESI) calcd for C₃₁H₂₉ClFN₃O₃S⁺ [M+H]⁺: 596.16, found, 596.3.

### Example 33: Preparation of 3-(5-((1-(4′-chloro-[1,1′-biphenyl]-2-carbonyl)pyrrolidin-3-yl)thio)-1-oxoisoindolin-2-yl)piperidine-2,6-dione (GT-06181)

The target compound (GT-06181) was prepared as a white solid (32 mg, yield: 41%) according to the method described in synthetic method III. ¹H NMR (400 MHz, DMSO-d6) δ 10.92 (s, 1H), 7.60 (d, J=8.0 Hz, 1H), 7.47-7.42 (m, 3H), 7.42-7.36 (m, 4H), 7.35-7.29 (m, 3H), 5.06-5.01 (m, 1H), 4.41-4.20 (m, 2H), 4.01 (s, 1H), 3.66-3.64 (m, 1H), 2.95-2.90 (m, 1H), 2.85-2.77 (m, 1H), 2.53 (d, J=17.2 Hz, 1H), 2.36-2.27 (m, 1H), 2.29-2.04 (m, 1H), 1.95-1.90 (m, 1H), 1.74-1.66 (m, 1H), 1.64-1.48 (m, 1H). LCMS (ESI) calcd for C₃₀H₂₇ClN₃O₄S⁺ [M+H]⁺: 560.14, found, 560.2.

### Example 34: Preparation of 3-(5-((1-(4′-chloro-[1,1′-biphenyl]-2-carbonyl)piperidin-4-yl)thio)-1-oxoisoindolin-2-yl)piperidine-2,6-dione (GT-06182)

The target compound (GT-06182) was prepared as a white solid (48 mg, yield: 62%) according to the method described in synthetic method III. ¹HNMR (400 MHz, DMSO-d6) δ 10.91 (s, 1H), 7.56 (d, J=8.0 Hz, 1H), 7.53-7.37 (m, 8H), 7.32 (d, J=8.1 Hz, 2H), 5.02 (dd, J=13.3, 5.1 Hz, 1H), 4.40-4.08 (m, 3H), 3.51-3.37 (m, 1H), 3.02-2.92 (m, 1H), 2.91-2.84 (m, 1H), 2.82-2.72 (m, 2H), 2.57-2.46 (m, 2H), 2.36-2.26 (m, 1H), 2.00-1.87 (m, 1H), 1.83-1.80 (m, 1H), 1.55-1.25 (m, 2H). LCMS (ESI) calcd for C₃₁H₂₉ClN₃O₄S⁺ [M+H]⁺: 574.16, found, 574.2.

### Example 35: Preparation of 3-(5-((1-(4′-chloro-[1,1′-biphenyl]-2-carbonyl)azepan-4-yl)thio)-1-oxoisoindolin-2-yl)piperidine-2,6-dione (GT-06644)

The target compound (52 mg, yield: 68%) was prepared as a white solid (52 mg, yield: 68%) according to the method described in synthetic method III. ¹H NMR (400 MHz, DMSO-d6) δ 10.99 (s, 1H), 7.71-7.60 (m, 1H), 7.52-7.41 (m, 9H), 7.34 (d, J=7.5 Hz, 1H), 5.11 (dd, J=13.2, 4.7 Hz, 1H), 4.47-4.28 (m, 2H), 3.70-3.46 (m, 2H), 3.21-3.06 (m, 1H), 2.91-2.79 (m, 2H), 2.79-2.74 (m, 1H), 2.60 (d, J=16.6 Hz, 1H), 2.44-2.34 (m, 1H), 2.08-1.99 (m, 2H), 1.93-1.80 (m, 1H), 1.79-1.39 (m, 4H). LCMS (ESI) calcd for C₃₂H₃₁ClN₃O₄S⁺ [M+H]⁺: 588.17, found, 588.2.

### Example 36: Preparation of 3-(5-(((3aS,6aS)-2-(4′-chloro-[1,1′-biphenyl]-2-carbonyl)octahydrocyclopenta[c]pyrrol-5-yl)thio)-1-oxoisoindolin-2-yl)piperidine-2,6-dione (GT-07023)

The target compound (GT-07023) was prepared as a white solid (13 mg, yield: 22%) according to the method described in synthetic method III. ¹HNMR (400 MHz, DMSO-d6) δ 10.91 (s, 1H), 7.55 (d, J=8.0 Hz, 1H), 7.46-7.42 (m, 3H), 7.42-7.38 (m, 5H), 7.32-7.29 (m, 1H), 7.28-7.26 (m, 1H), 5.02 (dd, J=13.3, 5.1 Hz, 1H), 4.35 (d, J=16.1 Hz, 1H), 4.22 (d, J=16.9 Hz, 1H), 3.63-3.47 (m, 1H), 3.35-3.28 (m, 3H), 3.02-2.97 (m, 1H), 2.90-2.76 (m, 1H), 2.64-2.45 (m, 4H), 2.37-2.26 (m, 3H), 2.12-2.05 (m, 1H), 2.00-1.85 (m, 1H). LCMS (ESI) calcd for C₃₃H₃₁ClN₃O₄S⁺ [M+H]⁺: 600.17, found, 600.2.

### Example 37: Preparation of 3-(5-(((3aR,6aS)-2-(4′-chloro-[1,1′-biphenyl]-2-carbonyl)octahydrocyclopenta[c]pyrrol-5-yl)thio)-1-oxoisoindolin-2-yl)piperidine-2,6-dione (GT-07024)

The target compound (GT-07024) was prepared as a white solid (15 mg, yield: 25%) according to the method described in synthetic method III. ¹HNMR (400 MHz, DMSO-d6) δ 10.91 (s, 1H), 7.55 (d, J=8.0 Hz, 1H), 7.46-7.42 (m, 3H), 7.42-7.38 (m, 5H), 7.32-7.29 (m, 1H), 7.28-7.26 (m, 1H), 5.02 (dd, J=13.3, 5.1 Hz, 1H), 4.35 (d, J=16.1 Hz, 1H), 4.22 (d, J=16.9 Hz, 1H), 3.63-3.47 (m, 1H), 3.35-3.28 (m, 3H), 3.02-2.97 (m, 1H), 2.90-2.76 (m, 1H), 2.64-2.45 (m, 4H), 2.37-2.26 (m, 3H), 2.12-2.05 (m, 1H), 2.00-1.85 (m, 1H). LCMS (ESI) calcd for C₃₃H₃₁ClN₃O₄S⁺ [M+H]⁺: 600.17, found, 600.2.

### Example 38: Preparation of 3-(5-((1-(4′-chloro-[1,1′-biphenyl]-2-carbonyl)-3,3-difluoropiperidin-4-yl)thio)-1-oxoisoindolin-2-yl)piperidine-2,6-dione (GT-06925)

The target compound (GT-06925) was prepared as a white solid (20 mg, yield: 27%) according to the method described in synthetic method III. ¹H NMR (400 MHz, DMSO-d6) δ 10.90 (s, 1H), 7.58 (d, J=7.9 Hz, 2H), 7.50-7.40 (m, 6H), 7.35-7.28 (m, 3H), 5.02 (dd, J=13.1, 4.8 Hz, 1H), 4.70-4.48 (m, 1H), 4.35 (d, J=16.9 Hz, 1H), 4.22 (d, J=17.3 Hz, 1H), 4.13-4.02 (m, 1H), 3.18-3.03 (m, 2H), 2.88-2.79 (m, 1H), 2.73-2.57 (m, 1H), 2.52 (d, J=17.3 Hz, 1H), 2.39-2.22 (m, 1H), 1.98-1.84 (m, 1H), 1.78-1.66 (m, 1H), 1.58-1.45 (m, 1H). LCMS (ESI) calcd for C₃₁H₂₇ClF₂N₃O₄S⁺ [M+H]⁺: 610.14, found, 610.2.

### Example 39: Preparation of 3-(5-((1-((4′-chloro-[1,1′-biphenyl]-3-yl)methyl)azetidin-3-yl)thio)-1-oxoisoindolin-2-yl)piperidine-2,6-dione (GT-06225)

The target compound (GT-06225) was prepared as a white solid (10 mg, yield: 13%) according to the method described in synthetic method I. ¹H NMR (400 MHz, DMSO-d6) δ 10.92 (s, 1H), 7.81 (s, 1H), 7.73-7.64 (m, 3H), 7.60 (d, J=8.2 Hz, 1H), 7.50-7.42 (m, 5H), 7.33 (d, J=7.8 Hz, 1H), 5.03 (dd, J=13.2, 5.0 Hz, 1H), 4.62-4.44 (m, 3H), 4.39-4.32 (m, 2H), 4.27-4.21 (m, 2H), 4.11-3.93 (m, 2H), 2.91-2.76 (m, 1H), 2.54-2.50 (m, 1H), 2.35-2.26 (m, 1H), 1.96-1.90 (m, 1H). LCMS (ESI) calcd for C₂₉H₂₇ClN₃O₃S⁺ [M+H]⁺: 532.15, found, 532.1.

### Example 40: Preparation of 3-(5-((1-((4′-chloro-[1,1′-biphenyl]-4-yl)methyl)azetidin-3-yl)thio)-1-oxoisoindolin-2-yl)piperidine-2,6-dione (GT-06226)

The target compound (GT-06226) was prepared as a white solid (12 mg, yield: 16%) according to the method described in synthetic method I. ¹H NMR (400 MHz, DMSO-d6) δ 10.92 (s, 1H), 7.73-7.65 (m, 4H), 7.62 (d, J=8.0 Hz, 1H), 7.56 (d, J=8.1 Hz, 2H), 7.47 (d, J=8.6 Hz, 3H), 7.34 (d, J=7.6 Hz, 1H), 5.05-5.01 (m, 1H), 4.64-4.44 (m, 3H), 4.40-4.32 (m, 2H), 4.27-4.22 (m, 2H), 3.99 (brs, 2H), 2.90-2.78 (m, 1H), 2.55-2.51 (m, 1H), 2.38-2.24 (m, 1H), 1.97-1.90 (m, 1H). LCMS (ESI) calcd for C₂₉H₂₇ClN₃O₃S⁺ [M+H]⁺: 532.15, found, 532.2.

### Example 41: Preparation of 3-(1-oxo-5-((1-((5-phenylpyrazin-2-yl)methyl)azetidin-3-yl)thio)isoindolin-2-yl)piperidine-2,6-dione (GT-06350)

The target compound (GT-06350) was prepared as a white solid (15 mg, yield: 26%) according to the method described in synthetic method II. ¹H NMR (400 MHz, DMSO-d6) δ 11.00 (s, 1H), 9.29 (s, 1H), 8.84 (s, 1H), 8.28-8.11 (m, 3H), 7.61-7.50 (m, 5H), 5.11 (dd, J=12.4, 4.1 Hz, 1H), 4.71 (d, J=16.1 Hz, 4H), 4.64-4.50 (m, 1H), 4.45 (d, J=17.8 Hz, 1H), 4.33 (d, J=17.3 Hz, 1H), 4.27-4.18 (m, 2H), 2.96-2.87 (m, 1H), 2.60 (d, J=17.9 Hz, 1H), 2.46-2.33 (m, 1H), 2.05-1.97 (m, 1H). LCMS (ESI) calcd for C₂₇H₂₆N₅O₃S⁺ [M+H]⁺: 500.18, found, 500.2.

### Example 42: Preparation of 3-(5-((1-([2,4′-bipyridin]-5-ylmethyl)azetidin-3-yl)thio)-1-oxoisoindolin-2-yl)piperidine-2,6-dione (GT-06808)

The target compound (GT-06808) was prepared as a white solid (27 mg, yield: 46%) according to the method described in synthetic method II. ¹H NMR (400 MHz, DMSO-d6) δ 11.00 (s, 1H), 9.00 (d, J=1.7 Hz, 1H), 8.97 (d, J=6.5 Hz, 2H), 8.55 (d, J=6.5 Hz, 1H), 8.43 (d, J=8.2 Hz, 1H), 8.32 (dd, J=8.3, 2.1 Hz, 1H), 7.68 (dd, J=8.0, 1.9 Hz, 1H), 7.53 (d, J=5.0 Hz, 1H), 7.40 (t, J=6.5 Hz, 1H), 5.16-5.05 (m, 1H), 4.67 (s, 2H), 4.56-4.27 (m, 5H), 4.27-4.14 (m, 1H), 3.89-3.83 (m, 1H), 2.99-2.84 (m, 1H), 2.60 (d, J=16.6 Hz, 1H), 2.46-2.33 (m, 1H), 2.05-1.93 (m, 1H). LCMS (ESI) calcd for C₂₇H₂₆N₅O₃S⁺ [M+H]⁺: 500.18, found, 500.2.

### Example 43: Preparation of 3-(5-((1-((5-(furan-2-yl)thiophen-2-yl)methyl)azetidin-3-yl)thio)-1-oxoisoindolin-2-yl)piperidine-2,6-dione (GT-06517)

The target compound (GT-06517) was prepared as a white solid (27 mg, yield: 46%) according to the method described in synthetic method I. ¹H NMR (400 MHz, DMSO-d6) δ 11.01 (s, 1H), 7.74 (s, 1H), 7.69 (d, J=8.0 Hz, 1H), 7.52 (s, 1H), 7.41 (d, J=7.9 Hz, 1H), 7.37-7.28 (m, 2H), 6.83 (d, J=3.4 Hz, 1H), 6.61 (s, 1H), 5.11 (dd, J=13.2, 4.9 Hz, 1H), 4.77-4.47 (m, 5H), 4.44 (d, J=17.5 Hz, 1H), 4.32 (d, J=17.4 Hz, 1H), 4.09-3.95 (m, 2H), 3.01-2.84 (m, 1H), 2.60 (d, J=16.5 Hz, 1H), 2.45-2.33 (m, 1H), 2.03-1.98 (m, 1H). LCMS (ESI) calcd for C₂₅H₂₄N₃O₄S2⁺ [M+H]⁺: 494.12, found, 494.2.

### Example 44: Preparation of 3-(5-((1-((4′-fluoro-3,4,5,6-tetrahydro-[1,1′-biphenyl]-2-yl)methyl)azetidin-3-yl)thio)-1-oxoisoindolin-2-yl)piperidine-2,6-dione (GT-06301)

The target compound (GT-06301) was prepared as a white solid (11 mg, yield: 18%) according to the method described in synthetic method I. ¹H NMR (400 MHz, DMSO-d6) δ 11.00 (s, 1H), 7.72-7.66 (m, 1H), 7.46-7.37 (m, 1H), 7.33-7.07 (m, 5H), 5.11 (dd, J=13.2, 5.1 Hz, 1H), 4.64 (brs, 1H), 4.53-4.19 (m, 4H), 4.18-3.95 (m, 1H), 3.81-3.64 (m, 2H), 3.00-2.81 (m, 1H), 2.67-2.58 (m, 2H), 2.41 (brs, 2H), 2.25 (brs, 2H), 2.13 (brs, 2H), 2.03-1.97 (m, 1H), 1.66 (brs, 3H). LCMS (ESI) calcd for C₂₉H₃₁FN₃O₃S⁺ [M+H]⁺: 520.21, found, 520.1.

### Example 45: Preparation of 3-(5-((1-((4-(4-chlorophenyl)-5,6-dihydro-2H-pyran-3-yl)methyl)azetidin-3-yl)thio)-1-oxoisoindolin-2-yl)piperidine-2,6-dione (GT-06351)

The target compound (GT-06351) was prepared as a white solid (10 mg, yield: 16%) according to the method described in synthetic method I. ¹H NMR (400 MHz, DMSO-d6) δ 10.92 (s, 1H), 7.61 (t, J=8.7 Hz, 1H), 7.45-7.28 (m, 3H), 7.29-7.12 (m, 3H), 5.03 (dd, J=13.3, 5.1 Hz, 1H), 4.67-4.41 (m, 1H), 4.36 (d, J=17.5 Hz, 1H), 4.24 (d, J=17.5 Hz, 2H), 4.12 (s, 2H), 3.82-3.65 (m, 3H), 3.60-3.37 (m, 4H), 2.92-2.77 (m, 1H), 2.53 (d, J=17.1 Hz, 1H), 2.41-2.31 (m, 1H), 2.32-2.23 (m, 2H), 1.99-1.87 (m, 1H). LCMS (ESI) calcd for C₂₈H₂₉ClN₃O₄S⁺ [M+H]⁺: 538.16, found, 538.2.

### Example 46: Preparation of 3-(5-((1-((4-(4-fluorophenyl)-5,6-dihydro-2H-pyran-3-yl)methyl)azetidin-3-yl)thio)-1-oxoisoindolin-2-yl)piperidine-2,6-dione (GT-06513)

The target compound (GT-06513) was prepared as a white solid (16 mg, yield: 26%) according to the method described in synthetic method I. ¹H NMR (400 MHz, DMSO-d6) δ 11.00 (s, 1H), 7.71-7.63 (m, 1H), 7.43-7.36 (m, 1H), 7.33-7.20 (m, 5H), 5.11 (dd, J=13.3, 5.1 Hz, 1H), 4.67-4.60 (m, 1H), 4.45-4.18 (m, 6H), 4.13-4.05 (m, 1H), 3.91-3.76 (m, 3H), 3.68-3.61 (m, 1H), 2.99-2.85 (m, 1H), 2.65-2.53 (m, 2H), 2.46-2.31 (m, 3H), 2.08-1.95 (m, 1H). LCMS (ESI) calcd for C₂₈H₂₉FN₃O₄S⁺ [M+H]⁺: 522.19, found, 522.2.

### Example 47: Preparation of 3-(5-((1-((4′-chloro-4,4-difluoro-3,4,5,6-tetrahydro-[1,1′-biphenyl]-2-yl)methyl)azetidin-3-yl)thio)-1-oxoisoindolin-2-yl)piperidine-2,6-dione (GT-06516)

The target compound (GT-06516) was prepared as a white solid (13 mg, yield: 20%) according to the method described in synthetic method I. ¹H NMR (400 MHz, DMSO-d6) δ 10.92 (s, 1H), 7.60 (d, J=7.8 Hz, 1H), 7.48-7.35 (m, 2H), 7.33-7.29 (m, 1H), 7.23-7.10 (m, 3H), 5.04 (dd, J=13.2, 5.1 Hz, 1H), 4.66-4.50 (m, 1H), 4.45-4.15 (m, 4H), 4.11-3.96 (m, 1H), 3.82-3.73 (m, 2H), 3.64-3.56 (m, 1H), 2.94-2.68 (m, 3H), 2.53 (d, J=16.9 Hz, 1H), 2.48-2.46 (m, 2H), 2.35-2.31 (m, 1H), 2.13-2.04 (m, 2H), 1.94-1.90 (m, 1H). LCMS (ESI) calcd for C₂₉H₂₉ClF₂N₃O₃S⁺ [M+H]⁺: 572.16, found, 572.2.

### Example 48: Preparation of 3-(5-((1-((4′-chloro-4-methoxy-4-methyl-3,4,5,6-tetrahydro-[1,1′-biphenyl]-2-yl)methyl)azetidin-3-yl)thio)-1-oxoisoindolin-2-yl)piperidine-2,6-dione (GT-07064)

The target compound (GT-07064) was prepared as a white solid (10 mg, yield: 14%) according to the method described in synthetic method I. ¹H NMR (400 MHz, DMSO) δ 10.92 (s, 1H), 7.60 (d, J=8.0 Hz, 1H), 7.40 (d, J=8.4 Hz, 1H), 7.37-7.24 (m, 3H), 7.17 (d, J=8.2 Hz, 1H), 7.13 (d, J=8.4 Hz, 1H), 5.04 (dd, J=13.3, 5.0 Hz, 1H), 4.59-4.53 (m, 1H), 4.37 (d, J=17.6 Hz, 1H), 4.27-4.22 (m, 2H), 4.04-4.00 (m, 1H), 3.71-3.64 (m, 2H), 3.57-3.51 (m, 1H), 3.09 (s, 3H), 2.92-2.77 (m, 1H), 2.53 (d, J=16.3 Hz, 1H), 2.49-2.44 (m, 1H), 2.40-2.22 (m, 2H), 2.20-2.13 (m, 3H), 1.97-1.92 (m, 1H), 1.73-1.68 (m, 1H), 1.58-1.51 (m, 1H), 1.11 (s, 3H). LCMS (ESI) calcd for C₃₁H₃₅ClN₃O₄S⁺ [M+H]⁺: 580.20, found, 580.3.

### Example 49: Preparation of 3-(5-((1-((4-(4-chlorophenyl)-6,6-dimethyl-5,6-dihydro-2H-pyran-3-yl)methyl)azetidin-3-yl)thio)-1-oxoisoindolin-2-yl)piperidine-2,6-dione (GT-06514)

The target compound (GT-06514) was prepared as a white solid (21 mg, yield: 32%) according to the method described in synthetic method I. ¹H NMR (400 MHz, DMSO-d6) δ 11.00 (s, 1H), 7.67 (d, J=8.0 Hz, 1H), 7.50 (d, J=8.3 Hz, 1H), 7.44 (d, J=8.4 Hz, 1H), 7.39 (s, 1H), 7.35-7.22 (m, 3H), 5.11 (dd, J=13.3, 5.1 Hz, 1H), 4.65-4.60 (m, 1H), 4.50-4.15 (m, 6H), 4.13-4.10 (m, 1H), 3.85 (s, 2H), 3.73-3.65 (m, 1H), 2.99-2.85 (m, 1H), 2.61 (d, J=16.7 Hz, 1H), 2.46-2.35 (m, 1H), 2.23 (s, 2H), 2.06-1.94 (m, 1H), 1.21 (s, 6H). LCMS (ESI) calcd for C₃₀H₃₃ClN₃O₄S⁺ [M+H]⁺: 566.19, found, 566.2.

### Example 50: Preparation of 3-(5-((1-benzhydrylazetidin-3-yl)thio)-1-oxoisoindolin-2-yl)piperidine-2,6-dione (GT-06138)

The target compound (GT-06138) was prepared as a white solid (24 mg, yield: 33%) according to the method described in synthetic method II. ¹H NMR (400 MHz, DMSO-d6) δ 10.91 (s, 1H), 7.64-7.52 (m, 5H), 7.40-7.24 (m, 8H), 5.02 (dd, J=13.3, 5.1 Hz, 1H), 4.64-4.52 (m, 2H), 4.36-4.32 (m, 2H), 4.25-4.19 (m, 2H), 4.10-3.95 (m, 2H), 2.91-2.77 (m, 1H), 2.52 (d, J=17.0 Hz, 1H), 2.37-2.21 (m, 1H), 1.96-1.90 (m, 1H). LCMS (ESI) calcd for C₂₉H₂₈N₃O₃S⁺ [M+H]⁺: 498.18, found, 498.2.

### Example 51: Preparation of 3-(5-((1-benzhydrylpyrrolidin-3-yl)thio)-1-oxoisoindolin-2-yl)piperidine-2,6-dione (GT-06196)

The target compound (GT-06196) was prepared as a white solid (11 mg, yield: 15%) according to the method described in synthetic method II. ¹H NMR (400 MHz, DMSO-d6) δ 10.92 (s, 1H), 7.81-7.70 (m, 3H), 7.61-7.55 (m, 2H), 7.41-7.24 (m, 8H), 5.69-5.60 (m, 1H), 5.02 (dd, J=13.1, 5.0 Hz, 1H), 4.38-4.21 (m, 3H), 3.69-3.58 (m, 1H), 3.23-3.10 (m, 2H), 3.05-2.94 (m, 1H), 2.91-2.76 (m, 1H), 2.59-2.50 (m, 2H), 2.37-2.26 (m, 1H), 2.13-1.98 (m, 1H), 1.94-1.85 (m, 1H). LCMS (ESI) calcd for C₃₀H₃₀N₃O₃S⁺ [M+H]⁺: 512.20, found, 512.2.

### Example 52: Preparation of 3-(5-((1-benzhydrylpiperidin-4-yl)thio)-1-oxoisoindolin-2-yl)piperidine-2,6-dione (GT-06184)

The target compound (GT-06184) was prepared as a white solid (12 mg, yield: 17%) according to the method described in synthetic method II. ¹H NMR (400 MHz, DMSO-d6) δ 10.92 (s, 1H), 7.84 (d, J=7.5 Hz, 1H), 7.79 (d, J=7.5 Hz, 3H), 7.61-7.55 (m, 2H), 7.48-7.41 (m, 1H), 7.42-7.34 (m, 4H), 7.33-7.21 (m, 2H), 5.03 (dd, J=12.8, 4.5 Hz, 1H), 4.36 (d, J=17.5 Hz, 1H), 4.23 (d, J=17.3 Hz, 1H), 3.52-3.45 (m, 1H), 3.20-3.13 (m, 2H), 3.08-2.97 (m, 2H), 2.88-2.79 (m, 1H), 2.52 (d, J=16.4 Hz, 1H), 2.36-2.25 (m, 2H), 2.11-2.06 (m, 3H), 1.99-1.84 (m, 2H). LCMS (ESI) calcd for C₃₁H₃₂N₃O₃S⁺[M+H]⁺: 526.22, found, 526.2.

### Example 53: Preparation of 3-(5-((1-benzhydrylazepan-4-yl)thio)-1-oxoisoindolin-2-yl)piperidine-2,6-dione (GT-06645)

The target compound (GT-06645) was prepared as a white solid (7 mg, yield: 10%) according to the method described in synthetic method II. ¹H NMR (400 MHz, DMSO-d6) δ 10.91 (s, 1H), 7.80-7.75 (m, 4H), 7.57 (t, J=7.9 Hz, 1H), 7.52 (s, 1H), 7.41-7.35 (m, 5H), 7.32-7.23 (m, 2H), 5.61 (dd, J=28.7, 9.4 Hz, 1H), 5.04-5.01 (m, 1H), 4.34 (d, J=16.8 Hz, 1H), 4.22 (d, J=17.1 Hz, 1H), 3.84-3.71 (m, 1H), 3.10-2.98 (m, 1H), 2.95-2.80 (m, 2H), 2.52 (d, J=18.3 Hz, 2H), 2.37-2.25 (m, 2H), 2.22-2.14 (m, 2H), 2.08-2.02 (m, 1H), 1.95-1.86 (m, 4H), 1.71-1.67 (m, 1H), 1.57-1.48 (m, 1H). LCMS (ESI) calcd for C₃₂H₃₄N₃O₃S⁺ [M+H]⁺: 540.23, found, 540.2.

### Example 54: Preparation of 3-(5-((1-benzhydryl-3,3-difluoropiperidin-4-yl)thio)-1-oxoisoindolin-2-yl)piperidine-2,6-dione (GT-06926)

The target compound (GT-06926) was prepared as a white solid (12 mg, yield: 17%) according to the method described in synthetic method II. ¹H NMR (400 MHz, DMSO-d6) δ 10.91 (s, 1H), 7.62 (s, 1H), 7.58 (d, J=8.0 Hz, 1H), 7.49 (d, J=8.0 Hz, 1H), 7.33 (d, J=7.3 Hz, 4H), 7.28-7.23 (m, 4H), 7.20-7.11 (m, 2H), 5.02 (dd, J=13.3, 4.9 Hz, 1H), 4.52 (s, 1H), 4.35 (d, J=17.3 Hz, 1H), 4.22 (d, J=17.5 Hz, 1H), 3.98-3.77 (m, 1H), 2.99-2.92 (m, 1H), 2.91-2.77 (m, 1H), 2.71 (d, J=11.5 Hz, 1H), 2.52 (d, J=16.7 Hz, 1H), 2.40-2.23 (m, 2H), 2.15 (t, J=10.9 Hz, 1H), 2.10-1.99 (m, 1H), 1.99-1.87 (m, 1H), 1.79-1.70 (m, 1H). LCMS (ESI) calcd for C₃₁H₃₀F₂N₃O₃S⁺ [M+H]⁺: 562.20, found, 562.2.

### Example 55: Preparation of 3-(5-((7-benzhydryl-7-azaspiro[3.5]nonan-2-yl)thio)-1-oxoisoindolin-2-yl)piperidine-2,6-dione (GT-06920)

The target compound (GT-06920) was prepared as a white solid (7 mg, yield: 15%) according to the method described in synthetic method II. ¹H NMR (400 MHz, DMSO-d6) δ 11.37 (s, 1H), 10.91 (s, 1H), 7.87-7.73 (m, 4H), 7.58-7.55 (m, 1H), 7.45-7.32 (m, 5H), 7.33-7.27 (m, 2H), 7.25-7.22 (m, 1H), 5.50 (d, J=9.3 Hz, 1H), 5.02 (dd, J=13.4, 5.0 Hz, 1H), 4.34 (d, J=17.4 Hz, 1H), 4.21 (d, J=17.4 Hz, 1H), 4.13-3.93 (m, 1H), 3.57-3.44 (m, 1H), 3.09-2.75 (m, 4H), 2.55-2.44 (m, 2H), 2.40-2.20 (m, 2H), 2.09-2.00 (m, 1H), 1.98-1.87 (m, 2H), 1.83-1.75 (m, 3H), 1.69-1.65 (m, 1H). LCMS (ESI) calcd for C₃₄H₃₆N₃O₃S⁺ [M+H]⁺: 566.25, found, 566.3.

### Example 56: Preparation of 3-(5-((3-benzhydryl-3-azaspiro[5.5]undecan-9-yl)thio)-1-oxoisoindolin-2-yl)piperidine-2,6-dione (GT-06958)

The target compound (GT-06958) was prepared as a white solid (6 mg, yield: 14%) according to the method described in synthetic method II. ¹H NMR (400 MHz, DMSO) δ 10.92 (s, 1H), 7.72-7.70 (m, 3H), 7.60-7.56 (m, 1H), 7.52-7.50 (m, 1H), 7.42-7.36 (m, 5H), 7.32-7.28 (m, 2H), 7.20-7.11 (m, 1H), 5.04-5.01 (m, 1H), 4.36 (d, J=17.3 Hz, 1H), 4.23 (d, J=17.8 Hz, 1H), 3.59-3.52 (m, 1H), 3.44-3.36 (m, 1H), 3.01-2.95 (m, 2H), 2.88-2.81 (m, 1H), 2.53 (d, J=16.1 Hz, 1H), 2.33-2.25 (m, 1H), 1.97-1.90 (m, 2H), 1.83-1.75 (m, 3H), 1.69-1.63 (m, 2H), 1.56-1.52 (m, 2H), 1.46-1.37 (m, 4H), 1.32-1.24 (m, 2H). LCMS (ESI) calcd for C₃₆H₄₀N₃O₃S⁺ [M+H]⁺: 594.28, found, 594.3.

### Example 57: Preparation of 3-(5-((2-benzhydryloctahydrocyclopenta[c]pyrrol-5-yl)thio)-1-oxoisoindolin-2-yl)piperidine-2,6-dione (GT-06922)

The target compound (GT-06922) was prepared as a white solid (6 mg, yield: 12%) according to the method described in synthetic method II. ¹H NMR (400 MHz, DMSO-d6) δ 10.92 (s, 1H), 7.85-7.69 (m, 4H), 7.62-7.48 (m, 2H), 7.43-7.35 (m, 5H), 7.32-7.27 (m, 2H), 5.65-5.50 (m, 1H), 5.08-4.94 (m, 1H), 4.36 (dd, J=17.4, 7.6 Hz, 1H), 4.23 (dd, J=17.4, 8.7 Hz, 1H), 3.99-3.86 (m, 1H), 3.12-3.09 (m, 1H), 2.94-2.78 (m, 4H), 2.53 (d, J=16.0 Hz, 1H), 2.37-2.26 (m, 3H), 2.08-1.87 (m, 2H), 1.82-1.73 (m, 1H), 1.62-1.54 (m, 1H), 1.49-1.42 (m, 1H). LCMS (ESI) calcd for C₃₃H₃₄N₃O₃S⁺ [M+H]⁺: 552.23, found, 552.3.

### Example 58: Preparation of 3-(5-((1-(bis(2-fluorophenyl)methyl)azetidin-3-yl)thio)-1-oxoisoindolin-2-yl)piperidine-2,6-dione (GT-06758)

The target compound (GT-06758) was prepared as a white solid (25 mg, yield: 40%) according to the method described in synthetic method II. ¹H NMR (400 MHz, DMSO-d6) δ 10.91 (s, 1H), 7.69-7.49 (m, 3H), 7.48-7.24 (m, 4H), 7.25-7.04 (m, 4H), 5.02 (dd, J=13.2, 5.0 Hz, 1H), 4.49-4.14 (m, 4H), 3.99 (s, 1H), 3.57-3.33 (m, 3H), 2.94-2.77 (m, 1H), 2.52 (d, J=16.3 Hz, 1H), 2.32-2.29 (m, 1H), 1.99-1.87 (m, 1H). LCMS (ESI) calcd for C₂₉H₂₆F₂N₃O₃S⁺ [M+H]⁺: 534.17, found, 534.3.

### Example 59: Preparation of 3-(5-((1-(bis(3-fluorophenyl)methyl)azetidin-3-yl)thio)-1-oxoisoindolin-2-yl)piperidine-2,6-dione (GT-06349)

The target compound (GT-06349) was prepared as a white solid (21 mg, yield: 34%) according to the method described in synthetic method II. ¹H NMR (400 MHz, DMSO-d6) δ 10.92 (s, 1H), 7.69-7.47 (m, 2H), 7.47-7.20 (m, 7H), 7.18-7.01 (m, 2H), 5.11-4.95 (m, 1H), 4.52-4.13 (m, 4H), 3.71-3.53 (m, 2H), 2.92-2.76 (m, 2H), 2.59-2.49 (m, 2H), 2.37-2.23 (m, 1H), 2.02-1.86 (m, 1H). LCMS (ESI) calcd for C₂₉H₂₆F₂N₃O₃S⁺ [M+H]⁺: 534.17, found, 534.3.

### Example 60: Preparation of 3-(5-((1-(bis(4-fluorophenyl)methyl)azetidin-3-yl)thio)-1-oxoisoindolin-2-yl)piperidine-2,6-dione (GT-06503)

The target compound (GT-06503) was prepared as a white solid (17 mg, yield: 27%) according to the method described in synthetic method II. ¹H NMR (400 MHz, DMSO-d6) δ 10.93 (s, 1H), 7.83-7.50 (m, 5H), 7.50-7.34 (m, 2H), 7.34-7.14 (m, 4H), 5.02 (dd, J=13.3, 5.0 Hz, 1H), 4.58-4.39 (m, 3H), 4.34 (d, J=17.5 Hz, 1H), 4.22 (d, J=17.6 Hz, 1H), 4.08-3.95 (m, 2H), 2.90-2.76 (m, 1H), 2.58-2.49 (m, 2H), 2.34-2.26 (m, 1H), 1.98-1.88 (m, 1H). LCMS (ESI) calcd for C₂₉H₂₆F₂N₃O₃S⁺ [M+H]⁺: 534.17, found, 534.2.

### Example 61: Preparation of 3-(5-((1-(bis(4-chlorophenyl)methyl)azetidin-3-yl)thio)-1-oxoisoindolin-2-yl)piperidine-2,6-dione (GT-06504)

The target compound (GT-06504) was prepared as a white solid (26 mg, yield: 40%) according to the method described in synthetic method II. ¹H NMR (400 MHz, DMSO-d6) δ 10.92 (d, J=11.3 Hz, 1H), 7.82-7.51 (m, 5H), 7.50-7.31 (m, 6H), 5.02 (dd, J=13.4, 5.0 Hz, 1H), 4.64-4.32 (m, 3H), 4.34 (d, J=17.6 Hz, 1H), 4.21 (d, J=17.6 Hz, 1H), 4.15-3.88 (m, 2H), 2.90-2.77 (m, 1H), 2.58-2.50 (m, 2H), 2.37-2.25 (m, 1H), 1.98-1.88 (m, 1H). LCMS (ESI) calcd for C₂₉H₂₆Cl₂N₃O₃S⁺ [M+H]⁺: 566.11, found, 566.1.

### Example 62: Preparation of 3-(5-((1-(bis(4-methoxyphenyl)methyl)azetidin-3-yl)thio)-1-oxoisoindolin-2-yl)piperidine-2,6-dione (GT-06923)

The target compound (GT-06923) was prepared as a white solid (9 mg, yield: 14%) according to the method described in synthetic method II. ¹H NMR (400 MHz, DMSO-d6) δ 10.92 (s, 1H), 7.61-7.58 (m, 1H), 7.52-7.47 (m, 4H), 7.42-7.24 (m, 2H), 6.95-6.90 (m, 4H), 5.69 (dd, J=81.4, 9.8 Hz, 1H), 5.02 (dd, J=13.3, 5.1 Hz, 1H), 4.60-4.16 (m, 5H), 3.99-3.93 (m, 2H), 3.67 (s, 6H), 2.95-2.74 (m, 1H), 2.52 (d, J=16.9 Hz, 1H), 2.39-2.23 (m, 1H), 1.95-1.90 (m, 1H). LCMS (ESI) calcd for C₃₁H₃₁N₃NaO₅S⁺ [M+Na]⁺: 580.19, found, 580.3.

### Example 63: Preparation of 3-(1-oxo-5-((1-(phenyl(pyridin-2-yl)methyl)azetidin-3-yl)thio)isoindolin-2-yl)piperidine-2,6-dione (GT-06518)

The target compound (GT-06518) was prepared as a white solid (5 mg, yield: 9%) according to the method described in synthetic method II. ¹H NMR (400 MHz, DMSO-d6) δ 10.99 (s, 1H), 8.73-8.60 (m, 1H), 7.92-7.81 (m, 1H), 7.75-7.62 (m, 2H), 7.62-7.49 (m, 3H), 7.49-7.29 (m, 5H), 5.21-5.05 (m, 1H), 4.64-4.13 (m, 4H), 3.71-3.67 (m, 1H), 3.60-3.40 (m, 3H), 2.97-2.88 (m, 1H), 2.61 (d, J=18.4 Hz, 1H), 2.44-2.37 (m, 1H), 2.05-1.98 (m, 1H). LCMS (ESI) calcd for C₂₈H₂₇N₄O₃S⁺ [M+H]⁺: 499.18, found, 499.2.

### Example 64: Preparation of 3-(1-oxo-5-((1-(phenyl(pyridin-3-yl)methyl)azetidin-3-yl)thio)isoindolin-2-yl)piperidine-2,6-dione (GT-06807)

The target compound (GT-06807) was prepared as a white solid (18 mg, yield: 31%) according to the method described in synthetic method II. ¹H NMR (400 MHz, DMSO-d6) δ 10.95 (s, 1H), 8.98-8.82 (m, 1H), 8.59-8.54 (m, 1H), 8.40-8.16 (m, 1H), 8.07-8.95 (m, 1H), 7.84-7.44 (m, 4H), 7.43-7.17 (m, 4H), 5.13-4.95 (m, 1H), 4.85-4.58 (m, 1H), 4.50-3.95 (m, 4H), 3.89-3.59 (m, 2H), 2.94-2.77 (m, 2H), 2.54 (d, J=16.5 Hz, 1H), 2.40-2.20 (m, 1H), 2.04-1.85 (m, 1H). LCMS (ESI) calcd for C₂₈H₂₇N₄O₃S⁺ [M+H]⁺: 499.18, found, 499.2.

### Example 65: Preparation of 3-(5-((1-(di(pyridin-2-yl)methyl)azetidin-3-yl)thio)-1-oxoisoindolin-2-yl)piperidine-2,6-dione (GT-06811)

The target compound (GT-06811) was prepared as a white solid (20 mg, yield: 34%) according to the method described in synthetic method II. ¹H NMR (400 MHz, DMSO-d6) δ 11.02 (s, 1H), 8.66-8.53 (m, 2H), 7.96-7.83 (m, 2H), 7.78-7.64 (m, 1H), 7.62-7.55 (m, 3H), 7.51-7.32 (m, 3H), 5.20-5.01 (m, 1H), 4.65-4.61 (m, 1H), 4.51-4.42 (m, 1H), 4.36-4.30 (m, 1H), 4.29-4.00 (m, 2H), 3.67-3.56 (m, 2H), 3.02-2.89 (m, 1H), 2.62 (d, J=16.7 Hz, 1H), 2.45-2.38 (m, 1H), 2.07-1.94 (m, 1H).

LCMS (ESI) calcd for C₂₇H₂₆N₅O₃S⁺ [M+H]⁺: 500.18, found, 500.2.

### Example 66: Preparation of 3-(4-((1-((4′-chloro-5,5-dimethyl-3,4,5,6-tetrahydro-[1,1′-biphenyl]-2-yl)methyl)azetidin-3-yl)oxy)-1-oxoisoindolin-2-yl)piperidine-2,6-dione (GT-06952)

The target compound (GT-06952) was prepared as a white solid (31 mg, yield: 46%) according to the method described in synthetic method I. ¹H NMR (400 MHz, DMSO-d6) δ 10.96 (s, 1H), 7.47-7.29 (m, 4H), 7.23 (d, J=8.0 Hz, 1H), 7.09-7.06 (m, 2H), 6.91-6.83 (m, 1H), 5.23-4.90 (m, 2H), 4.68-4.57 (m, 1H), 4.34-4.01 (m, 3H), 3.86-3.56 (m, 3H), 3.46 (d, J=5.1 Hz, 1H), 2.93-2.82 (m, 1H), 2.63-2.52 (m, 2H), 2.21-2.10 (m, 2H), 2.06-1.84 (m, 3H), 1.43-1.27 (m, 2H), 0.88 (s, 6H).

LCMS (ESI) calcd for C₃₁H₃₅ClN₃O₄⁺ [M+H]⁺: 548.23, found, 548.3.

### Example 67: Preparation of 3-(6-((1-((4′-chloro-5,5-dimethyl-3,4,5,6-tetrahydro-[1,1′-biphenyl]-2-yl)methyl)azetidin-3-yl)oxy)-1-oxoisoindolin-2-yl)piperidine-2,6-dione (GT-06872)

The target compound (GT-06872) was prepared as a white solid (19 mg, yield: 28%) according to the method described in synthetic method I. ¹H NMR (400 MHz, DMSO-d6) δ 10.99 (s, 1H), 7.58-7.56 (m, 1H), 7.51-7.36 (m, 3H), 7.22 (d, J=8.0 Hz, 1H), 7.20-7.11 (m, 2H), 5.11 (dd, J=13.2, 4.7 Hz, 1H), 5.07-5.00 (m, 1H), 4.73-4.68 (m, 1H), 4.42-4.25 (m, 3H), 3.84-3.76 (m, 2H), 2.95-2.87 (m, 1H), 2.61 (d, J=17.2 Hz, 1H), 2.45-2.33 (m, 1H), 2.25-2.15 (m, 3H), 2.10-1.94 (m, 4H), 1.48-1.44 (m, 2H), 0.95 (s, 6H). LCMS (ESI) calcd for C₃₁H₃₅ClN₃O₄⁺ [M+H]⁺: 548.23, found, 548.3.

### Example 68: Preparation of 3-(7-((1-((4′-chloro-5,5-dimethyl-3,4,5,6-tetrahydro-[1,1′-biphenyl]-2-yl)methyl))zetidine-3-yl)oxy)-1-oxoisoindolin-2-yl)piperidine-2,6-dione (GT-07063)

The target compound (GT-07063) was prepared as a white solid (6 mg, yield: 10%) according to the method described in synthetic method I. ¹H NMR (400 MHz, DMSO-d6) δ 10.91 (s, 1H), 10.49 (s, 1H), 7.46 (t, J=7.8 Hz, 1H), 7.39 (t, J=8.1 Hz, 1H), 7.28 (d, J=8.1 Hz, 1H), 7.22-7.04 (m, 3H), 6.69 (d, J=8.2 Hz, 1H), 5.03-4.85 (m, 2H), 4.58 (t, J=17.0 Hz, 1H), 4.34 (d, J=17.6 Hz, 1H), 4.24-4.19 (m, 2H), 3.92-3.63 (m, 3H), 2.90-2.73 (m, 1H), 2.56-2.50 (m, 1H), 2.48-2.45 (m, 1H), 2.36-2.28 (m, 1H), 2.16-2.07 (m, 2H), 2.02-1.86 (m, 3H), 1.40-1.35 (m, 2H), 0.88 (s, 6H). LCMS (ESI) calcd for C₃₁H₃₅ClN₃O₄⁺ [M+H]⁺: 548.23, found, 548.3.

### Example 69: Preparation of 3-(5-((1-((4′-chloro-5,5-dimethyl-3,4,5,6-tetrahydro-[1,1′-biphenyl]-2-yl)methyl)pyrrolidin-3-yl)oxy)-1-oxoisoindolin-2-yl)piperidine-2,6-dione (GT-06815)

The target compound (GT-06815) was prepared as a white solid (41 mg, yield: 63%) according to the method described in synthetic method I. ¹H NMR (400 MHz, DMSO-d6) δ 10.98 (s, 1H), 7.46 (t, J=7.9 Hz, 1H), 7.40-7.37 (m, 2H), 7.16-7.12 (m, 2H), 6.84 (d, J=5.9 Hz, 1H), 6.63 (d, J=8.4 Hz, 1H), 5.17-5.08 (m, 2H), 4.41 (d, J=19.5 Hz, 1H), 4.29 (d, J=17.3 Hz, 1H), 3.87-3.74 (m, 2H), 3.58-3.54 (m, 2H), 3.16 (d, J=14.4 Hz, 1H), 2.95-2.89 (m, 2H), 2.61 (d, J=17.2 Hz, 1H), 2.44-2.40 (m, 2H), 2.29-2.20 (m, 2H), 2.12-1.98 (m, 3H), 1.54-1.41 (m, 3H), 0.97 (s, 6H). LCMS (ESI) calcd for C₃₂H₃₇ClN₃O₄⁺ [M+H]⁺: 562.25, found, 562.3.

### Example 70: Preparation of 3-(5-((1-((4′-chloro-5,5-dimethyl-3,4,5,6-tetrahydro-[1,1′-biphenyl]-2-yl)methyl)piperidin-4-yl)oxy)-1-oxoisoindolin-2-yl)piperidine-2,6-dione (GT-07061)

The target compound (GT-07061) was prepared as a white solid (4 mg, yield: 8%) according to the method described in synthetic method I. ¹H NMR (400 MHz, DMSO-d6) δ 10.97 (s, 1H), 7.66-7.62 (m, 1H), 7.52-7.42 (m, 2H), 7.22-7.12 (m, 3H), 7.09-7.00 (m, 1H), 5.14-5.02 (m, 1H), 4.82-4.63 (m, 1H), 4.37 (d, J=16.9 Hz, 1H), 4.25 (d, J=17.4 Hz, 1H), 3.62-3.55 (m, 3H), 2.95-2.88 (m, 1H), 2.82-2.74 (m, 2H), 2.62-2.57 (m, 3H), 2.36-2.31 (m, 2H), 2.25-2.16 (m, 2H), 2.05 (s, 2H), 2.02-1.92 (m, 3H), 1.52-1.47 (m, 2H), 0.97 (s, 6H). LCMS (ESI) calcd for C₃₃H₃₉ClN₃O₄⁺ [M+H]⁺: 576.26, found, 576.3.

### Example 71: Preparation of 3-(5-((1-((4′-chloro-5,5-dimethyl-3,4,5,6-tetrahydro-[1,1′-biphenyl]-2-yl)methyl)azepan-4-yl)oxy)-1-oxoisoindolin-2-yl)piperidine-2,6-dione (GT-06929)

The target compound (GT-06929) was prepared as a white solid (17 mg, yield: 27%) according to the method described in synthetic method I. ¹H NMR (400 MHz, DMSO-d6) δ 10.90 (s, 1H), 7.55 (d, J=8.4 Hz, 1H), 7.40-7.36 (m, 2H), 7.13-7.03 (m, 3H), 6.98-6.88 (m, 1H), 5.00 (dd, J=13.3, 5.0 Hz, 1H), 4.70-4.61 (m, 1H), 4.31 (d, J=17.2 Hz, 1H), 4.19 (d, J=17.3 Hz, 1H), 3.52-3.46 (m, 2H), 3.41-3.30 (m, 1H), 3.19-3.07 (m, 1H), 3.00-2.95 (m, 1H), 2.88-2.79 (m, 3H), 2.52 (d, J=16.7 Hz, 1H), 2.39-2.16 (m, 3H), 2.16-2.05 (m, 1H), 2.01-1.97 (m, 3H), 1.94-1.86 (m, 2H), 1.84-1.79 (m, 2H), 1.76-1.60 (m, 2H), 1.44-1.31 (m, 2H), 0.89 (s, 6H). LCMS (ESI) calcd for C₃₄H₄₁ClN₃O₄⁺ [M+H]⁺: 590.28, found, 590.3.

### Example 72: Preparation of 3-(5-(((3aS,6aS)-2-((4′-chloro-5,5-dimethyl-3,4,5,6-tetrahydro-[1,1′-biphenyl]-2-yl)methyl)octahydrocyclopenta[c]pyrrol-5-yl)oxy)-1-oxoisoindolin-2-yl)piperidine-2,6-dione (GT-06930)

The target compound (GT-06930) was prepared as a white solid (28 mg, yield: 44%) according to the method described in synthetic method I. ¹H NMR (400 MHz, DMSO-d6) δ 10.99 (s, 1H), 10.78 (s, 1H), 7.59 (d, J=8.4 Hz, 1H), 7.53 (d, J=8.4 Hz, 2H), 7.46 (d, J=8.4 Hz, 1H), 7.23 (d, J=8.4 Hz, 2H), 7.20 (d, J=8.5 Hz, 1H), 5.11-5.01 (m, 2H), 4.38 (d, J=17.2 Hz, 1H), 4.27 (d, J=17.2 Hz, 1H), 3.66-2.53 (m, 4H), 3.20-3.16 (m, 1H), 2.99-2.92 (m, 3H), 2.63-2.59 (m, 1H), 2.43-2.28 (m, 4H), 2.07 (s, 2H), 2.03-1.90 (m, 3H), 1.86-1.83 (m, 2H), 1.48 (t, J=6.0 Hz, 2H), 0.97 (s, 6H). LCMS (ESI) calcd for C₃₅H₄₁ClN₃O₄⁺ [M+H]⁺: 602.28, found, 602.3.

### Example 73: Preparation of 3-(5-(((3aR,6aS)-2-((4′-chloro-5,5-dimethyl-3,4,5,6-tetrahydro-[1,1′-biphenyl]-2-yl)methyl)octahydrocyclopenta[c]pyrrol-5-yl)oxy)-1-oxoisoindolin-2-yl)piperidine-2,6-dione (GT-06931)

The target compound (GT-06931) was prepared as a white solid (11 mg, yield: 17%) according to the method described in synthetic method I. ¹H NMR (400 MHz, DMSO-d6) δ 10.89 (s, 1H), 7.54 (dd, J=8.3, 5.4 Hz, 1H), 7.38 (d, J=8.4 Hz, 2H), 7.11 (d, J=8.4 Hz, 2H), 7.06 (d, J=14.5 Hz, 1H), 6.94 (d, J=8.1 Hz, 1H), 5.00 (dd, J=12.7, 5.2 Hz, 2H), 4.31 (d, J=17.2 Hz, 1H), 4.19 (d, J=17.0 Hz, 1H), 3.57-3.52 (m, 3H), 3.15 (d, J=11.8 Hz, 1H), 2.91-2.81 (m, 3H), 2.72-2.67 (m, 1H), 2.54-2.51 (m, 2H), 2.38-2.19 (m, 3H), 2.09-1.87 (m, 5H), 1.85-1.77 (m, 2H), 1.39 (brs, 2H), 0.90 (s, 6H). LCMS (ESI) calcd for C₃₅H₄₁ClN₃O₄⁺ [M+H]⁺: 602.28, found, 602.3.

### Example 74: Preparation of 3-(5-((7-((4′-chloro-5,5-dimethyl-3,4,5,6-tetrahydro-[1,1′-biphenyl]-2-yl)methyl)-7-azaspiro[3.5]nonan-2-yl)oxy)-1-oxoisoindolin-2-yl)piperidine-2,6-dione (GT-06933)

The target compound (GT-06933) was prepared as a white solid (15 mg, yield: 29%) according to the method described in synthetic method I. ¹H NMR (400 MHz, DMSO-d6) δ 10.89 (s, 1H), 9.64 (s, 1H), 7.54 (d, J=8.4 Hz, 1H), 7.37 (d, J=8.3 Hz, 2H), 7.07 (d, J=8.3 Hz, 2H), 6.95 (s, 1H), 6.87 (dd, J=8.4, 2.0 Hz, 1H), 4.99 (dd, J=13.3, 5.1 Hz, 1H), 4.80-4.63 (m, 1H), 4.29 (dd, J=17.2, 5.6 Hz, 1H), 4.17 (dd, J=17.2, 4.7 Hz, 1H), 3.46 (brs, 2H), 3.08 (dd, J=27.5, 11.6 Hz, 2H), 2.93-2.74 (m, 1H), 2.54-2.47 (m, 3H), 2.38-2.16 (m, 5H), 2.07-1.81 (m, 6H), 1.77-1.61 (m, 2H), 1.58 (d, J=13.7 Hz, 1H), 1.40 (t, J=5.9 Hz, 2H), 0.89 (s, 6H). LCMS (ESI) calcd for C₃₆H₄₃ClN₃O₄⁺ [M+H]⁺: 616.29, found, 616.3.

### Example 75: Preparation of 3-(5-((1-(4′-chloro-5,5-dimethyl-3,4,5,6-tetrahydro-[1,1′-biphenyl]-2-carbonyl)azetidin-3-yl)oxy)-1-oxoisoindolin-2-yl)piperidine-2,6-dione (GT-06180)

The target compound (GT-06180) was prepared as a white solid (26 mg, yield: 31%) according to the method described in synthetic method III. ¹H NMR (400 MHz, DMSO-d6) δ 10.97 (s, 1H), 7.62 (d, J=8.4 Hz, 1H), 7.37 (dd, J=8.6, 2.3 Hz, 2H), 7.26 (dd, J=8.5, 1.7 Hz, 2H), 6.93 (s, 1H), 6.85 (dd, J=8.4, 1.9 Hz, 1H), 5.07 (dd, J=13.2, 3.7 Hz, 1H), 4.97-4.82 (m, 1H), 4.38 (d, J=17.3 Hz, 1H), 4.25 (d, J=17.1 Hz, 1H), 4.22-4.04 (m, 2H), 3.60-3.50 (m, 2H), 2.99-2.83 (m, 1H), 2.59 (d, J=17.6 Hz, 1H), 2.44-2.32 (m, 2H), 2.25-2.18 (m, 2H), 2.06 (s, 1H), 2.01-1.97 (m, 1H), 1.42 (t, J=6.4 Hz, 2H), 0.98 (s, 3H), 0.96 (s, 3H). LCMS (ESI) calcd for C₃₁H₃₃ClN₃O₅[M+H]⁺: 562.21, found, 562.3.

### Example 76: Preparation of 3-(5-((1-(4′-chloro-5,5-dimethyl-3,4,5,6-tetrahydro-[1,1′-biphenyl]-2-carbonyl)pyrrolidin-3-yl)oxy)-1-oxoisoindolin-2-yl)piperidine-2,6-dione (GT-06816)

The target compound (GT-06816) was prepared as a white solid (33 mg, yield: 49%) according to the method described in synthetic method III. ¹H NMR (400 MHz, DMSO-d6) δ 10.98 (s, 1H), 7.63-7.60 (m, 1H), 7.37-7.34 (m, 1H), 7.28-7.25 (m, 2H), 7.22-7.17 (m, 1H), 7.01-6.93 (m, 1H), 6.91-6.76 (m, 1H), 5.11-5.07 (m, 1H), 4.96 (d, J=28.8 Hz, 1H), 4.43-4.38 (m, 1H), 4.30-4.24 (m, 1H), 3.27-3.19 (m, 2H), 2.99-2.82 (m, 2H), 2.60 (d, J=17.1 Hz, 1H), 2.46-2.32 (m, 2H), 2.29-2.20 (m, 2H), 2.16-1.76 (m, 5H), 1.53-1.33 (m, 2H), 1.01 (s, 3H), 0.99 (s, 3H). LCMS (ESI) calcd for C₃₂H₃₅ClN₃O₅⁺ [M+H]⁺: 576.23, found, 576.3.

### Example 77: Preparation of 3-(5-((1-(4′-chloro-5,5-dimethyl-3,4,5,6-tetrahydro-[1,1′-biphenyl]-2-carbonyl)piperidin-4-yl)oxy)-1-oxoisoindolin-2-yl)piperidine-2,6-dione (GT-06192)

The target compound (GT-06192) was prepared as a white solid (27 mg, yield: 33%) according to the method described in synthetic method III. ¹H NMR (400 MHz, DMSO-d6) δ 10.96 (s, 1H), 7.59 (d, J=8.4 Hz, 1H), 7.41 (d, J=6.4 Hz, 2H), 7.28 (d, J=6.4 Hz, 2H), 7.18-7.10 (m, 1H), 7.05-6.95 (m, 1H), 5.06 (dd, J=13.3, 4.9 Hz, 1H), 4.54 (s, 1H), 4.36 (d, J=17.2 Hz, 1H), 4.23 (d, J=17.1 Hz, 1H), 3.90-3.87 (m, 1H), 3.73-3.62 (m, 2H), 2.97-2.82 (m, 2H), 2.59 (d, J=17.8 Hz, 2H), 2.46-2.34 (m, 3H), 2.18-2.03 (m, 1H), 2.03-1.81 (m, 3H), 1.69-1.56 (m, 1H), 1.56-1.41 (m, 3H), 1.00 (s, 3H), 0.97 (s, 3H). LCMS (ESI) calcd for C₃₃H₃₇ClN₃O₅⁺ [M+H]⁺: 590.24, found, 590.3.

### Example 78: Preparation of 3-(5-((1-(4′-chloro-5,5-dimethyl-3,4,5,6-tetrahydro-[1,1′-biphenyl]-2-carbonyl)azepan-4-yl)oxy)-1-oxoisoindolin-2-yl)piperidine-2,6-dione (GT-07018)

The target compound (GT-07018) was prepared as a white solid (23 mg, yield: 35%) according to the method described in synthetic method III. ¹H NMR (400 MHz, DMSO-d6) δ 10.98 (s, 1H), 7.63-7.59 (m, 1H), 7.46-7.26 (m, 4H), 7.08-7.02 (m, 1H), 6.98-6.92 (m, 1H), 5.07 (dd, J=13.2, 5.0 Hz, 1H), 4.38 (d, J=17.3 Hz, 1H), 4.33-4.15 (m, 1H), 3.29-3.22 (m, 2H), 3.12-3.03 (m, 2H), 3.01-2.81 (m, 1H), 2.59 (d, J=16.2 Hz, 1H), 2.47-2.29 (m, 3H), 2.21-2.11 (m, 1H), 2.01-1.88 (m, 2H), 1.84-1.67 (m, 2H), 1.67-1.51 (m, 2H), 1.51-1.31 (m, 3H), 1.18-1.12 (m, 1H), 1.01 (s, 3H), 0.98 (s, 3H).

LCMS (ESI) calcd for C₃₄H₃₉ClN₃O₅⁺ [M+H]⁺: 604.26, found, 604.3.

### Example 79: 3-(5-(((3aS,6aS)-2-(4′-chloro-5,5-dimethyl-3,4,5,6-tetrahydro-[1,1′-biphenyl]-2-carbonyl)octahydrocyclopenta[c]pyrrol-5-yl)oxy)-1-oxoisoindolin-2-yl)piperidine-2,6-dione (GT-07025) Preparation of

The target compound (GT-07025) was prepared as a white solid (22 mg, yield: 34%) according to the method described in synthetic method III. ¹H NMR (400 MHz, DMSO-d6) δ 10.91 (s, 1H), 7.54 (d, J=8.4 Hz, 1H), 7.27 (d, J=8.4 Hz, 2H), 7.21 (d, J=8.5 Hz, 2H), 6.96 (s, 1H), 6.83-6.81 (m, 1H), 5.00 (dd, J=13.4, 5.0 Hz, 1H), 4.74 (s, 1H), 4.31 (d, J=17.2 Hz, 1H), 4.19 (d, J=17.2 Hz, 1H), 3.27-2.96 (m, 4H), 2.92-2.64 (m, 3H), 2.52 (d, J=17.1 Hz, 1H), 2.40-2.25 (m, 3H), 2.25-2.13 (m, 2H), 2.13-1.97 (m, 3H), 1.92-1.88 (m, 2H), 1.35 (t, J=6.2 Hz, 2H), 0.91 (s, 6H), 0.90 (s, 3H). LCMS (ESI) calcd for C₃₅H₃₉ClN₃O₅₊ [M+H]⁺: 616.26, found, 616.3.

### Example 80: Preparation of 3-(5-(((3aR,6aS)-2-(4′-chloro-5,5-dimethyl-3,4,5,6-tetrahydro-[1,1′-biphenyl]-2-carbonyl)octahydrocyclopenta[c]pyrrol-5-yl)oxy)-1-oxoisoindolin-2-yl)piperidine-2,6-dione (GT-07019)

The target compound (GT-07019) was prepared as a white solid (11 mg, yield: 17%) according to the method described in synthetic method III. ¹H NMR (400 MHz, DMSO-d6) δ 10.90 (s, 1H), 7.53 (d, J=8.7 Hz, 1H), 7.34 (d, J=8.4 Hz, 2H), 7.23 (d, J=8.4 Hz, 2H), 6.94 (s, 1H), 6.84 (d, J=8.7 Hz, 1H), 5.00 (dd, J=13.3, 5.1 Hz, 1H), 4.78-4.46 (m, 1H), 4.32 (d, J=17.2 Hz, 1H), 4.19 (d, J=17.3 Hz, 1H), 3.21-3.15 (m, 2H), 3.06-2.99 (m, 2H), 2.92-2.77 (m, 2H), 2.77-2.62 (m, 1H), 2.54-2.50 (m, 3H), 2.38-2.25 (m, 2H), 2.20-2.08 (m, 3H), 1.94-1.90 (m, 1H), 1.86-1.62 (m, 2H), 1.37 (t, J=6.2 Hz, 2H), 0.92 (s, 3H), 0.91 (s, 3H). LCMS (ESI) calcd for C₃₅H₃₉ClN₃O₅⁺ [M+H]⁺: 616.26, found, 616.3.

### Example 81: Preparation of 3-(5-((7-(4′-chloro-5,5-dimethyl-3,4,5,6-tetrahydro-[1,1′-biphenyl]-2-carbonyl)-7-azaspiro[3.5]nonan-2-yl)oxy)-1-oxoisoindolin-2-yl)piperidine-2,6-dione (GT-07020)

The target compound (GT-07020) was prepared as a white solid (42 mg, yield: 66%) according to the method described in synthetic method III. ¹H NMR (400 MHz, DMSO-d6) δ 10.89 (s, 1H), 7.52 (dd, J=8.4, 1.3 Hz, 1H), 7.30 (dd, J=8.4, 5.9 Hz, 2H), 7.19 (dd, J=8.5, 6.6 Hz, 2H), 6.93 (d, J=5.1 Hz, 1H), 6.85 (d, J=8.4 Hz, 1H), 4.99 (dd, J=13.3, 5.1 Hz, 1H), 4.76-4.60 (m, 1H), 4.29 (d, J=17.2 Hz, 1H), 4.16 (d, J=17.2 Hz, 1H), 3.50-3.33 (m, 2H), 3.17-2.91 (m, 3H), 2.88-2.78 (m, 1H), 2.52 (d, J=16.7 Hz, 1H), 2.40-2.23 (m, 4H), 2.09-1.98 (m, 2H), 1.95-1.79 (m, 2H), 1.71-1.62 (m, 2H), 1.45-1.31 (m, 3H), 1.27-1.15 (m, 1H), 1.15-1.02 (m, 1H), 0.92 (s, 3H), 0.90 (s, 3H). LCMS (ESI) calcd for C₃₆H₄₁ClN₃O₅⁺ [M+H]⁺: 630.27, found, 630.3.

### Example 82: Preparation of 3-(5-((1-((4′-chloro-[1,1′-biphenyl]-2-yl)methyl)pyrrolidin-3-yl)oxy)-1-oxoisoindolin-2-yl)piperidine-2,6-dione (GT-06817)

The target compound (GT-06817) was prepared as a white solid (40 mg, yield: 65%) according to the method described in synthetic method I. ¹H NMR (400 MHz, DMSO-d6) δ 11.58 (s, 1H), 10.99 (s, 1H), 8.04 (d, J=7.0 Hz, 1H), 7.64 (t, J=8.0 Hz, 1H), 7.61-7.45 (m, 5H), 7.42 (d, J=8.3 Hz, 1H), 7.36-7.33 (m, 3H), 5.10-5.06 (m, 2H), 4.52-4.43 (m, 1H), 4.49-4.35 (m, 2H), 4.29-6.26 (m, 1H), 3.83-3.71 (m, 1H), 3.61-3.41 (m, 1H), 3.19-3.16 (m, 1H), 2.97-2.88 (m, 2H), 2.61 (d, J=17.5 Hz, 1H), 2.46-2.37 (m, 1H), 2.30-2.14 (m, 1H), 2.06-1.91 (m, 2H). LCMS (ESI) calcd for C₃₀H₂₉ClN₃O₄⁺ [M+H]⁺: 530.18, found, 530.2.

### Example 83: Preparation of 3-(5-((1-((4′-chloro-[1,1′-biphenyl]-2-yl)methyl)piperidin-4-yl)oxy)-1-oxoisoindolin-2-yl)piperidine-2,6-dione (GT-06193)

The target compound (GT-06193) was prepared as a white solid (52 mg, yield: 68%) according to the method described in synthetic method I. ¹H NMR (400 MHz, DMSO-d6) δ 10.89 (s, 1H), 8.00-7.95 (m, 1H), 7.55-7.47 (m, 5H), 7.33 (d, J=8.3 Hz, 2H), 7.28-7.25 (m, 1H), 7.09 (s, 1H), 6.97-6.94 (m, 1H), 5.01-4.97 (m, 1H), 4.33-4.12 (m, 4H), 3.19 (d, J=12.2 Hz, 1H), 3.07 (d, J=10.1 Hz, 1H), 2.93-2.87 (m, 1H), 2.86-2.68 (m, 3H), 2.52 (d, J=16.3 Hz, 1H), 2.38-2.24 (m, 1H), 2.20-2.08 (m, 1H), 2.08-1.98 (m, 1H), 1.98-1.79 (m, 3H). LCMS (ESI) calcd for C₃₁H₃₁ClN₃O₄⁺ [M+H]⁺: 544.20, found, 544.2.

### Example 84: Preparation of 3-(5-((1-((4′-chloro-[1,1′-biphenyl]-2-yl)methyl)azepan-4-yl)oxy)-1-oxoisoindolin-2-yl)piperidine-2,6-dione (GT-06934)

The target compound (GT-06934) was prepared as a white solid (30 mg, yield: 50%) according to the method described in synthetic method I. ¹H NMR (400 MHz, DMSO-d6) δ 10.89 (s, 1H), 8.01-7.94 (m, 1H), 7.54 (dd, J=8.4, 2.3 Hz, 1H), 7.51-7.43 (m, 4H), 7.38-7.29 (m, 2H), 7.27-7.25 (m, 1H), 7.04 (s, 1H), 6.97-6.82 (m, 1H), 5.00 (dd, J=13.2, 5.0 Hz, 1H), 4.66 (d, J=30.3 Hz, 1H), 4.32-4.15 (m, 4H), 3.06-2.98 (m, 1H), 2.94-2.73 (m, 3H), 2.52 (d, J=16.9 Hz, 1H), 2.36-2.26 (m, 1H), 2.16-1.69 (m, 5H), 1.66-1.58 (m, 2H), 1.54-1.47 (m, 1H). LCMS (ESI) calcd for C₃₂H₃₃ClN₃O₄⁺ [M+H]⁺: 558.22, found, 558.2.

### Example 85: Preparation of 3-(5-((2-((4′-chloro-[1,1′-biphenyl]-2-yl)methyl)octahydrocyclopenta[c]pyrrol-5-yl)oxy)-1-oxoisoindolin-2-yl)piperidine-2,6-dione (GT-06953)

The target compound (GT-06953) was prepared as a white solid (44 mg, yield: 59%) according to the method described in synthetic method I. ¹H NMR (400 MHz, DMSO-d6) δ 10.92 (s, 1H), 7.84-7.82 (m, 1H), 7.54 (m, 3H), 7.50-7.45 (m, 3H), 7.36-7.33 (m, 3H), 7.32-7.27 (m, 1H), 5.02 (dd, J=13.3, 5.1 Hz, 1H), 4.99-4.94 (m, 1H), 4.33-4.20 (m, 4H), 3.46-3.34 (m, 2H), 3.16-2.98 (m, 1H), 2.94-2.78 (m, 3H), 2.54 (d, J=16.3 Hz, 2H), 2.41-2.28 (m, 3H), 1.94-1.81 (m, 3H). LCMS (ESI) calcd for C₃₃H₃₃ClN₃O₄⁺ [M+H]⁺: 570.22, found, 570.2.

### Example 86: Preparation of 3-(5-((1-(4′-chloro-[1,1′-biphenyl]-2-carbonyl)azetidin-3-yl)oxy)-1-oxoisoindolin-2-yl)piperidine-2,6-dione (GT-06183)

The target compound (GT-06183) was prepared as a white solid (37 mg, yield: 46%) according to the method described in synthetic method I. ¹H NMR (400 MHz, MeOD) δ 7.60 (d, J=8.9 Hz, 1H), 7.50-7.44 (m, 1H), 7.42-7.36 (m, 3H), 7.34 (d, J=1.9 Hz, 4H), 6.78-6.75 (m, 2H), 5.01 (dd, J=13.3, 5.2 Hz, 1H), 4.85-480 (m, 1H), 4.41-4.24 (m, 3H), 4.00-3.96 (m, 1H), 3.85-3.74 (m, 1H), 3.46-3.35 (m, 1H), 2.87-2.74 (m, 1H), 2.73-2.62 (m, 1H), 2.41-2.32 (m, 1H), 2.12-2.00 (m, 1H). LCMS (ESI) calcd for C₂₉H₂₅ClN₃O₅₊ [M+H]⁺: 530.15, found, 530.2.

### Example 87: Preparation of 3-(5-((1-(4′-chloro-[1,1′-biphenyl]-2-carbonyl)pyrrolidin-3-yl)oxy)-1-oxoisoindolin-2-yl)piperidine-2,6-dione (GT-06818)

The target compound (GT-06818) was prepared as a white solid (33 mg, yield: 52%) according to the method described in synthetic method I. ¹H NMR (400 MHz, DMSO-d6) δ 10.97 (s, 1H), 7.65-7.59 (m, 1H), 7.55-7.53 (m, 1H), 7.52-7.46 (m, 3H), 7.46-7.41 (m, 2H), 7.41-7.31 (m, 2H), 7.03-6.99 (m, 1H), 6.90 (d, J=7.7 Hz, 1H), 5.17-4.90 (m, 2H), 4.45-4.36 (m, 1H), 4.28 (t, J=15.8 Hz, 1H), 3.71-3.49 (m, 2H), 3.06-3.00 (m, 1H), 2.88-2.78 (m, 1H), 2.60 (d, J=15.3 Hz, 1H), 2.47-2.30 (m, 1H), 2.13-1.80 (m, 3H). LCMS (ESI) calcd for C₃₀H₂₇ClN₃O₅⁺ [M+H]⁺: 544.16, found, 544.2.

### Example 88: Preparation of 3-(5-((1-(4′-chloro-[1,1′-biphenyl]-2-carbonyl)piperidin-4-yl)oxy)-1-oxoisoindolin-2-yl)piperidine-2,6-dione (GT-06204)

The target compound (GT-06204) was prepared as a white solid (36 mg, yield: 46%) according to the method described in synthetic method I. ¹H NMR (400 MHz, DMSO-d6) δ 10.89 (s, 1H), 7.52-7.44 (m, 4H), 7.42-7.36 (m, 4H), 7.31 (t, J=7.7 Hz, 1H), 7.11-7.04 (m, 1H), 6.98-6.90 (m, 1H), 5.01-4.96 (m, 1H), 4.55-4.49 (m, 1H), 4.28 (d, J=16.6 Hz, 1H), 4.16 (d, J=17.3 Hz, 1H), 3.93-3.77 (m, 1H), 2.96-2.89 (m, 1H), 2.85-2.78 (m, 1H), 2.72-2.54 (m, 3H), 2.32-2.26 (m, 1H), 1.94-1.86 (m, 2H), 1.60-1.52 (m, 1H), 1.39-1.30 (m, 1H), 1.24-1.17 (m, 1H). LCMS (ESI) calcd for C₃₁H₂₉ClN₃O₅⁺ [M+H]⁺: 558.18, found, 558.3.

### Example 89: Preparation of 3-(5-((1-(4′-chloro-[1,1′-biphenyl]-2-carbonyl)azepan-4-yl)oxy)-1-oxoisoindolin-2-yl)piperidine-2,6-dione (GT-06935)

The target compound (GT-06935) was prepared as a white solid (39 mg, yield: 63%) according to the method described in synthetic method I. ¹H NMR (400 MHz, DMSO-d6) δ 10.89 (s, 1H), 7.54 (d, J=8.4 Hz, 1H), 7.51-7.39 (m, 7H), 7.31-7.28 (m, 1H), 7.02 (d, J=16.4 Hz, 1H), 6.95-6.88 (m, 1H), 5.00 (dd, J=13.2, 4.9 Hz, 1H), 4.64-3.92 (m, 4H), 3.47-3.39 (m, 1H), 3.12-2.94 (m, 1H), 2.93-2.71 (m, 2H), 2.52 (d, J=16.4 Hz, 1H), 2.36-2.25 (m, 1H), 1.98-1.86 (m, 1H), 1.83-1.73 (m, 1H), 1.69-1.52 (m, 3H), 1.47-1.27 (m, 1H), 1.23-1.11 (m, 1H). LCMS (ESI) calcd for C₃₂H₃₁ClN₃O₅⁺ [M+H]⁺: 572.19, found, 572.2.

### Example 90: Preparation of 3-(5-((2-(4′-chloro-[1,1′-biphenyl]-2-carbonyl)octahydrocyclopenta[c]pyrrol-5-yl)oxy)-1-oxoisoindolin-2-yl)piperidine-2,6-dione (GT-06954)

The target compound (GT-06954) was prepared as a white solid (43 mg, yield: 56%) according to the method described in synthetic method I. ¹H NMR (400 MHz, DMSO-d6) δ 10.90 (s, 1H), 7.56 (d, J=8.5 Hz, 1H), 7.49-7.41 (m, 2H), 7.41-7.34 (m, 5H), 7.29 (dd, J=6.7, 5.3 Hz, 1H), 6.99 (s, 1H), 6.86 (d, J=8.3 Hz, 1H), 5.01 (dd, J=13.3, 5.0 Hz, 1H), 4.84-4.71 (m, 1H), 4.32 (d, J=17.2 Hz, 1H), 4.20 (d, J=17.2 Hz, 1H), 3.53-3.43 (m, 1H), 3.22-3.15 (m, 1H), 3.04-2.95 (m, 1H), 2.91-2.77 (m, 1H), 2.71-2.62 (m, 1H), 2.57-2.50 (m, 2H), 2.39-2.26 (m, 2H), 2.17-2.10 (m, 1H), 2.08-2.01 (m, 1H), 1.99-1.85 (m, 1H), 1.48-1.28 (m, 1H), 1.16-1.03 (m, 1H). LCMS (ESI) calcd for C₃₃H₃₁ClN₃O₅⁺ [M+H]⁺: 584.19, found, 584.2.

### Example 91: Preparation of 3-(5-((1-((4′-chloro-[1,1′-biphenyl]-3-yl)methyl)azetidin-3-yl)oxy)-1-oxoisoindolin-2-yl)piperidine-2,6-dione (GT-06227)

The target compound (GT-06227) was prepared as a white solid (17 mg, yield: 22%) according to the method described in synthetic method I. ¹H NMR (400 MHz, DMSO-d6) δ 10.90 (s, 1H), 7.75-7.65 (m, 4H), 7.60 (d, J=8.4 Hz, 1H), 7.50-7.44 (m, 4H), 7.03 (bts, 1H), 6.97-6.94 (m, 1H), 5.00 (dd, J=13.3, 5.0 Hz, 1H), 4.49-4.41 (m, 3H), 4.35-4.29 (m, 2H), 4.26-4.17 (m, 2H), 4.14-3.99 (m, 2H), 2.90-2.77 (m, 1H), 2.54-2.49 (m, 1H), 2.38-2.24 (m, 1H), 1.97-1.85 (m, 1H). LCMS (ESI) calcd for C₂₉H₂₇ClN₃O₄⁺ [M+H]⁺: 516.17, found, 516.2.

### Example 92: Preparation of 3-(5-((1-((4′-chloro-[1,1′-biphenyl]-4-yl)methyl)azetidin-3-yl)oxy)-1-oxoisoindolin-2-yl)piperidine-2,6-dione (GT-06230)

The target compound (GT-06230) was prepared as a white solid (16 mg, yield: 20%) according to the method described in synthetic method I. ¹H NMR (400 MHz, DMSO-d6) δ 10.90 (s, 1H), 7.70-7.65 (m, 4H), 7.62-7.57 (m, 3H), 7.47 (dd, J=9.0, 2.4 Hz, 2H), 7.03 (s, 1H), 6.96 (dd, J=8.4, 2.1 Hz, 1H), 5.01 (dd, J=13.3, 5.0 Hz, 1H), 4.53-4.22 (m, 7H), 4.06 (brs, 2H), 2.90-2.77 (m, 1H), 2.55-2.50 (m, 1H), 2.33-2.29 (m, 1H), 1.99-1.85 (m, 1H). LCMS (ESI) calcd for C₂₉H₂₇ClN₃O₄⁺ [M+H]⁺: 516.17, found, 516.2.

### Example 93: Preparation of 3-(5-((1-([2,4′-bipyridin]-5-ylmethyl)azetidin-3-yl)oxy)-1-oxoisoindolin-2-yl)piperidine-2,6-dione (GT-06809)

The target compound (GT-06809) was prepared as a white solid (18 mg, yield: 30%) according to the method described in synthetic method II. ¹H NMR (400 MHz, DMSO-d6) δ 10.98 (s, 1H), 9.04 (s, 1H), 8.99 (d, J=6.3 Hz, 2H), 8.58 (d, J=6.3 Hz, 2H), 8.45 (d, J=8.2 Hz, 1H), 8.36 (d, J=7.4 Hz, 1H), 7.67 (d, J=8.4 Hz, 1H), 7.12 (s, 1H), 7.04 (d, J=8.2 Hz, 1H), 5.08 (dd, J=13.3, 5.0 Hz, 1H), 4.78-4.56 (m, 4H), 4.42-4.45 (m, 5H), 3.02-2.82 (m, 1H), 2.60 (d, J=16.9 Hz, 1H), 2.41-2.37 (m, 1H), 2.03-1.97 (m, 1H). LCMS (ESI) calcd for C₂₇H₂₆N₅O₄⁺ [M+H]⁺: 484.20, found, 484.2.

### Example 94: Preparation of 3-(1-oxo-5-((1-((5-phenylpyrazin-2-yl)methyl)azetidin-3-yl)oxy)isoindolin-2-yl)piperidine-2,6-dione (GT-06353)

The target compound (GT-06353) was prepared as a white solid (33 mg, yield: 56%) according to the method described in synthetic method II. ¹H NMR (400 MHz, DMSO-d6) δ 10.98 (s, 1H), 9.30 (d, J=1.3 Hz, 1H), 8.86 (d, J=1.3 Hz, 1H), 8.18 (dd, J=7.9, 1.6 Hz, 2H), 7.62-7.50 (m, 4H), 7.12 (d, J=1.8 Hz, 1H), 7.03 (dd, J=8.4, 2.2 Hz, 1H), 5.22 (brs, 1H), 5.08 (dd, J=13.3, 5.1 Hz, 1H), 4.74-4.69 (m, 4H), 4.41 (dd, J=17.4, 8.6 Hz, 1H), 4.35-4.20 (m, 3H), 2.99-2.85 (m, 1H), 2.60 (d, J=16.6 Hz, 1H), 2.44-2.33 (m, 1H), 2.04-1.96 (m, 1H). LCMS (ESI) calcd for C₂₇H₂₆N₅O₄⁺ [M+H]⁺: 484.20, found, 484.2.

### Example 95: Preparation of 3-(5-((1-((5-(furan-2-yl)thiophen-2-yl)methyl)azetidin-3-yl)oxy)-1-oxoisoindolin-2-yl)piperidine-2,6-dione (GT-06562)

The target compound (GT-06562) was prepared as a white solid (18 mg, yield: 31%) according to the method described in synthetic method II. ¹H NMR (400 MHz, DMSO-d6) δ 10.90 (s, 1H), 7.67 (d, J=1.4 Hz, 1H), 7.60 (d, J=8.4 Hz, 1H), 7.28 (dd, J=10.5, 3.6 Hz, 2H), 7.03 (s, 1H), 6.96 (dd, J=8.4, 2.2 Hz, 1H), 6.75 (d, J=3.3 Hz, 1H), 6.54 (dd, J=3.4, 1.8 Hz, 1H), 5.17-5.07 (m, 1H), 5.01 (dd, J=13.3, 5.1 Hz, 2H), 4.60 (brs, 2H), 4.49-4.43 (m, 2H), 4.32 (d, J=17.6 Hz, 2H), 4.20 (d, J=17.4 Hz, 1H), 4.10-4.04 (m, 2H), 2.90-2.76 (m, 1H), 2.53 (d, J=16.5 Hz, 1H), 2.34-2.27 (m, 1H), 1.97-1.84 (m, 1H). LCMS (ESI) calcd for C₂₅H₂₄N₃O₅S⁺ [M+H]⁺: 478.14, found, 478.1.

### Example 96: Preparation of 3-(5-((1-((4′-fluoro-5,5-dimethyl-3,4,5,6-tetrahydro-[1,1′-biphenyl]-2-yl)methyl)piperidin-4-yl)oxy)-1-oxoisoindolin-2-yl)piperidine-2,6-dione (GT-07062)

The target compound (GT-07062) was prepared as a white solid (7 mg, yield: 10%) according to the method described in synthetic method I. ¹H NMR (400 MHz, DMSO-d6) δ 10.97 (s, 1H), 7.66-7.60 (m, 1H), 7.30-7.21 (m, 2H), 7.21-7.15 (m, 3H), 7.09-7.02 (m, 1H), 5.14-5.02 (m, 1H), 4.82-4.63 (m, 1H), 4.37 (d, J=16.9 Hz, 1H), 4.25 (d, J=17.4 Hz, 1H), 3.62-3.55 (m, 3H), 2.95-2.88 (m, 1H), 2.82-2.74 (m, 2H), 2.62-2.57 (m, 3H), 2.36-2.31 (m, 2H), 2.25-2.16 (m, 2H), 2.05 (s, 2H), 2.02-1.92 (m, 3H), 1.52-1.47 (m, 2H), 0.97 (s, 6H). LCMS (ESI) calcd for C₃₃H₃₉FN₃O₄⁺ [M+H]⁺: 560.29, found, 560.3.

### Example 97: Preparation of 3-(5-((1-((4′-chloro-[1,1′-biphenyl]-3-yl)methyl)piperidin-4-yl)oxy)-1-oxoisoindolin-2-yl)piperidine-2,6-dione (GT-06228)

The target compound (GT-06228) was prepared as a white solid (12 mg, yield: 16%) according to the method described in synthetic method I. ¹H NMR (400 MHz, DMSO-d6) δ 10.90 (s, 1H), 7.96-7.93 (m, 1H), 7.75-7.67 (m, 4H), 7.51-7.46 (m, 4H), 7.22-7.13 (m, 1H), 7.09-7.03 (m, 1H), 5.00 (dd, J=13.2, 5.0 Hz, 1H), 4.42-4.16 (m, 5H), 3.24-3.10 (m, 3H), 3.08-2.96 (m, 1H), 2.90-2.76 (m, 1H), 2.53 (d, J=17.5 Hz, 1H), 2.34-2.29 (m, 1H), 2.23-2.13 (m, 2H), 2.06-2.00 (m, 1H), 1.99-1.84 (m, 2H). LCMS (ESI) calcd for C₃₁H₃₁ClN₃O₄⁺ [M+H]⁺: 544.20, found, 544.2.

### Example 98: Preparation of 3-(5-((1-((4′-fluoro-3,4,5,6-tetrahydro-[1,1′-biphenyl]-2-yl)methyl)azetidin-3-yl)oxy)-1-oxoisoindolin-2-yl)piperidine-2,6-dione (GT-06302)

The target compound (GT-06302) was prepared as a white solid (15 mg, yield: 24%) according to the method described in synthetic method I. ¹H NMR (400 MHz, DMSO-d6) δ 10.98 (s, 1H), 7.70-7.64 (m, 1H), 7.45-7.30 (m, 1H), 7.26-7.11 (m, 5H), 5.20-4.96 (m, 2H), 4.72-4.61 (m, 1H), 4.42-4.21 (m, 3H), 3.83-3.69 (m, 2H), 2.95-2.86 (m, 1H), 2.74-2.62 (m, 3H), 2.33-2.22 (m, 3H), 2.19-2.11 (m, 2H), 2.07-1.92 (m, 2H), 1.67 (s, 3H). LCMS (ESI) calcd for C₂₉H₃₁FN₃O₄⁺ [M+H]⁺: 504.23, found, 504.2.

### Example 99: Preparation of 3-(5-((1-((4-(4-fluorophenyl)-5,6-dihydro-2H-pyran-3-yl)methyl)azetidin-3-yl)oxy)-1-oxoisoindolin-2-yl)piperidine-2,6-dione (GT-06560)

The target compound (GT-06560) was prepared as a white solid (20 mg, yield: 32%) according to the method described in synthetic method I. ¹H NMR (400 MHz, DMSO-d6) δ 10.91 (s, 1H), 7.65-7.53 (m, 1H), 7.32-7.17 (m, 3H), 7.12-7.02 (m, 2H), 6.95-6.84 (m, 1H), 5.02 (dd, J=13.3, 5.1 Hz, 1H), 4.93-4.90 (m, 1H), 4.5863-4.54 (m, 1H), 4.32 (d, J=17.5 Hz, 1H), 4.22 (d, J=17.5 Hz, 1H), 4.18-4.15 (m, 3H), 3.90-3.60 (m, 5H), 2.91-2.77 (m, 1H), 2.53 (d, J=16.8 Hz, 1H), 2.46-2.44 (m, 1H), 2.39-2.25 (m, 3H), 1.99-1.86 (m, 1H). LCMS (ESI) calcd for C₂₈H₂₉FN₃O₅₊ [M+H]⁺: 506.21, found, 506.2.

### Example 100: Preparation of 3-(5-((1-((4′-chloro-4,4-difluoro-3,4,5,6-tetrahydro-[1,1′-biphenyl]-2-yl)methyl)azetidin-3-yl)oxy)-1-oxoisoindolin-2-yl)piperidine-2,6-dione (GT-06563)

The target compound (GT-06563) was prepared as a white solid (15 mg, yield: 22%) according to the method described in synthetic method I. ¹H NMR (400 MHz, DMSO-d6) δ 11.06 (s, 1H), 10.91 (s, 1H), 7.61-7.56 (m, 1H), 7.49-7.41 (m, 1H), 7.34 (d, J=8.1 Hz, 1H), 7.26-7.11 (m, 2H), 7.00-6.81 (m, 1H), 6.74 (d, J=7.4 Hz, 1H), 5.01 (dd, J=13.3, 5.1 Hz, 1H), 4.92-4.88 (m, 1H), 4.68-4.49 (m, 1H), 4.35-4.18 (m, 3H), 3.83-3.61 (m, 3H), 2.91-2.68 (m, 3H), 2.53 (d, J=16.7 Hz, 1H), 2.46-2.44 (m, 2H), 2.37-2.28 (m, 1H), 2.17-2.00 (m, 2H), 1.94-1.89 (m, 1H). LCMS (ESI) calcd for C₂₉H₂₉ClF₂N₃O₄⁺ [M+H]⁺: 556.18, found, 556.2.

### Example 101: Preparation of 3-(5-((1-((4′-chloro-4-methoxy-4-methyl-3,4,5,6-tetrahydro-[1,1′-biphenyl]-2-yl)methyl)azetidin-3-yl)oxy)-1-oxoisoindolin-2-yl)piperidine-2,6-dione (GT-07065)

The target compound (GT-07065) was prepared as a white solid (12 mg, yield: 18%) according to the method described in synthetic method I. ¹H NMR (400 MHz, DMSO-d6) δ 10.98 (s, 1H), 7.64 (d, J=8.4 Hz, 1H), 7.49 (d, J=8.4 Hz, 1H), 7.38 (d, J=8.4 Hz, 2H), 7.26 (d, J=8.4 Hz, 1H), 7.19 (d, J=8.4 Hz, 2H), 5.09 (dd, J=13.2, 5.1 Hz, 1H), 5.04-4.95 (m, 1H), 4.71-4.56 (m, 1H), 4.40 (d, J=17.6 Hz, 1H), 4.29 (d, J=17.6 Hz, 1H), 4.23-4.18 (m, 1H), 3.85-3.66 (m, 3H), 3.17 (s, 3H), 2.98-2.85 (m, 1H), 2.60 (d, J=17.4 Hz, 1H), 2.55-2.51 (m, 1H), 2.45-2.32 (m, 2H), 2.32-2.17 (m, 3H), 2.02-1.98 (m, 1H), 1.81-1.76 (m, 1H), 1.67-1.60 (m, 1H), 1.20 (s, 3H). LCMS (ESI) calcd for C₃₁H₃₅ClN₃O₅+[M+H]⁺: 564.23, found, 564.3.

### Example 102: Preparation of 3-(5-((1-((4-(4-chlorophenyl)-6,6-dimethyl-5,6-dihydro-2H-pyran-3-yl)methyl)azetidin-3-yl)oxy)-1-oxoisoindolin-2-yl)piperidine-2,6-dione (GT-06561)

The target compound (GT-06561) was prepared as a white solid (21 mg, yield: 31%) according to the method described in synthetic method I. ¹H NMR (400 MHz, DMSO-d6) δ 10.98 (s, 1H), 7.69-7.60 (m, 1H), 7.51-7.47 (m, 1H), 7.46-7.36 (m, 2H), 7.36-7.21 (m, 2H), 7.00-6.94 (m, 1H), 5.13-5.08 (m, 2H), 4.97 (brs, 1H), 4.72-4.60 (m, 1H), 4.44-4.17 (m, 5H), 3.98-3.81 (m, 2H), 3.78-3.72 (m, 1H), 2.96-2.87 (m, 1H), 2.65-2.57 (m, 1H), 2.46-2.35 (m, 1H), 2.24 (s, 2H), 2.04-1.98 (m, 1H), 1.22 (s, 6H). LCMS (ESI) calcd for C₃₀H₃₃ClN₃O₅⁺ [M+H]⁺: 550.21, found, 550.2.

### Example 103: Preparation of 3-(5-((1-benzhydrylazetidin-3-yl)oxy)-1-oxoisoindolin-2-yl)piperidine-2,6-dione (GT-06185)

The target compound (GT-06185) was prepared as a white solid (21 mg, yield: 29%) according to the method described in synthetic method II. ¹H NMR (400 MHz, DMSO-d6) δ 10.89 (s, 1H), 7.66-7.53 (m, 5H), 7.43-7.33 (m, 6H), 7.05-6.92 (m, 2H), 6.00-5.82 (m, 1H), 5.04-4.96 (m, 1H), 4.62-4.39 (m, 2H), 4.36-4.04 (m, 5H), 2.87-2.75 (m, 1H), 2.51 (d, J=18.4 Hz, 1H), 2.34-2.23 (m, 1H), 1.94-1.86 (m, 1H). LCMS (ESI) calcd for C₂₉H₂₈N₃O₄⁺ [M+H]⁺: 482.21, found, 482.2.

### Example 104: Preparation of 3-(5-((1-benzhydrylpyrrolidin-3-yl)oxy)-1-oxoisoindolin-2-yl)piperidine-2,6-dione (GT-06819)

The target compound (GT-06819) was prepared as a white solid (16 mg, yield: 28%) according to the method described in synthetic method II. ¹H NMR (400 MHz, DMSO-d6) δ 10.90 (s, 1H), 7.93-7.68 (m, 4H), 7.58 (t, J=7.6 Hz, 1H), 7.41-7.30 (m, 7H), 7.16 (d, J=27.1 Hz, 1H), 5.70-5.62 (m, 1H), 5.22-5.18 (m, 1H), 5.00 (dd, J=13.2, 4.5 Hz, 1H), 4.32 (d, J=17.6 Hz, 1H), 4.20 (d, J=17.2 Hz, 1H), 3.95-3.76 (m, 1H), 3.60-3.42 (m, 1H), 3.24-3.14 (m, 1H), 3.09-3.04 (m, 1H), 2.92-2.77 (m, 1H), 2.74-2.58 (m, 1H), 2.52 (d, J=17.4 Hz, 1H), 2.35-2.26 (m, 1H), 2.25-2.07 (m, 1H), 1.92-1.86 (m, 1H). LCMS (ESI) calcd for C₃₀H₃₀N₃O₄⁺ [M+H]⁺: 496.22, found, 496.2.

### Example 105: Preparation of 3-(5-((1-benzhydrylpiperidin-4-yl)oxy)-1-oxoisoindolin-2-yl)piperidine-2,6-dione (GT-06195)

The target compound (GT-06195) was prepared as a white solid (15 mg, yield: 21%) according to the method described in synthetic method II. ¹H NMR (400 MHz, DMSO-d6) δ 10.89 (s, 1H), 7.87-7.79 (m, 4H), 7.64-7.53 (m, 1H), 7.44-7.36 (m, 4H), 7.32-7.28 (m, 2H), 7.18 (d, J=16.3 Hz, 1H), 7.13-7.00 (m, 1H), 5.02-4.97 (m, 1H), 4.84 (s, 1H), 4.32 (d, J=17.0 Hz, 1H), 4.20 (d, J=17.0 Hz, 1H), 3.04-3.03 (m, 3H), 2.91-2.77 (m, 1H), 2.63-2.49 (m, 4H), 2.35-2.29 (m, 2H), 2.21-2.12 (m, 1H), 2.05-1.99 (m, 1H), 1.96-1.83 (m, 1H). LCMS (ESI) calcd for C₃₁H₃₂N₃O₄⁺ [M+H]⁺: 510.24, found, 510.3.

### Example 106: Preparation of 3-(5-((1-benzhydrylazepan-4-yl)oxy)-1-oxoisoindolin-2-yl)piperidine-2,6-dione (GT-06928)

The target compound (GT-06928) was prepared as a white solid (12 mg, yield: 26%) according to the method described in synthetic method II. ¹H NMR (400 MHz, DMSO-d6) δ 11.82 (s, 1H), 10.89 (s, 1H), 7.89-7.77 (m, 4H), 7.54 (dd, J=8.4, 1.9 Hz, 1H), 7.41-7.34 (m, 4H), 7.32-7.23 (m, 2H), 7.12 (d, J=12.1 Hz, 1H), 7.03-6.95 (m, 1H), 5.70-5.62 (m, 1H), 4.99 (dd, J=13.4, 4.9 Hz, 1H), 4.86-4.68 (m, 1H), 4.30 (dd, J=17.3, 4.1 Hz, 1H), 4.18 (d, J=17.2 Hz, 1H), 3.48 (q, J=7.1 Hz, 1H), 3.08-2.91 (m, 2H), 2.93-2.77 (m, 1H), 2.52 (d, J=16.7 Hz, 1H), 2.39-2.18 (m, 3H), 2.16-2.05 (m, 1H), 2.01-1.79 (m, 4H), 1.78-1.67 (m, 1H). LCMS (ESI) calcd for C₃₂H₃₄N₃O₄⁺ [M+H]⁺: 524.25, found, 524.3.

### Example 107: Preparation of 3-(5-((7-benzhydryl-7-azaspiro[3.5]nonan-2-yl)oxy)-1-oxoisoindolin-2-yl)piperidine-2,6-dione (GT-06956)

The target compound (GT-06956) was prepared as a white solid (14 mg, yield: 20%) according to the method described in synthetic method II. ¹H NMR (400 MHz, DMSO-d6) δ 10.90 (s, 1H), 7.83-7.71 (m, 4H), 7.55 (d, J=8.4 Hz, 1H), 7.41-7.36 (m, 4H), 7.31-7.20 (m, 2H), 6.98 (s, 1H), 6.91-6.88 (m, 1H), 5.54 (d, J=9.3 Hz, 1H), 5.00 (dd, J=13.2, 5.0 Hz, 1H), 4.84-4.66 (m, 1H), 4.30 (d, J=17.3 Hz, 1H), 4.18 (d, J=17.3 Hz, 1H), 3.06-2.94 (m, 3H), 2.91-2.79 (m, 2H), 2.55-2.50 (m, 2H), 2.38-2.23 (m, 2H), 2.07-1.99 (m, 2H), 1.92-1.83 (m, 5H). LCMS (ESI) calcd for C₃₄H₃₆N₃O₄⁺ [M+H]⁺: 550.27, found, 550.3.

### Example 108: Preparation of 3-(5-((2-benzhydryloctahydrocyclopenta[c]pyrrol-5-yl)oxy)-1-oxoisoindolin-2-yl)piperidine-2,6-dione (GT-06955)

The target compound (GT-06955) was prepared as a white solid (9 mg, yield: 12%) according to the method described in synthetic method II. ¹H NMR (400 MHz, DMSO) δ 10.90 (s, 1H), 7.77-7.74 (m, 4H), 7.61 (d, J=8.4 Hz, 1H), 7.38 (t, J=7.4 Hz, 4H), 7.32-7.28 (m, 2H), 7.22 (s, 1H), 7.16-7.07 (m, 1H), 5.10-4.95 (m, 2H), 4.35 (d, J=17.3 Hz, 1H), 4.23 (d, J=17.2 Hz, 1H), 3.48-3.40 (m, 2H), 3.22-3.17 (m, 1H), 2.97-2.83 (m, 4H), 2.55-2.49 (m, 1H), 2.40-2.21 (m, 2H), 2.09-1.99 (m, 2H), 1.93-1.81 (m, 3H). LCMS (ESI) calcd for C₃₃H₃₄N₃O₄⁺ [M+H]⁺: 536.25, found, 536.3.

### Example 109: Preparation of 3-(5-((1-(bis(2-fluorophenyl)methyl)azetidin-3-yl)oxy)-1-oxoisoindolin-2-yl)piperidine-2,6-dione (GT-06759)

The target compound (GT-06759) was prepared as a white solid (24 mg, yield: 38%) according to the method described in synthetic method II. ¹H NMR (400 MHz, DMSO-d6) δ 10.96 (s, 1H), 8.01-7.73 (m, 1H), 7.70-7.56 (m, 2H), 7.55-7.09 (m, 7H), 7.12-6.93 (m, 1H), 5.36-4.96 (m, 2H), 4.43-4.22 (m, 2H), 3.99 (dd, J=34.3, 10.9 Hz, 1H), 3.64-3.39 (m, 3H), 3.17-3.02 (m, 1H), 2.99-2.87 (m, 1H), 2.60 (d, J=17.6 Hz, 1H), 2.45-2.30 (m, 1H), 2.08-1.90 (m, 1H). LCMS (ESI) calcd for C₂₉H₂₆F₂N₃O₄⁺ [M+H]⁺: 518.19, found, 518.2.

### Example 110: Preparation of 3-(5-((1-(bis(3-fluorophenyl)methyl)azetidin-3-yl)oxy)-1-oxoisoindolin-2-yl)piperidine-2,6-dione (GT-06352)

The target compound (GT-06352) was prepared as a white solid (21 mg, yield: 33%) according to the method described in synthetic method II. ¹H NMR (400 MHz, DMSO-d6) δ 10.90 (d, J=7.9 Hz, 1H), 7.59-7.55 (m, 1H), 7.49-7.32 (m, 6H), 7.20-7.08 (m, 2H), 7.00 (s, 1H), 6.94 (d, J=8.1 Hz, 1H), 5.38-5.16 (m, 1H), 5.04-4.97 (m, 1H), 4.37-4.14 (m, 5H), 3.97-3.88 (m, 1H), 2.89-2.78 (m, 1H), 2.54-2.48 (m, 2H), 2.37-2.22 (m, 1H), 1.99-1.82 (m, 1H). LCMS (ESI) calcd for C₂₉H₂₆F₂N₃O₄⁺ [M+H]⁺: 518.19, found, 518.2.

### Example 111: Preparation of 3-(5-((1-(bis(4-fluorophenyl)methyl)azetidin-3-yl)oxy)-1-oxoisoindolin-2-yl)piperidine-2,6-dione (GT-06519)

The target compound (GT-06519) was prepared as a white solid (11 mg, yield: 17%) according to the method described in synthetic method II. ¹H NMR (400 MHz, DMSO-d6) δ 10.98 (s, 1H), 7.95-7.54 (m, 5H), 7.43-7.21 (m, 4H), 7.16-6.89 (m, 2H), 6.23-5.88 (m, 1H), 5.19-4.92 (m, 2H), 4.65-3.49 (m, 2H), 4.35-4.11 (m, 4H), 2.95-2.86 (m, 1H), 2.34-2.25 (m, 2H), 2.04-1.91 (m, 1H). LCMS (ESI) calcd for C₂₉H₂₆F₂N₃O₄⁺ [M+H]⁺: 518.19, found, 518.2.

### Example 112: Preparation of 3-(5-((1-(bis(4-chlorophenyl)methyl)azetidin-3-yl)oxy)-1-oxoisoindolin-2-yl)piperidine-2,6-dione (GT-06520)

The target compound (GT-06520) was prepared as a white solid (20 mg, yield: 30%) according to the method described in synthetic method II. ¹H NMR (400 MHz, DMSO-d6) δ 10.89 (s, 1H), 7.80-7.49 (m, 5H), 7.49-7.17 (m, 4H), 7.00-6.93 (m, 2H), 6.12-5.79 (m, 1H), 5.00 (dd, J=13.3, 5.0 Hz, 1H), 4.58-4.46 (m, 1H), 4.29 (d, J=17.5 Hz, 1H), 4.18 (d, J=17.4 Hz, 1H), 4.13-4.00 (m, 1H), 3.29-3.23 (m, 3H), 2.87-2.78 (m, 1H), 2.52 (d, J=16.6 Hz, 1H), 2.36-2.24 (m, 1H), 1.92-1.88 (m, 1H).

LCMS (ESI) calcd for C₂₉H₂₆Cl₂N₃O₄⁺ [M+H]⁺: 550.13, found, 550.2.

### Example 113: Preparation of 3-(5-((1-(bis(4-methoxyphenyl)methyl)azetidin-3-yl)oxy)-1-oxoisoindolin-2-yl)piperidine-2,6-dione (GT-06959)

The target compound (GT-06959) was prepared as a white solid (10 mg, yield: 14%) according to the method described in synthetic method II. ¹H NMR (400 MHz, DMSO) δ 10.89 (s, 1H), 7.58-7.46 (m, 4H), 7.36-7.15 (m, 1H), 7.02-6.84 (m, 6H), 5.78-5.70 (m, 1H), 5.02-4.97 (m, 1H), 4.45-4.37 (m, 2H), 4.30 (d, J=17.5 Hz, 1H), 4.18 (d, J=17.9 Hz, 1H), 4.09-4.01 (m, 2H), 3.68 (s, 6H), 2.92-2.76 (m, 1H), 2.52 (d, J=17.2 Hz, 1H), 2.37-2.24 (m, 1H), 2.22-2.11 (m, 1H), 1.97-1.85 (m, 1H).

LCMS (ESI) calcd for C₃₁H₃₁N₃NaO₆⁺ [M+H]⁺: 564.21, found, 564.2.

### Example 114: Preparation of 3-(1-oxo-5-((1-(phenyl(pyridin-2-yl)methyl)azetidin-3-yl)oxy)isoindolin-2-yl)piperidine-2,6-dione (GT-06559)

The target compound (GT-06559) was prepared as a white solid (30 mg, yield: 51%) according to the method described in synthetic method II. ¹H NMR (400 MHz, DMSO-d6) δ 10.90 (s, 1H), 10.21 (s, 1H), 8.60 (s, 1H), 7.82-7.78 (m, 1H), 7.64-7.54 (m, 3H), 7.48-7.28 (m, 6H), 7.14-7.10 (m, 1H), 5.25-5.11 (m, 1H), 5.07-4.93 (m, 1H), 4.48-4.12 (m, 3H), 4.04-3.87 (m, 2H), 3.21-3.11 (m, 2H), 2.89-2.78 (m, 1H), 2.53 (d, J=17.0 Hz, 1H), 2.40-2.21 (m, 1H), 2.00-1.81 (m, 1H). LCMS (ESI) calcd for C₂₈H₂₇N₄O₄⁺ [M+H]⁺: 483.20, found, 483.2.

### Example 115: Preparation of 3-(1-oxo-5-((1-(phenyl(pyridin-3-yl)methyl)azetidin-3-yl)oxy)isoindolin-2-yl)piperidine-2,6-dione (GT-06760)

The target compound (GT-06760) was prepared as a white solid (16 mg, yield: 27%) according to the method described in synthetic method II. ¹H NMR (400 MHz, DMSO-d6) δ 10.99 (s, 1H), 9.00 (s, 1H), 8.67 (t, J=4.8 Hz, 1H), 8.44-8.40 (m, 1H), 7.81-7.74 (m, 2H), 7.72-7.61 (m, 2H), 7.52-7.31 (m, 4H), 7.24-7.21 (m, 1H), 5.10 (dd, J=13.7, 8.7 Hz, 1H), 4.52-4.39 (m, 2H), 4.35-4.27 (m, 1H), 4.13-3.93 (m, 2H), 3.38-3.21 (m, 3H), 2.97-2.87 (m, 1H), 2.60 (d, J=17.4 Hz, 1H), 2.45-2.34 (m, 1H), 2.07-1.89 (m, 1H). LCMS (ESI) calcd for C₂₈H₂₇N₄O₄⁺ [M+H]⁺: 483.20, found, 483.2.

### Example 116: Preparation of 3-(5-((1-(di(pyridin-2-yl)methyl)azetidin-3-yl)oxy)-1-oxoisoindolin-2-yl)piperidine-2,6-dione (GT-06814)

The target compound (GT-06814) was prepared as a white solid (19 mg, yield: 32%) according to the method described in synthetic method II. ¹H NMR (400 MHz, DMSO-d6) δ 10.99 (s, 1H), 8.64 (s, 2H), 7.98-7.83 (m, 2H), 7.71-7.65 (m, 2H), 7.60 (d, J=7.6 Hz, 1H), 7.51-7.38 (m, 2H), 7.30 (brs, 1H), 7.22-7.18 (m, 1H), 6.02 (brs, 1H), 5.29 (brs, 1H), 5.10 (dd, J=13.4, 5.1 Hz, 1H), 4.45-4.29 (m, 2H), 4.09-3.91 (m, 2H), 3.00-2.85 (m, 2H), 2.61 (d, J=17.2 Hz, 2H), 2.43-2.26 (m, 2H), 2.04-1.93 (m, 1H). LCMS (ESI) calcd for C₂₇H₂₆N₅O₄⁺ [M+H]⁺: 484.20, found, 484.2.

### Biological Activity Assay

### Reagents and Materials:

| Reagents and materials | Suppliers or Source |
|---|---|
| Human T cell acute lymphoblastic | Dalian Meilun Biotech Co., Ltd. |
| leukemia cell line: CCRF-CEM | |
| Human Burkitt's lymphoma cells: Daudi | Dalian Meilun Biotech Co., Ltd. |
| Human acute lymphoblastic leukemia cell: Kasumi-1 | ATCC (American Type Culture Collection) |
| Human breast cancer cell: MDA-MB-231 | Dalian Meilun Biotech Co., Ltd. |
| Human multiple myeloma cells: MM.1S | ATCC |
| Human mantle cell lymphoma cells: JEKO-1 | ATCC |
| Human Monocyte Leukemia cells: THP-1 | ATCC |
| Human myelomonocytic leukemia cells: MV-4-11 | ATCC |
| Human multiple myeloma cell line resistant to | ATCC |
| dexamethasone: MM.1R | |
| Human thyroid carcinoma cells: CAL-62 | ATCC |
| RPMI-1640 Medium | Dalian Meilun Biotech Co., Ltd. |
| IMDM Medium | Gibco Company |
| DMEM Medium | Dalian Meilun Biotech Co., Ltd. |
| Meilun Cell Counting Kit-8 | Dalian Meilun Biotech Co., Ltd. |
| Fetal bovine serum (FBS) | Sunrise Science Products Company |
| Penicillin-Streptomycin | Beijing TransGen Biotech Co., Ltd. |
| Mycoplasma detection kit | Beijing TransGen Biotech Co., Ltd. |
| STR genotype detection | Shanghai Biowing Applied Biotechnology |
| | Co. Ltd. |

### Methods:

### Cell Cultures

The tumor cells used herein were cultured in a cell culture medium listed in table A at 37° C. in an incubator with 5% CO₂. Prior to experimentation, all cells used were correctly identified by STR profiling, and tested negative for mycoplasma through routine screenings.

TABLE A
Tumor cells and cell culture medium used in the present disclosure

| Cell line | Cell culture medium used |
|---|---|
| CCRF-CEM | RPMI 1640 culture medium supplemented with 10% |
| Daudi | FBS, as well as penicillin at a final |
| Kasumi-1 | concentration of 100 U/mL and streptomycin |
| MM.1S | at a final concentration of 100 μg/mL |
| JEKO-1 | |
| THP-1 | |
| MM.1R | |
| MDA-MB-231 | DMEM culture medium supplemented with 10% FBS, |
| CAL-62 | as well as penicillin at a final concentration |
| | of 100 U/mL and streptomycin at a final |
| | concentration of 100 μg/mL |
| MV-4-11 | IMDM culture medium supplemented with 10% FBS, |
| | as well as penicillin at a final concentration |
| | of 100 U/mL and streptomycin at a final |
| | concentration of 100 μg/mL |

### I. Determination of Half Inhibitory Concentration (IC₅₀) of the Compounds Against Tumor Cells:

IC₅₀ values of the compounds of the present disclosure (including the compounds in Tables 1 and 2, and the Examples A1-A20 and 1-116 compounds) were measured using the Meilun Cell Counting Kit-8 kit from Dalian Meilun Biotech Co., Ltd. Assay details are as follows: tumor cells were seeded in a 96-well plate at a density listed in Table B. After 24 h, 100 μL of the inoculated cells were treated with 0.5 μL of the compounds of the present disclosure (including the compounds in Tables 1 and 2, and the Examples A1-A20 and 1-116 compounds) at 10 different successively decreasing concentrations (starting at the highest concentration of 10 μM; 5-fold serial dilutions). After the cells were treated with the compounds of the present disclosure for a period of time, the cell viability detection reagent was added to the culture medium for cell viability assessment according to the instructions provided with the CCK-8 kit. DMSO served as the negative control, and corresponding commercial inhibitors immunomodulatory agents (including lenalidomide and pomalidomide) were used as the positive control, both of which treated the cells using the same protocol as that of the compounds of the present disclosure. The growth inhibition of the compounds of the present disclosure on cells was plotted by Prism Graphpad software, and the IC₅₀ values of the compounds of the present disclosure were calculated therefrom.

TABLE B
Seeding protocol and treatment time for tumor cells

| | | The times for treating cells |
|---|---|---|
| | | with the compounds of the |
| Cells | Cells seeding protocol | present disclosure (hours) |
| Kasumi-1 | cells were seeded at a density of 10,000 cells | 96 h |
| MM.1S | per well in 100 μL of serum-containing | |
| THP-1 | RPMI-1640 medium per well | |
| MM.1R | | |
| CAL-62 | cells were seeded at a density of 2,000 cells | 96 h |
| | per well in 100 μL of serum-containing | |
| | DMEM medium per well | |
| CCRF-CEM | cells were seeded at a density of 4,000 cells | 96 h |
| Daudi | per well in 100 μL of serum-containing | |
| JEKO-1 | RPMI-1640 medium per well | |
| MDA-MB-231 | cells were seeded at a density of 4,000 cells | 96 h |
| | per well in 100 μL of serum-containing | |
| | DMEM medium per well | |
| MV-4-11 | cells were seeded at a density of 10,000 cells | 96 h |
| | per well in 100 μL of serum-containing | |
| | IMDM medium per well | |

Results of inhibiting tumor cell proliferation were shown in Table C below.

TABLE C
The inhibitory activity (IC₅₀, nM) of the compounds of the present disclosure
designed based on immunomodulatory drugs against tumor cells proliferation

| | Name | | | | | | | | | |
|---|---|---|---|---|---|---|---|---|---|---|
| | CCRF- | MV-4- | | Kasumi- | JEKO- | THP- | MDA-MB- | | | CAL- |
| Comp. No. | CEM | 11 | Daudi | 1 | 1 | 1 | 231 | MM.1R | MM.1S | 62 |
| lenalidomide | F | F | F | F | F | F | F | F | F | F |
| pomalidomide | F | F | F | F | F | F | F | F | C | F |
| GT- | A | B | A+++ | B | B | A+ | B | A | B | A |
| 03360 | | | | | | | | | | |
| GT- | B | — | B | — | — | — | — | — | — | — |
| 03357 | | | | | | | | | | |
| GT- | B | — | — | — | B | — | — | — | — | — |
| 03363 | | | | | | | | | | |
| GT- | — | — | — | — | — | C | — | — | — | — |
| 04245 | | | | | | | | | | |
| GT- | C | — | — | — | — | — | — | — | — | B |
| 03462 | | | | | | | | | | |
| GT- | — | A+ | — | — | — | — | — | — | — | — |
| 06873 | | | | | | | | | | |
| GT- | — | B | — | — | — | — | — | — | — | — |
| 06874 | | | | | | | | | | |
| GT- | — | C | — | — | — | — | — | — | — | — |
| 06960 | | | | | | | | | | |
| GT- | — | B | — | — | — | — | — | — | — | — |
| 06918 | | | | | | | | | | |
| GT- | — | C | — | — | — | — | — | — | — | — |
| 07016 | | | | | | | | | | |
| GT- | C | B | — | — | — | — | — | — | — | — |
| 06924 | | | | | | | | | | |
| GT- | A+ | — | — | — | — | B | — | — | — | A+ |
| 03892 | | | | | | | | | | |
| GT- | — | — | — | — | — | — | — | — | A+ | — |
| 06182 | | | | | | | | | | |
| GT- | — | A++ | — | — | — | — | — | — | — | — |
| 06925 | | | | | | | | | | |
| GT- | — | — | — | — | — | — | — | — | — | — |
| 06226 | | | | | | | | | | |
| GT- | — | C | — | — | — | — | — | — | — | — |
| 06645 | | | | | | | | | | |
| GT- | A | A+ | — | B | — | — | — | — | — | — |
| 06926 | | | | | | | | | | |
| GT- | A | A | — | B | — | — | — | — | — | — |
| 06758 | | | | | | | | | | |
| GT- | — | C | — | — | — | — | — | — | — | — |
| 06815 | | | | | | | | | | |
| GT- | — | B | — | — | — | — | — | — | — | — |
| 06934 | | | | | | | | | | |
| GT- | — | — | — | — | — | — | — | — | A+ | — |
| 06204 | | | | | | | | | | |
| GT- | — | B | — | — | — | — | — | — | — | — |
| 06935 | | | | | | | | | | |
| GT- | — | A+ | — | — | — | — | — | — | — | — |
| 06928 | | | | | | | | | | |
| GT- | — | C | — | — | — | — | — | — | — | — |
| 06956 | | | | | | | | | | |
| GT- | — | A | — | — | — | — | — | — | — | — |
| 06955 | | | | | | | | | | |

Note:
In Table C, the symbols A+++, A++, A+, A, B, C, D, E, and F are defined as follows: 0 < A+++ ≤ 1 nM; 1 nM < A++ ≤ 10 nM; 10 nM < A+ ≤ 50 nM; 50 nM < A ≤ 100 nM; 100 nM < B ≤ 500 nM; 500 nM < C ≤ 1000 nM; 1000 nM < D ≤ 5000 nM; 5000 nM < E ≤ 10000 nM; F > 10000 nM. The symbol “—” indicates “not detected”.

The results demonstrated that the compounds of the present disclosure (including but not limited to those listed in Tables 1 and 2, and Examples A1-A20 and 1-116 compounds) exhibited significant inhibitory effects on the proliferation of various cancer cell lines, including CCRF-CEM (human T cell acute lymphoblastic leukemia cell line), MV-4-11 (human myelomonocytic leukemia cells), Daudi (human Burkitt's lymphoma cells), MM.1S (human multiple myeloma cells), MM.1R (dexamethasone-resistant human multiple myeloma cells), MDA-MB-231 (human breast cancer cells), Kasumi-1 (human acute lymphoblastic leukemia cells), CAL-62 (human thyroid carcinoma cells), JEKO-1 (human mantle cell lymphoma cells), and THP-1 (human monocytic leukemia cells), as shown in Table C. These compounds of the present disclosure demonstrated superior inhibitory potency compared to their corresponding positive control drugs.

### II. Determination of CRBN-Binding Affinity of Compounds:

The CEREBLON BINDING kits (specification: 10,000 tests; product number: Catalog #64BDCRBNPEH; CISBIO company) was used to determine the CRBN binding ability of the compounds to be tested through a HTRF method (homogeneous time-resolved fluorescence). The specific methods are as follows:
1. According to the instructions of the CEREBLON BINDING kits, the compounds to be tested of the present disclosure and lenalidomide were serially diluted using diluent #9 (1×) solution to obtain a final concentration of 2 μM for both the tested compounds and lenalidomide solution.
2. 2.5 μL of the above 2 μM of the tested compounds and lenalidomide solution, as well as the same volume of diluent #9 (1×) solution (solvent control group, Std0) were added to each well of a 96-well plate, respectively. Then, 2.5 μL of human Cereblon WT GST-tagged protein solution was added to each well. Finally, 5 μL of the thoroughly mixed Thalidomide-Re reagent and GST Eu antibody working solution were added to each of the aforementioned wells. The final concentration of the tested compounds and lenalidomide in each well is 0.5 M.
3. The blank control wells were sequentially added with 2.5 μL of diluent #9 (1×) solution, 2.5 μL of PROTAC binding buffer, and 5 μL of thoroughly mixed Thalidomide-Re reagent and GST Eu antibody working solution.
4. After sealing and incubating the solutions in the aforementioned wells at room temperature for 3 hours, the absorbance values at emission wavelengths of 620 nm and 665 nm were detected by the HTRF method using a Spark microplate reader (V3.1 SP1).

The corresponding CRBN binding inhibition rate was calculated using the following method: R_{compound}=OD 665 nm_{compound}/OD 620 nm_{compound}−OD 665 nm_{control}/OD 620 nm_{control} R_{Std0}=OD 665 nm_{Sta0}/OD 620 nm_{Std0}−OD 665 nm_{control}/OD 620 nm_{control} inhibition rate (%)=(1−R_{compound}/R_{Std0})*100

Note: OD 665 nm_{compound} is the absorbance value of each well of the compounds to be tested of the present disclosure at an emission wavelength of 665 nm.
OD 620 nm_{compound} is the absorbance value of each well of the compounds to be tested of the present disclosure at an emission wavelength of 620 nm.
OD 665 nm_{control} is the absorbance value of the blank control well at an emission wavelength of 665 nm.
OD 620 nm_{control} is the absorbance value of the blank control well at an emission wavelength of 620 nm.
OD 665 nm_{Std0} is the absorbance value of the solvent control well at an emission wavelength of 665 nm.
OD 620 nm_{Std0} is the absorbance value of the solvent control well at an emission wavelength of 620 nm.

The test results were shown in Table D below.

TABLE D
HTRF inhibitory activity (IC₅₀, μM) of the compounds of the
present disclosure (including but not limited to the compounds
in Tables 1 and 2, and the Examples A1-A20 and 1-116 compounds)

| | HTRF | | HTRF | | HTRF | | HTRF |
|---|---|---|---|---|---|---|---|
| | IC₅₀ | | IC₅₀ | | IC₅₀ | | IC₅₀ |
| Comp. No. | (μM) | Comp. No. | (μM) | Comp. No. | (μM) | Comp. No. | (μM) |
| lenalidomide | d | GT-06925 | b | GT-06872 | c | GT-06561 | a |
| GT-03464 | a | GT-06225 | a | GT-06815 | c | GT-06185 | a |
| GT-06515 | b | GT-06226 | b | GT-06929 | b | GT-06819 | a |
| GT-06757 | c | GT-06350 | a | GT-06933 | c | GT-06195 | a |
| GT-06812 | c | GT-06808 | b | GT-06816 | a | GT-06928 | b |
| GT-04245 | a | GT-06517 | a | GT-06192 | b | GT-06956 | a |
| GT-03462 | b | GT-03457 | a | GT-07018 | a | GT-06955 | a |
| GT-06641 | c | GT-06301 | a | GT-07025 | a | GT-06759 | a |
| GT-06813 | c | GT-06351 | a | GT-07019 | a | GT-06352 | b |
| GT-06873 | c | GT-06513 | a | GT-07020 | b | GT-06519 | b |
| GT-06874 | c | GT-06516 | c | GT-03363 | b | GT-06520 | c |
| GT-06137 | b | GT-06514 | b | GT-06817 | b | GT-06959 | a |
| GT-06139 | b | GT-06138 | b | GT-06193 | c | GT-06559 | a |
| GT-06140 | b | GT-06196 | b | GT-06934 | a | GT-06760 | a |
| GT-06642 | c | GT-06184 | b | GT-06953 | a | GT-06814 | a |
| GT-07022 | b | GT-06645 | a | GT-06183 | a | GT-03613 | a |
| GT-07015 | b | GT-06926 | c | GT-06818 | a | GT-03616 | c |
| GT-06918 | b | GT-06920 | a | GT-06204 | a | GT-06995 | c |
| GT-07016 | c | GT-06958 | a | GT-06935 | a | GT-06992 | b |
| GT-07017 | c | GT-06922 | a | GT-06954 | a | GT-06993 | c |
| GT-03360 | a | GT-06758 | a | GT-06230 | b | GT-06997 | b |
| GT-04195 | c | GT-06349 | b | GT-06809 | b | GT-06998 | c |
| GT-06643 | c | GT-06503 | b | GT-06353 | b | GT-06996 | c |
| GT-06914 | c | GT-06504 | b | GT-06562 | a | GT-06999 | b |
| GT-03892 | a | GT-06923 | a | GT-03459 | b | GT-03612 | c |
| GT-06181 | a | GT-06518 | a | GT-06302 | b | GT-07000 | a |
| GT-06182 | a | GT-06807 | a | GT-06354 | a | GT-07005 | c |
| GT-06644 | a | GT-06811 | a | GT-06560 | b | GT-07004 | c |
| GT-07023 | a | GT-03369 | b | GT-06563 | b | | |
| GT-07024 | a | GT-06952 | b | | | | |

In Table D, the symbols a, b, and c are defined as follows: 0 < a ≤ 0.5 μM, 0.5 μM < b ≤ 1 μM, 1 μM < c < 1.9 μM, 1.9 μM ≤ d.

The results show that the compounds of the present disclosure (including the compounds listed in Tables 1 and 2, and the Examples A1-A20 and 1-116 compounds) exhibit strong binding affinity to CRBN.

### III. Western-Blot Assay

The effect of the compounds of the present disclosure on the expression of target proteins in cells was detected using conventional Western blot analysis.

Human peripheral blood mononuclear cells (hPBMCs) at a density of 1×10⁶ cells/mL were thawed according to the PBMC Recovery Protocol (Milecell Biotechnologies Inc, Shanghai). The cells were then treated with the compounds of the present disclosure (including the compounds listed in Tables 1 and 2, and the Examples A1-A20 and 1-116 compounds) at concentrations of 50 nM and 500 nM.

Controls included a negative control (DMSO) and a positive control (the commercial immunomodulatory drug lenalidomide), both processed following the identical experimental protocol as the compounds of the present disclosure. After 24 hours of compounds treatment, cells were harvested and lysed in RIPA protein lysate containing phosphatase inhibitors on ice for 30-60 min, followed by centrifugation. The supernatant was aspirated as the extracted total cell protein. The protein concentration of the supernatant was determined using the conventional BCA (Bicinchoninic Acid) assay. The supernatant was then mixed with 4× SDS sample loading buffer, denatured at 95° C. for 5 minutes, and then subjected to SDS-PAGE electrophoresis on a polyacrylamide gel. Proteins were then transferred to a nitrocellulose (NC) membrane, blocked at room temperature for 1-2 h, and processed for antibody incubation and detection according to the manufacturer's instructions (Cell Signaling Technology).

Half-Maximal Degradation Concentration value (the drug concentration required for degrading proteins by 50%, abbreviated as DC₅₀) reads method: comparing the grayscale values of Western blot bands from drug-treated samples with those from blank DMSO-treated controls, and identifying the drug concentration range at which the grayscale value is half that of the DMSO-treated control bands.

DC₅₀ values can be calculated using ImageJ software to measure grayscale values of Western blot bands from drug-treated samples. The concentration-response curve is fitted to estimate the drug concentration corresponding to half the grayscale value.

Target protein remaining (%) refers to the percentage of target protein remaining in hPBMC cells after treatment with protein degrader compounds.

Calculation method for protein remaining (%): Use ImageJ software to measure grayscale values of Western blot bands from drug (protein degrader)-treated samples. The grayscale value of drug-treated bands is divided by that of internal control protein bands, and the resulting quotient is normalized to obtain the relative percentage of remaining target protein after protein degrader treatment.

Target protein degradation rate (%)=100%−target protein remaining (%).

The effects of the compounds of the present disclosure (including the compounds listed in Tables 1 and 2, and the Examples A1-A20 and 1-116 compounds) on substrate proteins expression in hPBMC cells were shown in Table E below.

TABLE E
Degradation results (substrate protein remaining %) of
the compounds of the present disclosure (including the
compounds listed in Tables 1 and 2, and the Examples
A1-A20 and 1-116 compounds) on substrate proteins

| Comp. No. | Wee1 | IKZF1 | IKZF2 | IKZF3 | GSPT1 | CK1α |
|---|---|---|---|---|---|---|
| GT-03464 | A | A | | | A | A |
| GT-06515 | A | A | | A | A | A |
| GT-06812 | A | A | | | A | |
| GT-04245 | | | A | | A | |
| GT-03462 | A | | | | A | |
| GT-06641 | | | A | | | |
| GT-06813 | A | | A | | A | |
| GT-06873 | A | A | A | A | A | A |
| GT-06874 | A | | A | A | A | A |
| GT-06960 | A | | | | A | A |
| GT-06137 | A | A | | | A | |
| GT-06140 | A | | | | | |
| GT-06642 | | | A | | A | |
| GT-07022 | | | | A | | A |
| GT-07015 | | | A | A | | |
| GT-06918 | A | A | A | A | | |
| GT-03360 | | A | A | A | A | A |
| GT-03357 | | | | A | A | |
| GT-06643 | A | | A | | | |
| GT-06914 | A | | A | | | |
| GT-06924 | | A | | | A | A |
| GT-03892 | A | A | A | A | A | |
| GT-06181 | A | | | A | A | |
| GT-06182 | A | A | | A | A | A |
| GT-06644 | | | | | A | |
| GT-07023 | A | | | | | |
| GT-07024 | A | | A | | | |
| GT-06925 | A | A | A | A | A | A |
| GT-06225 | | A | | | A | |
| GT-06226 | A | A | | | | |
| GT-06350 | | A | A | A | A | A |
| GT-06808 | A | A | A | | | A |
| GT-03457 | | A | A | A | A | A |
| GT-06301 | A | | A | | A | |
| GT-06351 | | A | A | A | A | A |
| GT-06513 | | | | | A | |
| GT-06516 | A | | A | A | A | A |
| GT-06514 | A | A | A | A | A | |
| GT-06138 | A | | | | A | |
| GT-06196 | | | A | | A | A |
| GT-06184 | | | | A | A | |
| GT-06645 | | | | | A | |
| GT-06926 | A | A | A | A | A | A |
| GT-06920 | A | A | | | | |
| GT-06958 | A | | | A | A | A |
| GT-06922 | A | A | | A | A | A |
| GT-06758 | | A | A | | A | |
| GT-06349 | | A | A | A | A | A |
| GT-06503 | A | A | | | A | A |
| GT-06518 | | | A | | A | A |
| GT-06811 | A | A | A | A | A | |
| GT-03369 | A | A | | A | A | |
| GT-06952 | | A | | A | A | A |
| GT-06872 | A | | A | A | A | A |
| GT-06815 | A | A | A | A | A | A |
| GT-06929 | A | | A | A | | |
| GT-06933 | A | A | A | A | | |
| GT-06816 | A | | A | | A | A |
| GT-06192 | A | | A | | | |
| GT-07025 | | | A | | | A |
| GT-07019 | A | | | | | |
| GT-07020 | A | | | | A | |
| GT-03363 | A | A | | A | A | A |
| GT-06817 | A | | A | A | A | A |
| GT-06193 | A | | A | | A | |
| GT-06934 | A | | A | | | A |
| GT-06953 | A | | | | | A |
| GT-06183 | A | | | | | |
| GT-06818 | A | | | | A | A |
| GT-06204 | | | A | A | A | A |
| GT-06935 | A | | | | A | A |
| GT-06954 | A | A | A | | A | |
| GT-06227 | A | | | | A | A |
| GT-06230 | A | | | | A | A |
| GT-06809 | A | A | A | A | A | A |
| GT-06353 | A | A | | A | A | A |
| GT-06562 | A | A | | | A | |
| GT-03459 | A | A | A | A | A | A |
| GT-06228 | A | | | | A | A |
| GT-06302 | A | A | A | A | A | A |
| GT-06354 | A | A | | A | A | A |
| GT-06560 | A | | | A | | |
| GT-06563 | A | | | | | |
| GT-06561 | A | | A | | | |
| GT-06185 | | | A | A | A | |
| GT-06819 | A | | A | A | A | A |
| GT-06195 | A | A | | A | A | A |
| GT-06928 | A | | A | | | |
| GT-06956 | A | A | A | A | | A |
| GT-06955 | | A | A | | | A |
| GT-06759 | | | A | | A | |
| GT-06352 | | A | | | A | A |
| GT-06519 | | | | | | A |
| GT-06520 | | | | | A | |
| GT-06959 | A | | | A | A | A |
| GT-06559 | A | | A | | A | A |
| GT-06760 | A | A | A | | | |
| GT-06814 | A | | A | A | A | A |
| GT-07061 | | | | | | A |
| GT-07062 | | | | | | A |
| GT-07063 | | | | | | A |
| GT-07064 | | | | | | A |
| GT-07065 | | | | | | A |
| GT-03613 | A | A | A | | A | A |
| GT-04309 | | | | | A | |
| GT-03616 | | A | A | | A | A |
| GT-06995 | | | | | | A |
| GT-06994 | | A | A | | | |
| GT-06997 | | | A | | A | |
| GT-03614 | A | | | | | |
| GT-03612 | A | A | A | A | A | A |
| GT-07003 | A | | | | | |
| GT-07005 | | | A | | | |

In Table E, A represents 0%≤ substrate protein remaining % ≤80%.

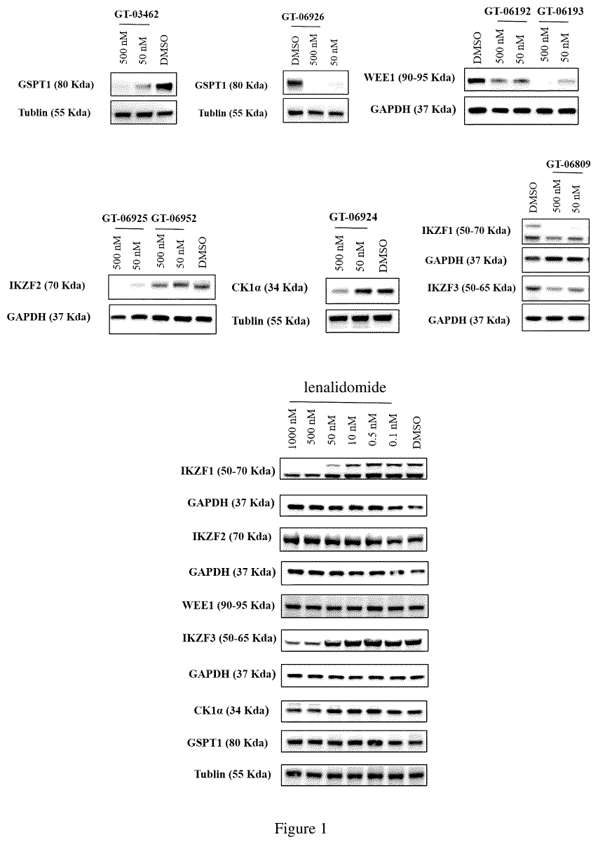

As demonstrated in Table E, the compounds of the present disclosure (including the compounds in Tables 1 and 2, and the Examples A1-A20 and 1-116 compounds) effectively degraded one or more substrate proteins (e.g., Wee1, IKZF1, IKZF2, IKZF3, GSPT1, CK1α, etc.), showing significantly superior activity compared to the marketed drug lenalidomide. Lenalidomide failed to effectively degrade Wee1, IKZF2, GSPT1, and CK1α proteins in hPBMC cells at concentrations ≤1000 nM (as shown in FIG. 1). Western blot results demonstrated that the compounds of the present disclosure exhibited significantly enhanced degradation efficiency against substrate proteins and a markedly expanded range of degradable substrates proteins compared to lenalidomide. In summary, the structural diversity of the compounds of the present disclosure enables effective degradation of multiple substrate proteins, making them potentially useful for treating diseases associated with these substrate proteins.

### IV. TNF-α Activity Inhibition Assay

PBMCs were cultured at 37° C. with 5% CO₂ and seeded in 96-well plates at 1×10\hat{ }7 cells/well. The compounds to be tested (including those listed in Tables 1 and 2, and Examples A1-A20 and 1-116) were dissolved in DMSO and diluted to appropriate concentrations, ensuring the final DMSO concentration in cell culture did not exceed 0.5%. After 1-hour incubation in medium with or without the compounds, the cells were stimulated with LPS (1 ng/ml) for 18-20 hours. Culture supernatants were then collected, diluted with serum-free medium, and analyzed for TNF-α levels using an ELISA kit. IC₅₀ values were calculated using GraphPad Prism 7.0.

TNF-α downregulation is a key mechanism of immunomodulatory agents' antitumor effects. The compounds of the present disclosure demonstrated dose-dependent inhibition of TNF-α levels.

### V. Pharmacokinetic Study of the Compounds of the Present Disclosure

The pharmacokinetic properties of the compounds of the present disclosure were evaluated using conventional pharmacokinetic experimental methods.

The test compounds of the present disclosure (including the compounds in Tables 1 and 2, and the Examples A1-A20 and 1-116 compounds) were orally administered to experimental animals to evaluate their pharmacokinetic properties. The experimental animals used may be those commonly used in pharmacokinetic experiments, e.g., adult and healthy rodents (e.g., mice, rats (such as Sprague-Dawley (SD) rats), guinea pigs) or non-rodents (rabbits, dogs, and monkeys). In the present disclosure, mice were used as the experimental animals.

The experimental animals were divided into two groups: a control group (for blank plasma collection) and an oral administration group (dosed at 10 mg/kg or 30 mg/kg). Blood samples were collected at predetermined time points after administration, including: 0.25 h, 0.5 h, 1 h, 2 h, 4 h, 8 h, and 24 h post-dose. Blank plasma was collected from the control group.

Prior to analysis, plasma samples were processed as follows: To 10 μL of plasma sample were added 10 μL of 50% acetonitrile and 200 μL of internal standard solution (containing 50 ng/mL dexamethasone in acetonitrile). For blank control plasma samples, no internal standard was added, instead an equivalent volume of acetonitrile was supplemented. The processed samples were vortex-mixed, centrifuged, and the supernatant was subjected to LC-MS/MS analysis.

After preparation of calibration curves and QC samples, the concentrations of compounds in plasma samples were determined by LC-MS/MS analysis. The pharmacokinetic parameters of the test compounds were calculated using the pharmacokinetic calculation software WinNonlin v8.1 with non-compartmental analysis based on the plasma concentration data obtained from LC-MS/MS.

The results demonstrated that the compounds of the present disclosure administered via oral route exhibited favorable drug metabolism and pharmacokinetic (DMPK) properties, indicating their potential as therapeutic agents for cancer patients.

VI. Effects of the Compounds of the Present Disclosure on TNF-α, IL-10, IL-6, IL-1β, IFN-γ, GM-CSF, IL-2, and IL-12p40 Release from PBMCs Induced by Lipopolysaccharide (LPS) or Phytohemagglutinin (PHA)

The following materials were used in this study:

VI.1 Reagents and Consumable materials

| Name | Supplier or Source |
|---|---|
| Human PBMC cell | Shanghai Sai Li |
| Lipopolysaccharide (LPS) | Sigma |
| Phytohemagglutinin (PHA) | MCE |
| RPMI 1640 Medium | Gibco |
| Human TNF-alpha DuoSet ELISA | R&D Systems |
| Human IL-6 DuoSet ELISA | R&D Systems |
| Human IL-10 DuoSet ELISA | R&D Systems |
| Human IFN-gamma DuoSet ELISA | R&D Systems |
| Human IL-2 DuoSet ELISA | R&D Systems |
| Human IL-1 beta ELISA Kit | Abclonal |
| Human GM-CSF ELISA Kit | Abclonal |
| Human IL-12/IL-23 p40 ELISA Kit | Abclonal |

### VI.2 Methods

### VI.2.1 Cell Thawing and Plating

A complete medium was prepared and pre-warmed to 37° C. Cryovials were retrieved from liquid nitrogen, and the area immediately below the cap-notch interface was firmly gripped with forceps. The cryovials were immersed in a 37° C. water bath, and shook occasionally to facilitate thawing. The cell suspension was aspirated and transferred to a centrifuge tube containing 10 mL complete medium, followed by thorough mixing and centrifugation at 400×g for 10 min at room temperature. The supernatant was discarded, and the pellet was resuspended in fresh complete medium. After cell counting and the cell density adjustment, the cell suspension was seeded in 96-well plates at a density of 1×10⁵ cells/well (75 L/well) and incubated for 2 h at 37° C. with 5% CO₂ under high humidity conditions.

### VI.2.2 Compound Preparation and Addition to Cell Plates

1) The test compounds were prepared as 10 mM stock solutions in DMSO.
2) The test compounds were serially diluted 5-fold in DMSO starting from 1 mM (highest concentration) to generate 9 concentration gradients.
3) 2) The test compounds were further diluted in culture medium to five times the final working concentration intended for use.
4) The diluted compounds (25 L/well) were added to designated wells according to the plate layout, achieving final concentrations starting from 1 M with 5-fold serial dilution (9 concentration points in total).
5) After 1 h incubation, LPS or PHA (25 L/well) was added to the cell wells to reach final concentrations of: 1 μg/mL LPS (for TNF-α, IL-10, IL-6, GM-CSF and IL-12p40), 5 μg/mL LPS (IL-1β), 50 μg/mL LPS (IFN-γ), or 1 μg/mL PHA (IL-2).
6) The plates were incubated in a incubator for 24 h at 37° C. with 5% CO₂ under high humidity.

### Cell Plate Layout:

| | 1 | 2 | 3 | 4 | 5 | 6 | 7 | 8 | 9 | 10 | 11 | 12 |
|---|---|---|---|---|---|---|---|---|---|---|---|---|
| A | H₂O | H₂O | H₂O | H₂O | H₂O | H₂O | H₂O | H₂O | H₂O | H₂O | H₂O | H₂O |
| B | H₂O | Cmpd 1# (1 μM Starting, 5-fold dilution, 9 points) | | | | | | | | | LPS/PHA | H₂O |
| C | H₂O | Cmpd 2# (1 μM Starting, 5-fold dilution, 9 points) | | | | | | | | | | H₂O |
| D | H₂O | | | | | | | | | | | H₂O |
| E | H₂O | | | | | | | | | | | H₂O |
| F | H₂O | Cmpd 3# (1 μM Starting, 5-fold dilution, 9 points) | | | | | | | | | Blank | H₂O |
| G | H₂O | | | | | | | | | | | H₂O |
| H | H₂O | H₂O | H₂O | H₂O | H₂O | H₂O | H₂O | H₂O | H₂O | H₂O | H₂O | H₂O |

### VI.2.3 Detection

(1) The cell plate was centrifuged and the culture supernatant was aspirated. TNF-α, IL-10, IL-6, IL-1β, IFN-γ, GM-CSF, IL-2, and IL-12p40 cytokines in the culture supernatant were detected according to the ELISA kit instructions.
The concentrations of TNF-α, IL-10, IL-6, IL-1β, IFN-γ, GM-CSF, IL-2, and IL-12p40 in the samples were calculated based on the standard curve. Inhibition rate %=[(Ac−As)/(Ac-Ab)]X 100%
As: OA of the samples (cells+LPS/PHA+test compound)
Ac: OA of positive cell control (cells+LPS/PHA+DMSO)
Ab: OA of blank control (cells+culture medium+DMSO)
(2) IC₅₀ value was calculated as follows: using GraphPad Prism 6 software and applying the calculation formula XY-analysis/Nonlinear regression (curve fit)/Dose response-Inhibition/log (inhibitor) vs. response-Variable slope (four parameters) to perform IC₅₀ curve fitting and determine the IC₅₀ values.

The experimental results demonstrated that the compounds of the present disclosure can modulate the production levels of cytokines associated with autoimmune diseases and may be used for the treatment of autoimmune diseases.

### VII. Efficacy Experiment of the Compounds of the Present Disclosure on Psoriasis-Like Skin Lesions Induced by Imiquimod in Mice

The compounds of the present disclosure (test compounds, including the compounds in Tables 1 and 2, and the Examples A1-A20 and 1-116 compounds) were administered orally via gavage once daily for 7 consecutive days to mice with psoriasis-like skin lesions induced by imiquimod, to investigate their efficacy on such lesions.

A total of 60 female C₅₇BL/6J mice (purchased from Shanghai SLAC Laboratory Animal Co., Ltd.) were randomly divided into 6 groups (n=10 per group) based on body weight:

| | | | | | Administration | |
|---|---|---|---|---|---|---|
| | | Number of | Dose | Administration | concentration | Route and |
| Group | Drug | Animals | (mg/kg) | volume (mL/kg) | (mg/mL) | Duration |
| 1 | Control: Vehicle | 10 | NA | 10 | NA | ig. qd. 7 days |
| 2 | Model: Vehicle | 10 | NA | 10 | NA | ig. qd. 7 days |
| 3 | Positive Control | 10 | 3 | 10 | 0.3 | sc. qd. 7 days |
| 4 | Low-dose test | 10 | 3 | 10 | 0.3 | ig. qd. 7 days |
| | compounds group | | | | | |
| 5 | Medium-dose test | 10 | 10 | 10 | 1 | ig. qd. 7 days |
| | compounds group | | | | | |
| 6 | High-dose test | 10 | 30 | 10 | 3 | ig. qd. 7 days |
| | compounds group | | | | | |

Note:
ig., oral gavage; sc., subcutaneous injection; qd., once daily.

After grouping the experimental animals, the backs of all mice were shaved. Except for the Control group, the remaining groups were topically treated with imiquimod cream once daily for 7 consecutive days. Simultaneously, each group was administered with a solvent control, a positive control drug, or the test compounds, according to the above table, once daily for 7 consecutive days.

All clinical symptoms of each animal were observed at the beginning and throughout the experiment. The animals' weights were measured and recorded every 2 days.

Scoring Gross Observation Scoring of Skin Lesions (PASI Method): during the last 3 days of modeling and drug administration, the changes in skin lesions of mice in each group were observed once daily. PASI Method: the erythema, scaling, and thickening/infiltration of the lesions were scored separately, and the scores were summed to obtain the total score. PASI Scoring Criteria: 0=none; 1=mild; 2=moderate; 3=severe; 4=very severe.

The skin lesions were photographed once daily during the last 3 days of modeling and drug administration.

After the experiment, the mice were euthanized by CO₂ inhalation. Three different areas of skin from the lesion site on the back were collected using a 6 mm punch, weighed, and recorded, and the average weight was calculated.

The skin tissue from the lesion site was homogenized and the levels of IL-23p19, IL-12p40, IL-17, and TNF-α were determined.

The local lesion tissue was fixed, stained with HE, and subjected to pathological scoring (based on epidermal thickness, degree of epidermal keratinization, thickness of the basal layer, and degree of dermal inflammatory cell infiltration). Scoring Criteria: 0=none; 1=mild; 2=moderate; 3=severe; 4=very severe.

Experimental data were presented as Mean±SD. Statistical analysis between two groups was performed using SPSS, with p<0.05 considered statistically significant.

The experimental results demonstrated that the compounds of the present disclosure improve psoriasis-like skin lesions on the back of mice induced by imiquimod and may be used for the treatment of psoriasis.

### VIII. Efficacy Experiment of the Compounds of the Present Disclosure on the CIA Model in Rats Induced by Type II Collagen

The compounds of the present disclosure (test compounds, including the compounds in Tables 1 and 2, and the Examples A1-A20 and 1-116 compounds) were administered orally via gavage once daily for 21 consecutive days to Wistar rats with collagen-induced arthritis (CIA) that was induced using bovine type II collagen (Chondrex, Inc.), in order to investigate their efficacy in the rat arthritis model.

Preparation of the Modeling Agent: 10 mg of CII (Immunization Grade Bovine Type II Collagen, purchased from Chondrex, Inc.) was dissolved in 5 mL of 0.05M acetic acid solution, allowing it to be fully dissolved, thereby preparing a solution with a concentration of 2 mg/mL, which was then stored at 4° C. in the dark overnight. On the day of the experiment, the 2 mg/mL CII solution was mixed with an equal volume of a 4 mg/mL solution of Complete Freund's Adjuvant (CFA, also purchased from Chondrex, Inc.) on ice, and was thoroughly emulsified into an emulsion.

On day 0, 10 Wistar rats (purchased from Beijing Vital River Laboratory Animal Technology Co., Ltd.) were randomly selected as the blank control group and were not immunized. The remaining 100 Wistar rats were intradermally injected at the base of the tail with an emulsion prepared by mixing equal volumes of CII (2 mg/mL) and CFA (4 mg/mL) for the first immunization. A second booster immunization was administered to them on day 7. Between days 10 and 14, i.e., 3 to 7 days after the second booster immunization, 50 rats with an arthritis index (AI) score ranging from 1 to 2 were selected from the modeled animals and were divided into 5 groups based on their body weight, foot volume, and A1 score, with 10 rats per group:

| | | | | | Administration | |
|---|---|---|---|---|---|---|
| | | Number of | Dose | Administration | concentration | Route and |
| Group | Drug | Animals | (mg/kg) | volume (mL/kg) | (mg/mL) | Duration |
| 1 | Control: Vehicle | 10 | NA | 10 | NA | ig. qd. 21 days |
| 2 | Model: Vehicle | 10 | NA | 10 | NA | ig. qd. 21 days |
| 3 | Positive Control | 10 | 0.1 | 10 | 0.01 | sc. qd. 21 days |
| 4 | Low-dose test | 10 | 3 | 10 | 0.3 | ig. qd. 21 days |
| | compounds | | | | | |
| | group | | | | | |
| 5 | Medium-dose | 10 | 10 | 10 | 1 | ig. qd. 21 days |
| | test compounds | | | | | |
| | group | | | | | |
| 6 | High-dose test | 10 | 30 | 10 | 3 | ig. qd. 21 days |
| | compounds | | | | | |
| | group | | | | | |

Note:
ig. = oral gavage;
sc. = subcutaneous injection

Between 3 and 7 days after the second booster immunization, when the A1 score of the affected feet reached 1 to 2, a solvent control, a positive control drug, or the test compounds were administered orally via gavage (or subcutaneously) to the animals in each group, according to the above table, once daily for 21 consecutive days.

Starting 3 days after the second booster immunization, the volume of both hind paws (or toes) was measured twice a week and recorded.

Foot Volume Measurement: Before the measurement, a line was marked on the rat's ankle joint with a marker pen to indicate the position. After clean water was added to the instrument, the instrument's reading was reset to zero in preparation for the measurement. The hind limb of the rat was placed in the water so that the marked line at the ankle joint was at the surface of the liquid. The reading obtained by pressing the foot pedal at this time was the foot volume of the rat. After the measurement was completed, the foot pedal was pressed again to reset the instrument to zero, preparing it for the measurement of the next rat.

### Scoring

Arthritis Index (A1): The foot volume was measured twice a week, and the swelling of the four toes was scored simultaneously. Each foot was scored on a scale from 0 to 4, with a maximum score of 16 for each rat.

### Arthritis Index (AI) Scoring Criteria

| Score | Degree |
|---|---|
| 0 | No swelling, normal appearance |
| 1 | Mild swelling or redness of the ankle, wrist, or finger joints |
| 2 | Moderate swelling or redness of the ankle, wrist, or finger joints |
| 3 | Severe swelling or redness of the ankle, wrist, or finger joints |
| 4 | Very severe swelling or redness of the ankle, wrist, or finger joints |

The day of starting drug administration is recorded as Day 0. Five days after starting drug administration, i.e., 2 hours after Day 5 administration, blood was collected from the jugular vein of the experimental animals, allowed to stand for more than 30 minutes, centrifuged to separate the serum, which was stored at −80° C. The levels of TNF-α, IL-1β, and IL-6 in serum were measured by ELISA.

After the experiment, the animals were euthanized by CO₂ inhalation. The spleen and thymus of the animals were collected and weighed, and the organ coefficients were calculated.

Pathological Examination: One side of the foot joint tissue was fixed in 10% neutral formalin solution, followed by decalcification and paraffin embedding to prepare pathological sections. The sections were then stained with hematoxylin and eosin (HE) for pathological observation.

### Observation Indices:

1. Infiltration of synovial inflammatory cells (lymphocytes, plasma cells);
2. Synovial tissue hyperplasia;
3. Synovial pannus;
4. Fibrinoid necrosis on the synovial surface.
   Scoring and Counting Method: 0, 1, 2, 3, and 4 levels, where “0” indicates no observation; “1” is mild; “2” is moderate; “3” is severe; and “4” is extremely severe.

Experimental data were presented as Mean±SEM. Data between two groups were analyzed using Excel-T test, with p<0.05 considered statistically significant. Scoring data were analyzed using the non-parametric Mann-Whitney U test in SPSS, with p<0.05 considered statistically significant.

The experimental results demonstrated that the compounds of the present disclosure can regulate the serum levels of inflammatory cytokines in rats with collagen-induced arthritis (CIA) model that was induced using bovine type II collagen, and significantly improve the swelling of rat limbs, and thus may be used for the treatment of arthritis.

The above descriptions represent only preferred embodiments of the present invention, but the scope of protection is not limited thereto. Any person skilled in the art, within the technical scope disclosed by the invention, may make equivalent replacements or modifications based on the technical solutions and inventive concepts of the present invention, and such changes shall fall within the protection scope of the invention.

1. A compound of Formula (I) 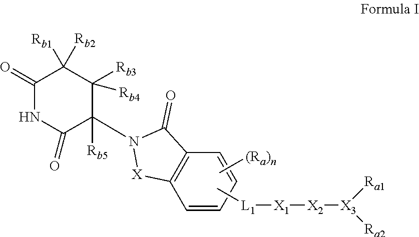
or salts, enantiomers, stereoisomer, polymorphs or solvates thereof,
wherein X is selected from the group consisting of CO or CH₂;
L₁ is selected from the group consisting of S or O;
X₁ is selected from the group consisting of unsubstituted or substituted linear or branched alkylene, or X₁ is absent;
X₂ represents a structure of Formula II: 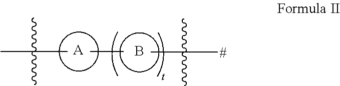
wherein ring A and ring B are each independently selected from the group consisting of unsubstituted or substituted arylene, unsubstituted or substituted heteroarylene, unsubstituted or substituted heterocyclylene, or unsubstituted or substituted cycloalkylene;
t represents an integer of 0, 1 or 2; and
symbol # indicates the point of attachment to X₃;
X₃ is selected from the group consisting of CR₁, N, or CO, wherein R₁ is selected from the group consisting of hydrogen, deuterium, halogen, nitro, cyano, hydroxy, mercapto, trifluoromethoxy, unsubstituted or substituted amino, unsubstituted or substituted silyl, unsubstituted or substituted boryl, unsubstituted or substituted linear or branched alkyl, or unsubstituted or substituted cycloalkyl;
Rₐ₁ is selected from the group consisting of hydrogen, deuterium, unsubstituted or substituted aryl, unsubstituted or substituted heteroaryl, unsubstituted or substituted cycloalkyl, or unsubstituted or substituted heterocyclyl;
Rₐ₂ is selected from the group consisting of hydrogen, deuterium, unsubstituted or substituted aryl, unsubstituted or substituted heteroaryl, unsubstituted or substituted cycloalkyl, or unsubstituted or substituted heterocyclyl, or Rₐ₂ is absent; and when X₃ represents CO, Rₐ₂ is absent and Rₐ₁ is neither hydrogen nor deuterium, and when Rₐ₂ represents hydrogen or deuterium, X₃ represents CR₁ or N, and Rₐ₁ is neither hydrogen nor deuterium;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl;
R_{b1}, R_{b2}, R_{b3}, R_{b4} and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl; and
n is an integer of 0, 1, 2 or 3, and when n is an integer of 2 or 3, each Rₐ is independently identical to or different from each other.

2. The compound or salts, enantiomers, stereoisomers, polymorphs or solvates thereof as claimed in claim 1, wherein X₁ represents unsubstituted or substituted linear or branched C₁-C₅ alkylene, or X₁ is absent;
and/or, X₂ represents the structure of Formula II: 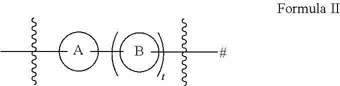
wherein ring A is selected from the group consisting of unsubstituted or substituted C₅-C₃₀ arylene, unsubstituted or substituted 5- to 30-membered heteroarylene, unsubstituted or substituted 4- to 30-membered heterocyclylene, or unsubstituted or substituted C₃-C₃₀ cycloalkylene; preferably, ring A is selected from the group consisting of unsubstituted or substituted C₅-C₂₀ arylene, unsubstituted or substituted 5- to 20-membered heteroarylene, unsubstituted or substituted 4- to 20-membered heterocyclylene, or unsubstituted or substituted C₃-C₂₀ cycloalkylene;
ring B is selected from the group consisting of unsubstituted or substituted C₅-C₃₀ arylene, unsubstituted or substituted 5- to 30-membered heteroarylene, unsubstituted or substituted 4- to 30-membered heterocyclylene, or unsubstituted or substituted C₃-C₃₀ cycloalkylene; preferably, ring B is selected from the group consisting of unsubstituted or substituted C₅-C₂₀ arylene, unsubstituted or substituted 5- to 20-membered heteroarylene, unsubstituted or substituted 4- to 20-membered heterocyclylene, or unsubstituted or substituted C₃-C₂₀ cycloalkylene;
t represents an integer of 0, 1 or 2; preferably, t represents an integer of 0 or 1; and
symbol # indicates the point of attachment to X₃;
and/or, R₁ is selected from the group consisting of hydrogen, deuterium, F, Cl, Br, I, nitro, cyano, hydroxy, mercapto, trifluoromethoxy, unsubstituted or substituted amino, unsubstituted or substituted silyl, unsubstituted or substituted boryl, unsubstituted or substituted linear or branched C₁-C₁₀ alkyl, or unsubstituted or substituted C₃-C₁₅ cycloalkyl;
and/or, Rₐ₁ is selected from the group consisting of hydrogen, deuterium, unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl; preferably, Rₐ₁ is selected from the group consisting of hydrogen, deuterium, unsubstituted or substituted C₅-C₂₀ aryl, unsubstituted or substituted 5- to 20-membered heteroaryl, unsubstituted or substituted C₃-C₂₀ cycloalkyl, or unsubstituted or substituted 4- to 20-membered heterocyclyl;
and/or, Rₐ₂ is selected from the group consisting of hydrogen, deuterium, unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl, or Rₐ₂ is absent; preferably, Rₐ₂ is selected from the group consisting of hydrogen, deuterium, unsubstituted or substituted C₅-C₂₀ aryl, unsubstituted or substituted 5- to 20-membered heteroaryl, unsubstituted or substituted C₃-C₂₀ cycloalkyl, or unsubstituted or substituted 4- to 20-membered heterocyclyl, or Rₐ₂ is absent;
and/or, Rₐ is selected from the group consisting of deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, unsubstituted or substituted linear or branched C₁-C₅ alkyl, unsubstituted or substituted C₃-C₁₅ cycloalkyl, unsubstituted or substituted 4- to 15-membered heterocyclyl, unsubstituted or substituted C₅-C₁₅ aryl, or unsubstituted or substituted 5- to 15-membered heteroaryl; preferably, Rₐ is selected from the group consisting of deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, unsubstituted or substituted linear or branched C₁-C₅ alkyl, unsubstituted or substituted C₃-C₁₅ cycloalkyl, unsubstituted or substituted 4 to 15-membered heterocyclyl, unsubstituted or substituted C₅-C₁₅ aryl, or unsubstituted or substituted 5- to 15-membered heteroaryl;
and/or, R_{b1}, R_{b2}, R_{b3}, R_{b4} and R_{b5} are each independently selected from the group consisting of hydrogen, deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, or unsubstituted or substituted linear or branched C₁-C₅ alkyl; preferably, R_{b1}, R_{b2}, R_{b3}, R_{b4} and R_{b5} are each independently selected from the group consisting of hydrogen, deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, or unsubstituted or substituted linear or branched C₁-C₅ alkyl.

3. The compound or salts, enantiomers, stereoisomers, polymorphs or solvates thereof as claimed in claim 1, wherein the structure of said compound is shown in Formula I-1 or Formula I-2: 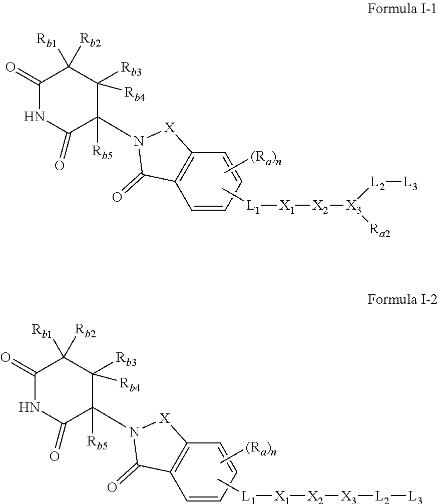
in Formula I-1 or Formula I-2, X, L₁, X₁, X₂, Rₐ, R_{b1}, R_{b2}, R_{b3}, R_{b4}, R_{b5}, and n are as defined in claim 1;
L₂ is selected from the group consisting of unsubstituted or substituted C₅-C₃₀ arylene, unsubstituted or substituted 5- to 30-membered heteroarylene, unsubstituted or substituted C₃-C₃₀ cycloalkylene, or unsubstituted or substituted 4- to 30-membered heterocyclylene, or L₂ is absent;
L₃ is selected from the group consisting of unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl;
Rₐ₂ is selected from the group consisting of unsubstituted or substituted C₅-C₃₀ aryl, unsubstituted or substituted 5- to 30-membered heteroaryl, unsubstituted or substituted C₃-C₃₀ cycloalkyl, or unsubstituted or substituted 4- to 30-membered heterocyclyl;
wherein in Formula I-1, X₃ is selected from the group consisting of CR₁ or N, and in Formula I-2, X₃ is selected from the group consisting of CR₁, N or CO, wherein R₁ is as defined in claim 1.

4. The compound or salts, enantiomers, stereoisomers, polymorphs or solvates thereof as claimed in claim 1, wherein (i) X₁ is selected from the group consisting of methylene, ethylene, propylene or isopropylene, or X₁ is absent; and/or
(ii) X₂ represents the structure of Formula II: 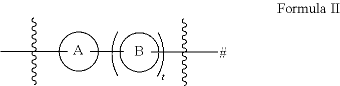
wherein ring A is selected from the group consisting of unsubstituted or substituted C₅-C₁₅ arylene, unsubstituted or substituted 5- to 15-membered heteroarylene, unsubstituted or substituted 4 to 15-membered heterocyclylene, or unsubstituted or substituted C₃-C₁₅ cycloalkylene, wherein the substituent of the substituted ring A is optionally one or more substituents selected from the group consisting of halogen, mercapto, hydroxy, amino, cyano, C₁-C₆ alkyl, C₁-C₆ alkoxy, halogenated C₁-C₆ alkyl or any combination thereof;
ring B is selected from the group consisting of unsubstituted or substituted C₅-C₁₅ arylene, unsubstituted or substituted 5- to 15-membered heteroarylene, unsubstituted or substituted 4- to 15-membered heterocyclylene, or unsubstituted or substituted C₃-C₁₅ cycloalkylene; wherein the substituent of the substituted ring B is optionally one or more substituents selected from the group consisting of halogen, mercapto, hydroxy, amino, cyano, C₁-C₆ alkyl, C₁-C₆ alkoxy, halogenated C₁-C₆ alkyl or any combination thereof;
t represents an integer of 0, 1 or 2; preferably, t represents an integer of 0 or 1; and
symbol # indicates the point of attachment to X₃;
and/or
(iii) when Rₐ₂ is present, X₃ is selected from the group consisting of CH or N;
when Rₐ₂ is absent, X₃ represents CO.

5. (canceled)

6. The compound or salts, enantiomers, stereoisomers, polymorphs or solvates thereof as claimed in claim 1, wherein the ring A and ring B are each independently selected from the group consisting of the following groups:
azetidinylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, azacyclooctylene, diazacycloheptanylene, diazacyclooctylene, bridged heterocyclylene (e.g., 5- to 15-membered bridged heterocyclylene, such as azabicyclohexylene, azabicycloheptanylene, or azabicyclooctylene), or spiro-heterocyclylene (e.g., 5- to 15-membered spiro-heterocyclylene, such as 3-azaspiro[5.5]undecanylene or 7-azaspiro[3.5]nonanylene); or
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene; or
cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₅₋₁₅ spiro-cycloalkylene, such as spiro[3.3]heptylene, spiro[2.5]octylene, spiro[3.5]nonylene, spiro[4.4]nonylene, spiro[4.5]decylene, or spiro[5.5]undecylene), or bridged cycloalkylene (e.g., C₅₋₁₅ bridged cycloalkylene, such as adamantanylene, noradamantanylene, bornylene, bicyclo[2.2.1]heptylene, 2-oxobicyclo[2.2.1]heptylene, or bicyclo[2.2.1]heptentylene); or
phenylene or naphthylene;
wherein the ring A and ring B are each independently optionally substituted by one or more substituents selected from the group consisting of deuterium, halogen, nitro, mercapto, hydroxy, cyano, C₁-C₆ alkyl, C₁-C₆ alkoxy, halogenated C₁-C₆ alkoxy, halogenated C₁-C₆ alkyl, unsubstituted or substituted amino, unsubstituted or substituted silyl, unsubstituted or substituted boryl or any combination thereof.

7. The compound or salts, enantiomers, stereoisomers, polymorphs or solvates thereof as claimed in claim 3, wherein X₂ is selected from the group consisting of: 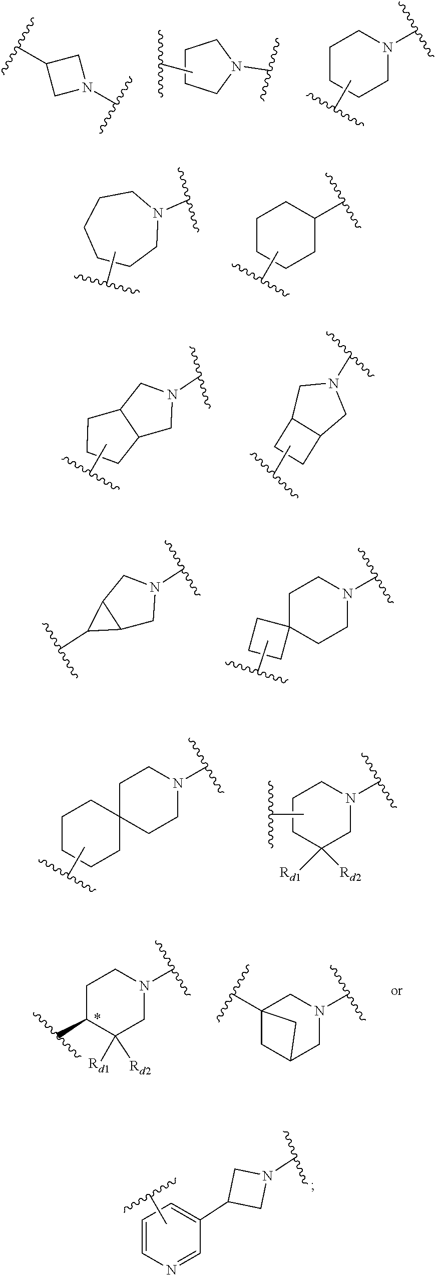
wherein R_{d1} and R_{d2} are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, and unsubstituted or substituted linear or branched C₁-C₅ alkyl; and R_{d1} and R_{d2} are not hydrogen at the same time.

8. (canceled)

9. The compound or salts, enantiomers, stereoisomers, polymorphs or solvates thereof as claimed in claim 3, wherein
(i) L₂ is selected from the group consisting of unsubstituted or substituted 5- to 20-membered heteroarylene (e.g., unsubstituted or substituted 5- to 15-membered heteroarylene); preferably, the heteroarylene is selected from the group consisting of pyridylene, pyrrolylene, pyrimidinylene, pyridazinylene, furanylene, thienylene, imidazolylene, pyrazolylene, oxazolylene, isoxazolylene, thiazolylene, isothiazolylene, triazolylene, oxadiazolylene, thiadiazolylene, tetrazolylene, pyranylene, thiopyranylene, pyrazinylene, pyridazinylene, thiazinylene, triazinylene, tetrazinylene, quinolinylene, isoquinolinylene, quinolinylene, quinazolinylene, quinoxalinylene, cinnolinylene, naphthyridinylene, acridinylene, xanthenylene, phenanthridinylene, diazanaphthylene, triazaindenylene, indolylene, indolinylene, indolizinylene, phthalazinylene, pyrazolopyridylene, pyrazolopyrimidinylene, pyridopyrimidinylene, pyridopyrazinylene, pyrazinopyrazinylene, benzothiazolylene, benzoxazolylene, benzimidazolylene, benzothienylene, benzofuranylene, isobenzofuranylene, dibenzothienylene, dibenzofuranylene, indazolylene, carbazolylene, benzocarbazolylene, dibenzocarbazolylene, indolocarbazolylene, indenocarbazolylene, phenazinylene, imidazopyridinylene, phenazinylene, phenanthridinylene, phenanthrolinylene, phenothiazinylene, imidazopyridinylene, imidazophenanthridinylene, pyrrolopyridinylene, pyrrolothiazolylene, imidazothiazolylene, benzimidazoquinazolinylene, benzimidazophenanthridinylene, pyrenylene, dinaphthofuranylene, naphthobenzofuranylene, dinaphthothienylene, or naphthobenzothienylene;
and/or, L₂ is selected from the group consisting of unsubstituted or substituted C₃-C₂₀ cycloalkylene (e.g., unsubstituted or substituted C₃-C₁₅ cycloalkylene); preferably, L₂ is selected from the group consisting of cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₅-C₁₅ spiro-cycloalkylene, such as spiro[3.3]heptylene, spiro[2.5]octylene, spiro[3.5]nonylene, spiro[4.4]nonylene, spiro[4.5]decylene, or spiro[5.5]undecylene), or bridged cycloalkylene (e.g., C₅-C₁₅ bridged cycloalkylene, adamantanylene, noradamantanylene, bornylene, bicyclo[2.2.1]heptylene, 2-oxobicyclo[2.2.1]heptylene, or bicyclo[2.2.1]heptentylene);
and/or, L₂ is selected from the group consisting of unsubstituted or substituted C₅-C₂₀ arylene (e.g., unsubstituted or substituted C₅-C₁₅ arylene); preferably, L₂ is selected from the group consisting of phenylene, biphenylene, terphenylene, naphthylene, anthrylene, phenanthrylene, fluorenylene, 9,9′-dimethylfluorenylene;
and/or, L₂ is selected from the group consisting of unsubstituted or substituted 4- to 20-membered heterocyclylene (e.g., unsubstituted or substituted 4- to 15-membered heterocyclylene); preferably, L₂ is selected from the group consisting of azetidinylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, azacyclooctylene, diazacycloheptanylene, diazacyclooctylene, bridged heterocyclylene (e.g., 5- to 15-membered bridged heterocyclylene, such as azabicyclohexylene, azabicycloheptanylene, diazabicycloheptanylene, azabicyclooctylene), or spiro-heterocyclylene (e.g., 5- to 15-membered spiro-heterocyclylene, such as 3-azaspiro[5.5]undecanylene or 7-azaspiro[3.5]nonanylene);
wherein the substituent of the substituted L₂ is optionally one or more substituents selected from the group consisting of deuterium, halogen, nitro, mercapto, hydroxy, cyano, halogenated C₁-C₆ alkyl linear or branched C₁-C₆ alkyl, halogenated C₁-C₆ alkoxy, C₁-C₆ alkoxy, halogenated C₁-C₆ alkyl, unsubstituted or substituted amino, unsubstituted or substituted silyl, unsubstituted or substituted boryl or any combination thereof;
or L₂ is absent; and/or
(ii) L₃ and Rₐ₂ are each independently selected from the group consisting of unsubstituted or substituted C₅-C₂₀ aryl (e.g., unsubstituted or substituted C₅-C₁₅ aryl); preferably, L₃ and Rₐ₂ are each independently selected from the group consisting of biphenyl, terphenyl, naphthyl, anthryl, phenanthryl, fluorenyl, and 9,9′-dimethylfluorenyl;
and/or, L₃ and Rₐ₂ are each independently selected from the group consisting of unsubstituted or substituted 5- to 25-membered heteroaryl (preferably, unsubstituted or substituted 5- to 20-membered heteroaryl, or unsubstituted or substituted 5- to 15-membered heteroaryl); more preferably, L₃ and Rₐ₂are each independently selected from the group consisting of pyridyl, pyrrolyl, pyrimidinyl, pyridazinyl, furanyl, thienyl, imidazolyl, pyrazolyl, oxazolyl, isoxazolyl, thiazolyl, isothiazolyl, triazolyl, oxadiazolyl, thiadiazolyl, tetrazolyl, pyranyl, thiopyranyl, pyrazinyl, oxazinyl, thiazinyl, triazinyl, tetrazinyl, quinolinyl, isoquinolinyl, quinazolinyl, quinoxalinyl, cinnolinyl, naphthyridinyl, acridinyl, xanthenyl, phenanthridinyl, diazanaphthyl, triazaindenyl, indolyl, indolinyl, indolizinyl, phthalazinyl, pyridopyrimidinyl, pyridopyrazinyl, pyrazinopyrazinyl, benzothiazolyl, benzoxazolvl, benzimidazolyl, benzothienyl, benzofuranyl, dibenzothienyl, dibenzofuranyl, carbazolyl, benzocarbazolyl, dibenzocarbazolyl, indolocarbazolyl, indenocarbazolyl, phenazinyl, imidazopyridinyl, phenanthrolinyl, phenothiazinyl, imidazopyridinyl, imidazophenanthridinyl, benzimidazoquinazolinyl, benzimidazophenanthridinyl, spiro[fluorene-9,9′-xanthene], pyrenyl, dinaphthofuranyl, naphthobenzofuranyl, dinaphthothienyl, or naphthobenzothienyl;
and/or, L₃ and Rₐ₂ are each independently selected from the group consisting of unsubstituted or substituted 4- to 20-membered heterocyclyl (e.g., unsubstituted or substituted 4- to 15-membered heterocyclyl); preferably, L₃ and Rₐ₂ are each independently selected from the group consisting of azetidinyl, oxetanyl, pyrrolidinyl, imidazolidinyl, pyrazolidyl, tetrahydrofuranyl, tetrahydropyranyl, dihydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, dioxacyclohexenyl, azacycloheptanyl, azacyclooctyl, diazacycloheptanyl, diazacyclooctyl, bridged heterocyclyl (e.g., 5- to 15-membered bridged heterocyclyl, such as azabicyclohexyl, azabicycloheptanyl, or azabicyclooctyl), or spiro-heterocyclyl (e.g., 5- to 15-membered spiro-heterocyclyl, such as 3-azaspiro[5.5]undecanyl or 7-azaspiro[3.5]nonanyl):
and/or, L₃ and Rₐ₂ are each independently selected from the group consisting of unsubstituted or substituted C₃-C₂₀ cycloalkyl (e.g., unsubstituted or substituted C₃-C₁₅ cycloalkyl); preferably, L₃ and Rₐ₂ are each independently selected from the group consisting of cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, spiro-cycloalkyl (e.g., C₅-C₁₅ spiro-cycloalkyl, such as spiro[3.3]heptyl, spiro[2.5]octyl, spiro[3.5]nonyl, spiro[4.4]nonyl, spiro[4.5]decyl, or spiro[5.5]undecyl), or bridged cycloalkyl (e.g., C₅-C₁₅ bridged cycloalkyl, such as adamantanyl, noradamantanyl, bornyl, bicyclo[2.2.1]heptyl, 2-oxobicyclo[2.2.1]heptyl or bicyclo[2.2.1]heptentyl);
wherein the substituents of the substituted L₃ and Rₐ₂ are each independently optionally one or more substituents selected from the group consisting of deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, linear or branched C₁-C₅ alkyl or halogenated C₁-C₅ alkyl.

10. The compound or salts, enantiomers, stereoisomers, polymorphs or solvates thereof as claimed in claim 3, wherein
(i)L₂ is selected from the group consisting of: 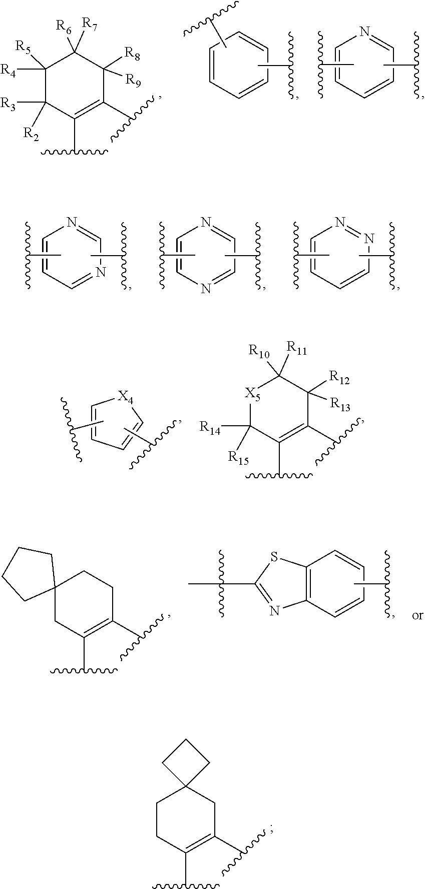
wherein X₄ is selected from the group consisting of O, S, or NH;
X₅ is selected from the group consisting of O, S, or NR₁₆, wherein R₁₆ represents hydrogen or C₁-C₃ alkyl;
R₂, R₃, R₄, R₅, R₆, R₇, R₈, R₉, R₁₀, R₁₁, R₁₂, R₁₃, R₁₄ and R₁₅ are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, linear or branched C₁-C₅ alkyl or halogenated C₁-C₅ alkyl, wherein preferably, the halogenated C₁-C₆ alkoxy is selected from the group consisting of trifluoromethoxy; or
L₂ is absent; and/or
(ii) L₃ and Rₐ₂ are each independently selected from the group consisting of: 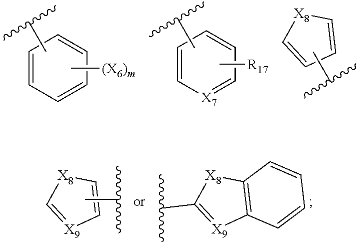
wherein X₆ is selected from the group consisting of deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, linear or branched C₁-C₆ alkyl or halogenated C₁-C₆ alkyl:
m is an integer of 0, 1, 2, 3, 4 or 5, and when m is an integer of 2, 3, 4 or 5, each X₆ is independently identical to or different from each other;
X₇ and X₉ are each independently selected from the group consisting of CH or N;
each X₈ is independently selected from the group consisting of O, S or NH;
R₁₇ is selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, linear or branched C₁-C₅ alkyl or halogenated C₁-C₅ alkyl, wherein the halogenated C₁-C₆ alkoxy is preferably selected from the group consisting of trifluoromethoxy.

11. (canceled)

12. (canceled)

13. The compound or salts, enantiomers, stereoisomers, polymorphs or solvates thereof as claimed in claim 1, wherein the chemical structure of said compound is selected from the group consisting of any one of the following compounds:

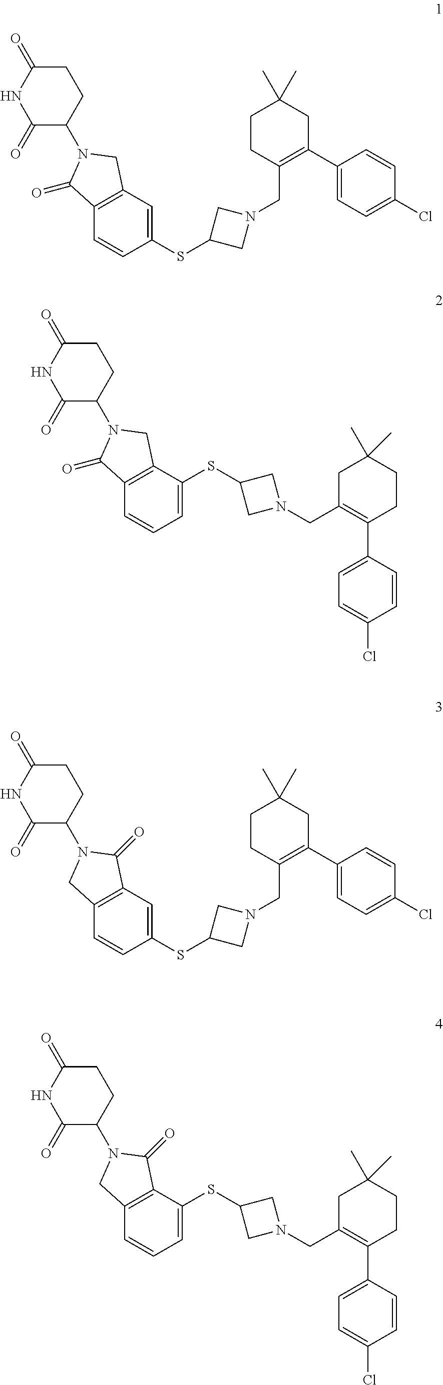 [SMILES: CC1(C)CCC(CN2CC(Sc3ccc4c(c3)CN(C3CCC(=O)NC3=O)C4=O)C2)=C(c2ccc(Cl)cc2)C1 ; CC1(C)CCC(c2ccc(Cl)cc2)=C(CN2CC(Sc3cccc4c3CN(C3CCC(=O)NC3=O)C4=O)C2)C1 ; CC1(C)CCC(CN2CC(Sc3ccc4c(c3)C(=O)N(C3CCC(=O)NC3=O)C4)C2)=C(c2ccc(Cl)cc2)C1 ; CC1(C)CCC(c2ccc(Cl)cc2)=C(CN2CC(Sc3cccc4c3C(=O)N(C3CCC(=O)NC3=O)C4)C2)C1]

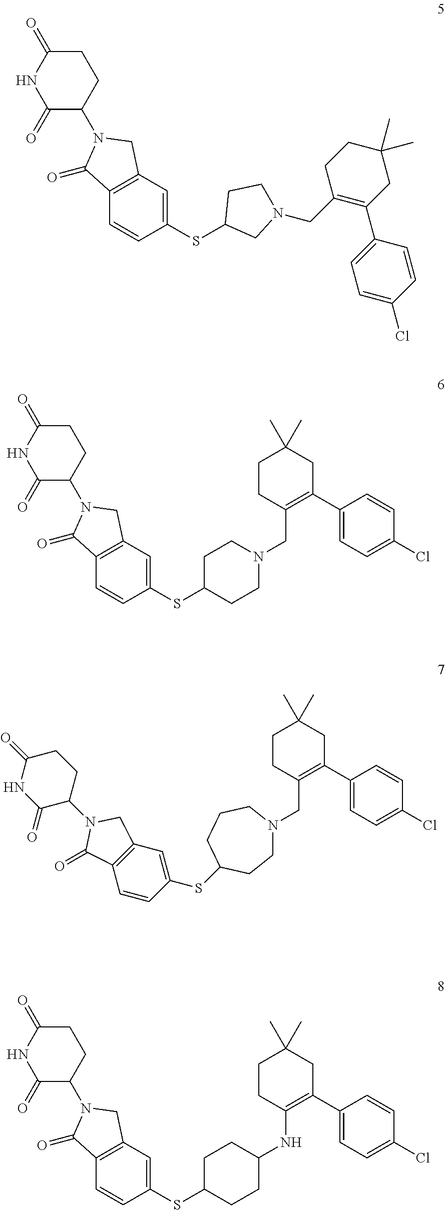 [SMILES: CC1(C)CCC(CN2CCC(Sc3ccc4c(c3)CN(C3CCC(=O)NC3=O)C4=O)C2)=C(c2ccc(Cl)cc2)C1 ; CC1(C)CCC(CN2CCC(Sc3ccc4c(c3)CN(C3CCC(=O)NC3=O)C4=O)CC2)=C(c2ccc(Cl)cc2)C1 ; CC1(C)CCC(CN2CCCC(Sc3ccc4c(c3)CN(C3CCC(=O)NC3=O)C4=O)CC2)=C(c2ccc(Cl)cc2)C1 ; CC1(C)CCC(NC2CCC(Sc3ccc4c(c3)CN(C3CCC(=O)NC3=O)C4=O)CC2)=C(c2ccc(Cl)cc2)C1]

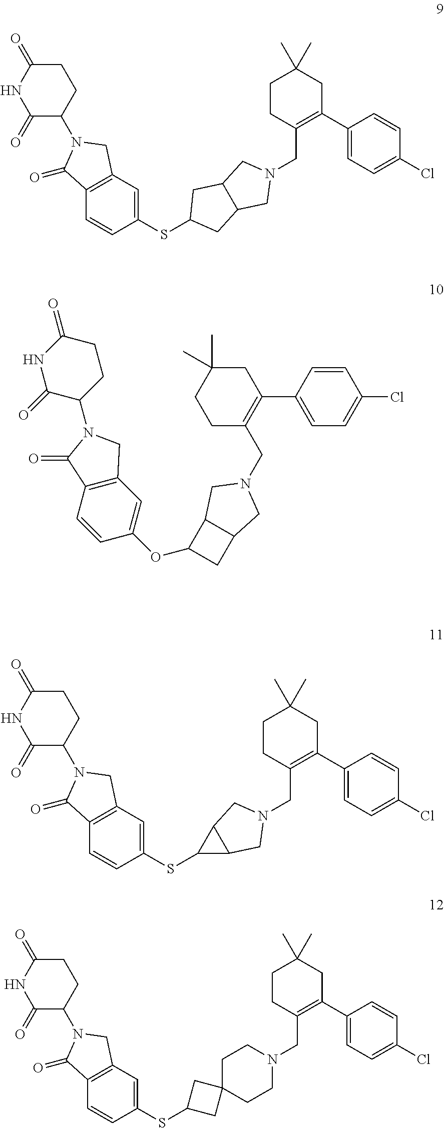 [SMILES: CC1(C)CCC(CN2CC3CC(Sc4ccc5c(c4)CN(C4CCC(=O)NC4=O)C5=O)CC3C2)=C(c2ccc(Cl)cc2)C1 ; CC1(C)CCC(CN2CC3CC(Oc4ccc5c(c4)CN(C4CCC(=O)NC4=O)C5=O)C3C2)=C(c2ccc(Cl)cc2)C1 ; CC1(C)CCC(CN2CC3C(C2)C3Sc2ccc3c(c2)CN(C2CCC(=O)NC2=O)C3=O)=C(c2ccc(Cl)cc2)C1 ; CC1(C)CCC(CN2CCC3(CC2)CC(Sc2ccc4c(c2)CN(C2CCC(=O)NC2=O)C4=O)C3)=C(c2ccc(Cl)cc2)C1]

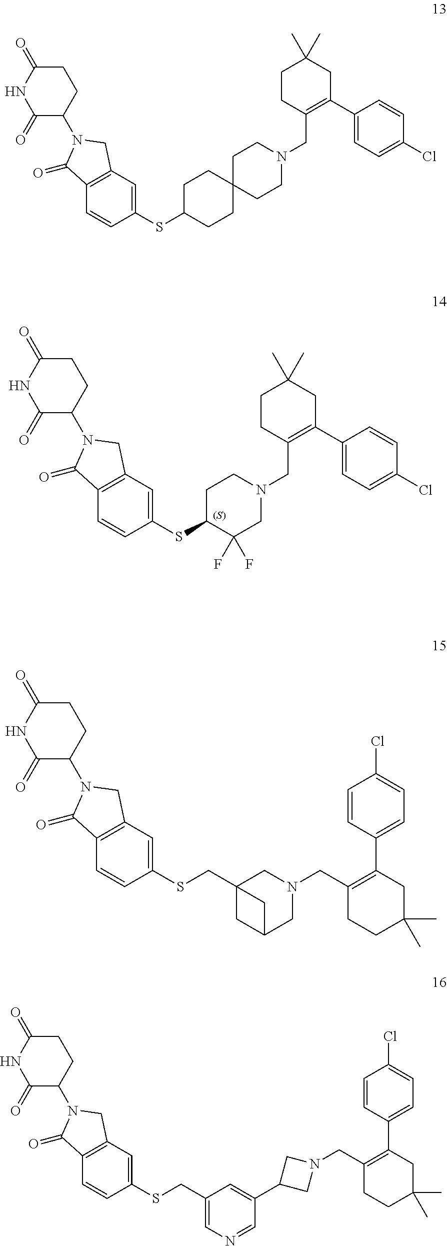

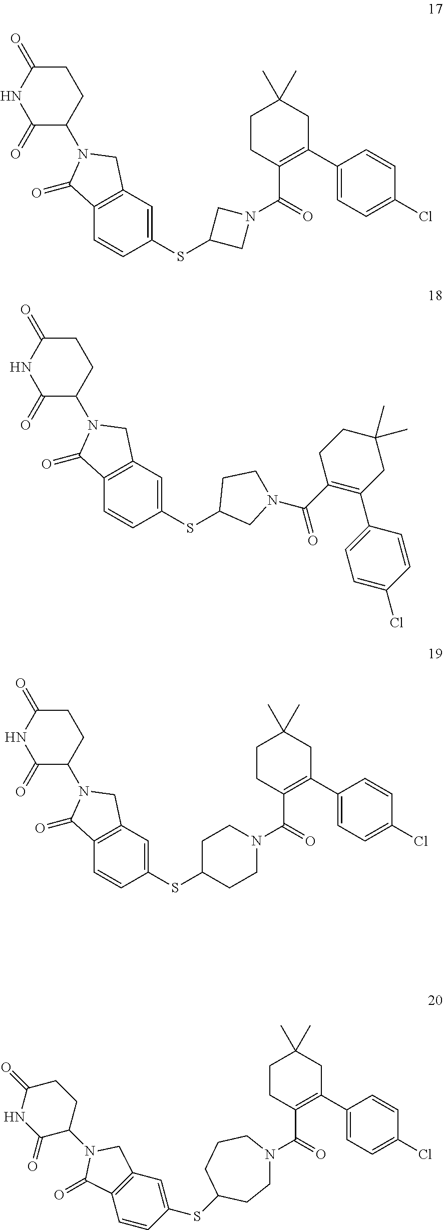 [SMILES: CC1(C)CCC(C(=O)N2CC(Sc3ccc4c(c3)CN(C3CCC(=O)NC3=O)C4=O)C2)=C(c2ccc(Cl)cc2)C1 ; CC1(C)CCC(C(=O)N2CCC(Sc3ccc4c(c3)CN(C3CCC(=O)NC3=O)C4=O)C2)=C(c2ccc(Cl)cc2)C1 ; CC1(C)CCC(C(=O)N2CCC(Sc3ccc4c(c3)CN(C3CCC(=O)NC3=O)C4=O)CC2)=C(c2ccc(Cl)cc2)C1 ; CC1(C)CCC(C(=O)N2CCCC(Sc3ccc4c(c3)CN(C3CCC(=O)NC3=O)C4=O)CC2)=C(c2ccc(Cl)cc2)C1]

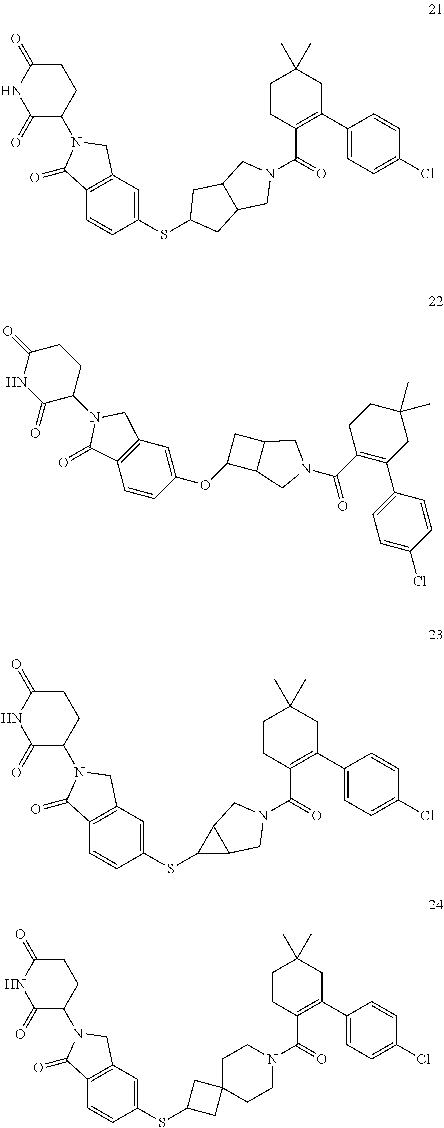 [SMILES: CC1(C)CCC(C(=O)N2CC3CC(Sc4ccc5c(c4)CN(C4CCC(=O)NC4=O)C5=O)CC3C2)=C(c2ccc(Cl)cc2)C1 ; CC1(C)CCC(C(=O)N2CC3CC(Oc4ccc5c(c4)CN(C4CCC(=O)NC4=O)C5=O)C3C2)=C(c2ccc(Cl)cc2)C1 ; CC1(C)CCC(C(=O)N2CC3C(C2)C3Sc2ccc3c(c2)CN(C2CCC(=O)NC2=O)C3=O)=C(c2ccc(Cl)cc2)C1 ; CC1(C)CCC(C(=O)N2CCC3(CC2)CC(Sc2ccc4c(c2)CN(C2CCC(=O)NC2=O)C4=O)C3)=C(c2ccc(Cl)cc2)C1]

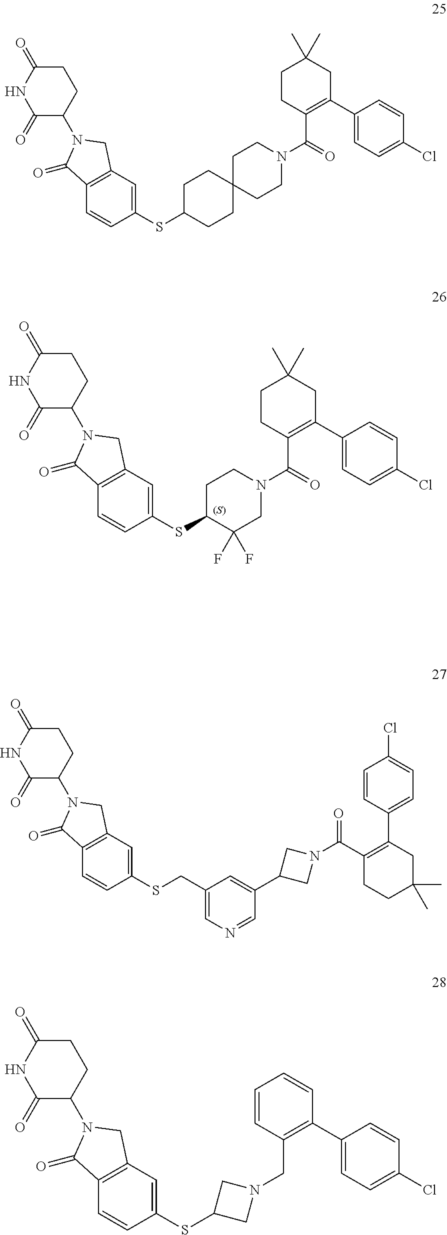

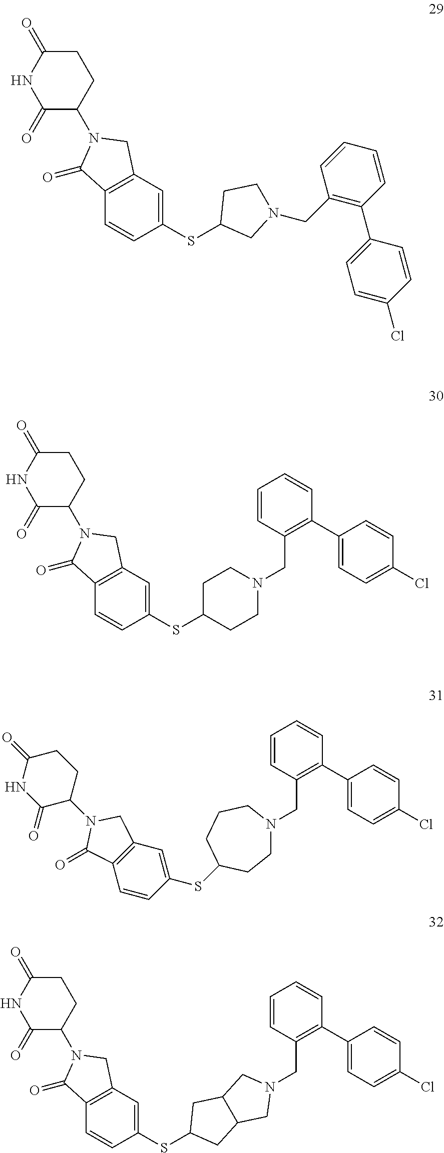 [SMILES: O=C1CCC(N2Cc3cc(SC4CCN(Cc5ccccc5-c5ccc(Cl)cc5)C4)ccc3C2=O)C(=O)N1 ; O=C1CCC(N2Cc3cc(SC4CCN(Cc5ccccc5-c5ccc(Cl)cc5)CC4)ccc3C2=O)C(=O)N1 ; O=C1CCC(N2Cc3cc(SC4CCCN(Cc5ccccc5-c5ccc(Cl)cc5)CC4)ccc3C2=O)C(=O)N1 ; O=C1CCC(N2Cc3cc(SC4CC5CN(Cc6ccccc6-c6ccc(Cl)cc6)CC5C4)ccc3C2=O)C(=O)N1]

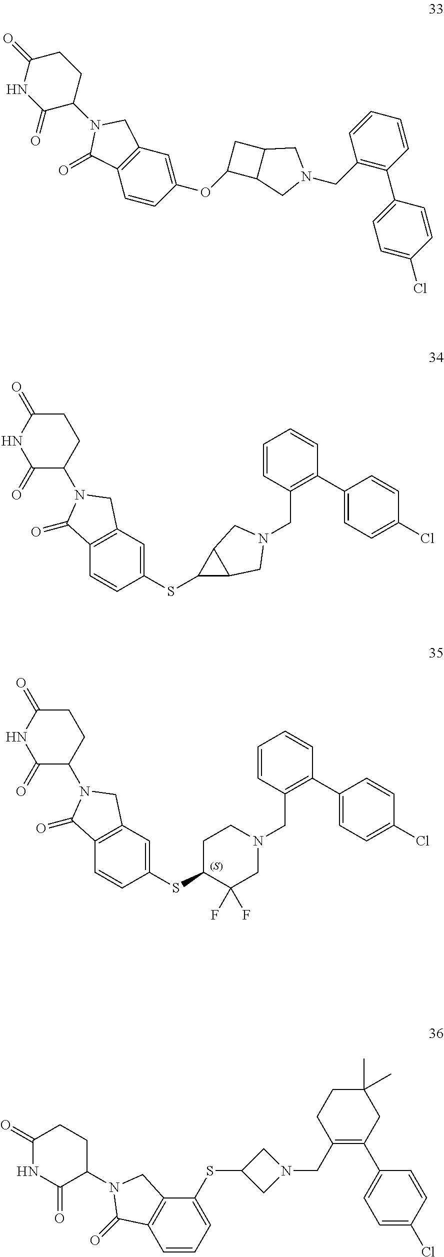

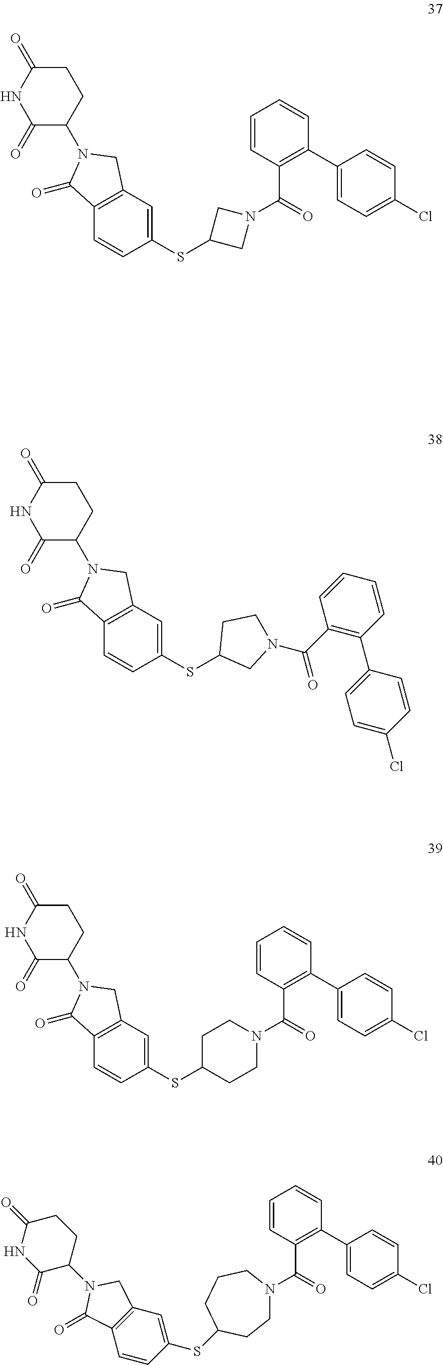 [SMILES: O=C1CCC(N2Cc3cc(SC4CN(C(=O)c5ccccc5-c5ccc(Cl)cc5)C4)ccc3C2=O)C(=O)N1 ; O=C1CCC(N2Cc3cc(SC4CCN(C(=O)c5ccccc5-c5ccc(Cl)cc5)C4)ccc3C2=O)C(=O)N1 ; O=C1CCC(N2Cc3cc(SC4CCN(C(=O)c5ccccc5-c5ccc(Cl)cc5)CC4)ccc3C2=O)C(=O)N1 ; O=C1CCC(N2Cc3cc(SC4CCCN(C(=O)c5ccccc5-c5ccc(Cl)cc5)CC4)ccc3C2=O)C(=O)N1]

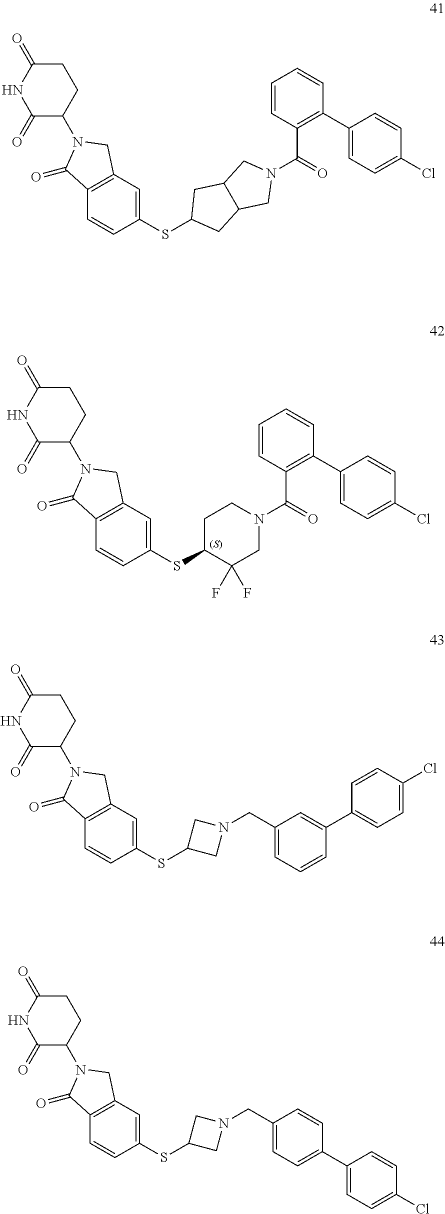

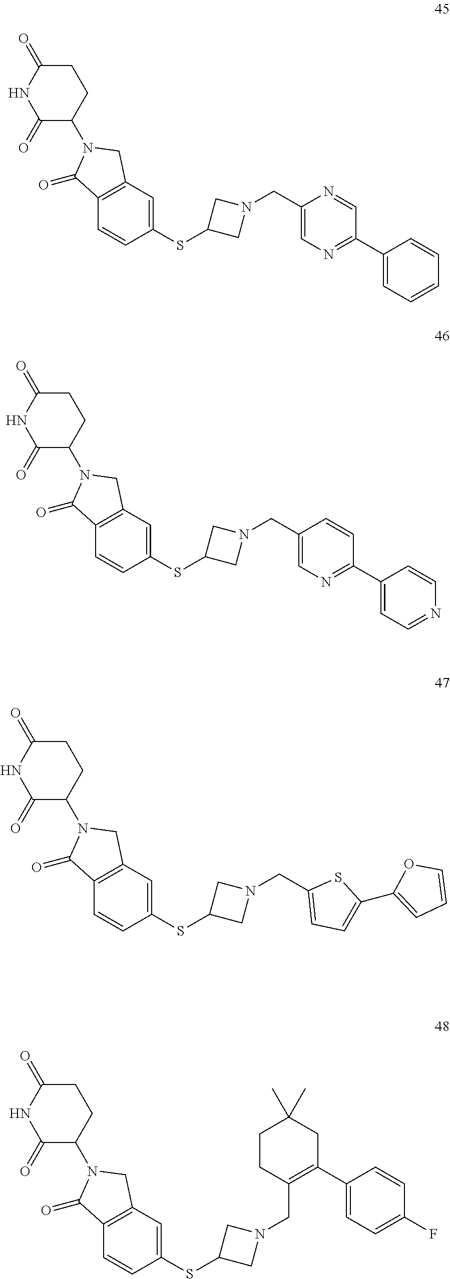 [SMILES: O=C1CCC(N2Cc3cc(SC4CN(Cc5cnc(-c6ccccc6)cn5)C4)ccc3C2=O)C(=O)N1 ; O=C1CCC(N2Cc3cc(SC4CN(Cc5ccc(-c6ccncc6)nc5)C4)ccc3C2=O)C(=O)N1 ; O=C1CCC(N2Cc3cc(SC4CN(Cc5ccc(-c6ccco6)s5)C4)ccc3C2=O)C(=O)N1 ; CC1(C)CCC(CN2CC(Sc3ccc4c(c3)CN(C3CCC(=O)NC3=O)C4=O)C2)=C(c2ccc(F)cc2)C1]

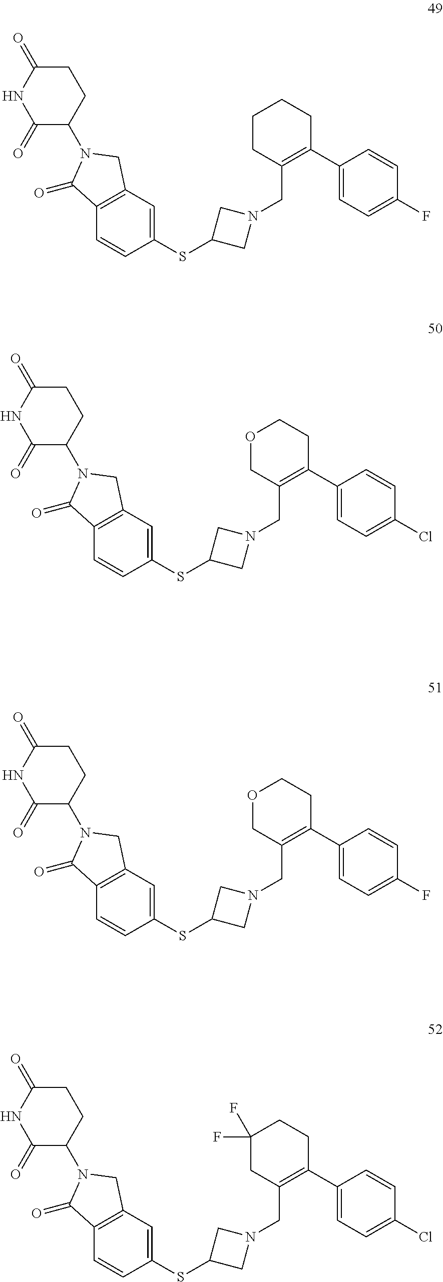 [SMILES: O=C1CCC(N2Cc3cc(SC4CN(CC5=C(c6ccc(F)cc6)CCCC5)C4)ccc3C2=O)C(=O)N1 ; O=C1CCC(N2Cc3cc(SC4CN(CC5=C(c6ccc(Cl)cc6)CCOC5)C4)ccc3C2=O)C(=O)N1 ; O=C1CCC(N2Cc3cc(SC4CN(CC5=C(c6ccc(F)cc6)CCOC5)C4)ccc3C2=O)C(=O)N1 ; O=C1CCC(N2Cc3cc(SC4CN(CC5=C(c6ccc(Cl)cc6)CCC(F)(F)C5)C4)ccc3C2=O)C(=O)N1]

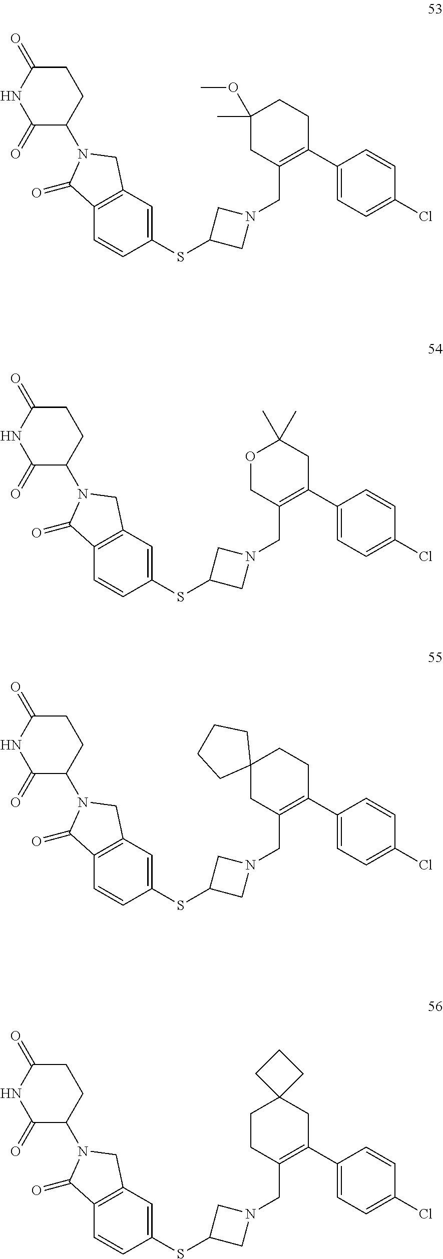 [SMILES: COC1(C)CCC(c2ccc(Cl)cc2)=C(CN2CC(Sc3ccc4c(c3)CN(C3CCC(=O)NC3=O)C4=O)C2)C1 ; CC1(C)CC(c2ccc(Cl)cc2)=C(CN2CC(Sc3ccc4c(c3)CN(C3CCC(=O)NC3=O)C4=O)C2)CO1 ; O=C1CCC(N2Cc3cc(SC4CN(CC5=C(c6ccc(Cl)cc6)CCC6(CCCC6)C5)C4)ccc3C2=O)C(=O)N1 ; O=C1CCC(N2Cc3cc(SC4CN(CC5=C(c6ccc(Cl)cc6)CC6(CCC6)CC5)C4)ccc3C2=O)C(=O)N1]

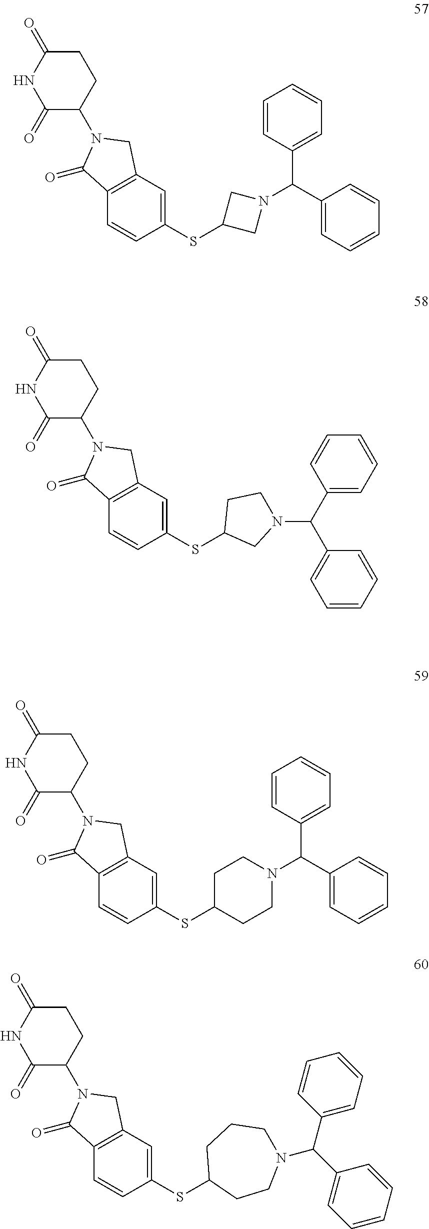 [SMILES: O=C1CCC(N2Cc3cc(SC4CN(C(c5ccccc5)c5ccccc5)C4)ccc3C2=O)C(=O)N1 ; O=C1CCC(N2Cc3cc(SC4CCN(C(c5ccccc5)c5ccccc5)C4)ccc3C2=O)C(=O)N1 ; O=C1CCC(N2Cc3cc(SC4CCN(C(c5ccccc5)c5ccccc5)CC4)ccc3C2=O)C(=O)N1 ; O=C1CCC(N2Cc3cc(SC4CCCN(C(c5ccccc5)c5ccccc5)CC4)ccc3C2=O)C(=O)N1]

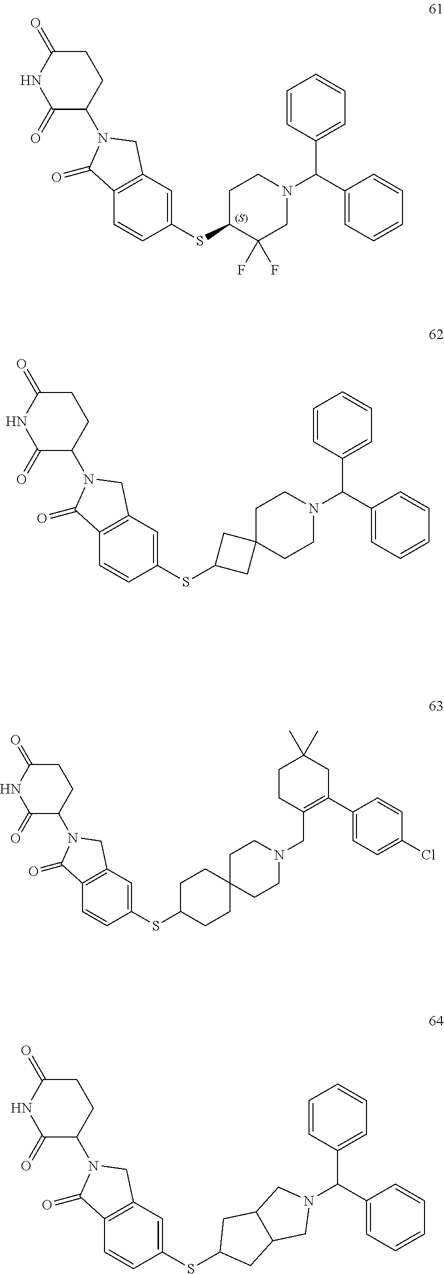

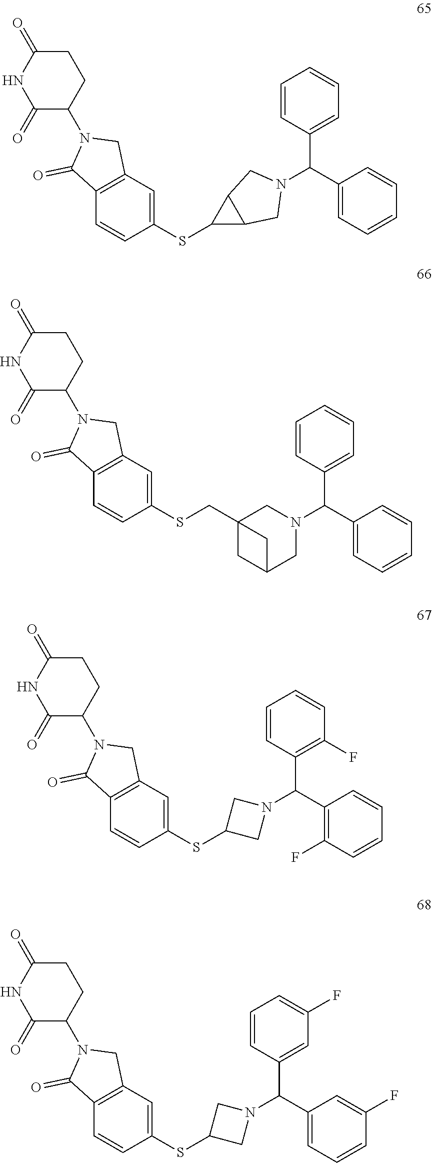 [SMILES: O=C1CCC(N2Cc3cc(SC4C5CN(C(c6ccccc6)c6ccccc6)CC54)ccc3C2=O)C(=O)N1 ; O=C1CCC(N2Cc3cc(SCC45CC(CN(C(c6ccccc6)c6ccccc6)C4)C5)ccc3C2=O)C(=O)N1 ; O=C1CCC(N2Cc3cc(SC4CN(C(c5ccccc5F)c5ccccc5F)C4)ccc3C2=O)C(=O)N1 ; O=C1CCC(N2Cc3cc(SC4CN(C(c5cccc(F)c5)c5cccc(F)c5)C4)ccc3C2=O)C(=O)N1]

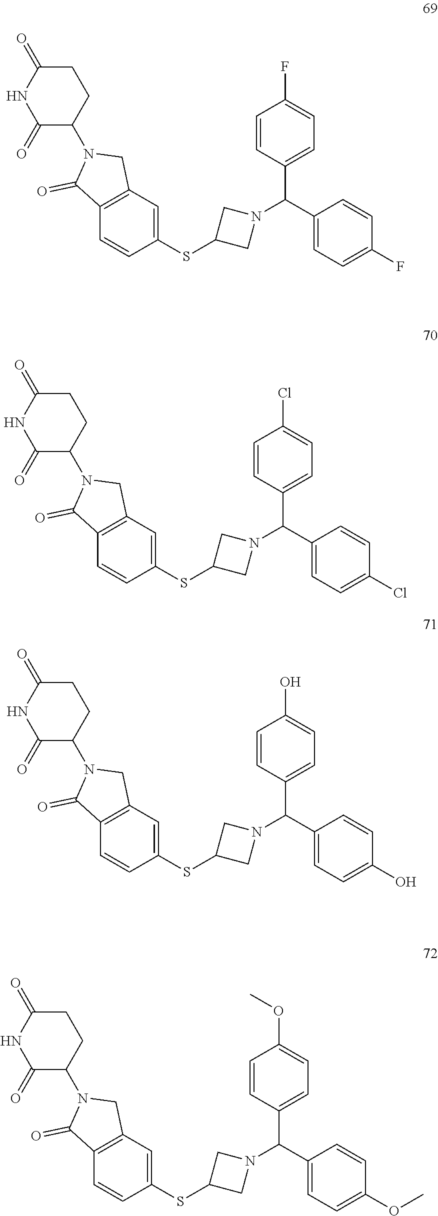 [SMILES: O=C1CCC(N2Cc3cc(SC4CN(C(c5ccc(F)cc5)c5ccc(F)cc5)C4)ccc3C2=O)C(=O)N1 ; O=C1CCC(N2Cc3cc(SC4CN(C(c5ccc(Cl)cc5)c5ccc(Cl)cc5)C4)ccc3C2=O)C(=O)N1 ; O=C1CCC(N2Cc3cc(SC4CN(C(c5ccc(O)cc5)c5ccc(O)cc5)C4)ccc3C2=O)C(=O)N1 ; COc1ccc(C(c2ccc(OC)cc2)N2CC(Sc3ccc4c(c3)CN(C3CCC(=O)NC3=O)C4=O)C2)cc1]

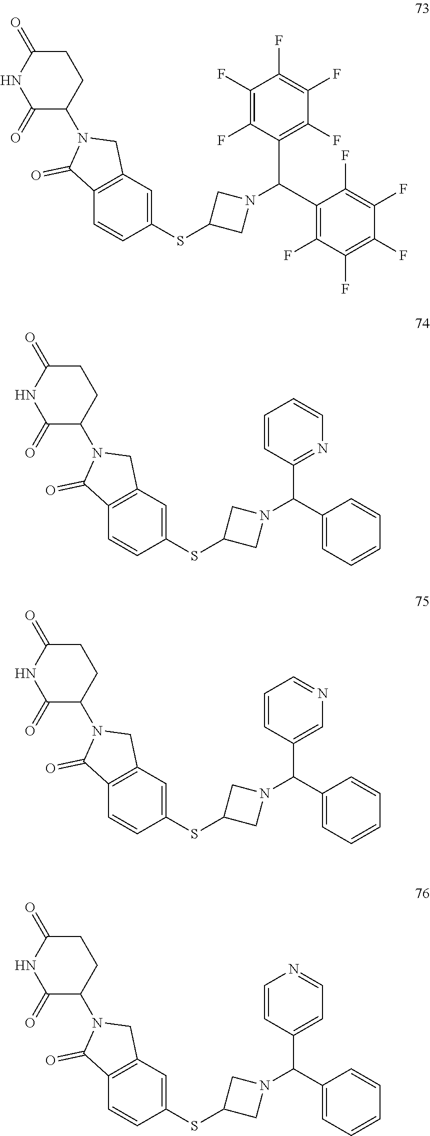 [SMILES: O=C1CCC(N2Cc3cc(SC4CN(C(c5c(F)c(F)c(F)c(F)c5F)c5c(F)c(F)c(F)c(F)c5F)C4)ccc3C2=O)C(=O)N1 ; O=C1CCC(N2Cc3cc(SC4CN(C(c5ccccc5)c5ccccn5)C4)ccc3C2=O)C(=O)N1 ; O=C1CCC(N2Cc3cc(SC4CN(C(c5ccccc5)c5cccnc5)C4)ccc3C2=O)C(=O)N1 ; O=C1CCC(N2Cc3cc(SC4CN(C(c5ccccc5)c5ccncc5)C4)ccc3C2=O)C(=O)N1]

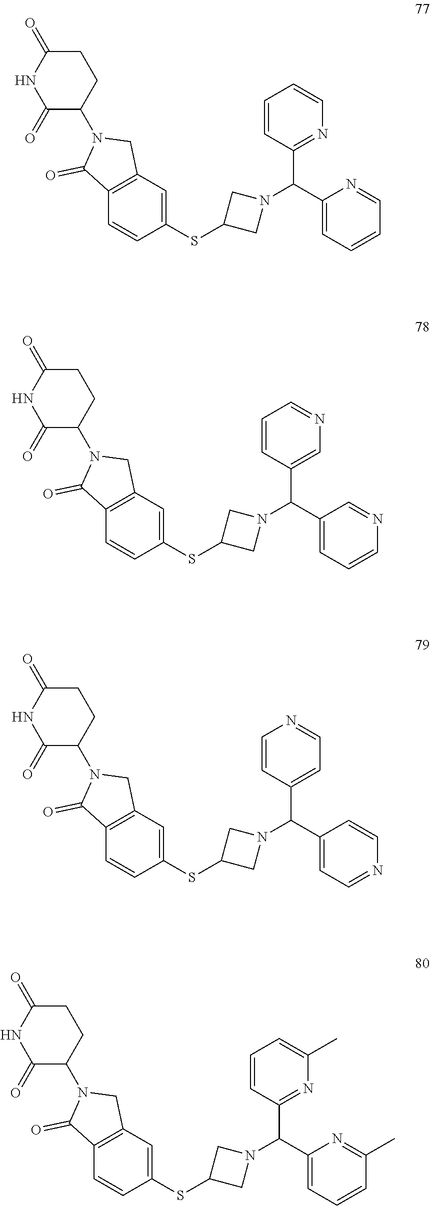 [SMILES: O=C1CCC(N2Cc3cc(SC4CN(C(c5ccccn5)c5ccccn5)C4)ccc3C2=O)C(=O)N1 ; O=C1CCC(N2Cc3cc(SC4CN(C(c5cccnc5)c5cccnc5)C4)ccc3C2=O)C(=O)N1 ; O=C1CCC(N2Cc3cc(SC4CN(C(c5ccncc5)c5ccncc5)C4)ccc3C2=O)C(=O)N1 ; Cc1cccc(C(c2cccc(C)n2)N2CC(Sc3ccc4c(c3)CN(C3CCC(=O)NC3=O)C4=O)C2)n1]

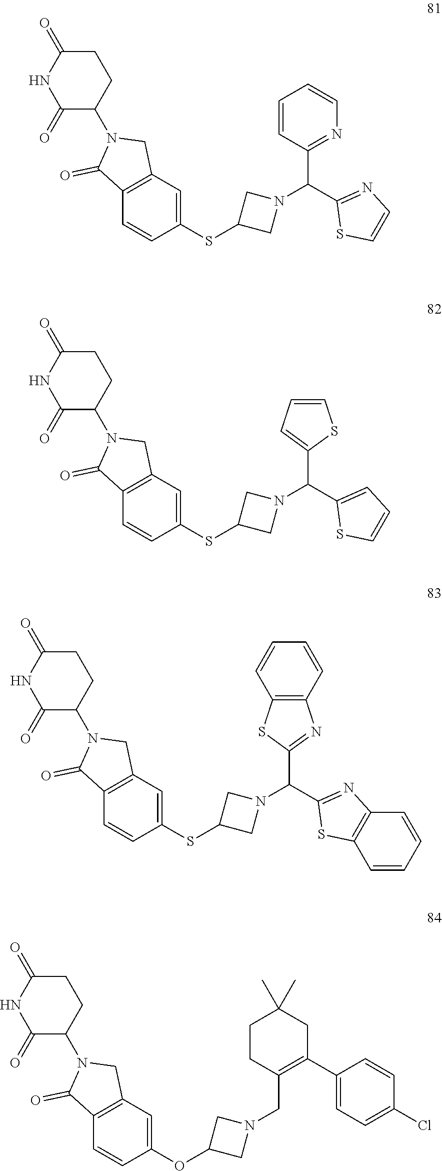 [SMILES: O=C1CCC(N2Cc3cc(SC4CN(C(c5ccccn5)c5nccs5)C4)ccc3C2=O)C(=O)N1 ; O=C1CCC(N2Cc3cc(SC4CN(C(c5cccs5)c5cccs5)C4)ccc3C2=O)C(=O)N1 ; O=C1CCC(N2Cc3cc(SC4CN(C(c5nc6ccccc6s5)c5nc6ccccc6s5)C4)ccc3C2=O)C(=O)N1 ; CC1(C)CCC(CN2CC(Oc3ccc4c(c3)CN(C3CCC(=O)NC3=O)C4=O)C2)=C(c2ccc(Cl)cc2)C1]

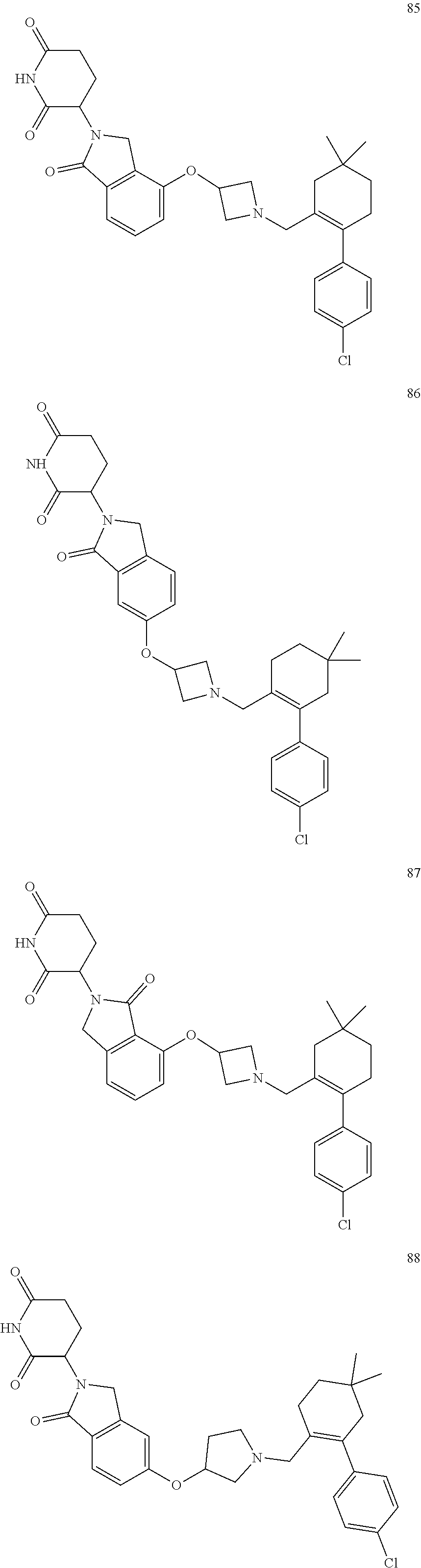 [SMILES: CC1(C)CCC(c2ccc(Cl)cc2)=C(CN2CC(Oc3cccc4c3CN(C3CCC(=O)NC3=O)C4=O)C2)C1 ; CC1(C)CCC(CN2CC(Oc3ccc4c(c3)C(=O)N(C3CCC(=O)NC3=O)C4)C2)=C(c2ccc(Cl)cc2)C1 ; CC1(C)CCC(c2ccc(Cl)cc2)=C(CN2CC(Oc3cccc4c3C(=O)N(C3CCC(=O)NC3=O)C4)C2)C1 ; CC1(C)CCC(CN2CCC(Oc3ccc4c(c3)CN(C3CCC(=O)NC3=O)C4=O)C2)=C(c2ccc(Cl)cc2)C1]

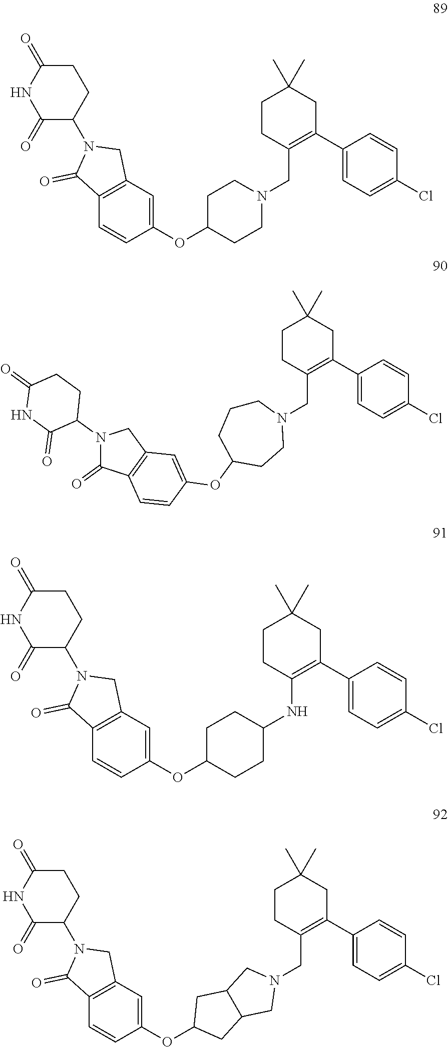 [SMILES: CC1(C)CCC(CN2CCC(Oc3ccc4c(c3)CN(C3CCC(=O)NC3=O)C4=O)CC2)=C(c2ccc(Cl)cc2)C1 ; CC1(C)CCC(CN2CCCC(Oc3ccc4c(c3)CN(C3CCC(=O)NC3=O)C4=O)CC2)=C(c2ccc(Cl)cc2)C1 ; CC1(C)CCC(NC2CCC(Oc3ccc4c(c3)CN(C3CCC(=O)NC3=O)C4=O)CC2)=C(c2ccc(Cl)cc2)C1 ; CC1(C)CCC(CN2CC3CC(Oc4ccc5c(c4)CN(C4CCC(=O)NC4=O)C5=O)CC3C2)=C(c2ccc(Cl)cc2)C1]

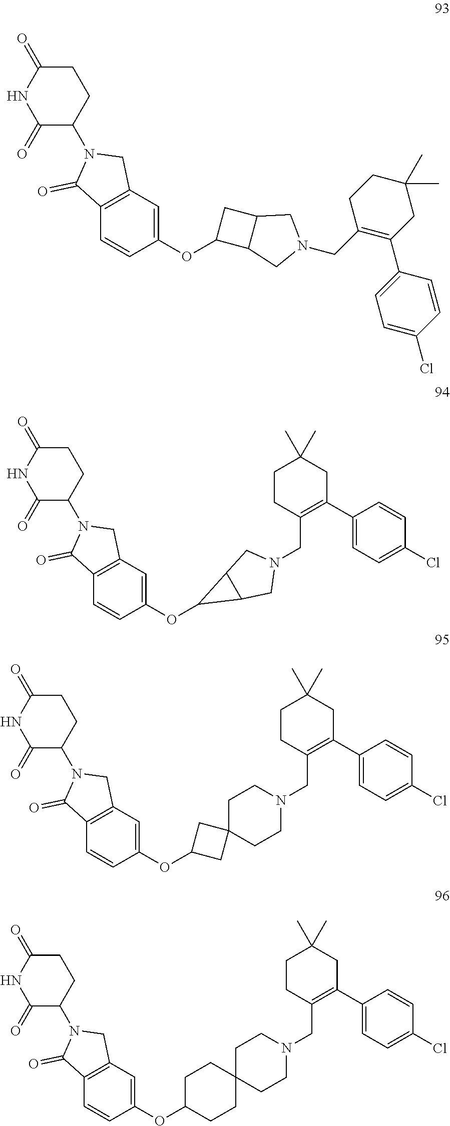 [SMILES: CC1(C)CCC(CN2CC3CC(Oc4ccc5c(c4)CN(C4CCC(=O)NC4=O)C5=O)C3C2)=C(c2ccc(Cl)cc2)C1 ; CC1(C)CCC(CN2CC3C(C2)C3Oc2ccc3c(c2)CN(C2CCC(=O)NC2=O)C3=O)=C(c2ccc(Cl)cc2)C1 ; CC1(C)CCC(CN2CCC3(CC2)CC(Oc2ccc4c(c2)CN(C2CCC(=O)NC2=O)C4=O)C3)=C(c2ccc(Cl)cc2)C1 ; CC1(C)CCC(CN2CCC3(CCC(Oc4ccc5c(c4)CN(C4CCC(=O)NC4=O)C5=O)CC3)CC2)=C(c2ccc(Cl)cc2)C1]

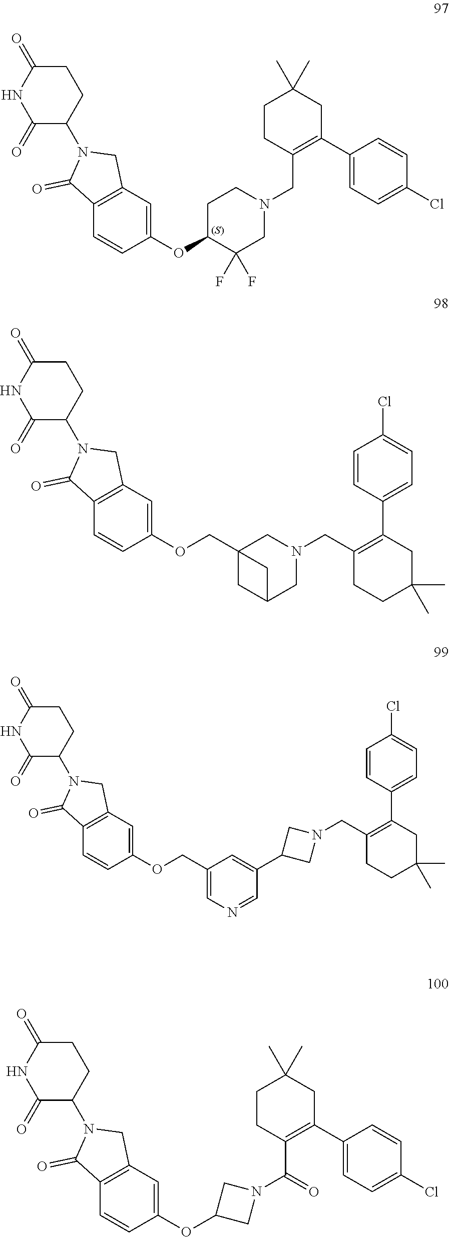

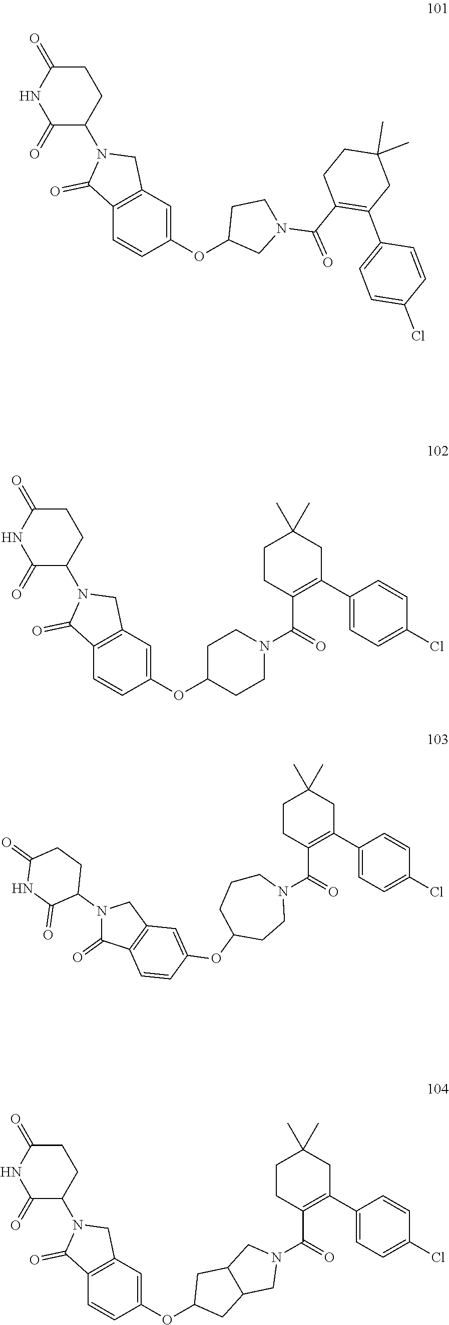 [SMILES: CC1(C)CCC(C(=O)N2CCC(Oc3ccc4c(c3)CN(C3CCC(=O)NC3=O)C4=O)C2)=C(c2ccc(Cl)cc2)C1 ; CC1(C)CCC(C(=O)N2CCC(Oc3ccc4c(c3)CN(C3CCC(=O)NC3=O)C4=O)CC2)=C(c2ccc(Cl)cc2)C1 ; CC1(C)CCC(C(=O)N2CCCC(Oc3ccc4c(c3)CN(C3CCC(=O)NC3=O)C4=O)CC2)=C(c2ccc(Cl)cc2)C1 ; CC1(C)CCC(C(=O)N2CC3CC(Oc4ccc5c(c4)CN(C4CCC(=O)NC4=O)C5=O)CC3C2)=C(c2ccc(Cl)cc2)C1]

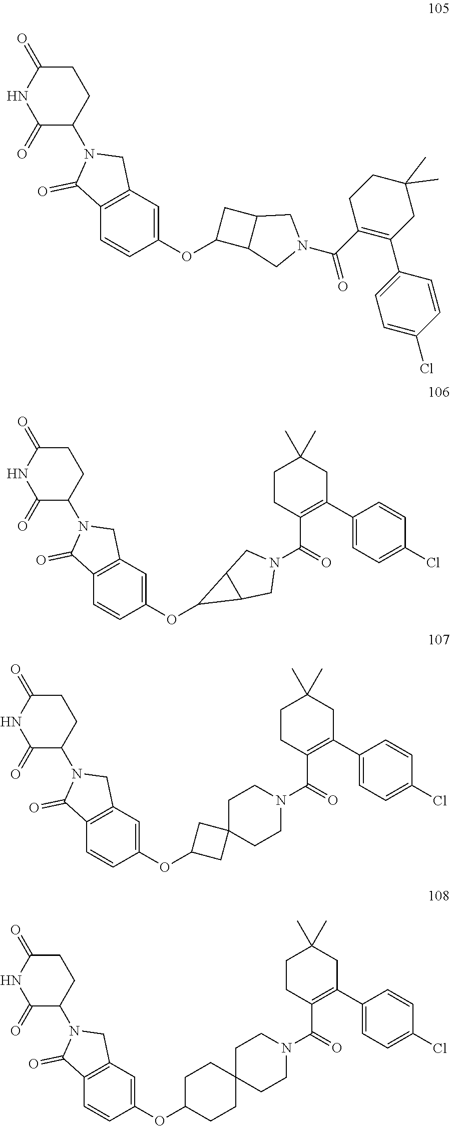 [SMILES: CC1(C)CCC(C(=O)N2CC3CC(Oc4ccc5c(c4)CN(C4CCC(=O)NC4=O)C5=O)C3C2)=C(c2ccc(Cl)cc2)C1 ; CC1(C)CCC(C(=O)N2CC3C(C2)C3Oc2ccc3c(c2)CN(C2CCC(=O)NC2=O)C3=O)=C(c2ccc(Cl)cc2)C1 ; CC1(C)CCC(C(=O)N2CCC3(CC2)CC(Oc2ccc4c(c2)CN(C2CCC(=O)NC2=O)C4=O)C3)=C(c2ccc(Cl)cc2)C1 ; CC1(C)CCC(C(=O)N2CCC3(CCC(Oc4ccc5c(c4)CN(C4CCC(=O)NC4=O)C5=O)CC3)CC2)=C(c2ccc(Cl)cc2)C1]

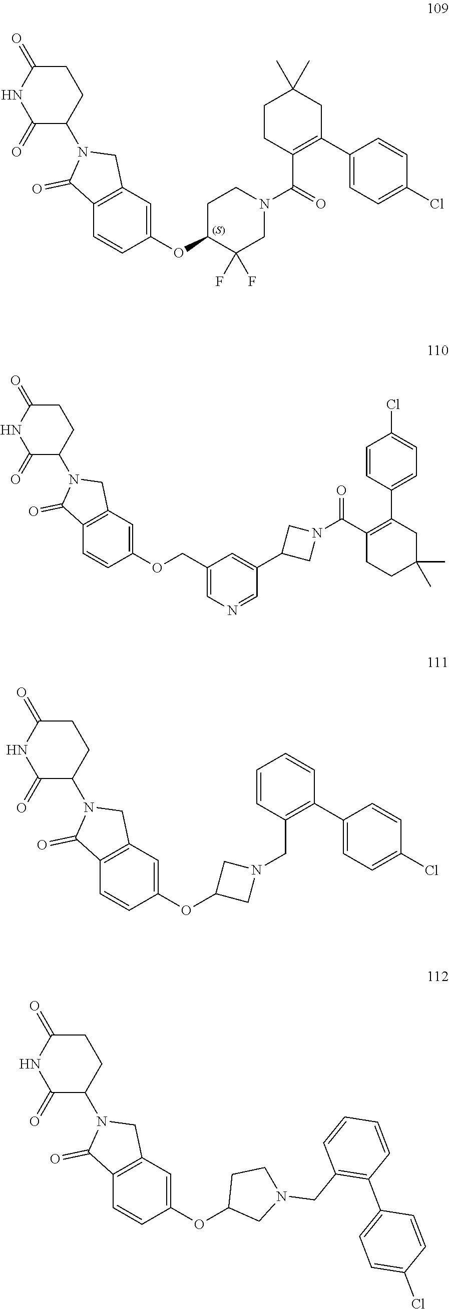

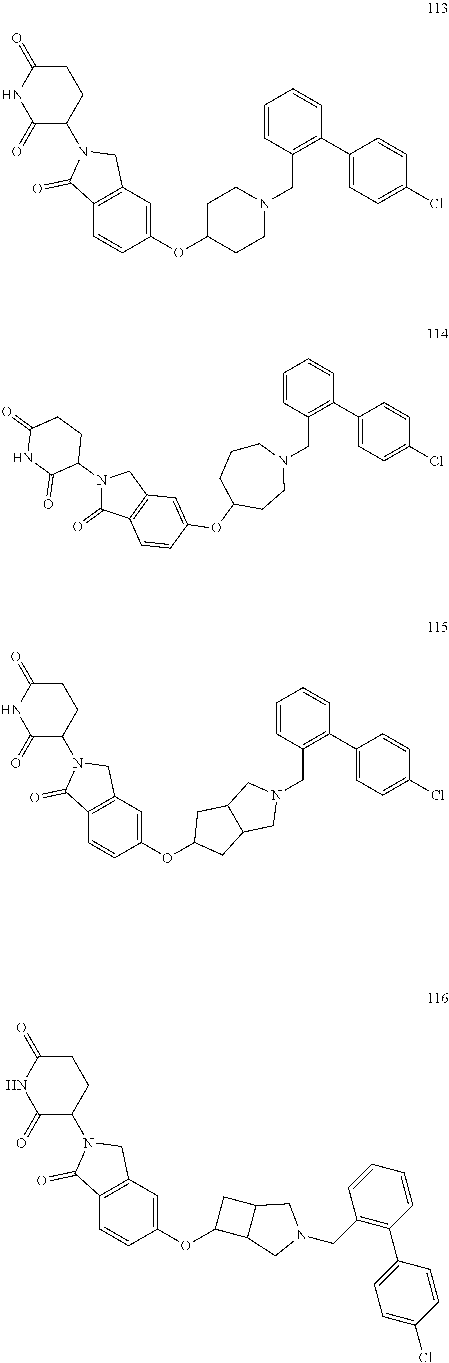 [SMILES: O=C1CCC(N2Cc3cc(OC4CCN(Cc5ccccc5-c5ccc(Cl)cc5)CC4)ccc3C2=O)C(=O)N1 ; O=C1CCC(N2Cc3cc(OC4CCCN(Cc5ccccc5-c5ccc(Cl)cc5)CC4)ccc3C2=O)C(=O)N1 ; O=C1CCC(N2Cc3cc(OC4CC5CN(Cc6ccccc6-c6ccc(Cl)cc6)CC5C4)ccc3C2=O)C(=O)N1 ; O=C1CCC(N2Cc3cc(OC4CC5CN(Cc6ccccc6-c6ccc(Cl)cc6)CC54)ccc3C2=O)C(=O)N1]

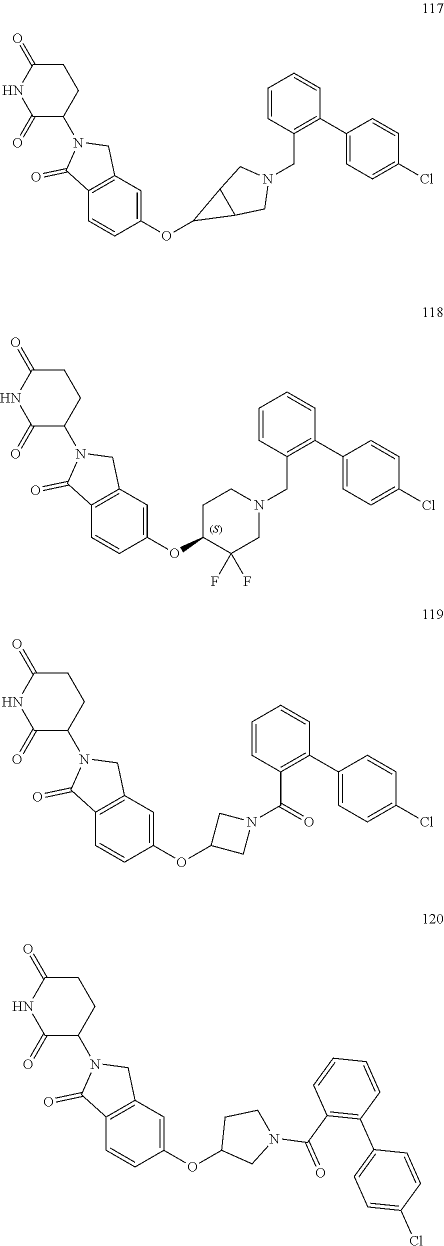

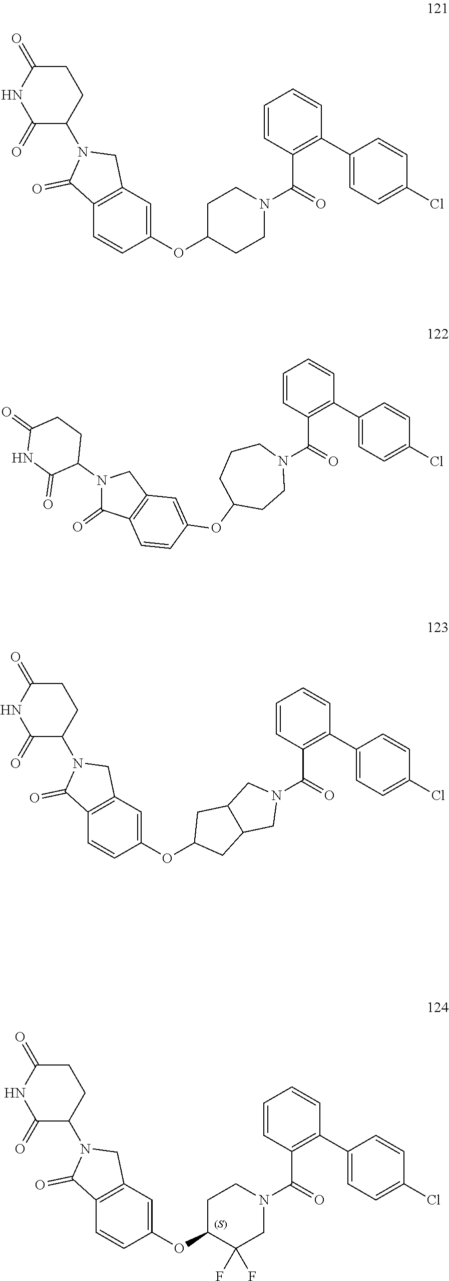

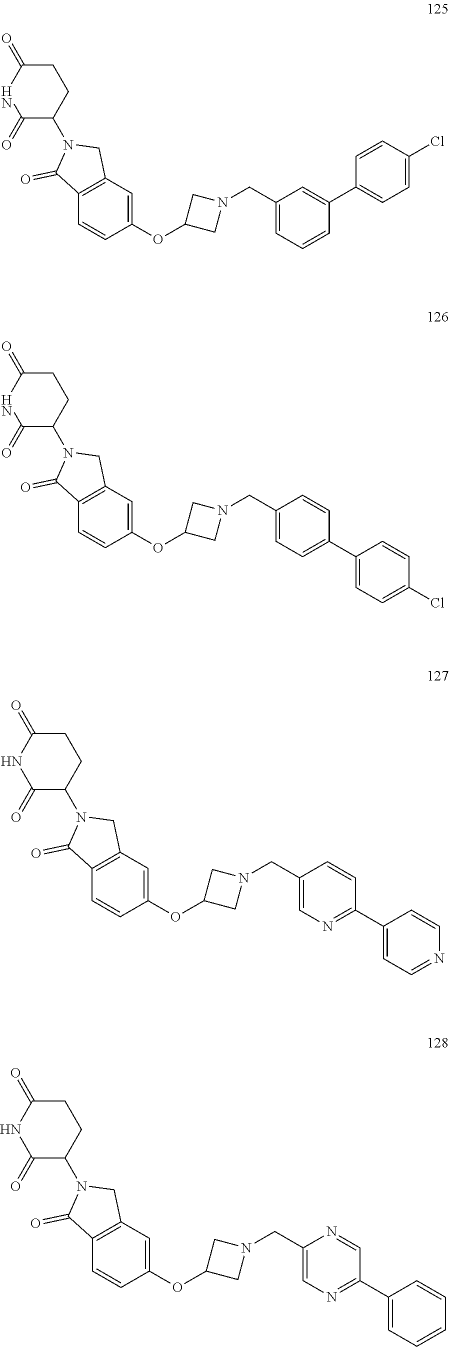 [SMILES: O=C1CCC(N2Cc3cc(OC4CN(Cc5cccc(-c6ccc(Cl)cc6)c5)C4)ccc3C2=O)C(=O)N1 ; O=C1CCC(N2Cc3cc(OC4CN(Cc5ccc(-c6ccc(Cl)cc6)cc5)C4)ccc3C2=O)C(=O)N1 ; O=C1CCC(N2Cc3cc(OC4CN(Cc5ccc(-c6ccncc6)nc5)C4)ccc3C2=O)C(=O)N1 ; O=C1CCC(N2Cc3cc(OC4CN(Cc5cnc(-c6ccccc6)cn5)C4)ccc3C2=O)C(=O)N1]

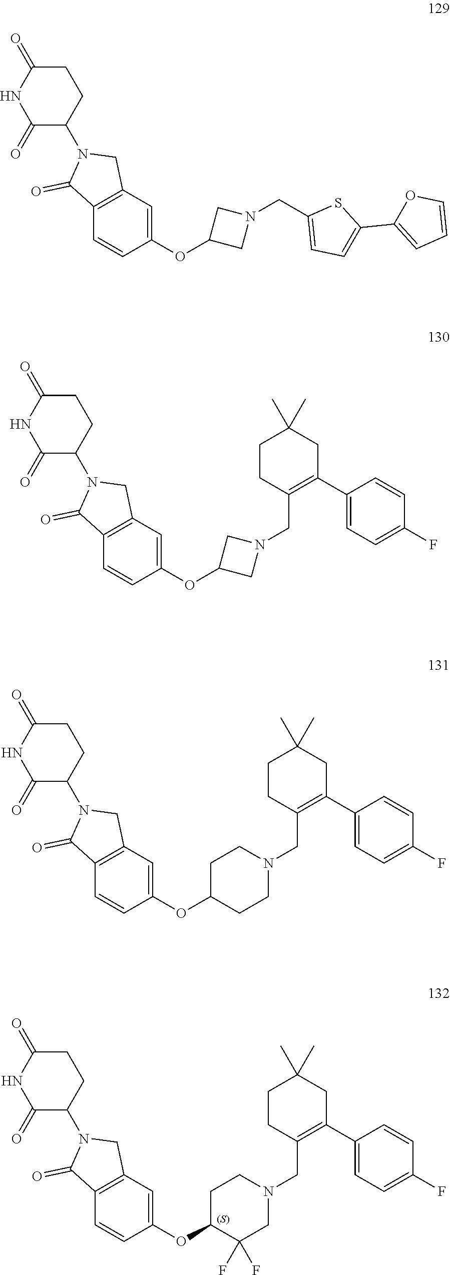

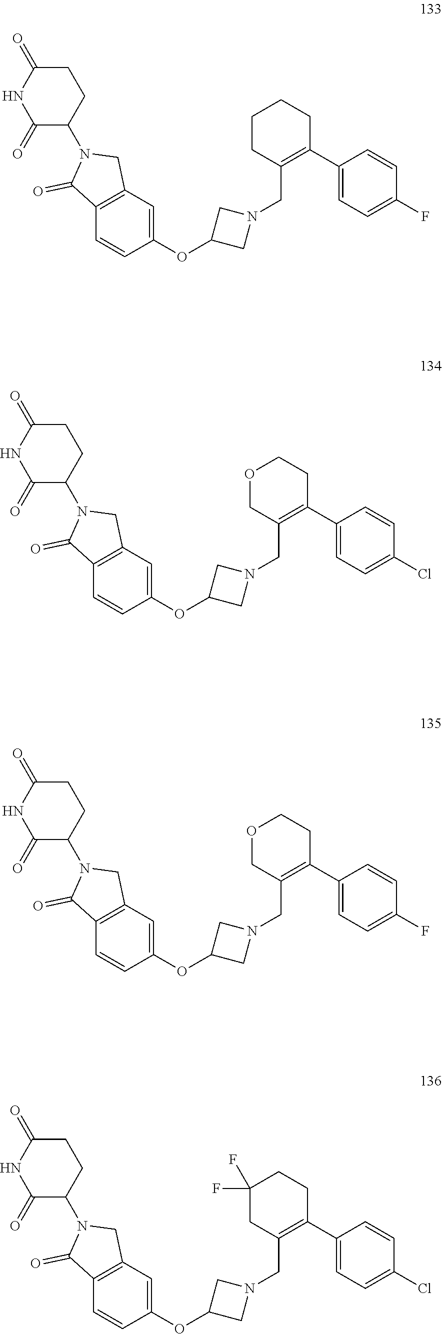 [SMILES: O=C1CCC(N2Cc3cc(OC4CN(CC5=C(c6ccc(F)cc6)CCCC5)C4)ccc3C2=O)C(=O)N1 ; O=C1CCC(N2Cc3cc(OC4CN(CC5=C(c6ccc(Cl)cc6)CCOC5)C4)ccc3C2=O)C(=O)N1 ; O=C1CCC(N2Cc3cc(OC4CN(CC5=C(c6ccc(F)cc6)CCOC5)C4)ccc3C2=O)C(=O)N1 ; O=C1CCC(N2Cc3cc(OC4CN(CC5=C(c6ccc(Cl)cc6)CCC(F)(F)C5)C4)ccc3C2=O)C(=O)N1]

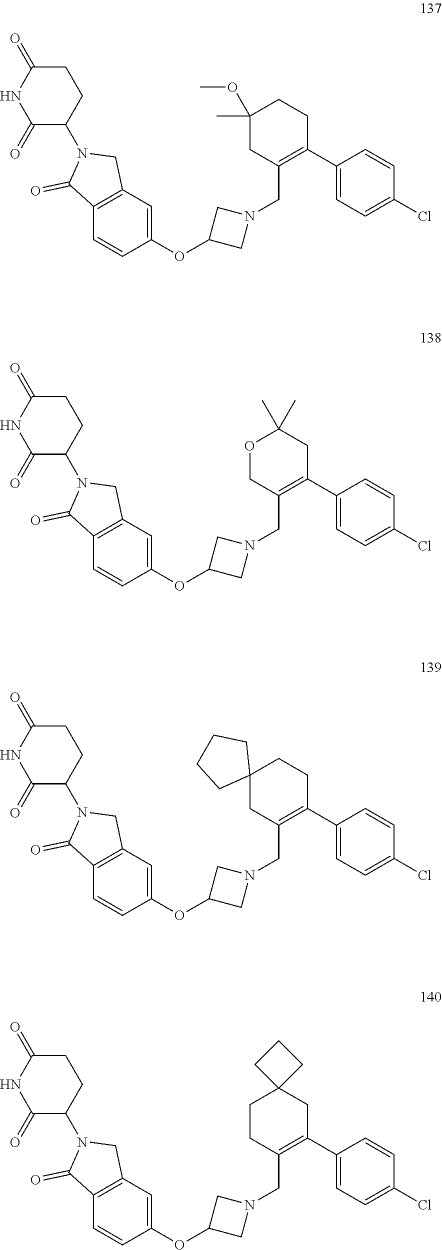 [SMILES: COC1(C)CCC(c2ccc(Cl)cc2)=C(CN2CC(Oc3ccc4c(c3)CN(C3CCC(=O)NC3=O)C4=O)C2)C1 ; CC1(C)CC(c2ccc(Cl)cc2)=C(CN2CC(Oc3ccc4c(c3)CN(C3CCC(=O)NC3=O)C4=O)C2)CO1 ; O=C1CCC(N2Cc3cc(OC4CN(CC5=C(c6ccc(Cl)cc6)CCC6(CCCC6)C5)C4)ccc3C2=O)C(=O)N1 ; O=C1CCC(N2Cc3cc(OC4CN(CC5=C(c6ccc(Cl)cc6)CC6(CCC6)CC5)C4)ccc3C2=O)C(=O)N1]

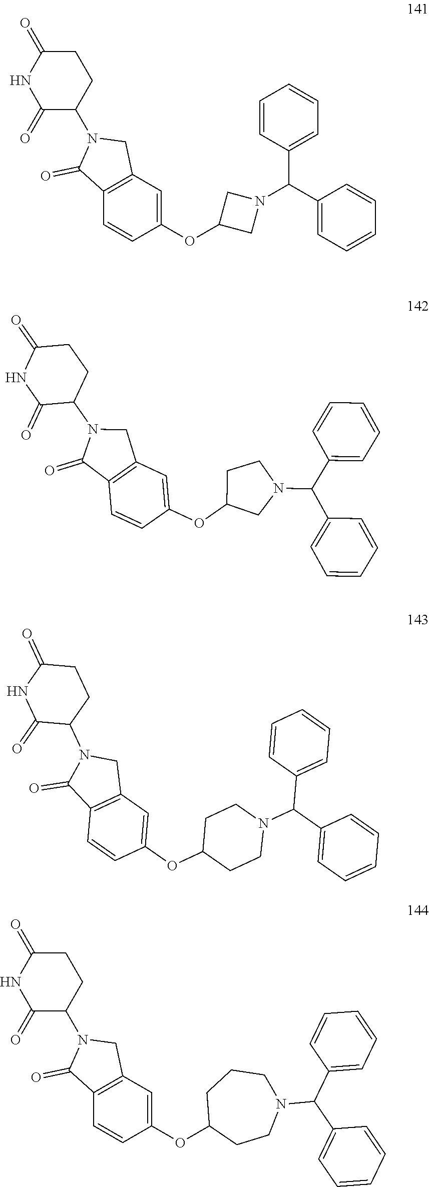 [SMILES: O=C1CCC(N2Cc3cc(OC4CN(C(c5ccccc5)c5ccccc5)C4)ccc3C2=O)C(=O)N1 ; O=C1CCC(N2Cc3cc(OC4CCN(C(c5ccccc5)c5ccccc5)C4)ccc3C2=O)C(=O)N1 ; O=C1CCC(N2Cc3cc(OC4CCN(C(c5ccccc5)c5ccccc5)CC4)ccc3C2=O)C(=O)N1 ; O=C1CCC(N2Cc3cc(OC4CCCN(C(c5ccccc5)c5ccccc5)CC4)ccc3C2=O)C(=O)N1]

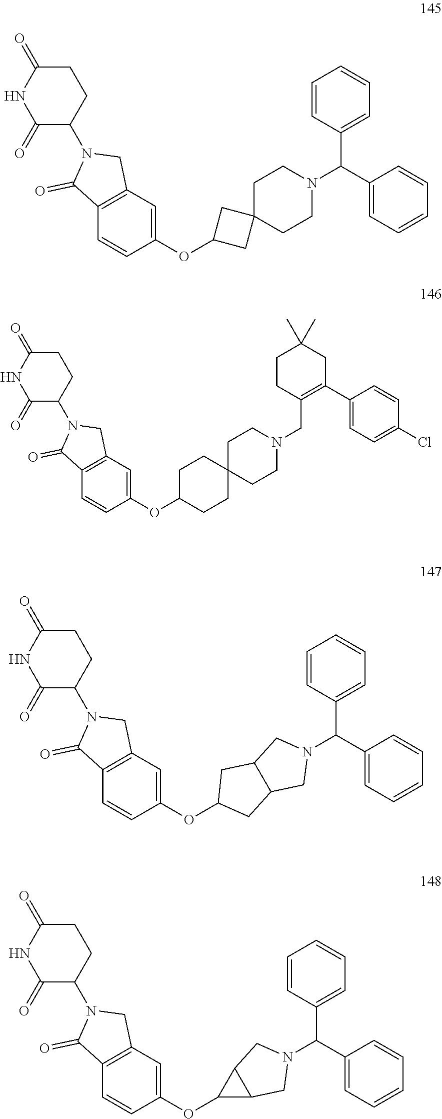 [SMILES: O=C1CCC(N2Cc3cc(OC4CC5(CCN(C(c6ccccc6)c6ccccc6)CC5)C4)ccc3C2=O)C(=O)N1 ; CC1(C)CCC(CN2CCC3(CCC(Oc4ccc5c(c4)CN(C4CCC(=O)NC4=O)C5=O)CC3)CC2)=C(c2ccc(Cl)cc2)C1 ; O=C1CCC(N2Cc3cc(OC4CC5CN(C(c6ccccc6)c6ccccc6)CC5C4)ccc3C2=O)C(=O)N1 ; O=C1CCC(N2Cc3cc(OC4C5CN(C(c6ccccc6)c6ccccc6)CC54)ccc3C2=O)C(=O)N1]

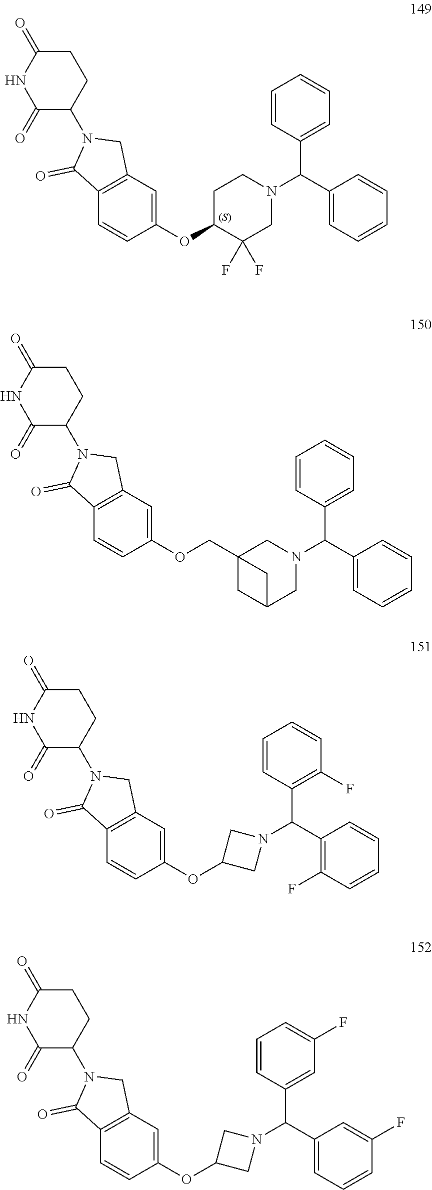

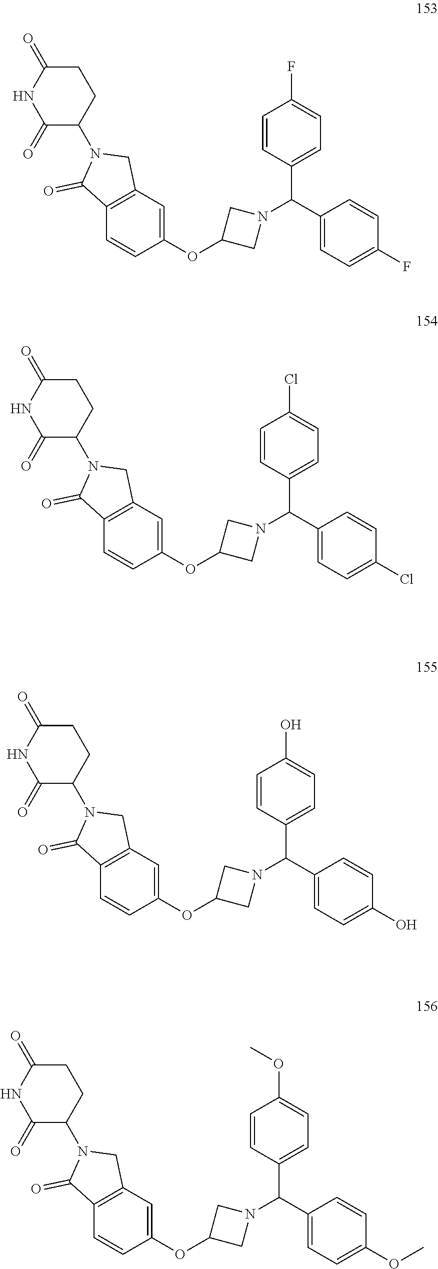 [SMILES: O=C1CCC(N2Cc3cc(OC4CN(C(c5ccc(F)cc5)c5ccc(F)cc5)C4)ccc3C2=O)C(=O)N1 ; O=C1CCC(N2Cc3cc(OC4CN(C(c5ccc(Cl)cc5)c5ccc(Cl)cc5)C4)ccc3C2=O)C(=O)N1 ; O=C1CCC(N2Cc3cc(OC4CN(C(c5ccc(O)cc5)c5ccc(O)cc5)C4)ccc3C2=O)C(=O)N1 ; COc1ccc(C(c2ccc(OC)cc2)N2CC(Oc3ccc4c(c3)CN(C3CCC(=O)NC3=O)C4=O)C2)cc1]

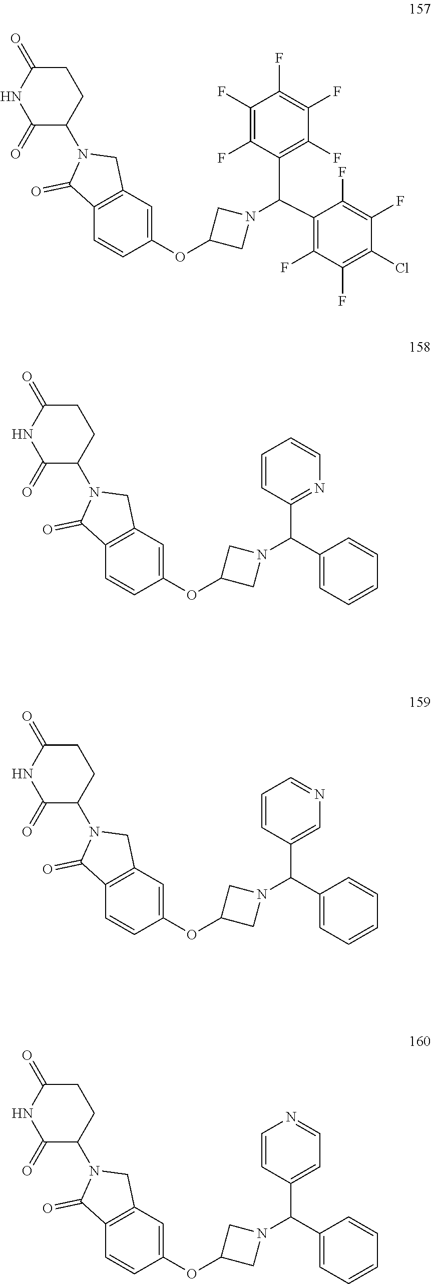 [SMILES: O=C1CCC(N2Cc3cc(OC4CN(C(c5c(F)c(F)c(F)c(F)c5F)c5c(F)c(F)c(Cl)c(F)c5F)C4)ccc3C2=O)C(=O)N1 ; O=C1CCC(N2Cc3cc(OC4CN(C(c5ccccc5)c5ccccn5)C4)ccc3C2=O)C(=O)N1 ; O=C1CCC(N2Cc3cc(OC4CN(C(c5ccccc5)c5cccnc5)C4)ccc3C2=O)C(=O)N1 ; O=C1CCC(N2Cc3cc(OC4CN(C(c5ccccc5)c5ccncc5)C4)ccc3C2=O)C(=O)N1]

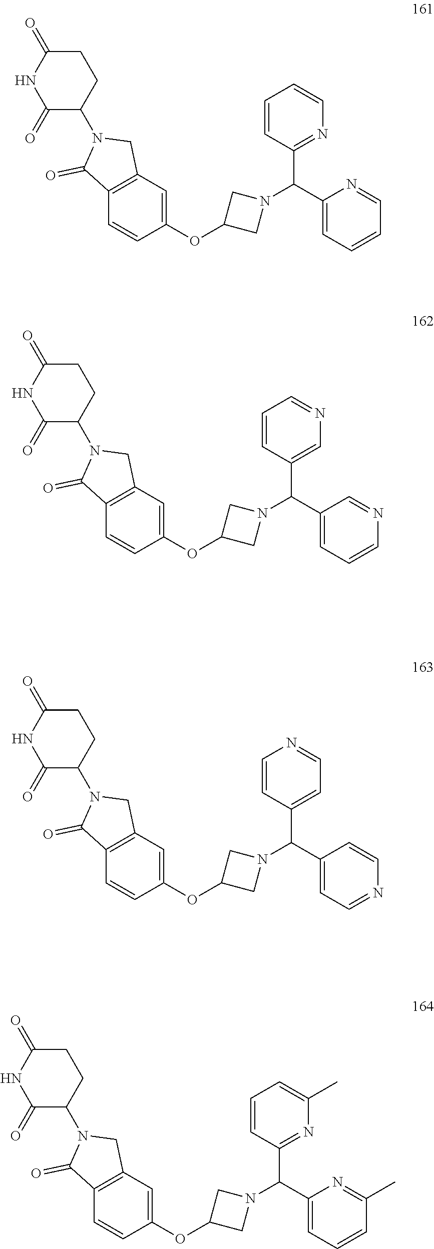 [SMILES: O=C1CCC(N2Cc3cc(OC4CN(C(c5ccccn5)c5ccccn5)C4)ccc3C2=O)C(=O)N1 ; O=C1CCC(N2Cc3cc(OC4CN(C(c5cccnc5)c5cccnc5)C4)ccc3C2=O)C(=O)N1 ; O=C1CCC(N2Cc3cc(OC4CN(C(c5ccncc5)c5ccncc5)C4)ccc3C2=O)C(=O)N1 ; Cc1cccc(C(c2cccc(C)n2)N2CC(Oc3ccc4c(c3)CN(C3CCC(=O)NC3=O)C4=O)C2)n1]

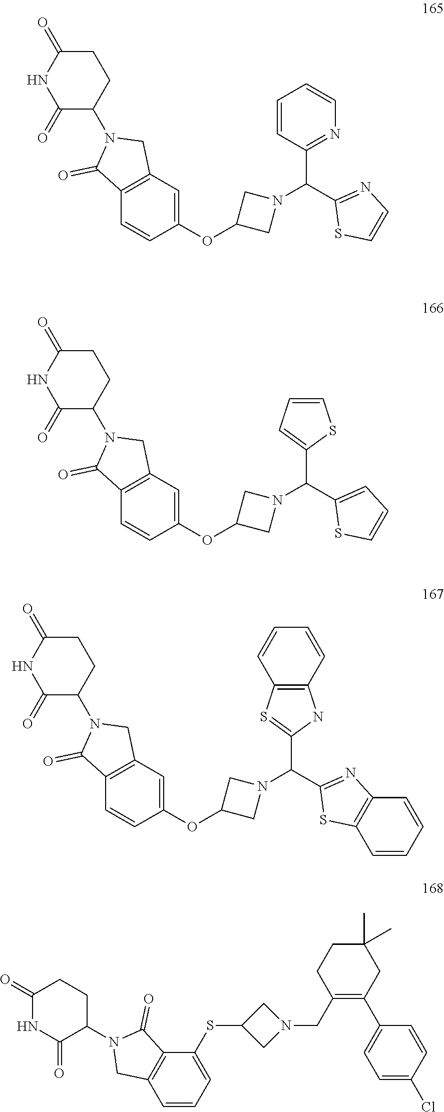

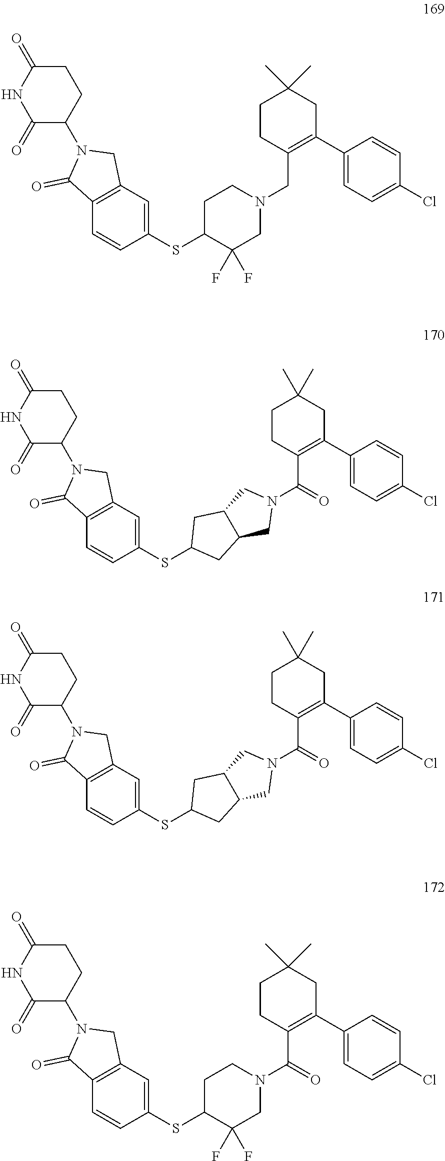 [SMILES: CC1(C)CCC(CN2CCC(Sc3ccc4c(c3)CN(C3CCC(=O)NC3=O)C4=O)C(F)(F)C2)=C(c2ccc(Cl)cc2)C1 ; CC1(C)CCC(C(=O)N2C[C@H]3CC(Sc4ccc5c(c4)CN(C4CCC(=O)NC4=O)C5=O)C[C@@H]3C2)=C(c2ccc(Cl)cc2)C1 ; CC1(C)CCC(C(=O)N2C[C@H]3CC(Sc4ccc5c(c4)CN(C4CCC(=O)NC4=O)C5=O)C[C@H]3C2)=C(c2ccc(Cl)cc2)C1 ; CC1(C)CCC(C(=O)N2CCC(Sc3ccc4c(c3)CN(C3CCC(=O)NC3=O)C4=O)C(F)(F)C2)=C(c2ccc(Cl)cc2)C1]

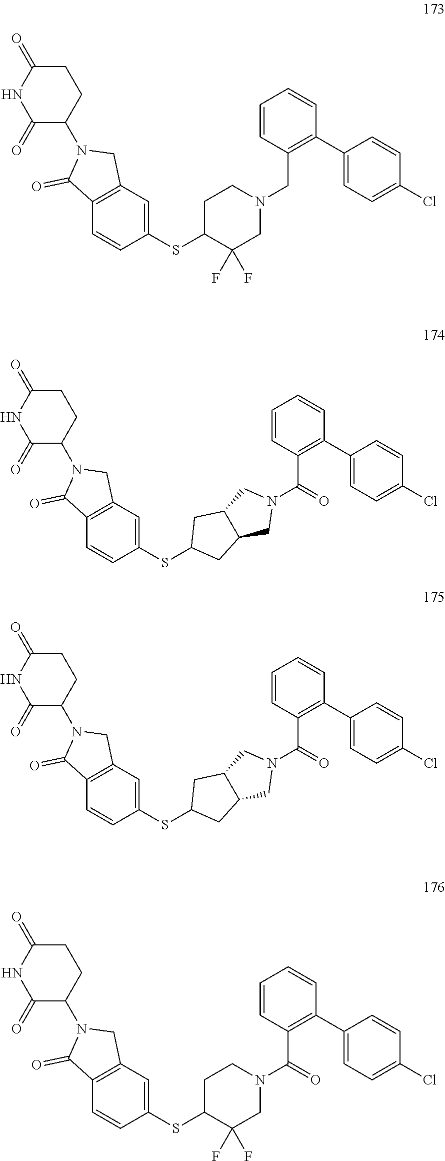 [SMILES: O=C1CCC(N2Cc3cc(SC4CCN(Cc5ccccc5-c5ccc(Cl)cc5)CC4(F)F)ccc3C2=O)C(=O)N1 ; O=C1CCC(N2Cc3cc(SC4C[C@@H]5CN(C(=O)c6ccccc6-c6ccc(Cl)cc6)C[C@H]5C4)ccc3C2=O)C(=O)N1 ; O=C1CCC(N2Cc3cc(SC4C[C@@H]5CN(C(=O)c6ccccc6-c6ccc(Cl)cc6)C[C@@H]5C4)ccc3C2=O)C(=O)N1 ; O=C1CCC(N2Cc3cc(SC4CCN(C(=O)c5ccccc5-c5ccc(Cl)cc5)CC4(F)F)ccc3C2=O)C(=O)N1]

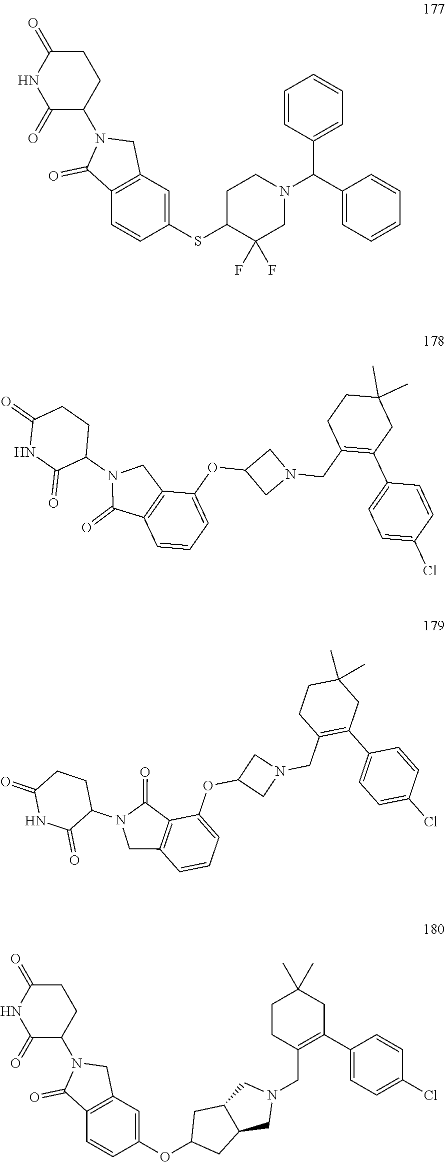 [SMILES: O=C1CCC(N2Cc3cc(SC4CCN(C(c5ccccc5)c5ccccc5)CC4(F)F)ccc3C2=O)C(=O)N1 ; CC1(C)CCC(CN2CC(Oc3cccc4c3CN(C3CCC(=O)NC3=O)C4=O)C2)=C(c2ccc(Cl)cc2)C1 ; CC1(C)CCC(CN2CC(Oc3cccc4c3C(=O)N(C3CCC(=O)NC3=O)C4)C2)=C(c2ccc(Cl)cc2)C1 ; CC1(C)CCC(CN2C[C@H]3CC(Oc4ccc5c(c4)CN(C4CCC(=O)NC4=O)C5=O)C[C@@H]3C2)=C(c2ccc(Cl)cc2)C1]

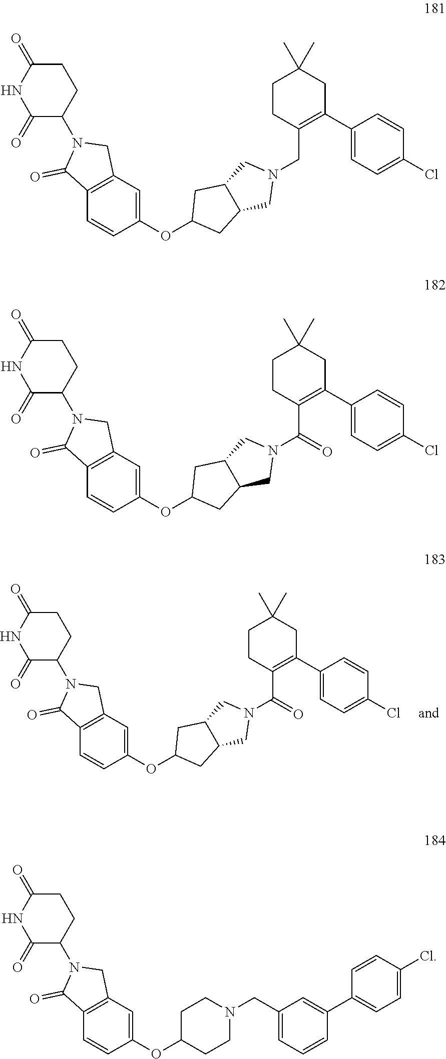 [SMILES: CC1(C)CCC(CN2C[C@H]3CC(Oc4ccc5c(c4)CN(C4CCC(=O)NC4=O)C5=O)C[C@H]3C2)=C(c2ccc(Cl)cc2)C1 ; CC1(C)CCC(C(=O)N2C[C@H]3CC(Oc4ccc5c(c4)CN(C4CCC(=O)NC4=O)C5=O)C[C@@H]3C2)=C(c2ccc(Cl)cc2)C1 ; CC1(C)CCC(C(=O)N2C[C@H]3CC(Oc4ccc5c(c4)CN(C4CCC(=O)NC4=O)C5=O)C[C@H]3C2)=C(c2ccc(Cl)cc2)C1 ; O=C1CCC(N2Cc3cc(OC4CCN(Cc5cccc(-c6ccc(Cl)cc6)c5)CC4)ccc3C2=O)C(=O)N1]

14. The compound of Formula I or salts, enantiomers, stereoisomers, polymorphs or solvates thereof as claimed in claim 1, which is hydrohalides (including hydrochlorides and hydrobromates), sulfates, citrates, maleates, mesylates, citrates, lactates, L-tartrates, fumarates, L-malates, phosphates, dihydrophosphates, pyrophosphates, metaphosphates, oxalates, malonates, benzoates, mandelates, succinates, trifluoroacetates, hippurates, glycolates, or p-toluenesulfonates of the compound of Formula I.

15. A compound of Formula A or salts, enantiomers, stereoisomers, polymorphs or solvates thereof, 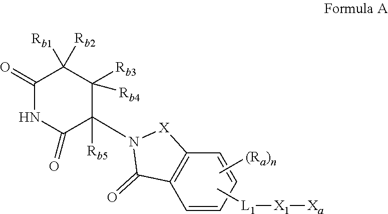
wherein X is selected from the group consisting of CO or CH₂;
L₁ is selected from the group consisting of S or O;
X₁ is selected from the group consisting of unsubstituted or substituted linear or branched alkylene, or X₁ is absent;
Xₐ represents the structure of Formula A1: 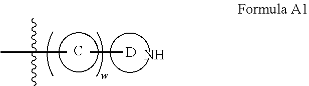
wherein ring C is selected from the group consisting of unsubstituted or substituted arylene, unsubstituted or substituted heteroarylene, unsubstituted or substituted heterocyclylene, or unsubstituted or substituted cycloalkylene, and w represents an integer of 0 or 1; and
ring D represents unsubstituted or substituted nitrogen-containing heterocyclyl;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl;
R_{b1}, R_{b2}, R_{b3}, R_{b4} and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl; and
n is an integer of 0, 1, 2 or 3, and when n is an integer of 2 or 3, each Rₐ is independently identical to or different from each other.

16. The compound of Formula A or salts, enantiomers, stereoisomers, polymorphs or solvates thereof as claimed in claim 15, wherein X₁ represents unsubstituted or substituted linear or branched C₁-C₅ alkylene, or X₁ is absent;
and/or, Xₐ represents the structure of Formula A1: 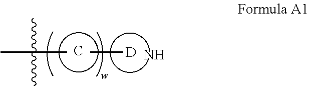
wherein ring C is selected from the group consisting of unsubstituted or substituted C₅-C₃₀ arylene, unsubstituted or substituted 5- to 30-membered heteroarylene, unsubstituted or substituted 4- to 30-membered heterocyclylene, or unsubstituted or substituted C₃-C₃₀ cycloalkylene; preferably, ring C is selected from the group consisting of unsubstituted or substituted C₅-C₂₀ arylene, unsubstituted or substituted 5- to 20-membered heteroarylene, unsubstituted or substituted 4- to 20-membered heterocyclylene, or unsubstituted or substituted C₃-C₂₀ cycloalkylene;
w represents an integer of 0 or 1; and
ring D represents unsubstituted or substituted 4- to 30-membered nitrogen-containing heterocyclyl; preferably, ring D represents unsubstituted or substituted 4- to 20-membered nitrogen-containing heterocyclyl;
and/or, Rₐ is selected from the group consisting of deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, unsubstituted or substituted linear or branched C₁-C₅ alkyl, unsubstituted or substituted C₃-C₁₅ cycloalkyl, unsubstituted or substituted 4- to 15-membered heterocyclyl, unsubstituted or substituted C₅-C₁₅ aryl, or unsubstituted or substituted 5- to 15-membered heteroaryl; preferably, Rₐ is selected from the group consisting of deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, unsubstituted or substituted linear or branched C₁-C₅ alkyl, unsubstituted or substituted C₃-C₁₅ cycloalkyl, unsubstituted or substituted 4 to 15-membered heterocyclyl, unsubstituted or substituted C₅-C₁₅ aryl, or unsubstituted or substituted 5- to 15-membered heteroaryl;
and/or, R_{b1}, R_{b2}, R_{b3}, R_{b4} and R_{b5} are each independently selected from the group consisting of hydrogen, deuterium, F, Cl, Br, I, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, or unsubstituted or substituted linear or branched C₁-C₅ alkyl; preferably, R_{b1}, R_{b2}, R_{b3}, R_{b4} and R_{b5} are each independently selected from the group consisting of hydrogen, deuterium, F, Cl, Br, I, nitro, cyano, hydroxy, mercapto, C₁-C₆ alkoxy, trifluoromethoxy, unsubstituted or substituted amino, unsubstituted or substituted silyl, unsubstituted or substituted boryl, or unsubstituted or substituted linear or branched C₁-C₅ alkyl.

17. The compound of Formula A or salts, enantiomers, stereoisomers, polymorphs or solvates thereof as claimed in claim 15, wherein (i) X₁ represents methylene, ethylene, propylene or isopropylene, or X₁ is absent; and/or
(ii) Xₐ represents the structure of Formula A1: 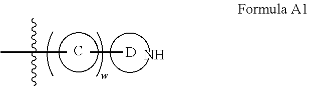
wherein the ring C is selected from the group consisting of unsubstituted or substituted C₅-C₁₅ arylene, unsubstituted or substituted 5- to 15-membered heteroarylene, unsubstituted or substituted 4 to 15-membered heterocyclylene, or unsubstituted or substituted C₃-C₁₅ cycloalkylene, wherein the substituent of the substituted ring C is optionally one or more substituents selected from the group consisting of halogen, mercapto, hydroxy, amino, cyano, C₁-C₆ alkyl, C₁-C₆ alkoxy, halogenated C₁-C₆ alkyl or any combination thereof;
w represents an integer of 0 or 1; and
the ring D represents unsubstituted or substituted 4- to 15-membered nitrogen-containing heterocyclyl.

18. (canceled)

19. The compound of Formula A or salts, enantiomers, stereoisomers, polymorphs or solvates thereof as claimed in claim 15, wherein
(1) the ring C is selected from the group consisting of the following groups:
azetidinylene, oxetanylene, pyrrolidinylene, imidazolidinylene, pyrazolidylene, tetrahydrofuranylene, tetrahydropyranylene, dihydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, dioxacyclohexenylene, azacycloheptanylene, azacyclooctylene, diazacycloheptanylene, diazacyclooctylene, bridged heterocyclylene (e.g., 5- to 15-membered bridged heterocyclylene, such as azabicyclohexylene, azabicycloheptanylene or azabicyclooctylene), or spiro-heterocyclylene (e.g., 5- to 15-membered spiro-heterocyclylene, such as 3-azaspiro[5.5]undecanylene or 7-azaspiro[3.5]nonanylene); or
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene; or
cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, spiro-cycloalkylene (e.g., C₅-C₁₅ spiro-cycloalkylene, such as spiro[3.3]heptylene, spiro[2.5]octylene, spiro[3.5]nonylene, spiro[4.4]nonylene, spiro[4.5]decylene, or spiro[5.5]undecylene), or bridged cycloalkylene (e.g., C₅-C₁₅ bridged cycloalkylene, adamantanylene, noradamantanylene, bornylene, bicyclo[2.2.1]heptylene, 2-oxobicyclo[2.2.1]heptylene, or bicyclo[2.2.1]heptentylene); or
phenylene or naphthylene;
wherein the ring C is optionally substituted by one or more substituents selected from the group consisting of deuterium, halogen, nitro, mercapto, hydroxy, cyano, C₁-C₆ alkyl, C₁-C₆ alkoxy, halogenated C₁-C₆ alkoxy, halogenated C₁-C₆ alkyl, unsubstituted or substituted amino, unsubstituted or substituted silyl, unsubstituted or substituted boryl or any combination thereof;
and/or, (2) the ring D is selected from the group consisting of the following groups: azetidinyl, pyrrolidinyl, imidazolidinyl, pyrazolidyl, oxazolidinyl, thiazolidinyl, piperidinyl, piperazinyl, morpholinyl, thiomorpholinyl, azacycloheptanyl, azacyclooctyl, diazacycloheptanyl, diazacyclooctyl, nitrogen-containing bridged heterocyclyl (e.g., 5- to 15-membered bridged heterocyclyl, such as azabicyclohexyl, azabicycloheptanyl or azabicyclooctyl), or nitrogen-containing spiro-heterocyclyl (e.g., 5- to 15-membered spiro-heterocyclyl, such as 3-azaspiro[5.5]undecanyl or 7-azaspiro[3.5]nonanyl);
wherein the ring D is optionally substituted by one or more substituents selected from the group consisting of deuterium, halogen, nitro, mercapto, hydroxy, cyano, oxo, C₁-C₆ alkyl, C₁-C₆ alkoxy, halogenated C₁-C₆ alkoxy, halogenated C₁-C₆ alkyl, unsubstituted or substituted amino, unsubstituted or substituted silyl, unsubstituted or substituted boryl or any combination thereof;
and/or Xₐ is selected from the group consisting of: 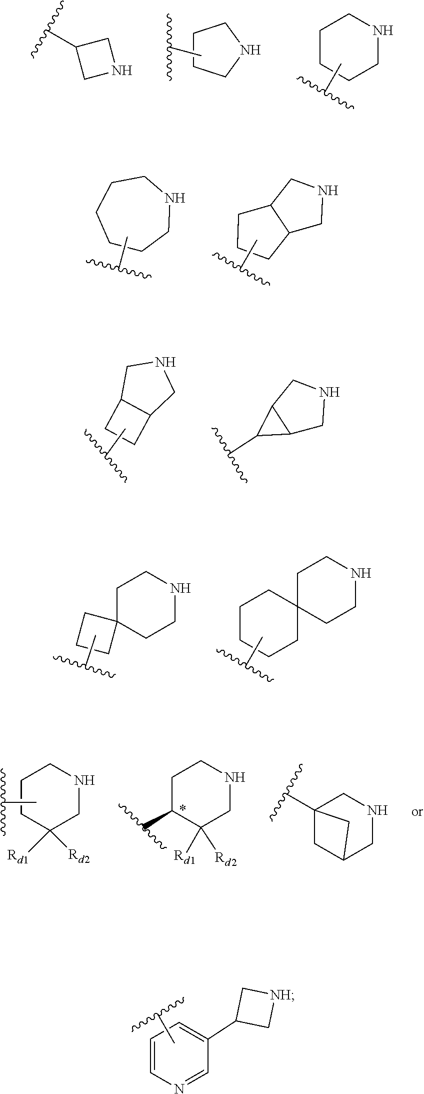
wherein R_{d1} and R_{d2} are each independently selected from the group consisting of hydrogen, deuterium, halogen, nitro, cyano, hydroxy, mercapto, C₁-C₆ alkoxy, halogenated C₁-C₆ alkoxy, unsubstituted or substituted amino, unsubstituted or substituted silyl, unsubstituted or substituted boryl, unsubstituted or substituted linear or branched C₁-C₅ alkyl; and R_{d1} and R_{d2} are not hydrogen at the same time.

20. (canceled)

21. The compound of Formula A or salts, enantiomers, stereoisomers, polymorphs or solvates thereof as claimed in claim 15, which is selected from the group consisting of:
3-(5-(azetidin-3-ylthio)-1-oxoisoindolin-2-yl)piperidine-2,6-dione;
3-(1-oxo-5-(pyrrolidin-3-ylthio)isoindolin-2-yl)piperidine-2,6-dione;
3-(1-oxo-5-(piperidin-4-ylthio)isoindolin-2-yl)piperidine-2,6-dione;
3-(5-(azepan-4-ylthio)-1-oxoisoindolin-2-yl)piperidine-2,6-dione;
3-(4-(azetidin-3-ylthio)-1-oxoisoindolin-2-yl)piperidine-2,6-dione;
3-(6-(azetidin-3-ylthio)-1-oxoisoindolin-2-yl)piperidine-2,6-dione;
3-(7-(azetidin-3-ylthio)-1-oxoisoindolin-2-yl)piperidine-2,6-dione;
3-(5-((7-azaspiro[3.5]nonan-2-yl)thio)-1-oxoisoindolin-2-yl)piperidine-2,6-dione;
3-(5-((3-azaspiro[5.5]undecan-9-yl)thio)-1-oxoisoindolin-2-yl)piperidine-2,6-dione;
3-(5-((octahydrocyclopenta[c]pyrrol-5-yl)thio)-1-oxoisoindolin-2-yl)piperidine-2,6-dione;
3-(5-((3,3-difluoropiperidin-4-yl)thio)-1-oxoisoindolin-2-yl)piperidine-2,6-dione;
3-(5-((3-azabicyclo[3.2.0]heptan-6-yl)thio)-1-oxoisoindolin-2-yl)piperidine-2,6-dione;
3-(5-((3-azabicyclo[3.1.0]hexan-6-yl)thio)-1-oxoisoindolin-2-yl)piperidine-2,6-dione;
3-(5-(((3-azabicyclo[3.1.1]heptan-1-yl)methyl)thio)-1-oxoisoindolin-2-yl)piperidine-2,6-dione;
3-(5-(((5-(azetidin-3-yl)pyridin-3-yl)methyl)thio)-1-oxoisoindolin-2-yl)piperidine-2,6-dione;
3-(5-(azetidin-3-yloxy)-1-oxoisoindolin-2-yl)piperidine-2,6-dione;
3-(1-oxo-5-(pyrrolidin-3-yloxy)isoindolin-2-yl)piperidine-2,6-dione;
3-(1-oxo-5-(piperidin-4-yloxy)isoindolin-2-yl)piperidine-2,6-dione;
3-(5-(azepan-4-yloxy)-1-oxoisoindolin-2-yl)piperidine-2,6-dione;
3-(4-(azetidin-3-yloxy)-1-oxoisoindolin-2-yl)piperidine-2,6-dione;
3-(6-(azetidin-3-yloxy)-1-oxoisoindolin-2-yl)piperidine-2,6-dione;
3-(7-(azetidin-3-yloxy)-1-oxoisoindolin-2-yl)piperidine-2,6-dione;
3-(5-((7-azaspiro[3.5]nonan-2-yl)oxy)-1-oxoisoindolin-2-yl)piperidine-2,6-dione;
3-(5-((octahydrocyclopenta[c]pyrrol-5-yl)oxy)-1-oxoisoindolin-2-yl)piperidine-2,6-dione;
3-(5-((3-azaspiro[5.5]undecan-9-yl)oxy)-1-oxoisoindolin-2-yl)piperidine-2,6-dione;
3-(5-((3,3-difluoropiperidin-4-yl)oxy)-1-oxoisoindolin-2-yl)piperidine-2,6-dione;
3-(5-((3-azabicyclo[3.2.0]heptan-6-yl)oxy)-1-oxoisoindolin-2-yl)piperidine-2,6-dione;
3-(5-((3-azabicyclo[3.1.0]hexan-6-yl)oxy)-1-oxoisoindolin-2-yl)piperidine-2,6-dione;
3-(5-((3-azabicyclo[3.1.1]heptan-1-yl)methoxy)-1-oxoisoindolin-2-yl)piperidine-2,6-dione; and
3-(5-((5-(azetidin-3-yl)pyridin-3-yl)methoxy)-1-oxoisoindolin-2-yl)piperidine-2,6-dione.

22. The compound of Formula A or salts, enantiomers, stereoisomers, polymorphs or solvates thereof as claimed in claim 15, which is hydrohalides (including hydrochlorides and hydrobromates), sulfates, citrates, maleates, mesylates, citrates, lactates, L-tartrates, fumarates, L-malates, phosphates, dihydrophosphates, pyrophosphates, metaphosphates, oxalates, malonates, benzoates, mandelates, succinates, trifluoroacetates, hippurates, glycolates, or p-toluenesulfonates of the compound of Formula A.

23. A pharmaceutical composition comprising:
the compound or salts, enantiomers, stereoisomers, polymorphs or solvates thereof as claimed in claim 1, and at least one pharmaceutically acceptable carrier or excipient;
and optionally comprising at least one additional therapeutic agent, e.g., an anticancer agent.

24. A medicine kit or reagent kit comprising:
the compound or a pharmaceutically acceptable salt thereof as claimed in claim 1; or
a pharmaceutical composition comprising the compound or pharmaceutically acceptable salt thereof.

25. (canceled)

26. (canceled)

27. (canceled)

28. (canceled)

29. A method for treating or preventing a disease or disorder associated with cereblon protein, comprising administering to a subject a therapeutically effective amount of the compound as claimed in claim 1, or salts, enantiomers, stereoisomers, polymorphs or solvates thereof, or a pharmaceutical composition comprising the compound or pharmaceutically acceptable salt thereof.

30. The method as claimed in claim 29, wherein the disease or disorder associated with cereblon protein is selected from the group consisting of tumor, infectious disease, inflammatory disease, immune system diseases, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes.

31. The method as claimed in claim 30, wherein the disease or disorder associated with cereblon protein is selected from the group consisting of: myeloma (e.g., multiple myeloma, plasma cell myeloma, smoldering myeloma, smoldering multiple myeloma); myelofibrosis; bone marrow disease; myelodysplastic syndrome (MDS); previously treated myelodysplastic syndrome; transplantation-related cancer; neutropenia; leukemia (e.g., acute myeloid leukemia, chronic myelogenous leukemia, B-cell chronic lymphocytic leukemia, leukemia-associated anemia, acute myeloid leukemia (AML), monocytic leukemia, acute lymphoblastic leukemia (ALL), acute B-cell lymphoblastic leukemia, acute T-cell lymphoblastic leukemia, chronic lymphocytic leukemia, lymphoma cell leukemia, T-cell lymphoblastic leukemia, myelomonocytic leukemia); lymphoma (e.g., diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, follicular lymphoma (FL), Burkitt lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma); thyroid cancer; melanoma; lung cancer (e.g., lung adenocarcinoma, lung squamous cell carcinoma, non-small cell lung cancer, and small cell lung cancer); inflammatory myofibroblastoma; colorectal cancer; intestinal cancer; brain glioma; astroblastoma; neuroglioma; peripheral neuroepithelioma; ovarian cancer; bronchial cancer; prostate cancer; breast cancer (e.g., triple negative breast cancer, sporadic breast cancer, and patients with Cowden syndrome); pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma (e.g., rhabdomyosarcoma, various lipogenic tumors, Ewing's sarcoma/primitive neuroectodermal tumors (Ewing/PNETs), and leiomyosarcoma); urothelial carcinoma; basal cell carcinoma; oral squamous cell carcinoma; cholangiocarcinoma; bone cancer; cervical cancer; skin cancer; bone tumors; brain tumors; lung or mesothelium tumor; colonic tumors; ureteral tumors; Richter syndrome (RS); sepsis syndrome; autoimmune diseases (e.g., rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, atopic dermatitis, vitiligo, experimental autoimmune encephalomyelitis, and Arthus reaction); keratoconjunctivitis; inflammatory diseases (e.g., Crohn's disease and ulcerative colitis, pneumonia, osteoarthritis, synovitis, systemic inflammatory response syndrome, airway inflammation, bronchitis, rheumatoid spondylitis, gouty arthritis, eczema, psoriasis, dermatitis, uveitis, conjunctivitis, acute respiratory distress syndrome, bacteremia, endotoxemia, aphthous ulcer, gingivitis, pancreatitis, regional ileitis, atrophic gastritis, peritonitis, pepticulcer, or irritable bowel syndrome); cerebral malaria; infectious diseases (e.g., viral pneumonia, Acquired immunodeficiency syndrome (AIDS), COVID-19 novel coronavirus infection, gram-negative bacteria infection, gram-positive bacteria infection, tuberculosis); septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.

32. The compound of Formula A or salts, enantiomers, stereoisomers, polymorphs or solvates thereof as claimed in claim 15, for use as a Cereblon protein (CRBN)-binding agent.

33. A method, comprising: preparing the compound or salts, enantiomers, polymorphs or solvates thereof as claimed in claim 1 from a compound of Formula A: 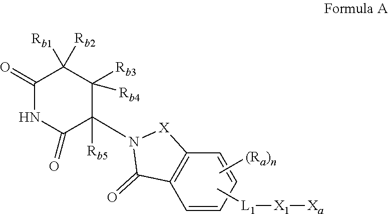
wherein X is selected from the group consisting of CO or CH₂;
L₁ is selected from the group consisting of S or O;
X₁ is selected from the group consisting of unsubstituted or substituted linear or branched alkylene, or X₁ is absent;
Xₐ represents the structure of Formula A1: 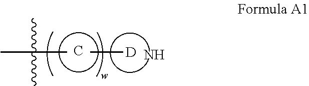
wherein ring C is selected from the group consisting of unsubstituted or substituted arylene, unsubstituted or substituted heteroarylene, unsubstituted or substituted heterocyclylene, or unsubstituted or substituted cycloalkylene, and w represents an integer of 0 or 1; and
ring D represents unsubstituted or substituted nitrogen-containing heterocyclyl;
Rₐ is selected from the group consisting of deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl, unsubstituted or substituted cycloalkyl, unsubstituted or substituted heterocyclyl, unsubstituted or substituted aryl, or unsubstituted or substituted heteroaryl:
R_{b1}, R_{b2}, R_{b3}, R_{b4} and R_{b5} are each independently selected from the group consisting of hydrogen, deuterium, halogen, nitro, cyano, hydroxy, mercapto, alkoxy, halogenated alkoxy, unsubstituted or substituted amino, unsubstituted or substituted silyl, unsubstituted or substituted boryl, unsubstituted or substituted linear or branched alkyl; and
n is an integer of 0, 1, 2 or 3, and when n is an integer of 2 or 3, each Rₐ is independently identical to or different from each other;
or salts, enantiomers, stereoisomers, polymorphs or solvates thereof.

34. A pharmaceutical composition comprising:
the compound of Formula A or salts, enantiomers, stereoisomers, polymorphs or solvates thereof as claimed in claim 15, and at least one pharmaceutically acceptable carrier or excipient;
and optionally comprising at least one additional therapeutic agent, e.g., an anticancer agent.

35. A medicine kit or reagent kit comprising:
the compound of Formula A or a pharmaceutically acceptable salt thereof as claimed in claim 15: or
a pharmaceutical composition comprising the compound of Formula A or pharmaceutically acceptable salt thereof.

36. A method for treating or preventing a disease or disorder associated with cereblon protein, comprising administering to a subject a therapeutically effective amount of the compound of Formula A or salts, enantiomers, stereoisomers, polymorphs or solvates thereof as claimed in claim 15, or a pharmaceutical composition comprising the compound of Formula A or pharmaceutically acceptable salt thereof.

37. The method as claimed in claim 36, wherein the disease or disorder associated with cereblon protein is selected from the group consisting of tumor, infectious disease, inflammatory disease, immune system diseases, anemia, hemorrhagic shock, transplant rejection, multiple organ dysfunction syndrome (MODS), sarcoidosis, adult respiratory distress syndrome, cardiovascular disease, Richter syndrome (RS), acute liver failure, or diabetes.

38. The method as claimed in claim 37, wherein the disease or disorder associated with cereblon protein is selected from the group consisting of: myeloma (e.g., multiple myeloma, plasma cell myeloma, smoldering myeloma, smoldering multiple myeloma); myelofibrosis; bone marrow disease; myelodysplastic syndrome (MDS); previously treated myelodysplastic syndrome; transplantation-related cancer; neutropenia; leukemia (e.g., acute myeloid leukemia, chronic myelogenous leukemia, B-cell chronic lymphocytic leukemia, leukemia-associated anemia, acute myeloid leukemia (AML), monocytic leukemia, acute lymphoblastic leukemia (ALL), acute B-cell lymphoblastic leukemia, acute T-cell lymphoblastic leukemia, chronic lymphocytic leukemia, lymphoma cell leukemia, T-cell lymphoblastic leukemia, myelomonocytic leukemia); lymphoma (e.g., diffuse large B-cell lymphoma, non-Hodgkin's lymphoma, Hodgkin's lymphoma, anaplastic lymphoma, anaplastic large cell lymphoma, CD20 positive lymphoma, mantle cell lymphoma, follicular lymphoma (FL), Burkitt lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma); thyroid cancer; melanoma; lung cancer (e.g., lung adenocarcinoma, lung squamous cell carcinoma, non-small cell lung cancer, and small cell lung cancer); inflammatory myofibroblastoma; colorectal cancer; intestinal cancer; brain glioma; astroblastoma; neuroglioma; peripheral neuroepithelioma; ovarian cancer; bronchial cancer; prostate cancer; breast cancer (e.g., triple negative breast cancer, sporadic breast cancer, and patients with Cowden syndrome); pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma (e.g., rhabdomyosarcoma, various lipogenic tumors, Ewing's sarcoma/primitive neuroectodermal tumors (Ewing/PNETs), and leiomyosarcoma); urothelial carcinoma; basal cell carcinoma; oral squamous cell carcinoma; cholangiocarcinoma; bone cancer; cervical cancer; skin cancer; bone tumors; brain tumors; lung or mesothelium tumor; colonic tumors; ureteral tumors; Richter syndrome (RS); sepsis syndrome; autoimmune diseases (e.g., rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, atopic dermatitis, vitiligo, experimental autoimmune encephalomyelitis, and Arthus reaction); keratoconjunctivitis; inflammatory diseases (e.g., Crohn's disease and ulcerative colitis, pneumonia, osteoarthritis, synovitis, systemic inflammatory response syndrome, airway inflammation, bronchitis, rheumatoid spondylitis, gouty arthritis, eczema, psoriasis, dermatitis, uveitis, conjunctivitis, acute respiratory distress syndrome, bacteremia, endotoxemia, aphthous ulcer, gingivitis, pancreatitis, regional ileitis, atrophic gastritis, peritonitis, pepticulcer, or irritable bowel syndrome); cerebral malaria; infectious diseases (e.g., viral pneumonia, Acquired immunodeficiency syndrome (AIDS), COVID-19 novel coronavirus infection, gram-negative bacteria infection, gram-positive bacteria infection, tuberculosis); septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; or acute liver failure.
